(19) United States
(12) Patent Application Publication
(10) Pub. No.: US 2006/0063784 A1
(43) Pub. Date: Mar. 23, 2006

(54) Method of using substituted pyrazolo [1,5-a] pyrimidines

(76) Inventors: Yanong Daniel Wang, Green Brook, NJ (US); Ariamala Gopalsamy, Mahwah, NJ (US); Erick Eduardo Honores, Hoboken, NJ (US); Lee Dalton Jennings, Chestnut Ridge, NY (US); Steven Lawrence Johnson, Amherst, NY (US); Dennis William Powell, Cortlandt Manor, NY (US); Fuk-Wah Sum, Pomona, NY (US); Hwei-Ru Tsou, New City, NY (US); Biqi Wu, Nanuet, NY (US); Nan Zhang, Bayside, NY (US)

Correspondence Address: FITZPATRICK CELLA (WYETH), 30 ROCKEFELLER PLAZA, NEW YORK, NY, 10112-3800, US

(21) Appl. No.: 11/221,846

(22) Filed: Sep. 9, 2005

Related U.S. Application Data
(60) Provisional application No. 60/610,550, filed on Sep. 17, 2004.

(51) Int. Cl. A61K 31/519; C07D 487/04

(52) U.S. Cl. 514/259.3; 544/281

(57) ABSTRACT

This invention relates to novel methods of use of certain pyrazolo[1,5-a]pyrimidine compounds and the therapeutically acceptable salts thereof. This invention also relates to novel methods of using these compounds as anti-proliferative agents in mammals, including humans.

This application claims the benefit of U.S. Provisional Application No. 60/610,550, filed Sep. 17, 2004, which is incorporated by reference herein.

### BACKGROUND OF THE INVENTION

This invention relates to the novel methods of use of certain pyrazolo[1,5-a]pyrimidine compounds and the pharmaceutically acceptable salts thereof. The invention relates to the novel methods of using these compounds as anti-proliferative agents in mammals, including humans.

Deregulation of cell proliferation, or a lack of appropriate cell death, has a wide range of clinical implications, including cancers, restenosis, angiogenesis, hyperplasia, endometriosis, lymphoproliferative disorders, graft rejection and the like. Such cells may lack the normal regulatory control of cell division, and therefore fail to undergo appropriate cell death.

Progression from one phase of the cell division cycle to the next phase is controlled by a series of sensors and arresting mechanisms called cell cycle checkpoints [Zhou, B. B, et al Nature 408, 433 (2000) and Weinert, T. A., et al, Genes Dev., 8, 652 (1994)]. Through regulation of the cyclin-dependent kinases and their obligate activating partners, the cyclins, checkpoints ensure that each step in the cell cycle has been successfully completed before the onset of the next phase. At each checkpoint, the cell determines whether it is ready for progression to the next phase or halts the progression if conditions are unfavorable, for example, if the nutrients are insufficient or if DNA damage has not been repaired [Keith, C. T., et al, Science, 270, 50 (1995)]. Deregulation of a cell cycle phase transition may occur as a consequence of the aberrant expression of positive regulators, such as the cyclins, loss of negative regulators (CDK inhibitors), e.g., p21, p27, p15, p16, p18, and p19, or the inactivation of tumor suppressor genes, such as p53 and pRb. Loss of cell cycle checkpoint control is a hallmark of tumor cells, as it increases the mutation rate and allows a more rapid progression to the tumorigenic state. Inactivation of these checkpoints can result in aberrant responses to cellular damage. For example, a cell with intact DNA damage control checkpoints will arrest at the G1/S and G2/M boundaries of the cell cycle in response to low levels of DNA damaging agents. Disruption of the checkpoint leads to the failure of the cell to arrest, multiple rounds of DNA synthesis in the presence of damaged DNA, and ultimately, apoptosis. This failure of cell cycle arrest responses in malignant cells can be exploited therapeutically in an innovative screening approach: identification of compounds that by selectively killing checkpoint-deficient cells compared with checkpoint-proficient cells can be expected to preferentially target tumor cells, while sparing normal cells. Novel anti-tumor agents identified by these screening methods are likely to be more effective and safer than current therapies for cancer. The publication WO 97/34640 describes this strategy for drug screening that was developed based on isogenic human cancer cell lines in which key checkpoint regulators have been deleted by targeted homologous recombination. These isogenic cell lines can then be used in parallel with the corresponding unmodified cells to screen for therapeutic compounds with selective toxicity toward any desired genotype [Torrance, C. J., et al, Nature Biotech., 19, 940 (2001)].

A major cell cycle checkpoint regulator, the protein p21^{Waf1/Cip1/Sdi1} (hereafter referred to as p21) was originally isolated as a general inhibitor of CDKs [El-Deiry, W. S., et al, Cell, 75, 817 (1993) and Harper, J. W., et al, Cell, 75, 805 (1993)]. p21 inhibits progression of the cell cycle by inhibiting the activity of G1 kinases (cyclin D/cdk4 and cyclin E-cdk2) and the G2 kinase (cyclin B/cdk1) in response to DNA damage or abnormal DNA content [Xiong, Y., et al, Nature, 366, 701 (1993) and Sherr, C. J., et al, Genes Dev., 9, 1149 (1995)]. Regulation of p21 levels occurs transcriptionally by p53-dependent and p53-independent mechanisms. Upon DNA damage, p21 is strongly up-regulated, reaching the levels that completely arrest proliferation. Cells derived from p21-null mice arrest proliferation inefficiently after p53 activation [Brugarolas, J., et al, Nature, 377, 552, (1995) and Brugarolas, J., et al, Proc. Natl. Acad. Sci. USA, 96, 1002 (1999)]. The expression of p21 is often low in human cancer cells due to frequent loss of the upstream activator, p53, and is associated with poor prognosis in some cancer patients [Kaijalainen, J. M., Br. J. Cancer, 79, 895 (1999) and Komiya, T., et al, Clin. Cancer Res., 3, 1831 (1997)]. A colon cancer cell line and the isogenic p21-deficient cells generated by targeted gene deletion [Waldman, T. et al. Cancer Res., 55, 5187-5190 (1995); Waldman T. et al. Nature, 381, 713-716 (1996)] was used to identify compounds that preferentially inhibit the p21-deficient cells. It would be useful to have a method of selectively inhibiting the growth of tumor cells over the growth of normal cells. Therefore, small drug molecules that can selectively seek out the p21 deficient cell and inhibit its proliferation should represent an important and useful approach to treat and cure cancer and other conditions resulting in aberrant cell growth.

Pyrazolo[1,5-a]pyrimidines have been described as having anxiolytic activity (U.S. Pat. No. 4,654,347; U.S. Pat. No. 4,236,005; U.S. Pat. No. 4,521,422; U.S. Pat. No. 4,281,000; U.S. Pat. No. 4,626,538; U.S. Pat. No. 4,576,943; U.S. Pat. No. 5,059,691; U.S. Pat. No. 5,538,977; EP 0129847; and EP 0208846).

DE4333705 described substituted pyrazolo[1,5-a]pyrimidines as useful medicinal agents. These pyrazolopyrimidines however must contain a substituent consisting of an arylmethyl group at the C-3 position.

International patent publication WO 96/35690 described substituted pyrazolo[1,5-a]pyrimidines as pesticides and fungicides. These pyrazolopyrimidines however must contain phenyl substituents linked by O or S at the C-2 position.

EP0941994 described substituted pyrazolo[1,5-a]pyrimidines as having selective affinity to 5HT-6 receptors. However, these pyrazolopyrimidines must contain an arylsulphonyl or alkylsulphonyl group at the C-3 position.

International patent publication WO 02/12244 described alternative methods for making polymorphic, crystalline forms of substituted pyrazolopyrimidines in general, and more particularly, N-[3-cyanopyrazolo[1,5a]pyrimidin-7-yl)phenyl]-N-ethylacetamide (Zaleplon), commonly used as anxiolytic and antiepileptic agents.

### SUMMARY OF THE INVENTION

The present invention is directed to a novel method for treating cancer and aberrant cell growth, or neoplasms. Preferably, the method is directed to treating neoplasms of the colon, breast, kidney, bladder, mouth, larynx, esophagus, stomach, colon, ovary, and lung. The invention is directed to using certain substituted pyrazolo[1,5-a]pyrimidines, and the therapeutically acceptable salts thereof, to selectively inhibit the proliferation of p21 deficient cells. The compounds used in the method of the present invention, involving the pyrrazolo[1,5-a]pyrimidine system, will be numbered as indicated in the formula below:

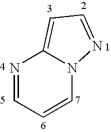

In accordance with this invention, the active compounds used in the method of this invention are represented by the following structural formula: 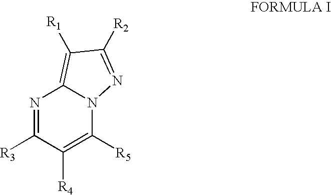
and the pharmaceutically acceptable salts and prodrugs thereof,

wherein R₁ is selected from the groups consisting of hydrogen, cyano, halogen, carbamoyl, formyl, carboxy, C(O)O-alkyl, C(O)O-cycloalkyl, C(O)cycloalkyl, R₆, C(O)R₆, and C(S)R₆;

R₆ is unsubstituted, monosubstituted or disubstituted aryl or heteroaryl wherein aryl or heteroaryl is phenyl, naphthalenyl, thiazolyl, biphenyl, thienyl, furanyl, or pyridinyl; and the substituents are selected from the groups consisting of halogen, nitro, cyano, CF₃, OCF₃, alkyl, alkoxy, trifluoromethyl, alkanol, alkylamino, alkylthio, dialkylamino, methylenedioxy, alkylsulfonyl and alkanoylamino.

R₂, R₃, and R₄ are hydrogen, CF₃, or alkyl.

R₅ is unsubstituted aryl or heteroaryl, or aryl or heteroaryl substituted by R₇, R₈, R₉, or R₁₀ with the proviso that R₁₀ must be present if the aryl or heteroaryl is substituted. The preferred moieties for R₅ are phenyl, naphthalenyl, thiazolyl, biphenyl, thienyl, furanyl and pyridinyl.

R₇, R₈, and R₉ are independently selected from the groups consisting of hydrogen, nitro, cyano, carbamoyl, halogen, N(CH₃)₂, CF₃, OCF₃, alkyl, alkoxy, and carboxy.

R₁₀ is selected from nitro, cyano, carboxy, carbamoyl, halogen, CF₃, OCF₃, alkyl, alkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₁₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl. R₁₀ is also unsubstituted, monosubstituted, or disubstituted aryl or heteroaryl which is a 5- or 6-membered aromatic ring moiety containing at least 1-4 heteroatoms selected from O, S, and N. Preferred aryl or heteroaryl groups for R₁₀ are phenyl, naphthalenyl, thiazolyl, biphenyl, thienyl, furanyl and pyridinyl.

R₁₁ and R₁₂ are independently selected from the groups consisting of hydrogen, alkyl, alkenyl, alkynyl, cycloalkyl, Q₁, Q₂, -L-Q₁ and -L-Q₂; wherein Q₁ is unsubstituted aryl or heteroaryl, monosubstituted or disubstituted aryl or heteroaryl. Preferred moieties for Q₁ are phenyl, naphthalenyl, biphenyl, thiazolyl, oxazolyl, pyrrolyl, pyrrazolyl, thienyl, furanyl, 1,2,3-triazolyl, 1,2,4-triazolyl, tetrazolyl, pyrrolidinyl, imidazolyl, and pyridinyl; and the substituents are selected from halogen, alkyl, CF₃, OCF₃, cyano, nitro, carboxy, hydroxy, alkoxy. Q₂ is alkyl or heterocycle containing at least one and up to 4 heteroatoms selected from O, S, and N, optionally including their common protecting groups. Q₂ further comprises alkyl or cycloalkyl containing or substituted by functional groups selected from halogen, carboxy, carbamoyl, hydroxy, carbonyl, and cycloalkyl with a site of unsaturation.

R₁₁ and R₁₂ together with the N to which they are attached may join to form a 3 to 8 membered ring.

R₁₃ is hydrogen, alkyl, alkenyl, alkynyl or cycloalkyl; and R₁₁ and R₁₃ together with the N to which they are attached may join to form a 3 to 8 membered ring.

Compounds for use in the method of this invention include pyrazolo[1,5-a]pyrimidines of Formula I wherein R₁ is selected from the groups consisting of hydrogen, cyano, halogen, carbamoyl, formyl, carboxy, C(O)O-alkyl, C(O)O-cycloalkyl, C(O)cycloalkyl, R₆, C(O)R₆, C(S)R₆; R₂, R₃, and R₄ are hydrogen, CF₃, or alkyl; R₅ is unsubstituted aryl or heteroaryl, or aryl or heteroaryl substituted by R₇, R₈, R₉, or R₁₀ with the proviso that R₁₀ must be present; and P6, R₇, R₈, R₉, and R₁₀ are as defined before.

Further compounds for use in the method of this invention include pyrazolo[1,5-a]pyrimidines of Formula I wherein R₁ is C(O)R₆, C(S)R₆; R₂, R₃, and R₄ are hydrogen, CF₃, or alkyl; R₅ is unsubstituted aryl or heteroaryl, or aryl or heteroaryl substituted by R₇, R₈, R₉, or R₁₀ with the proviso that R₁₀ must be present; and R₆, R₇, R₈, R₉, and R₁₀ are as defined before.

In another embodiment of the method of the present invention, the compounds used include pyrazolo[1,5-a]pyrimidines of Formula I wherein R₁ is C(O)R₆, C(S)R₆; R₂, R₃, and R₄ are hydrogen; R₅ is unsubstituted aryl or heteroaryl, or aryl or heteroaryl substituted by R₇, R₈, R₉, or R₁₀ with the proviso that R₁₀ must be present; and R₆, R₇, R₈, R₉, and R₁₀ are as defined before.

Another embodiment of the method of the present invention involves using compounds Formula I wherein R₁ is C(O)R₆; R₂, R₃, and R₄ are hydrogen;

R₅ is aryl or heteroaryl substituted by R₇, R₈, R₉, or R₁₀ with the proviso that R₁₀ must be present; and R₆, R₇, R₈, R₉, and R₁₀ are as defined before.

### DETAILED DESCRIPTION OF THE INVENTION

The definitions set forth below apply to the terms used herein unless otherwise defined.

Definitions

Halogen is defined as fluoro, chloro, bromo, and iodo.

In this specification the term “alkyl” includes straight, branched alkyl groups, such as iso-propyl, n-butyl, tert-butyl, and cycloalkyl groups. The length of an alkyl moiety can be from 1 to 12 carbon atoms, but is preferably 1 to 6 carbon atoms. Furthermore, branched alkyl moieties may contain 3 to 12 carbon atoms. These alkyl moieties may be unsubstituted or substituted. The term “alkenyl” refers to a substituted or unsubstituted radical aliphatic hydrocarbon containing one double bond and includes alkenyl moieties of both straight, preferably of 2 to 7 carbon atoms and branched, preferably of 3 to 7 carbon atoms. Such alkenyl moieties may exist in the E or Z configurations; the compounds of this invention include both configurations. The term “alkynyl” includes substituted and unsubstituted alkynyl moieties of both straight chain containing 2 to 7 carbon atoms and branched containing 4 to 7 carbon atoms having at least one triple bond.

An alkoxy group is defined as an alkyl group attached to an oxygen atom such as methoxy, t-butoxy and the like. It includes polyethers such as —O—(CH₂)₂OCH₃ and the like. It also includes cycloalkyl ethers, such as an epoxide, in which the oxygen atom is a member of the cyclic ring. The alkyl group is as defined as above (it can thus be straight, branched, or cyclic).

A substituted phenyl or heteroaryl ring may have substituents in the ortho, meta, or para positions. The heteroaryl ring is defined as an aromatic heterocyclic ring system, preferably with a 5 or 6 membered aromatic moiety, containing at least 1-4 heteroatoms selected from O, S, and N. The heteroaryl moieties are preferably selected from the group consisting of thiophene, furan, pyrrole, pyrazole, imidazole, 1,2,3-triazole, 1,2,4-triazole, tetrazole, thiazole, oxazole, isothiazole, isoxazole, 1,3,4-oxadiazole, 1,2,4-oxadiazole, 1,3,4-thiadiazole, pyridine, pyrimidine, pyrazine, pyridazine and 1,3,5-triazine. The heteroaryl ring may be oxidized on a nitrogen atom to provide the corresponding N-oxide, such as pyridine N-oxide, or the heterocyclic ring may contain a carbonyl group on one of the carbon atoms, such as 1,3,4-oxadiazol-2-one. Heteroatoms in any ring system can be protected with their known protecting groups common in the art. (Greene, T.; Wuts, P. Protective Groups in Organic Synthesis, 2^{nd} Ed., 1991).

Bicyclic ring systems include both bicyclic aryl and bicyclic heteroaryl and are preferably selected from naphthalene, 1,2,3,4-tetrahydronaphthalene, indan, indene, isoindene, indole, 2,3-dihydroindole, 2-indazole, isoindazole, quinoline, isoquinoline, tetrahydroquinoline, benzofuran, benzothiophene, benzimidazole, benzotriazole, benzothiazole, benzoxazole, benzisoxazole, 1,2-benzopyran, cinnoline, phthalazine, quinazoline, 1,8-naphthyridine, pyrido[3,2-b]pyridine, pyrido[3,4-b]pyridine, pyrido[4,3-b]pyridine, pyrido[2,3-d]pyrimidine, purine, pteridine and others. Nitrogen atoms contained in either or both rings of the bicyclic group may be oxidized to provide the corresponding N-oxide, such as quinoline N-oxide. The bicyclic ring system may be oxidized at the carbon atoms to provide a carbonyl group, such as 2-indanone.

Within the present invention, it is to be understood that a pyrazolo[1,5-a]pyrimidine compound of Formula I may exhibit the phenomenon of tautomerism and that the formula drawings within this specification can represent only one of the tautomeric forms. It is to be understood that this invention encompasses any tautomeric form and is not limited merely to any one tautomeric form utilized within the formula drawings.

The compounds used in the method of this invention may contain one or more stereogenic carbon atoms. In such cases, the compounds used in this invention include the individual diasteromers, the racemates and the enantiomers thereof. The compounds used in this invention may contain one or more double bonds. In such cases, the compounds used in this invention include each of the possible configuration isomers as well as mixtures of these isomers.

The subject invention also includes the use of pharmaceutically acceptable prodrugs of compounds of Formula I. A “pharmaceutically acceptable prodrug” is intended to mean a compound that may be converted under physiological conditions or by solvolysis to a compound or derivative of Formula I.

The subject invention also includes the use of isotopically-labelled compounds, which are identical to those recited in Formula I, but for the fact that one or more atoms are replaced by an atom having an atomic mass or mass number different from the atomic mass or mass number usually found in nature.

A “pharmaceutically acceptable salt” is intended to mean a salt that retains the biological effectiveness and properties of the free acids and bases of compounds and derivatives of Formula I, and that is not biologically or otherwise undesirable. Though not intending to be limiting in any way, the pharmaceutically acceptable salts of the compounds of Formula I with a basic moiety can be formed from organic and inorganic acids, such as acetic, citric, tartaric, succinic, maleic, malonic, gluconic, hydrochloric, hydrobromic, phosphoric, nitric, sulfuric, methanesulfonic, and similarly known acceptable acids. Similarly, when a compound of this invention contains a acidic moiety, salts can be formed form organic and inorganic bases. For example, alkali metal salts might include: sodium, lithium, potassium and N-tetraalkylammonium salts such as N-tetrabutylammonium. The literature of this art is replete with the possible salts and the methods for preparing them. One skilled in the art would be knowledgeable of the pharmaceutically acceptable salts and could easily prepare salts of the inventive compounds.

Solid or liquid pharmaceutically acceptable carriers, diluents, vehicles, or excipients may be employed in the pharmaceutical compositions. Illustrative solid carriers include starch, lactose, calcium sulphate dihydrate, terra alba, sucrose, talc, gelatin, pectin, acacia, magnesium stearate, and stearic acid. Illustrative liquid carriers may include syrup, peanut oil, olive oil, saline solution, and water.

A “therapeutically effective amount” is intended to mean that amount of a compound of Formula I that, when administered to a human or mammal in need thereof, is sufficient to effect treatment for cancer. The amount of a given compound of Formula I that will correspond to a “therapeutically effective amount” will vary depending upon factors such as the particular compound, the disease condition and the severity thereof, the identity of the human or mammal in need thereof, but it can nevertheless be readily determined by one of skill in the art.

A “neoplasm” is any new and abnormal growth; specifically a new growth of tissue in which the growth is uncontrolled and progressive. A neoplasm can be benign or malignant. A neoplasm furthermore can be the result or symptom of cancer.

“Treating” or “treatment” is intended to mean at least the slowing of the progression of a neoplastic cell in a mammal, such as a human; preferably stopping the progression of the neoplasm, and more preferably curing the condition. Treatment relates to the inhibition of proliferation of p21-deficient cells, and may include:

(a) prophylactic treatment in a mammal, particularly when the mammal is found to be predisposed to having the disease condition but not yet diagnosed as having it;

(b) inhibiting the disease condition; and/or

(c) alleviating, in whole or in part, the disease condition.

The compounds of this invention may be prepared by the procedures known in the art as detailed in the following references: U.S. Pat. No. 4,654,347; U.S. Pat. No. 4,236,005; U.S. Pat. No. 4,521,422; U.S. Pat. No. 4,281,000; U.S. Pat. No. 4,626,538; U.S. Pat. No. 4,576,943; U.S. Pat. No. 5,059,691; EP 0129847; and EP 0208846, the disclosures of which are hereby incorporated by reference.

In one embodiment of this invention the method is directed to inhibiting abnormal cell growth in a mammal by the step of administering to the mammal a therapeutically effective amount of at least one compound of Formula I or a pharmaceutically acceptable salt or prodrug thereof. Another embodiment of the invention is directed to a method of treating, inhibiting the progression of, or eradicating a neoplasm comprising administering to a mammal in need thereof an effective amount of at least one compound of Formula I or a pharmaceutically acceptable salt or prodrug thereof. In a preferred embodiment the neoplasm being treated is selected from the group consisting of colon, heart, kidney, bladder, mouth, larynx, esophagus, stomach, colon, ovary and lung. In a particularly preferred embodiment the neoplasm being treated is a colorectal neoplasm. It should be readily evident to one of ordinary skill that the method of this invention may be practiced using a single compound as described herein or a combination of the compounds described herein to achieve a therapeutically effective amount for treatment.

The compounds used in the present invention may be prepared as set forth in the following reaction scheme:

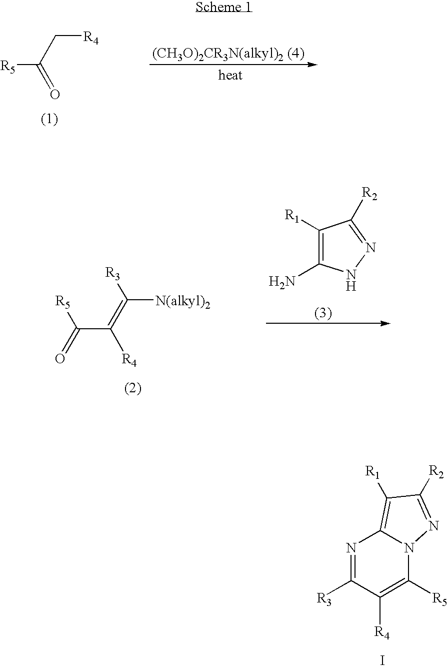

Referring to Scheme 1, the reaction of ketone (1) with acetals of N,N-dialkylformamides or acetals of N,N-dialkylacetamide can be carried out in an inert solvent or without a solvent. The reaction of the substituted 3-aminopyrazole (3) where R₁ and R₂ are herein before defined, and an appropriately substituted 3-dialkylamino-1-(aryl or heteroaryl)-2-propen-1-one (2) where R₃, R₄, and R₅ are herein before defined in weak acid such as glacial acetic acid or in an inert solvent such as toluene, acetonitrile or dimethoxyethane, at reflux temperature for several hours, produces the desired products I where R₁, R₂, R₃, R₄, and R₅ are herein before defined.

Substituted 3-dimethylamino-1-(3-heteroaryl)-2-propane-1-ones are disclosed in U.S. Pat. Nos. 4,281,000 and 3-dialkylamino-1-phenyl-2-propen-1-one are disclosed in U.S. Pat. Nos. 4,178,449 and 4,236,005.

The 3-amino-4-pyrazoles (3) where R₂ is H are disclosed in one or more U.S. Pat. Nos. 4,236,005; 4,281,000; 4,521,422; 4,626,538; 4,654347; and 4,900,836.

Pyrazolo[1,5-a]pyrimidines are prepared by condensation of 3-aminopyrazoles and substituted 3-aminopyrazoles with 1,3-dicarbonyl compounds as described in J. Med. Chem., 18, 645 (1974); J. Med. Chem. 18, 460 (1975); J. Med. Chem., 20, 386 (1977); Synthesis, 673 (1982) and references contained therein.

The preparation of the compounds used in this invention encompassed by Formula (7) is described below in Scheme 2 where R₁, R₂, R₃, R₄, R₇, R₈, and R₉ are as defined herein before. The reaction of (5) with substituted 3-aminopyrazole (3) in acetic acid at reflux for several hours gives compounds represented by Formula (6). The reduction of nitro compound (6) with reducing agents such as Fe, SnCl₂-xH₂O, catalytic hydrogenation and the like, gives compounds represented by Formula (7).

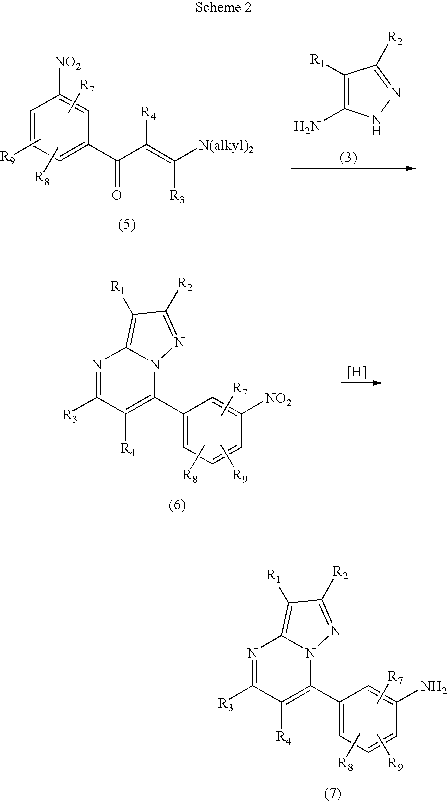

The preparation of the compounds used in this invention encompassed by Formula (9) is described below in Scheme 3 where R₁, R₂, R₃, R₄, R₇, R₈, R₉, R₁₁, and R₁₃ are as defined herein before. The reaction of aniline (7) with the acylating agents such as acyl chloride (8) or carboxylic acid anhydride, in the presence of a base such as pyridine, triethylamine, N-methylmorpholine, 4-dimethylaminopyridine and the like, gives compounds represented by Formula (9). Alternatively, (9) can be prepared from the reaction of aniline (7) with carbamate intermediate (12) generated in situ by treating carboxylic acid (10) with alkyl chloroformate (11) in the presence of base as defined above.

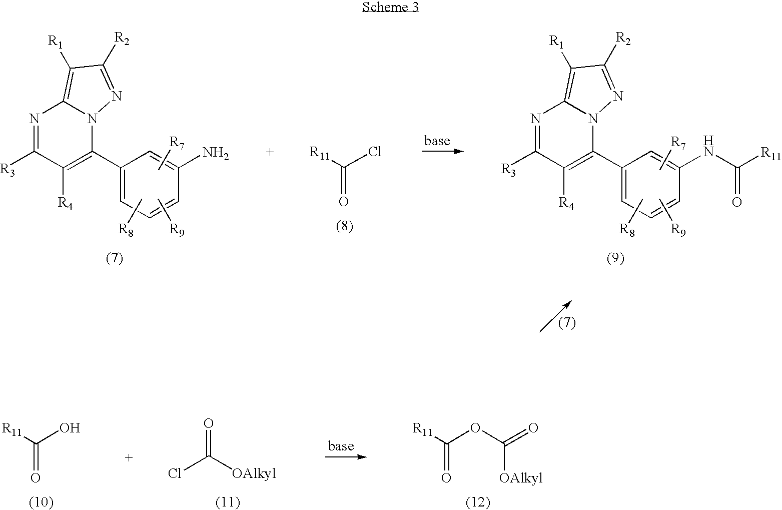

The preparation of the compounds used in this invention encompassed by Formula (14) is described below in Scheme 4 where R₁, R₂, R₃, R₄, R₇, R₈, R₉, R₁₁, R₁₂, and R₁₃ are as defined herein before. The reaction of aniline (7) with alkyl isocyanate or aryl isocyanate (13) in the presence of base, such as pyridine, triethylamine, N-methylmorpholine, 4-dimethylaminopyridine and the like, gives compounds represented by Formula (14). Alternatively, (14) can be prepared by treating aniline (7) with phenyl chloroformate or substituted phenyl chloroformate (15) to form carbamate intermediate (16) in the presence of base as defined above, followed by reaction with amine (17).

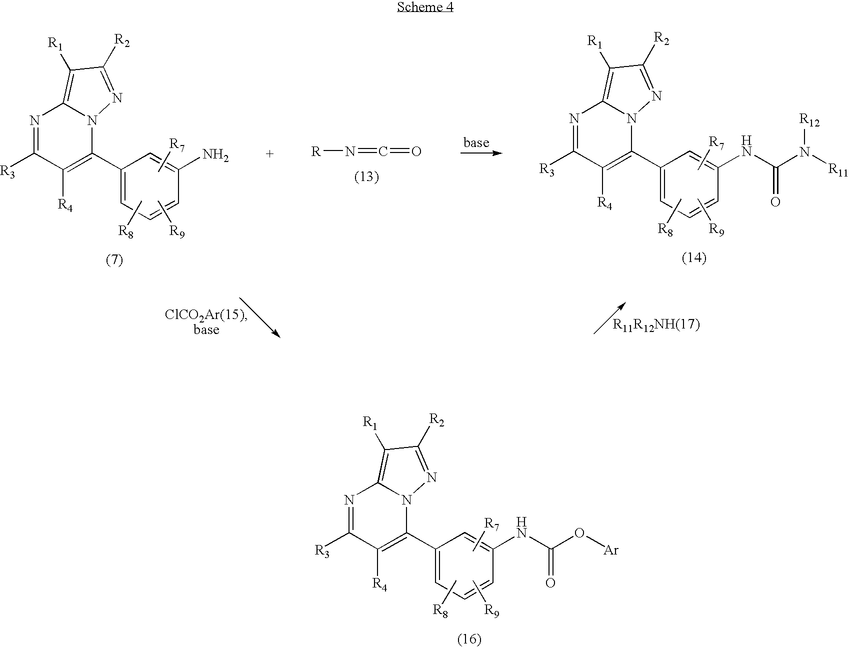

The preparation of the compounds used in this invention encompassed by Formula (19) is described below in Scheme 5 where R₁, R₂, R₃, R₄, R₇, R₈, R₉, R₁₁, and R₁₃ are as defined herein before. The reaction of aniline (7) with alkyl chloroformate or aryl chloroformate (18) in the presence of base, such as pyridine, triethylamine, N-methylmorpholine, 4-dimethylaminopyridine and the like, gives compounds represented by Formula (19). Alternatively, (19) can be prepared from the reaction of aniline (7) with carbamate intermediate (22) generated in situ by treating alcohol (20) with aryl chloroformate (21), such as 4-nitrophenyl chloroformate in the presence of base as defined above.

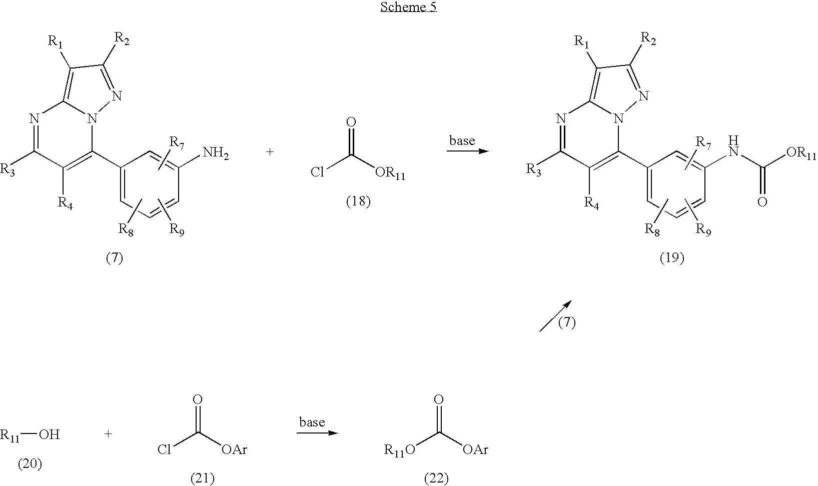

The preparation of the compounds used in this invention encompassed by Formula (24) and (25) is described below in Scheme 6 where R₁, R₂, R₃, R₄, R₇, R₈, R₉, R₁₁, and R₁₂ are as defined herein before, with the proviso that R₁₁ and R₁₂ are not an aryl or heteroaryl group. The reaction of aniline (7) with aldehydes or ketones using a reducing agents (such as sodium cyanoborohydride, sodium triacetoxylborohydride, and the like) and an inert solvent such as tetrohydrofuran, methylene chloride, N,N-dimethylformamide (“DMF”) and the like, gives compounds represented by Formula (24). Compound (25) can be prepared from (24) with the same or different aldehyde or ketone using the same procedure described above.

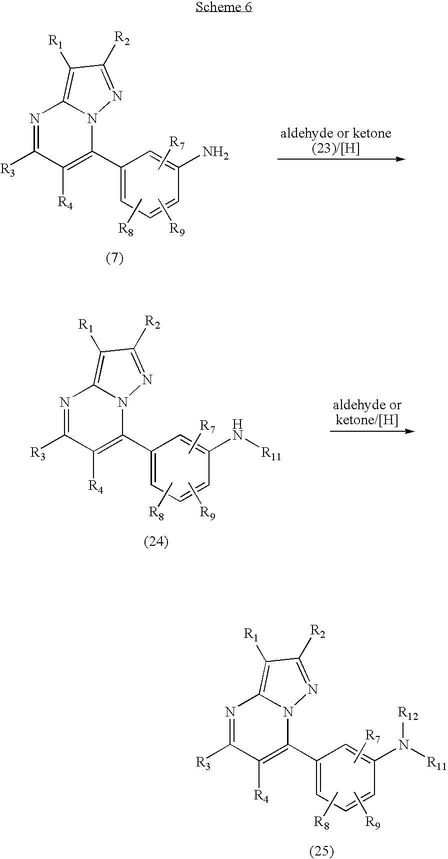

The preparation of the compounds used in this invention encompassed by Formula (27) is described below in Scheme 7 where R₁, R₂, R₃, R₄, R₇, R₉, R₉, R₁₁ and R₁₂ are as defined herein before. The reaction of aniline (7) with alkyl sulfonyl chloride or aryl sulfonyl chloride (26) in the presence of a base such as pyridine, triethylamine, N-methylmorpholine, 4-dimethylaminopyridine and the like, gives compounds represented by Formula (27).

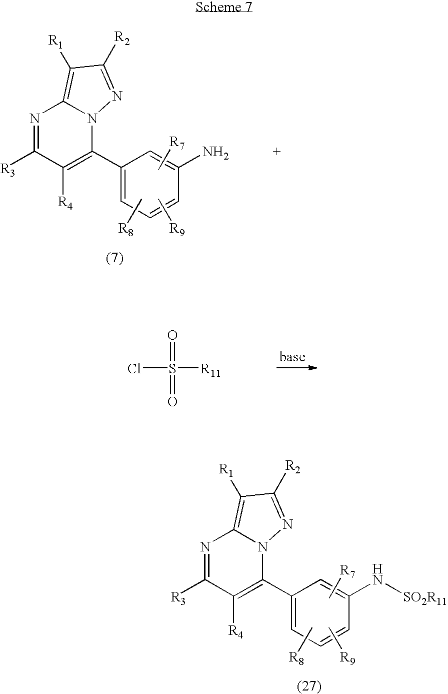

The preparation of the compounds used in this invention encompassed by Formula (29) is described below in Scheme 8 where R₁, R₂, R₃, R₄, R₇, R₉, R₉, R₁₁, and R₁₃ are as defined herein before. The reaction of aniline (7) with alkyl isothiocyanate or aryl isothiocyanate (28) in the presence of base such as pyridine, triethylamine, N-methylmorpholine, DMAP and the like, gives compounds represented by Formula (29).

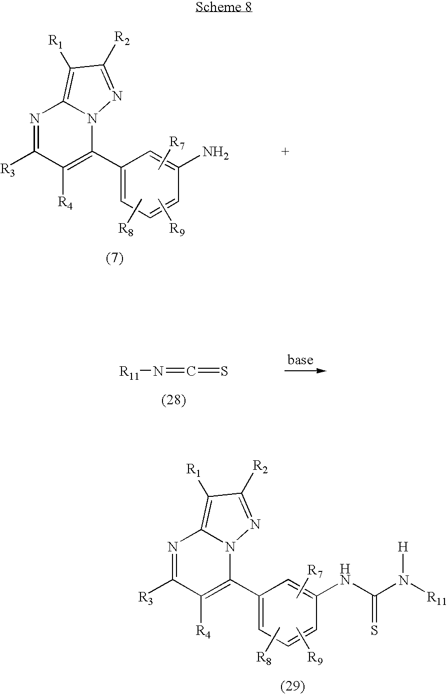

The preparation of the compounds used in this invention encompassed by Formula (32) is described below in Scheme 9 where R₁, R₂, R₃, R₄, R₇, R₈, R₉, R₁₁, R₁₂, and R₁₃ are as defined herein before. The reaction of aniline (7) with diarylcyanocarbonimidate, such as diphenylcyanocarbonimidate (30), in an inert solvent such as acetonitrile, DMF, methylene chloride, tetrahydrofuran and the like, gives compounds represented by Formula (31). Compound (31) was subsequently reacted with amine (17) in an inert solvent such as methanol, ethanol, isopropanol, acetonitrile, DMF, methylene chloride, tetrahydrofuran and the like to produce compounds represented by Formula (32).

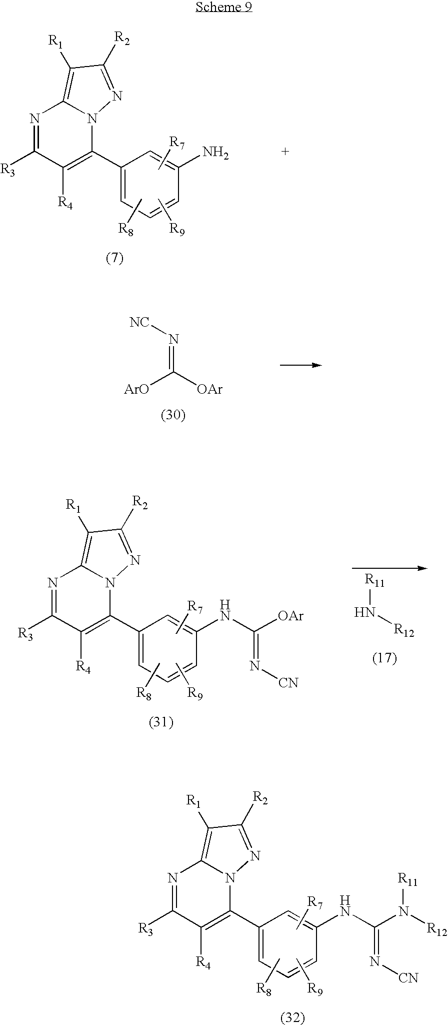

The preparation of the compounds used in this invention encompassed by Formula (37) is described below in Scheme 10 where R₁, R₂, R₃, R₄, R₇, R₈, R₉, R₁₁ and R₁₂ are as defined herein before. The reaction of ketone (33) with acetals of N,N-dialkylformamides or acetals of N,N-dialkylacetamide can be carried out in an inert solvent such as DMF, acetonitrile, toluene and the like, or without a solvent. The ketoester (33) where R₄, R₇, R₈, and R₉ are H is disclosed in J. Med. Chem., 13, 674 (1970).

The reaction of (34) with substituted 3-aminopyrazole (3) in acetic acid at reflux for several hours gives compounds represented by Formula (35). Compound (35) may be hydrolyzed in strong aqueous base, such as potassium hydroxide, sodium hydroxide and the like, at temperatures from 20° C.-100° C., to give compounds represented by Formula (36), which may be treated with amine (17) in an inert solvent such as methylene chloride, THF, DMF, acetonitrile and the like, in the presence of the organic base such as triethylamine, diisopropylethylamine, pyridine and the like, and a catalyst such as benzotriazol-1-yloxytripyrrolidinophosphonium hexafluorophosphate (Py-BOP) and the like, to produce the compounds represented by Formula (37).

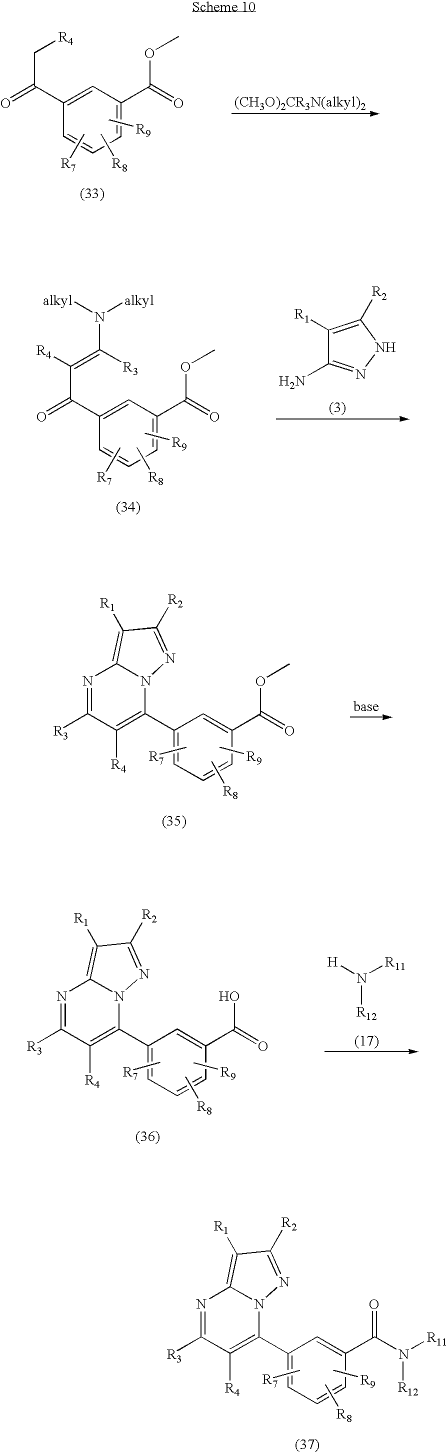

The preparation of the compounds used in this invention encompassed by Formulas (42)-(49) is described below in Scheme 11 where R₁, R₂, R₃, R₄, R₇, R₈, R₉, R₁₁, R₁₂, and R₁₃ are as defined herein before. The reaction of thiophene ketone (38) with acetals of N,N-dialkylformamides or acetals of N,N-dialkylacetamide can be carried out in an inert solvent such as DMF, acetonitrile, toluene and the like, or without a solvent. The reaction of (39) with substituted 3-aminopyrazole (3) in acetic acid at reflux for several hours gives compounds represented by Formula (40). The reduction of nitro compound (40) with reducing agents such as Fe, SnCl₂-xH₂O, catalytic hydrogenation and the like, gives compounds represented by Formula (41). Compound (41) may be converted to the corresponding amides, ureas, carbamates, substituted amines, sulphonamides, thioureas, thiocarbamates, cyanoimidates and the like as described in Scheme 3-9.

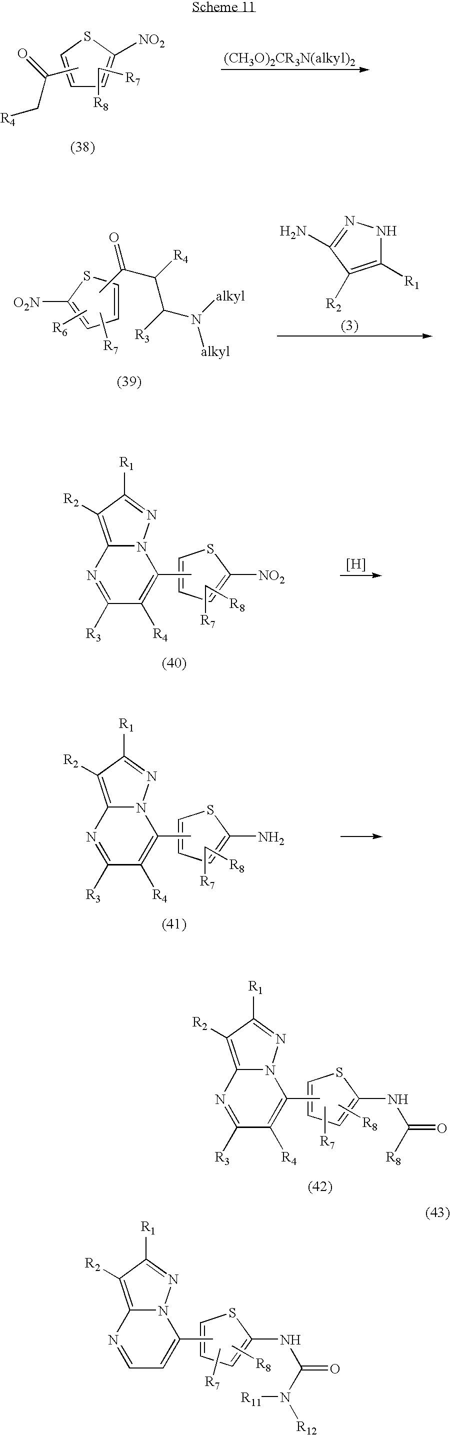

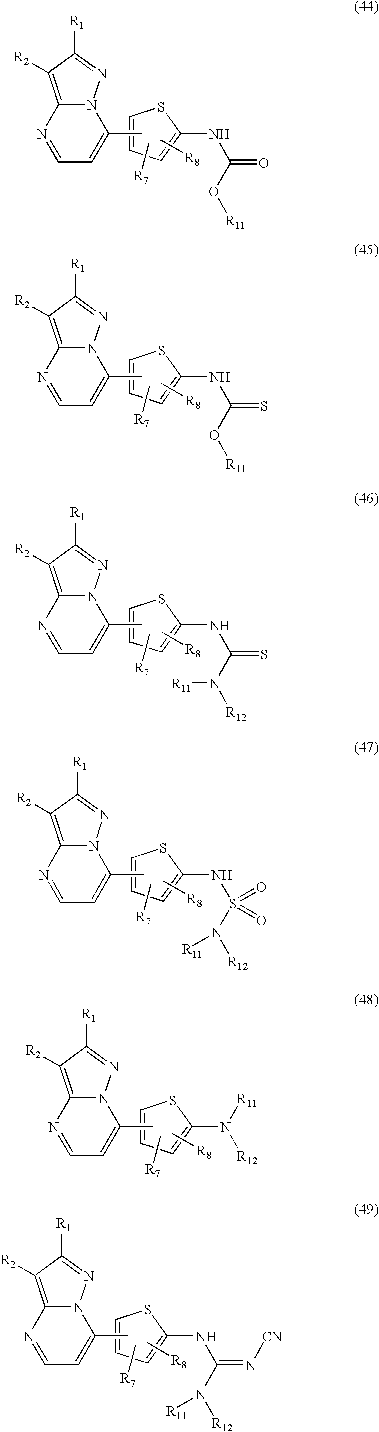

The preparation of the compounds used in this invention encompassed by Formulas (57)-(64) is described below in Scheme 12 where R₁, R₂, R₃, R₄, R₇, R₈, R₉, R₁₁, R₁₂, and R₁₃ are as defined herein before. The reaction of pyrimidin-4-one (50) with ketone (51) may be carried out in alcoholic solvent such as methanol, ethanol and the like, in the presence of NH3 at elevated temperature such as 100° C. in a sealed tube to give compounds represented by Formula (52). The pyrimidin-4-one (50) where R₇, and R₈, are H may be prepared by the procedures disclosed in J. Amer. Chem. Soc. 82, 486 (1960) and Bull. Chem. Soc. Jpn. 69, 1997 (1996). The 3-nitropyridine (52) where R₄, R₇, R₈, and R₉ are H is disclosed in Synthesis, 1277 (1997).

Oxidation of compounds (52) with oxidation agents such as CrO₃, KMnO₄ and the like in an inert solvent such as methylene chloride, chloroform and the like generate 3-acetylpyridine (53). The reaction of ketones (53) with acetals of N,N-dialkylformamides or acetals of N,N-dialkylacetamide can be carried out in an inert solvents such as DMF, acetonitrile, toluene and the like, or without a solvent. The reaction of (54) with substituted 3-aminopyrazole (3) in acetic acid at reflux for several hours gives compounds represented by Formula (55). The reduction of nitro compounds (55) with reducing agents such as Fe, SnCl₂-xH₂O, catalytic hydrogenation and the like, gives compounds represented by Formula (56). Compound (56) may be converted to the corresponding amides, ureas, carbamates, substituted amines, sulphonamides, thioureas, thiocarbamates, cyanoimidates and the like as described in Scheme 3-9.

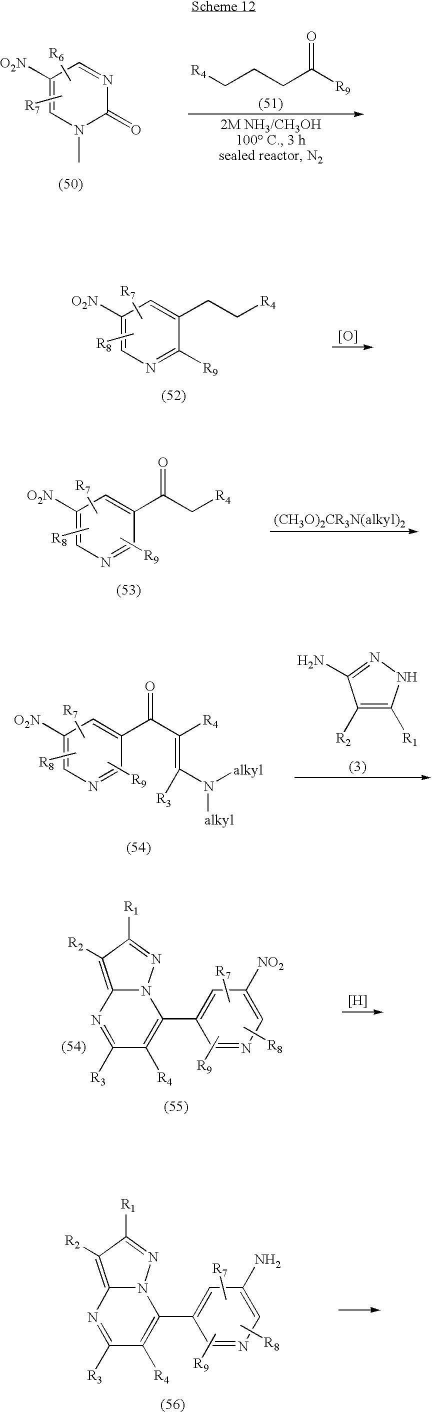

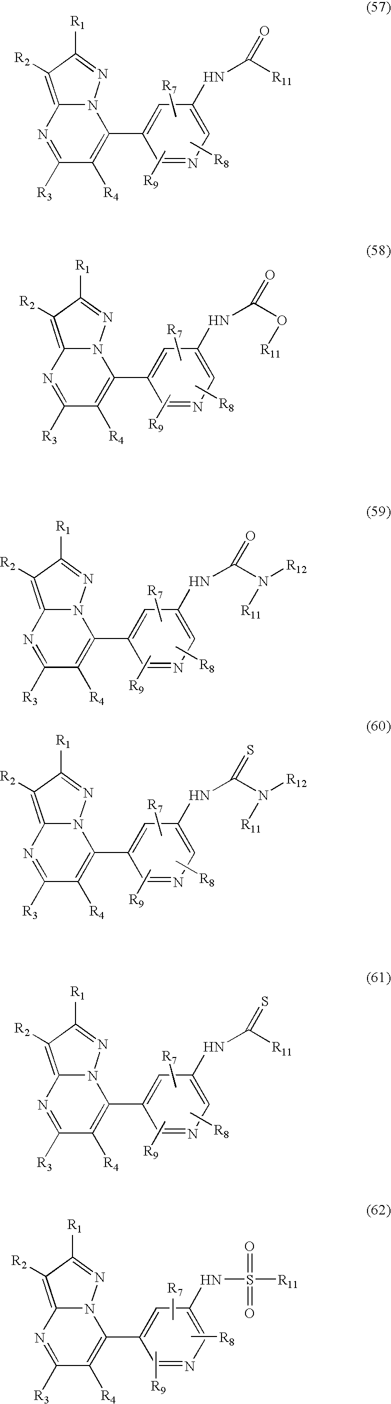

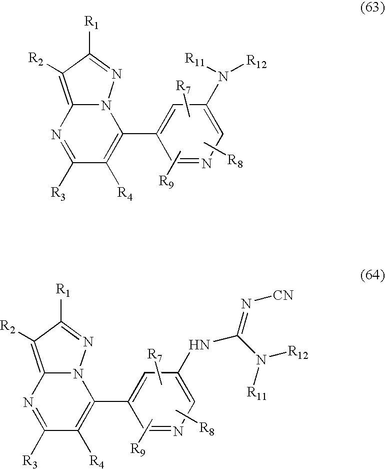

The preparation of the compounds used in this invention encompassed by Formula (69) is described below in Scheme 13 where R₁, R₂, R₃, R₄, R₇, R₈, and R₉ are as defined herein before. The reaction of ketones (66) with acetals of N,N-dialkylformamides or acetals of N,N-dialkylacetamide can be carried out in an inert solvents such as DMF, acetonitrile, toluene and the like, or without a solvent. The reaction of (66) with substituted 3-aminopyrazole (3) in acetic acid at reflux for several hours gives compounds represented by Formula (67). The Pd-mediated coupling reaction of (67) with organometallic agents (68), such as boronic acids (esters) and organostannane, gives compounds represented by Formula (69).

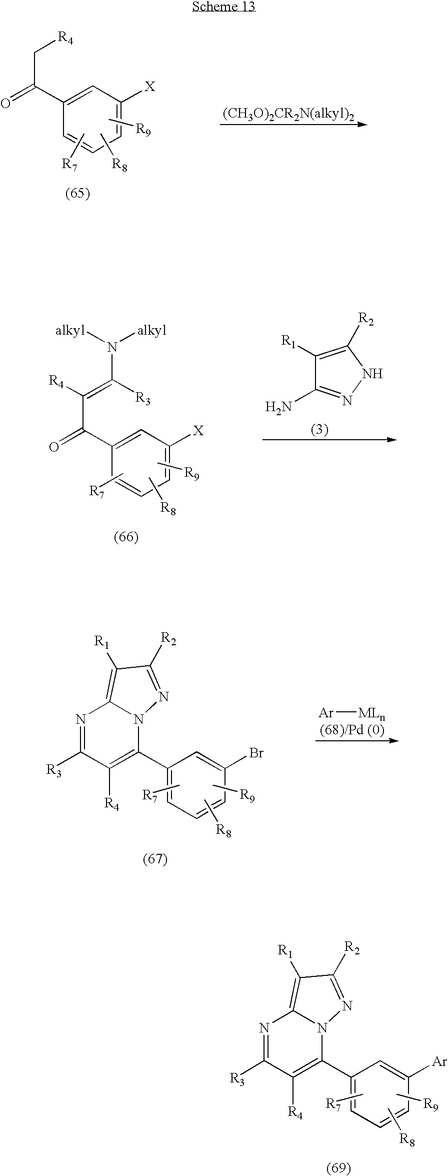

The preparation of the compounds used in this invention encompassed by Formulas (74) is described below in Scheme 14 where R₂, R₃, R₄, R₇, R₈, R₉, and R₁₀ are as defined herein before. The reaction of ketones (70) with acetals of N, N-dialkylformamides or acetals of N,N-dialkylacetamide can be carried out in an inert solvents such as DMF, acetonitrile, toluene and the like, or without a solvent. The reaction of (71) with substituted 3-aminopyrazole (72) in acetic acid at reflux for several hours gives compounds represented by Formula (73). The Pd-mediated coupling reaction of (73) with organometallic agents (68), such as boronic acids (esters) and organostannane, gives compounds represented by Formula (74).

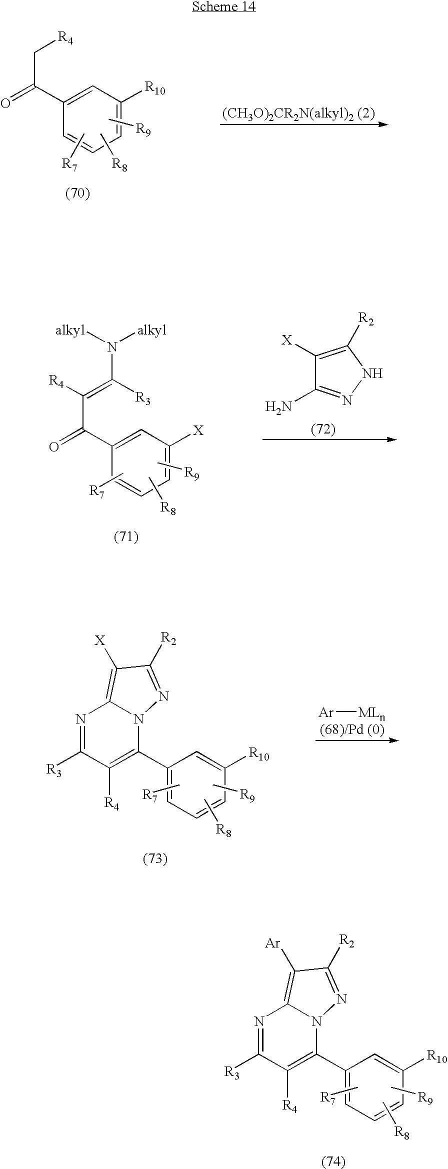

The compounds used in this invention may formulated neat or may be combined with one or more pharmaceutically acceptable carriers for administration. For examples, solvents, diluents and the like and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parentally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to 1000 mg/kg of animal body weight, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The compounds used in this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvant customarily employed in the preparation of pharmaceutical compositions may be advantageously included, such as flavoring agents, preserving agents and antioxidants, for example, vitamin E, ascorbic acid, BHT and BHA.

In some cases, it may be desirable to administer the compounds directly to the airways in the form of an aerosol.

The compounds used in this invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemperaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixture thereof, and vegetable oils.

For the treatment of cancer, the compounds used in this invention can be administered in combination with other antitumor substances or with radiation therapy. These other substances or radiation can be-given at the same or at different times as the compounds used in this invention. These combined therapies may effect synergy and result in improved efficacy. For example, the compounds of this invention can be used in combination with mitotic inhibitors such as taxol or vinblastine, alkylating agents such as cisplatin or cyclophosamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, signal transduction inhibitors such as EGFR (epidermal growth factor receptor) antibodies and EGFR inhibitors, and antiestrogens such as tamoxifen.

The methods of using these compounds can also include simultaneous administration with other agents useful in treating abnormal cell growth or cancer, including agents capable of enhancing antitumor immune responsed, such as CTLA4 (cytotoxic lymphocyte antigen 4) antibodies, and other agents capable of blocking CTLA4; and anti-proliferative agents such as farnesyl protein transferase inhibitors, and the like.

Representative compounds used in this invention were evaluated in several standard pharmaceutical test procedures that showed that the compounds used in this invention possess significant activity as inhibitors of the growth of various cancer cells. Based on the activity shown in the standard pharmacological test procedures, the compounds used in this invention are therefore useful as antineoplastic agents. In particular, these compounds are useful in treating, inhibiting the growth of, or eradicating neoplasms such as those of the breast, kidney, bladder, mouth, larynx, esophagus, stomach, colon, ovary, lung, pancreas, liver, prostate and skin.

The test procedures used and results obtained are shown below. Cytotoxicity Assay Using Isogenic Cell Lines:
1. 80S14 (p21-deficient) cells were cultured in RPMI 1640 medium (Gibco/Invitrogen Life technologies) supplemented with 10% fetal bovine serum (FBS, Gibco) and 10 μg/ml gentamycin (Gibco). Cells were maintained at 37° C. under 7% CO₂
2. Cells were plated at 6×10⁴/ml (0.2 ml/well; 12,000 cells/well) in 96-well microtiter plates (Falcon or Corning/Costar) and incubated at 37° C. overnight (18-24 hours).
3. 2-5 μl of the test agent [200 μg/ml stock in 20% DMSO/20 mM HEPES, pH 7.5 (Gibco)] was added to each well to achieve a final concentration of 2-5 μg/ml. Cells were incubated with the test compounds for 5 days at 37° C.
4. Surviving cells were fixed with 50 μl 50% trichloroacetic acid (Sigma; 10% final concentration) for 1 hour at 4° C.
5. Plates were rinsed twice with water (0.3 ml per wash using MRD8 robot), and allowed to dry.
6. Sulforhodamine B (SRB) (Sigma; 0.4% in 1% acetic acid, 50 μl per well; MRD8 robot) was added and plates kept at room temperature for 10 minutes.
7. Excess dye was removed by washing with 1% acetic acid (3 times, 0.3 ml per wash; MRD8 robot) and allowed to dry.
8. The dye was solubilized in Tris base (Sigma; 10 mM, 150 μl per well). Plates were kept at room temperature for 30 minutes to allow the dye to diffuse.
9. Absorbance was measured at 540 nm.
10. The percentage of cells surviving drug treatment relative to control wells (no drug) was determined.
11. The difference in % cell survival between the two cell lines was calculated.
12. Compounds producing a difference score of >50 were considered to be selective for the p21-deficient cells and were evaluated further:
   Determination of selectivity of compounds for p21−/− cells:
1. 80S14 cells were cultured as described above.
2. Cells were seeded in 96-well microtiter plates at 12,000 cells/well (either 0.15 ml/well of 8×10⁴ cells/ml suspension, or 0.2 ml/well of 6×10⁴ cells/ml suspension). Plates were incubated at 37° C. overnight (18-24 hours).
3. Serial dilutions of the test agent were added to each well. Dilutions were prepared either in 40% DMSO/20% methanol/20 mM HEPES, pH 7.5 (Gibco) or directly in growth medium. One of two dose ranges were used: 5 pM to 50 μM or 170 pg/ml to 10 μg/ml. Cells were incubated with the test compounds for 5 days at 37° C.
4. Surviving cells were fixed with 50 μl 50% trichloroacetic acid (Sigma; 10% final concentration) for 1 hour at 4° C.
5. Plates were rinsed extensively in water, and allowed to dry.
6. Sulforhodamine B (Sigma; 0.4% in 1% acetic acid, 70 μl per well) was added and plates kept at room temperature for 10 minutes.
7. Excess dye was removed by washing 5 times with 1% acetic acid and plates were allowed to dry.
8. The dye was solubilized in Tris base (Sigma; 10 mM, 150 μl per well). Plates were placed on a titer plate shaker (Lab Line Instruments) for 5 minutes to allow the dye to diffuse.
9. Absorbance was measured at 560 nm.

10. The drug concentration that inhibits cell proliferation by 50% relative to untreated controls (IC₅₀) was determined from cytotoxicity curves.

TABLE 1
Cellular activity in 80s14

| | | IC₅₀ for | | |
|---|---|---|---|---|
| Example | | 80SI4 | Mol | Procedure |
| Number | Chemical Name | (uM) | Ion | Number |
| 1 | N-phenyl-N′-{3-[3-(thien-2-ylcarbonyl)pyrazolo[1,5- | +++ | 449 | 1 |
| | a]pyrimidin-7-yl]phenyl}urea | | | |
| 2 | N-(2-phenylethyl)-N′-{3-[3-(thien-2- | ++ | 468 | 1 |
| | ylcarbonyl)pyrazolo[1,5-a]pyrimidin-7-yl]phenyl}urea | | | |
| 3 | N-cyclohexyl-N′-{3-[3-(thien-2-ylcarbonyl)pyrazolo[1,5- | +++ | 446 | 1 |
| | a]pyrimidin-7-yl]phenyl}urea | | | |
| 4 | methyl N-[({3-[3-(thien-2-ylcarbonyl)pyrazolo[1,5- | +++ | 478 | 1 |
| | a]pyrimidin-7-yl]phenyl}amino)carbonyl]-L-valinate | | | |
| 5 | N-ethyl-N′-{3-[3-(thien-2-ylcarbonyl)pyrazolo[1,5- | +++ | 392 | 1 |
| | a]pyrimidin-7-yl]phenyl}urea | | | |
| 6 | N-isopropyl-N′-{3-[3-(thien-2-ylcarbonyl)pyrazolo[1,5- | +++ | 406 | 1 |
| | a]pyrimidin-7-yl]phenyl}urea | | | |
| 7 | N-allyl-N′-{3-[3-(thien-2-ylcarbonyl)pyrazolo[1,5- | +++ | 404 | 1 |
| | a]pyrimidin-7-yl]phenyl}urea | | | |
| 8 | N-butyl-N′-{3-[3-(thien-2-ylcarbonyl)pyrazolo[1,5- | +++ | 420 | 1 |
| | a]pyrimidin-7-yl]phenyl}urea | | | |
| 9 | N-{3-[3-(thien-2-ylcarbonyl)pyrazolo[1,5-a]pyrimidin-7- | ++ | 509 | 1 |
| | yl]phenyl}-N′-(trichloroacetyl)urea | | | |
| 10 | ethyl N-[({3-[3-(thien-2-ylcarbonyl)pyrazolo[1,5- | + | 450 | 1 |
| | a]pyrimidin-7- | | | |
| | yl]phenyl}amino)carbonyl]glycinate | | | |
| 11 | N-[(1S)-1-phenylethyl]-N′-{3-[3-(thien-2- | ++ | 468 | 1 |
| | ylcarbon1)pyrazolo[1,5-a]pyrimidin-7-yl]phenyl}urea | | | |
| 12 | N-[(1S,2R)-2-phenylcyclopropyl]-N′-{3-[3-(thien-2- | +++ | 480 | 1 |
| | ylcarbonyl)pyrazolo[1,5-a]pyrimidin-7-yl]phenyl}urea | | | |
| 13 | butyl 3-[3-(thien-2-ylcarbonyl)pyrazolo[1,5-a]pyrimidin- | +++ | 421 | 3 |
| | 7-yl]phenylcarbamate | | | |
| 14 | allyl 3-[3-(thien-2-ylcarbonyl)pyrazolo[1,5-a]pyrimidin- | +++ | 405 | 3 |
| | 7-yl]phenylcarbamate | | | |
| 15 | isopropyl 3-[3-(thien-2-ylcarbonyl)pyrazolo[1,5- | +++ | 407 | 3 |
| | a]pyrimidin-7-yl]phenylcarbamate | | | |
| 16 | isobutyl 3-[3-(thien-2-ylcarbonyl)pyrazolo[1,5- | +++ | 421 | 3 |
| | a]pyrimidin-7-yl]phenylcarbamate | | | |
| 17 | neopentyl 3-[3-(thien-2-ylcarbonyl)pyrazolo[1,5- | +++ | 435 | 3 |
| | a]pyrimidin-7-yl]phenylcarbamate | | | |
| 18 | 2,2,2-trichlroethyl 3-[3-(thien-2- | +++ | 494, | 3 |
| | ylcarbonyl)pyrazolo[1,5-a]pyrimidin-7- | | 496 | |
| | yl]phenylcarbamate | | | |
| 19 | prop-2-ynyl 3-[3-(thien-2-ylcarbonyl)pyrazolo[1,5- | +++ | 403 | 3 |
| | a]pyrimidin-7-yl]phenylcarbanate | | | |
| 20 | 3-butenyl 3-[3-(2-thienylcarbonyl)pyrazolo[1,5- | +++ | 419 | 3 |
| | a]pyrimidin-7-yl]phenylcarbamate | | | |
| 21 | isopropenyl 3-[3-(thien-2-ylcarbonyl)pyrazolo[1,5- | +++ | 405 | 3 |
| | alpyrimidin-7-yl]phenylcarbamate | | | |
| 22 | 2-chloroethyl 3-[3-(thien-2-ylcarbonyl)pyrazolo[1,5- | +++ | 427, | 3 |
| | alpyrimidin-7-yl]phenylcarbamate | | 429 | |
| 23 | 4-methylphenyl 3-[3-(thien-2-ylcarbonyl)pyrazolo[1,5- | + | 455 | 3 |
| | a]pyrimidin-7-yl]phenylcarbamate | | | |
| 24 | benzyl 3-[3-(thien-2-ylcarbonyl)pyrazolo[1,5- | +++ | 455 | 3 |
| | a]pyrimidin-7-yl]phenylcarbamate | | | |
| 25 | ethyl 3-[3-(thien-2-ylcarbonyl)pyrazolo[1,5-a]pyrimidin- | +++ | 393 | 3 |
| | 7-yl]phenylcarbamate | | | |
| 26 | 4-chlorobutyl 3-[3-(thien-2-ylcarbonyl)pyrazolo[1,5- | +++ | 455, | 3 |
| | a]pyrimidin-7-yl]phenylcarbamate | | 457 | |
| 27 | N-{3-[3-(thien-2-ylcarbonyl)pyrazolo[1,5-a]pyrimidin-7- | ++ | 399 | 8 |
| | yl]phenyl} methanesulfonamide | | | |
| 28 | N-{3-[3-(thien-2-ylcarbonyl)pyrazolo[1,5-a]pyrimidin-7- | +++ | 413 | 8 |
| | yl]phenyl}ethanesulfonamide | | | |
| 29 | N-{3-[3-(thien-2-ylcarbonyl)pyrazolo[1,5-a]pyrimidin-7- | ++ | 441 | 8 |
| | yl]phenyl}butane-1-sulfonamide | | | |
| 30 | N-{3-[3-(thien-2-ylcarbonyl)pyrazolo[1,5-a]pyrimidin-7- | ++ | 427 | 8 |
| | yl]phenyl} propane-2-sulfonamide | | | |
| 31 | N-{3-[3-(thien-2-ylcarbonyl)pyrazolo[1,5-a]pyrimidin-7- | ++ | 461 | 8 |
| | yl]phenyl}benzenesulfonamide | | | |
| 32 | 4-methyl-N-{3-[3-(thien-2-ylcarbonyl)pyrazolo[1,5- | +++ | 475 | 8 |
| | a]pyrimidin-7-yl]phenyl}benzenesulfonamide | | | |
| 33 | 2-methyl-N-{3-[3-(thien-2-ylcarbonyl)pyrazolo[1,5- | +++ | 475 | 8 |
| | a]pyrimidin-7-yl]phenyl}benzenesulfonamide | | | |
| 34 | 4-fluoro-N-{3-[3-(thien-2-ylcarbonyl)pyrazolo[1,5- | +++ | 479 | 8 |
| | a]pyrimidin-7- | | | |
| | yl]phenyl}benzenesulfonamide | | | |
| 35 | 4-bromo-N-{3-[3-(thien-2-ylcarbonyl)pyrazolo[1,5- | ++ | 539, | 8 |
| | a]pyrimidin-7-yl]phenyl}benzenesulfonamide | | 541 | |
| 36 | 4-methoxy-N-{3-[3-(thien-2-ylcarbonyl)pyrazolo[1,5- | ++ | 491 | 8 |
| | a]pyrimidin-7-yl]phenyl}benzenesulfonamide | | | |
| 37 | (E)-2-phenyl-N-{3-[3-(thien-2-ylcarbonyl)pyrazolo[1,5- | + | 487 | 8 |
| | a]pyrimidin-7-yl]phenyl}ethenesulfonamide | | | |
| 38 | N-{3-[3-(thien-2-ylcarbonyl)pyrazolo[1,5-a]pyrimidin-7- | ++ | 467 | 8 |
| | yl]phenyl}thiophene-2-sulfonamide | | | |
| 39 | 1-methyl-N-{3-[3-(thien-2-ylcarbonyl)pyrazolo[1,5- | + | 465 | 8 |
| | a]pyrimidin-7-yl]phenyl}-1H-imidazole-4-sulfonamide | | | |
| 40 | N,N-dimethyl-N′-{3-[3-(thien-2-ylcarbonyl)pyrazolo[1,5- | ++ | 428 | 8 |
| | a]pyrimidin-7-yl]phenyl}sulfamide | | | |
| 41 | ethyl oxo({3-[3-(thien-2-ylcarbonyl)pyrazolo[1,5- | + | 421 | 5 |
| | a]pyrimidin-7- | | | |
| | yl]phenyl}amino)acetate | | | |
| 42 | methyl 4-oxo-4-({3-[3-(thien-2-ylcarbonyl)pyrazolo[1,5- | + | 435 | |
| | a]pyrimidin-7-yl]phenyl}amino)butanoate | | | |
| 43 | 2-methyl-N-{3-[3-(thien-2-ylcarbonyl)pyrazolo[1,5- | +++ | 389 | 5 |
| | a]pyrimidin-7-yl]phenyl}acrylamide | | | |
| 45 | 3-methyl-N-{3-[3-(thien-2-ylcarbonyl)pyrazolo[1,5- | +++ | 403 | 5 |
| | a]pyrimidin-7-yl]phenyl}but-2-enamide | | | |
| 46 | N,N-dimethyl-N′-{3-[3-(thien-2-ylcarbonyl)pyrazolo[1,5- | ++ | 392 | 2 |
| | a]pyrimidin-7-yl]phenyl}urea | | | |
| 47 | 3-methyl-N-{3-[3-(thien-2-ylcarbonyl)pyrazolo[1,5- | +++ | 405 | 5 |
| | a]pyrimidin-7-yl]phenyl}butanamide | | | |
| 48 | 2,2-dimethyl-N-{3-[3-(thien-2-ylcarbonyl)pyrazolo[1,5- | +++ | 405 | 5 |
| | a]pyrimidin-7-yl]phenyl}propanamide | | | |
| 49 | 2,2,2-trichloro-N-{3-[3-(thien-2-ylcarbonyl)pyrazolo[1,5- | ++ | 466 | 5 |
| | a]pyrimidin-7-yl]phenyl}acetamide | | | |
| 50 | 3-(methylthio)-N-{3-[3-(thien-2-ylcarbonyl)pyrazolo[1,5- | +++ | 423 | 5 |
| | a]pyrimidin-7-yl]phenyl}propanamide | | | |
| 51 | N-{3-[3-(thien-2-ylcarbonyl)pyrazolo[1,5-a]pyrimidin-7- | +++ | 431 | 5 |
| | yl]phenyl}thiophene-2-carboxamide | | | |
| 52 | N-{3-[3-(thien-2-ylcarbonyl)pyrazolo[1,5-a]]pyrimidin-7- | ++ | 514 | 5 |
| | yl]phenyl}-1-(trifluoroacetyl)-L-prolinamide | | | |
| 53 | N-{3-[3-(thien-2-ylcarbonyl)pyrazolo[1,5-a]pyrimidin-7- | +++ | 483 | 5 |
| | Yl]phenyl}adamantine-1-carboxamide | | | |
| 54 | 4,7,7-trimethyl-3-oxo-N-{3-[3-(thien-2- | +++ | 501 | 5 |
| | ylcarbonyl)pyrazolo[1,5-a]pyrimidin-7-yl]phenyl}-2- | | | |
| | oxabicyclo[2.2.1]heptane-1-carboxamide | | | |
| 55 | 3,3-dimethyl-N-{3-[3-(thien-2-ylcarbonyl)pyrazolo[1,5- | +++ | 419 | 7 |
| | a]pyrimidin-7-yl]phenyl}butanamide | | | |
| 56 | 4-methyl-N-{3-[3-(thien-2-ylcarbonyl)pyrazolo[1,5- | +++ | 419 | 7 |
| | a]pyrimidin-7-yl]phenyl}pentanamide | | | |
| 57 | N-{3-[3-(thien-2-ylcarbonyl)pyrazolo[1,5-a]pyrimidin-7- | +++ | 403 | 7 |
| | yl]phenyl}pent-4-enamide | | | |
| 58 | N-{3-[3-(thien-2-ylcarbonyl)pyrazolo[1,5-a]pyrimidin-7- | +++ | 301 | 7 |
| | yl]phenyl}pent-4-enamide | | | |
| 59 | 4,4,4-trifluoro-3-methyl-N-{3-[3-(thien-2- | +++ | 459 | 7 |
| | ylcarbonyl)pyrazolo[1,5-a]pyrimidin-7- | | | |
| | yl]phenyl}butanamide | | | |
| 60 | 2-cyano-N-{3-[3-(thien-2-ylcarbonyl)pyrazolo[1,5- | +++ | 388 | 7 |
| | a]pyrimidin-7-yl]phenyl]acetamide | | | |
| 61 | 2-methoxy-N-{3-[3-(thien-2-ylcarbonyl)pyrazolo[1,5- | ++ | 393 | 7 |
| | a]pyrimidin-7-yl]phenyl]acetamide | | | |
| 62 | 2-oxo-2-({3-[3-(thien-2-ylcarbonyl)pyrazolo[1.5- | ++ | 421 | 7 |
| | a]pyrimidin-7-yl]phenyl]amino)ethyl acetate | | | |
| 63 | 2-(2-methoxyethoxy)-N-{3-[3-(thien-2- | ++ | 437 | 7 |
| | ylcarbonyl)pyrazolo[1,5-a]pyrimidin-7- | | | |
| | yl]phenyl}acetamide | | | |
| 64 | N-2-N-2-dimethyl-N-1-{3-[3-(thien-2- | ++ | 406 | 7 |
| | ylcarbonyl)pyrazolo[1,5-a]pyrimidin-7- | | | |
| | yl]phenyl}glycinamide | | | |
| 65 | N-3,N-3-diethyl-N-1-{3-[3-(thien-2- | + | 448 | 7 |
| | ylcarbonyl)pyrazolo[1,5-a]pyrimidin-7-yl]phenyl}-beta- | | | |
| | alaninamide | | | |
| 66 | 2-thien-2-yl-N-{3-[3-(thien-2-ylcarbonyl)pyrazolo[1,5- | +++ | 445 | 7 |
| | a]pyrimidin-7-yl}acetamide | | | |
| 67 | 2-(2,3-dihydro-1H-inden-2-yl)-N-{3-[3-(thien-2- | +++ | 479 | 7 |
| | ylcarbonyl)pyrazolo[1,5-a]pyrimidin-7- | | | |
| | yl]phenyl}acetamide | | | |
| 68 | 2-(2-methylphenyl)-N-{3-[3-(thien-2- | +++ | 453 | 7 |
| | ylcarbonyl)pyrazolo[1,5-a]pyrimidin-7- | | | |
| | yl]phenyl}acetamide | | | |
| 69 | 2-(1,3-benzodioxol-5-yl)-N-{3-[3-(thien-2- | +++ | 483 | 7 |
| | ylcarbonyl)pyrazolo[1,5-a]pyrimidin-7- | | | |
| | yl]phenyl}acetamide | | | |
| 70 | N-{3-[3-(thien-2-ylcarbonyl)pyrazolo[1,5-a]pyrimidin-7- | ++ | 476 | 7 |
| | yl]phenyl}quinoline-3-carboxamide | | | |
| 71 | (2E)-3-phenyl-N-{3-[3-(thien-2-ylcarbonyl)pyrazolo[1,5- | ++ | 451 | 7 |
| | a]pyrimidin-7-yl]phenyl}prop-2-enamide | | | |
| 72 | 2-cyclopropyl-N-{3-[3-(thien-2-ylcarbonyl)pyrazolo[1,5- | +++ | 403 | 7 |
| | a]pyrimidin-7-yl]phenyl}acetamide | | | |
| 73 | 2-cyclopentyl-N-{3-[3-(thien-2-ylcarbonyl)pyrazolo[1,5- | +++ | 431 | 7 |
| | a]pyrimidin-7-yl]phenyl}acetamide | | | |
| 74 | 2-cyclohexyl-N-{3-[3-(thien-2-ylcarbonyl)pyrazolo[1,5- | +++ | 445 | 7 |
| | a]pyrimidin-7-yl]phenyl}acetamide | | | |
| 75 | 2-cycloheptyl-N-{3-[3-(thien-2-ylcarbonyl)pyrazolo[1,5- | +++ | 459 | 7 |
| | a]pyrimidin-7-yl]phenyl}acetamide | | | |
| 76 | 2-bicyclo[2.2.1]hept-2-yl-N-{3-[3-(thien-2- | +++ | 457 | 7 |
| | ylcarbonyl)pyrazolo[1,5-a]pyrimidin-7- | | | |
| | yl]phenyl}acetamide | | | |
| 77 | N-1-{3-[3-(thien-2-ylcarbonyl)pyrazolo[1,5- | + | 425 | 6 |
| | a]pyrimidin-7-yl]phenyl}gylcinamide | | | |
| 78 | N-1-{3-[3-(thien-2-ylcarbonyl)pyrazolo[1,5- | ++ | 439 | 6 |
| | a]pyrimidin-7-yl] phenyl}-L-alaninamide | | | |
| 79 | N-1-{3-[3-(thien-2-ylcarbonyl)pyrazolo[1,5- | + | 455 | 6 |
| | a]pyrimidin-7-yl]phenyl}-L-leucinamide | | | |
| 80 | N-1-{3-[3-(thien-2-ylcarbonyl)pyrazolo[1,5- | ++ | 434 | 6 |
| | a]pyrimidin-7-yl]phenyl}-L-leucinamide | | | |
| 81 | N-{3-[3-(thien-2-ylcarbonyl)pyrazolo[1,5-a]pyrimidin-7- | + | 468 | 6 |
| | yl]phenyl}-L-phenylalaninamide | | | |
| 82 | 3-cyclohexyl-N-1-[3-[3-(thien-2- | + | 474 | 6 |
| | ylcarbonyl)pyrazolo[1,5-a]pyrimidin-7-yl]phenyl}-L- | | | |
| | alaninamide | | | |
| 83 | N-1-{3-[3-(thien-2-ylcarbonyl)pyrazolo[1,5- | ++ | 420 | 6 |
| | a]pyrimidin-7-yl]phenyl}-L-valinamide | | | |
| 84 | 4-amino-N-{3-[3-(thien-2-ylcarbonyl)pyrazolo[1,5- | + | 406 | 6 |
| | a]pyrimidin-7-yl]phenyl}butanamide | | | |
| 85 | N-{3-[3-(thien-2-ylcarbonyl)pyrazolo[1,5-alpyrimidin-7- | ++ | 418 | 6 |
| | yl]phenyl}-L-prolinamide | | | |
| 86 | (2S)-2-amino-2-cyclohexyl-N-{3-(thien-2- | ++ | 460 | 6 |
| | ylcarbonyl)pyrazolo[1,5-a]pyrimidin-7- | | | |
| | yl}phenyl}ethanamide | | | |
| 87 | 4-methyl-N-{3-[3-(thien-2-ylcarbonyl)pyrazolo[1,5- | +++ | 439 | 5 |
| | a]pyrimidin-7-yl]phenyl]benzamide | | | |
| 88 | 4-methoxy-N-{3-[3-(thien-2-ylcarbonyl)pyrazolo[1,5- | +++ | 455 | 5 |
| | a]pyrimidin-7-yl]phenyl]benzamide | | | |
| 89 | 4-chloro-N-{3-[3-thien-2-ylcarbonyl)pyrazolo[1,5- | ++ | 459, | 5 |
| | a]pyrimidin-7-yl]phenyl]benzamide | | 461 | |
| 90 | 4-bromo-N-{3-[3-(thien-2-ylcarbonyl)pyrazolo[1,5- | ++ | 503, | 5 |
| | a]pyrimidin-7-yl]phenyl}benzamide | | 505 | |
| 91 | 4-(heptyloxy)-N-{3-[3-(thien-2-ylcarbonyl)pyrazolo[1,5- | + | 539 | 5 |
| | a]pyrimidin-7-yl]phenyl}benzamide | | | |
| 92 | 4-pentyl-N-{3-[3-(thien-2-ylcarbonyl)pyrazolo[1,5- | + | 495 | 5 |
| | a]pyrimidin-7-yl]phenyl}benzamide | | | |
| 93 | 2-methoxy-N-{3-[3-(thien-2-ylcarbonyl)pyrazolo[1,5- | +++ | 455 | 5 |
| | a]pyrimidin-7-yl]phenyl}benzamide | | | |
| 94 | 2-methyl-N-{3-[3-(thien-2-ylcarbonyl)pyrazolo[1,5- | +++ | 439 | 5 |
| | a]pyrimidin-7-yl]phenyl}benzamide | | | |
| 95 | 2-chloro-N-{3-[3-(thien-2-ylcarbonyl)pyrazolo[1,5- | +++ | 459, | 5 |
| | a]pyrimidin-7-yl]phenyl}benzamide | | 461 | |
| 96 | N-{3-[3-(thien-2-ylcarbonyl)pyrazolo[1,5-a]pyrimidin-7- | +++ | 493 | 5 |
| | yl]phenyl}-2-(trifluoromethyl)benzamide | | | |
| 97 | 3-methoxy-N-{3-[3-(thien-2-ylcarbonyl)pyrazolo[1,5- | +++ | 455 | 5 |
| | a]pyrimidin-7-yl]phenyl}benzamide | | | |
| 98 | 3-fluoro-N-{3-[3-(thien-2-ylcarbonyl)pyrazolo[1,5- | +++ | 443 | 5 |
| | a]pyrimidin-7-yl]phenyl}benzamide | | | |
| 99 | 3,4-dimethoxy-N-{3-[3-(thien-2- | ++ | 485 | 5 |
| | ylcarbonyl)pyrazolo[1,5-a]pyrimidin-7- | | | |
| | yl]phenyl}benzamide | | | |
| 100 | 3,4-dichloro-N-{3-[3-(thien-2-ylcarbonyl)pyrazolo[1,5- | + | 493, | 5 |
| | a]pyrimidin-7-yl]phenyl}benzamide | | 495 | |
| 101 | 2,6-dimethoxy-N-{3-[3-(thien-2- | +++ | 485 | 5 |
| | ylcarbonyl)pyrazolo[1,5-a]pyrimidin-7- | | | |
| | yl]phenyl}benzamide | | | |
| 102 | 3,4-difluoro-N-{3-[3-(thien-2-ylcarbonyl)pyrazolo[1,5- | ++ | 461 | 5 |
| | a]pyrimidin-7-yl]phenyl}benzamide | | | |
| 103 | N-{3-[3-(thien-2-ylcarbonyl)pyrazolo[1,5-a]pyrimidin-7- | +++ | 475 | 5 |
| | yl]phenyl}-1-naphthamide | | | |
| 104 | N-{3-[3-(thien-2-ylcarbonyl)pyrazolo[1,5-a]pyrimidin-7- | + | 475 | 5 |
| | yl]phenyl}-2-naphthamide | | | |
| 105 | 2-phenyl-N-{3-[3-(thien-2-ylcarbonyl)pyrazolo[1,5- | +++ | 439 | 5 |
| | a]pyrimidin-7-yl]phenyl}acetamide | | | |
| 106 | 2-(4-methoxyphenyl)-N-{3-[3-(thien-2- | +++ | 469 | 5 |
| | ylcarbonyl)pyrazolo[1,5-a]pyrimidin-7- | | | |
| | yl]phenyl}acetamide | | | |
| 107 | 2-(3-methoxyphenyl)-N-{3-[3-(thien-2- | +++ | 469 | 5 |
| | ylcarbonyl)pyrazolo[1,5-a]pyrimidin-7- | | | |
| | yl]phenyl}acetamide | | | |
| 108 | 2-(4-chlorophenyl)-N-{3-[3-(thien-2- | +++ | 473, | 5 |
| | ylcarbonyl)pyrazolo[1,5-a]pyrimidin-7- | | 475 | |
| | yl]phenyl}acetamide | | | |
| 109 | 2-(4-fluorophenyl)-N-{3-[3-(thien-2- | +++ | 457 | 5 |
| | ylcarbonyl)pyrazolo[1,5-a]pyrimidin-7- | | | |
| | yl]phenyl}acetamide | | | |
| 110 | 2-(2,5-dimethoxyphenyl)-N-{3-[3-(thien-2- | ++ | 499 | 5 |
| | ylcarbonyl)pyrazolo[1,5-a]pyrimidin-7- | | | |
| | yl]phenyl}acetamide | | | |
| 111 | 2-phenoxy-N-{3-[3-(thien-2-ylcarbonyl)pyrazolo[1,5- | ++ | 455 | 5 |
| | a]pyrimidin-7-yl]phenyl}acetamide | | | |
| 112 | 4-fluoro-N-{3-[3-(thien-2-ylcarbonyl)pyrazolo[1,5- | +++ | 443 | 5 |
| | a]pyrimidin-7-yl]phenyl}benzamide | | | |
| 113 | ethyl 2-oxo-2-({3-[3-(thien-2-ylcarbonyl)pyrazolo[1,5- | ++ | 450 | 5 |
| | a]pyrimidin-7-yl]phenyl}amino)ethylcarbamate | | | |
| 114 | propyl 2-oxo-2-({3-[3-(thien-2-ylcarbonyl)pyrazolo[1,5- | + | 464 | 5 |
| | a]pyrimidin-7-yl]phenyl}amino)ethylcarbamate | | | |
| 115 | isopropyl 2-oxo-2-({3-[3-(thien-2- | ++ | 464 | 5 |
| | ylcarbonyl)pyrazolo[1,5-a]pyrimidin-7- | | | |
| | yl]phenyl}amino)ethylcarbamate | | | |
| 116 | butyl 2-oxo-2-({3-[3-(thien-2-ylcarbonyl)pyrazolo[1,5- | +++ | 478 | 5 |
| | a]pyrimidin-7-yl]phenyl}amino)ethylcarbamate | | | |
| 117 | isobutyl 2-oxo-2-({3-[3-(thien-2- | ++ | 478 | 5 |
| | ylcarbonyl)pyrazolo[1,5-a]pyrimidin-7- | | | |
| | yl]phenyl}amino)ethylcarbamate | | | |
| 118 | but-3-enyl-2-oxo-2-({3-[3-(thien-2- | ++ | 476 | 5 |
| | ylcarbonyl)pyrazolo[1,5-a]pyrimidin-7- | | | |
| | yl]phenyl}amino)ethylcarbamate | | | |
| 119 | 4-chlorobutyl 2-oxo-2-({3-[3-(thien-2- | + | 512 | 5 |
| | ylcarbonyl)pyrazolo[1,5-a]pyrimidin-7- | | | |
| | yl]phenyl}amino)ethylcarbamate | | | |
| 120 | 2-chloroethyl-2-oxo-2-({3-[3-(thien-2- | ++ | 484 | 5 |
| | ylcarbonyl)pyrazolo[1,5-a]pyrimidin-7- | | | |
| | yl]phenyl}amino)ethylcarbamate | | | |
| 121 | neopentyl 2-oxo-2-({3-[3-(thien-2- | ++ | 492 | 5 |
| | ylcarbonyl)pyrazolo[1,5-a]pyrimidin-7- | | | |
| | yl]phenyl} amino)ethylcarbamate | | | |
| 122 | hexyl 2-oxo-2-({3-[3-(thien-2-ylcarbonyl)pyrazolo[1,5- | ++ | 506 | 5 |
| | a]pyrimidin-7-yl]phenyl}amino)ethylcarbamate | | | |
| 123 | prop-2-ynyl 2-oxo-2-({3-[3-(thien-2- | ++ | 460 | 5 |
| | ylcarbonyl)pyrazolo[1,5-a]pyrimidin-7- | | | |
| | yl]phenyl}amino)ethylcarbamate | | | |
| 124 | allyl 2-oxo-2-({3-[3-(thien-2-ylcarbonyl)pyrazolo[1,5- | + | 462 | 5 |
| | a)pyrimidin-7-yl]phenyl}amino)ethylcarbamate | | | |
| 125 | benzyl 2-oxo-2-({3-[3-(thien-2-ylcarbonyl)pyrazolo[1,5- | ++ | 512 | 5 |
| | a]pyrimidin-7-yl]phenyl}amino)ethylcarbamate | | | |
| 126 | N-[2-oxo-2-({3-[3-(thien-2-ylcarbonyl)pyrazolo[1,5- | + | 446 | 5 |
| | a]pyrimidin-7- | | | |
| | yl]phenyl}amino)ethyl]cyclopropanecarboxamide | | | |
| 127 | N-[2-oxo-2-({3-[3-(thien-2-ylcarbonyl)pyrazolo[1,5- | + | 460 | 5 |
| | a]pyrimidin-7- | | | |
| | yl]phenyl}amino)ethyl]cyclobutanecarboxamide | | | |
| 128 | N-[2-oxo-2-({3-[3-(thien-2-ylcarbonyl)pyrazolo[1,5- | ++ | 474 | 5 |
| | a]pyrimidin-7- | | | |
| | yl]phenyl}amino)ethyl]cyclopentanecarboxamide | | | |
| 129 | N-[2-oxo-2-({3-[3-(thien-2-ylcarbonyl)pyrazolo[1,5- | + | 488 | 5 |
| | a]pyrimidin-7- | | | |
| | yl]phenyl}amino)ethyl]cyclohexanecarboxamide | | | |
| 130 | 2-methyl-N-[2-oxo-2-({3-[3-(thien-2- | + | 462 | 5 |
| | ylcarbonyl)pyrazolo[1,5-a]pyrimidin-7- | | | |
| | yl]phenyl}amino)ethyl]butanamide | | | |
| 131 | 2-ethyl-N-[2-oxo-2-({3-[3-(thien-2- | + | 476 | 5 |
| | ylcarbonyl)pyrazolo[1,5-a]pyrimidin-7- | | | |
| | yl]phenyl}amino)ethyl]butanamide | | | |
| 132 | 2,2-dimethyl-N-[2-oxo-2-(13-[3-(thien-2- | ++ | 462 | 5 |
| | ylcarbonyl)pyrazolo[1,5-a]pyrimidin-7- | | | |
| | yl]phenyl}amino)ethyl]propanamide | | | |
| 133 | 3-methyl-N-[2-oxo-2-({3-[3-(thien-2- | ++ | 462 | 5 |
| | ylcarbonyl)pyrazolo[1,5-a]pyrimidin-7- | | | |
| | yl]phenyl}amino)ethyl]butanamide | | | |
| 134 | N-[2-oxo-2-({3-[3-(thien-2-ylcarbonyl)pyrazolo[1,5- | ++ | 540 | 5 |
| | a]pyrimidin-7-yl]phenyl}amino)ethyl]adamantine-1- | | | |
| | carboxamide | | | |
| 135 | N-[2-oxo-2-({3-[3-(thien-2-ylcarbonyl)pyrazolo[1,5- | ++ | 482 | 5 |
| | a]pyrimidin-7-yl]phenyl]amino)ethyl]benzamide | | | |
| 136 | N-2-[(pentylamino)carbonyl]-N-1-{3-[3-(thien-2- | + | 491 | 5 |
| | ylcarbonyl)pyrazolo[1,5-a]pyrimidin-7- | | | |
| | yl]phenyl}gylcinamide | | | |
| 137 | N-2-[(ethylamino)carbonyl]-N-1-{3-[3-(thien-2- | ++ | 449 | 5 |
| | ylcarbonyl)pyrazolo[1,5-a]pyrimidin-7- | | | |
| | yl]phenyl}glycinamide | | | |
| 138 | N-2-[(cyclohexylamino)carbonyl]-N1-{3-[3-(thien- | ++ | 503 | 5 |
| | 2-ylcarbonyl)pyrazolo[1,5-a]pyrimidin-7- | | | |
| | yl]phenyl}glycinamide | | | |
| 139 | 3-chloro-N-{3-[3-(thien-2-ylcarbonyl)pyrazolo[1,5- | +++ | 411, | 5 |
| | a]pyrimidin-7-yl]phenyl}propanamide | | 413 | |
| 140 | 3,5,5-trimethyl-N-{3-[3-(thien-2- | +++ | 461 | 5 |
| | ylcarbonyl)pyrazolo[1,5-a]pyrimidin-7- | | | |
| | yl]phenyl}hexanamide | | | |
| 141 | 3-chloro-2-(chloromethyl)-2-methyl-N-{3-[3-(thien-2- | +++ | 473, | 5 |
| | ylcarbonyl)pyrazolo[1,5-a]pyrimidin7- | | 475 | |
| | yl]phenyl}propanamide | | | |
| 142 | N-{3-[3-(thien-2-ylcarbonyl)pyrazolo[1,5-a]pyrimidin-7- | + | 489 | 5 |
| | yl]phenyl}undecanamide | | | |
| 143 | 1-methyl-N-{3-[3-(thien-2-ylcarbonyl)pyrazolo[1,5- | +++ | 445 | 5 |
| | a]pyrimidin-7-yl]phenyl}cyclohexanecarboxamide | | | |
| 144 | 2,3,3-trichloro-N-{3-[3-(thien-2-ylcarbonyl)pyrazolo[1,5- | +++ | 479, | 5 |
| | a]pyrimidin-7-yl]phenyl}acrylamide | | 481 | |
| 145 | 2-methyl-N-{3-[3-(thien-2-ylcarbonyl)pyrazolo[1,5- | +++ | 405 | 5 |
| | alpyrimidin-7-yl]phenyl}butanamide | | | |
| 146 | 3-cyclopentyl-N-{3-[3-(thien-2-ylcarbonyl)pyrazolo[1,5- | ++ | 445 | 5 |
| | a]pyrimidin-7-yl]phenyl}propanamide | | | |
| 147 | 2-ethyl-N-{3-[3-(thien-2-ylcarbonyl)pyrazolo[1,5- | ++ | 447 | 5 |
| | a]pyrimidin-7-yl}phenyl}hexanamide | | | |
| 148 | N-{3-[3-(thien-2-ylcarbonyl)pyrazolo[1,5-a]pyrimidin-7- | +++ | 391 | 5 |
| | yl]phenyl}butanamide | | | |
| 149 | (2E)-N-{3-[3-(thien-2-ylcarbonvl)pyrazolo[1,5- | ++ | 389 | 5 |
| | a]pyrimidin-7-yl]phenyl}but-2-enamide | | | |
| 150 | 2-ethyl-N-{3-[3-(thien-2-ylcarbonyl)pyrazolo[1,5- | ++ | 419 | 5 |
| | a]pyrimidin-7-yl]phenyl}butanamide | | | |
| 151 | N-{3-[3-(thien-2-ylcarbonyl)pyrazolo[1,5-a]pyrimidin-7- | ++ | 433 | 5 |
| | yl]phenyl}heptanamide | | | |
| 152 | N-3{3-[3-(thien-2-ylcarbonyl)pyrazolo[1,5-a]pyrimidin- | +++ | 405 | 5 |
| | 7-yl]phenyl}pentanamide | | | |
| 153 | N-{3-[thien-2-ylcarbonyl)pyrazolo[1,5-a]pyrimidin-7- | +++ | 419 | 5 |
| | yl]phenyl}hexanamide | | | |
| 154 | 2-methyl-N-{3-[3-(thien-2-ylcarbonyl)pyrazolo[1,5- | +++ | 419 | 5 |
| | a]pyrimidin-7-yl]phenyl}pentanamide | | | |
| 155 | N-{3-[3-(thien-2-ylcarbonyl)pyrazolo[1,5-a]pyrimidin-7- | +++ | 432 | 2 |
| | yl]phenyl}piperidine-1-carboxamide | | | |
| 156 | N-butyl-N-methyl-N′-{3-[3-(thien-2- | +++ | 434 | 2 |
| | ylcarbonyl)pyrazolo[1,5-a]primidin-7-yl]phenyl}urea | | | |
| 157 | N-{3-[3-(thien-2-ylcarbonyl)pyrazolo[1,5-a]pyrimidin-7- | ++ | 434 | 2 |
| | ylphenyl}morpholne-4-carboxamide | | | |
| 158 | N-(sec-butyl)-N′-{3-[3-(thien-2-ylcarbonyl)pyrazolo[1,5- | +++ | 420 | 2 |
| | a]pyrimidin-7-yl]phenyl}urea | | | |
| 159 | N,N-diethyl-N′-{3-[3-(thien-2-ylcarbonyl)pyrazolo[1,5- | ++ | 420 | 2 |
| | a]pyrimidin-7-yl]phenyl}urea | | | |
| 160 | N-(cyclohexylmethyl)-N′-{3-[3-(thien-2- | ++ | 460 | 2 |
| | ylcarbonyl)pyrazolo[1,5-a]pyrimidin-7-yl]phenyl}urea | | | |
| 161 | N-[2-(dimethylamino)ethyl]-N′-{3-[3-(thien-2- | + | 435 | 2 |
| | ylcarbonlyl)pyrazolo[1,5-a]pyrimidin-7-yl]phenyl}urea | | | |
| 162 | N-cyclopentyl-N′-{3-[3-(thien-2- | +++ | 432 | 2 |
| | ylcarbonyl)pyrazolo[1,5-a]pyrimidin-7-yl]phenyl}urea | | | |
| 163 | N-(tetrahydrofuran-2-ylmethyl)-N′-{3-[3-(thien-2- | +++ | 448 | 2 |
| | ylcarbonyl)pyrazolo[1,5-a]pyrimidin-7-yl]phenyl}urea | | | |
| 164 | N-neopentyl-N′-{3-[3-(thien-2-ylcarbonyl)pyrazolo[1,5- | +++ | 434 | 2 |
| | a]pyrimidin-7-yl]phenyl}urea | | | |
| 165 | N-pentyl-N′-{3-[3-(thien-2-ylcarbonyl)pyrazolo[1,5- | +++ | 434 | 2 |
| | a]pyrimidin-7-yl]phenyl}urea | | | |
| 166 | 4-methyl-N-{3-[3-(thien-2-ylcarbonyl)pyrazolo[1,5- | ++ | 447 | 2 |
| | a]pyrimidin-7-yl]phenyl}piperazine-1-carboxamide | | | |
| 167 | 4-methyl-N-{3-[3-(thien-2-ylcarbonyl)pyrazolo[1,5- | ++ | 461 | 2 |
| | a]pyrimidin-7-yl]phenyl}-1,4-diazepane-1-carboxamide | | | |
| 168 | N,N-bis(2-methoxyethyl)-N′-{3-[3-(thien-2- | + | 480 | 2 |
| | ylcarbonyl)pyrazolo[1,5-a]pyrimidin-7-yl]phenyl}urea | | | |
| 169 | N-cyclopropyl-N′-{3-[3-(thien-2- | ++ | 404 | 2 |
| | ylcarbonyl)pyrazolo[1,5-a]pyrimidin-7-yl]phenyl}urea | | | |
| 170 | N-{3-[3-(thien-2-ylcarbonyl)pyrazolo[1,5-a]pyrimidin-7- | ++ | 418 | 2 |
| | yl]phenyl}pyrrolidine-1-carboxamide | | | |
| 171 | cyclopentyl 3-[3-(thien-2-ylcarbonyl)pyrazolo[1,5- | +++ | 433 | 4 |
| | a]pyrimidin-7-yl]phenylcarbamate | | | |
| 172 | cyclohexyl 3-(3-(thien-2-ylcarbonyl)pyrazolo[1,5- | +++ | 447 | 4 |
| | a]pyrimidin-7-yl]phenylcarbamate | | | |
| 173 | cyclohex-2-en-1-yl 3-[3-(thien-2- | +++ | 445 | 4 |
| | ylcarbonyl)pyrazolo[1,5-a]pyrimidin-7- | | | |
| | yl]phenylcarbamate | | | |
| 174 | cyclobutylmethyl 3-[3-(thien-2-ylcarbonyl)pyrazolo[1,5- | +++ | 433 | 4 |
| | a]pyrimidin-7-yl]phenylcarbamate | | | |
| 175 | cyclohexylmethyl 3-[3-(thien-2-ylcarbonyl)pyrazolo[1,5- | +++ | 461 | 4 |
| | a]pyrimidin-7-yl]phenylcarbamate | | | |
| 176 | tetrahydro-2H-pyran-4-yl 3-[3-(thien-2- | +++ | 449 | 4 |
| | ylcarbonyl)pyrazolo[1,5-a]pyrimidin-7- | | | |
| | yl]phenylcarbamate | | | |
| 177 | 3-methylbut-2-enyl 3-[3-(thien-2- | ++ | 433 | 4 |
| | ylcarbonyl)pyrazolo[1,5-a]pyrimidin-7- | | | |
| | yl]phenylcarbamate | | | |
| 178 | 2-(methylthio)ethyl 3-[3-(thien-2- | +++ | 439 | 4 |
| | ylcarbonyl)pyrazolo[1,5-a]pyrimidin-7- | | | |
| | yl]phenylcarbamate | | | |
| 179 | 2-thien-3-ylethyl 3-[3-(thien-2-ylcarbonyl)pyrazolo[1,5- | ++ | 475 | 4 |
| | a]pyrimidin-7-yl]phenylcarbamate | | | |
| 180 | 2-piperidin-1-ylethyl 3-[3-(thien-2- | ++ | 476 | 4 |
| | ylcarbonyl)pyrazolo[1,5-a]pyrimidin-7- | | | |
| | yl]phenylcarbamate | | | |
| 181 | 2-morpholin-4-ylethyl 3-[3-(thien-2- | +++ | 478 | 4 |
| | ylcarbonyl)pyrazolo[1,5-a]pyrimidin-7- | | | |
| | yl]phenylcarbamate | | | |
| 182 | 1-methylprop-2-enyl 3-[3-(thien-2- | +++ | 419 | 4 |
| | ylcarbonyl)pyrazolo[1,5-a]pyrimidin-7- | | | |
| | yl]phenylcarbamate | | | |
| 183 | 1-methylbut-3-ynyl 3-[3-(thien-2- | +++ | 431 | 4 |
| | ylcarbonyl)pyrazolo[1,5-a]pyrimidin-7- | | | |
| | yl]phenylcarbamate | | | |
| 184 | 1-ethyl-2-methylpropyl 3-[3-(thien-2- | ++ | 449 | 4 |
| | ylcarbonyl)pyrazolo[1,5-a]pyrimidin-7- | | | |
| | yl]phenylcarbamate | | | |
| 185 | 1,3-dimethylbutyl3-[3-(thien-2-ylcarbonyl)pyrazolo[1,5- | ++ | 449 | 4 |
| | a]pyrimidin-7-yl]phenylcarbamate | | | |
| 186 | 1-ethylpropyl 3-[3-(thien-2-ylcarbonyl)pyrazolo[1,5- | +++ | 435 | 4 |
| | a]pyrimidin-7-yl]phenylcarbamate | | | |
| 187 | (2-methylcyclopropyl)methyl 3-[3-(thien-2- | +++ | 433 | 4 |
| | ylcarbonyl)pyrazolo[1,5-a]pyrimidin-7- | | | |
| | yl]phenylcarbamate | | | |
| 188 | 3-methylcyclopentyl 3-[3-(thien-2- | +++ | 447 | 4 |
| | ylcarbonyl)pyrazolo[1,5-a]pyrimidin-7- | | | |
| | yl]phenylcarbamate | | | |
| 189 | 1-methylbut-3-enyl 3-[3-(thien-2- | +++ | 433 | 4 |
| | ylcarbonyl)pyrazolo[1,5-a]pyrimidin-7- | | | |
| | yl]phenylcarbamate | | | |
| 190 | 1-cyclopropylethyl 3-[3-(thien-2- | +++ | 433 | 4 |
| | ylcarbonyl)pyrazolo[1,5-a]pyrimidin-7- | | | |
| | yl]phenylcarbamate | | | |
| 191 | 1,2-dimethylpropyl 3-[3-(thien-2- | +++ | 435 | 4 |
| | ylcarbonyl)pyrazolo[1,5-a]pyrimidin-7- | | | |
| | yl]phenylcarbamate | | | |
| 192 | 1-isopropylbutyl 3-[3-(thien-2-ylcarbonyl)pyrazolo[1,5- | ++ | 463 | 4 |
| | a]pyrimidin-7-yl]phenylcarbamate | | | |
| 193 | but-3-ynyl 3-[3-(thien-2-ylcarbonyl)pyrazolo[1,5- | +++ | 417 | 4 |
| | a]pyrimidin-7- | | | |
| | yl]phenylcarbamate | | | |
| 194 | propyl 3-[5-methyl-3-(thien-2-ylcarbonyl)pyrazolo[1,5- | +++ | 421 | 3 |
| | a]pyrimidin-7-yl]phenylcarbamate | | | |
| 195 | butyl 3-[5-methyl-3-(thien-2-ylcarbonyl)pyrazolo[1,5- | +++ | 435 | 3 |
| | a]pyrimidin-7-yl]phenylcarbamate | | | |
| 196 | allyl 3-[5-methyl-3-(thien-2-ylcarbonyl)pyrazolo[1,5- | +++ | 419 | 3 |
| | a]pyrimidin-7-yl]phenylcarbamate | | | |
| 197 | isopropyl 3-[5-methyl-3-(thien-2- | +++ | 421 | 3 |
| | ylcarbonyl)pyrazolo[1,5-a]pyrimidin-7- | | | |
| | yl]phenylcarbamate | | | |
| 198 | 2-chloroethyl 3-[5-methyl-3-(thien-2- | +++ | 441 | 3 |
| | ylcarbonyl)pyrazolo[1,5-a]pyrimidin-7- | | | |
| | yl]phenylcarbamate | | | |
| 199 | but-3-enyl 3-[5-methyl-3-(thien-2- | +++ | 433 | 3 |
| | ylcarbonyl)pyrazolo[1,5-a]pyrimidin-7- | | | |
| | yl]phenylcarbamate | | | |
| 200 | neopentyl 3-[5-methyl-3-(thien-2- | +++ | 449 | 3 |
| | ylcarbonyl)pyrazolo[1,5-a]pyrimidin-7- | | | |
| | yl]phenylcarbamate | | | |
| 201 | 3-methyl-N-{3-[5-methyl-3-(thien-2- | +++ | 419 | 5 |
| | ylcarbonyl)pyrazolo[1,5-a]pyrimidin-7- | | | |
| | yl]phenyl}butanamide | | | |
| 202 | N-{3-[5-methyl-3-(thien-2-ylcarbonyl)pyrazolo[1,5- | + | 439 | 5 |
| | a]pyrimidin-7-yl]phenyl}benzamide | | | |
| 204 | N-{3-[5-methyl-3-(thien-2-ylcarbonyl)pyrazolo[1,5- | +++ | 431 | 5 |
| | a]pyrimidin-7-yl]phenyl}cyclopentanecarboxamide | | | |
| 205 | N-{3-[5-methyl-3-(thien-2-ylcarbonyl)pyrazolo[1,5- | ++ | 459 | 5 |
| | a]pyrimidin-7-yl]phenyl}-2-thien-2-ylacetamide | | | |
| 206 | N-butyl-N′-{3-[5-methyl-3-(thien-2- | ++ | 434 | 7 |
| | ylcarbonyl)pyrazolo[1,5-a]pyrimidin-7-yl]phenyl}urea | | | |
| 207 | N-isopropyl-N′-{3-[5-methyl-3-(thien-2- | +++ | 420 | 7 |
| | ylcarbonyl)pyrazolo[1,5-a]pyrimidin-7-yl]phenyl}urea | | | |
| 208 | propyl 3-[6-methyl-3-(thien-2-ylcarbonyl)pyrazolo[1,5- | + | 421 | 4 |
| | a]pyrimidin-7-yl]phenylcarbamate | | | |
| 209 | butyl 3-[6-methyl-3-(thien-2-ylcarbonyl)pyrazolo[1,5- | + | 435 | 4 |
| | a]pyrimidin-7-yl]phenylcarbamate | | | |
| 210 | allyl 3-[6-methyl-3-(thien-2-ylcarbonyl)pyrazolo[1,5- | + | 419 | 4 |
| | a]pyrimidin-7-yl]phenylcarbamate | | | |
| 211 | isopropyl 3-[6-methyl-3-(thien-2- | + | 421 | 4 |
| | ylcarbonyl)pyrazolo[1,5-a]pyrimidin-7- | | | |
| | yl]phenylcarbamate | | | |
| 212 | 2-chloroethyl 3-[6-methyl-3-(thien-2- | + | 441 | 4 |
| | ylcarbonyl)pyrazolo[1,5-a]pyrimidin-7- | | | |
| | yl]phenylcarbamate | | | |
| 213 | but-3-enyl 3-[6-methyl-3-(thien-2- | + | 433 | 4 |
| | ylcarbonyl)pyrazolo[1,5-a]pyrimidin-7- | | | |
| | yl]phenylcarbamate | | | |
| 214 | neopentyl 3-[6-methyl-3-(thien-2- | + | 449 | 4 |
| | ylcarbonyl)pyrazolo[1,5-a]pyrimidin-7- | | | |
| | yl]phenylcarbamate | | | |
| 215 | 3-methyl-N-{3-[6-methyl-3-(thien-2- | + | 419 | 5 |
| | ylcarbonyl)pyrazolo[1,5-a]pyrimidin-7- | | | |
| | yl]phenyl}butanamide | | | |
| 216 | N-{3-[6-methyl-3-(thien-2-ylcarbonyl)pyrazolo[1,5- | + | 439 | 5 |
| | a]pyrimidin-7-yl]phenyl}benzamide | | | |
| 217 | N-{3-[6-methyl-3-(thien-2-ylcarbonyl)pyrazolo[1,5- | + | 403 | 5 |
| | a]pyrimidin-7-yl]phenyl}cycopropanecarboxamide | | | |
| 218 | N-{3-[6-methyl-3-(thien-2-ylcarbonyl)pyrazolo[1,5- | + | 431 | 5 |
| | a]pyrimidin-7-yl]phenyl}cyclopentanecarboxamide | | | |
| 219 | N-{3-[6-methyl-3-(thien-2-ylcarbonyl)pyrazolo[1,5- | + | 459 | 5 |
| | a]pyrimidin-7-yl]phenyl}-2-thien-2-ylacetamide | | | |
| 220 | N-{3-[6-methyl-3-(thien-2-ylcarbonyl)pyrazolo[1,5- | + | 440 | 5 |
| | a]pyrimidin-7-yl]phenyl}nicotinamide | | | |
| 221 | N-butyl-N′-{3-[6-methyl-3-(thien-2- | + | 434 | 7 |
| | ylcarbonyl)pyrazolo[1,5-a]pyrimidin-7-yl]phenyl}urea | | | |
| 222 | N-isopropyl-N′-{3-[6-methyl-3-(thien-2- | + | 420 | 7 |
| | ylcarbonyl)pyrazolo[1,5-a]pyrimidin-7-yl]phenyl}urea | | | |

+++, <1 μM;
++, 1-10 μM;
+; ⁻10-50 μM

Cytotoxicity Assay Using LoVo Colon Cell Line:
1. LoVo Colon cell lines were cultured in RPMI 1640 medium (Gibco/Invitrogen Life Sciences) supplemented with 10% fetal bovine serum (FBS, Gibco) and 50 μg/ml entamicin (Gibco). Cells were maintained at 37° C. under 7% CO₂.
2. Cells were seeded in 96-well microtiter plates (Corning/Costar) in a volume of 0.15 ml of growth medium (4000 cells/well). Plates were incubated at 37° C. overnight (18-24 hours).
3. Serial dilutions of the test agent were added to each well. Dilutions were prepared in growth medium. One of two dose ranges was used: 5 pM to 20 μM or 170 pg/ml to 10 μg/ml. Cells were incubated with the test compounds for 4 days at 37° C.
4. Surviving cells were processed using the SRB asay, as described above.

5. The drug concentration that inhibits cell proliferation by 50% relative to untreated controls (IC₅₀) was determined from cytotoxicity curves using the LSW Toolbox graphing application.

TABLE 2
Cellular activity in LoVo cell

| Example | | Inhibition of |
|---|---|---|
| Number | Chemical Name | LoVo |
| 2 | N-(2-phenylethyl)-N′-{3-[3-(thien-2- | ++ |
| | ylcarbonyl)pyrazolo[1,5-a]pyrimidin-7-yl]phenyl}urea | |
| 3 | N-cyclohexyl-N′-{3-[3-(thien-2-ylcarbonyl)pyrazolo[1,5- | +++ |
| | a]pyrimidin-7-yl]phenyl}urea | |
| 8 | N-butyl-N′-{3-[3-(thien-2-ylcarbonyl)pyrazolo[1,5- | +++ |
| | a]pyrimidin-7-yl]phenvl}urea | |
| 13 | butyl 3-[3-(thien-2-ylcarbonyl)pyrazolo[1,5-a]pyrimidin- | +++ |
| | 7-yl]phenylcarbamate | |
| 14 | allyl 3-[3-(thien-2-ylcarbonyl)pyrazolo[1,5-a]pyrimidin- | +++ |
| | 7-yl]phenylcarbamate | |
| 17 | neopentyl 3-[3-(thien-2-ylcarbonyl)pyrazolo[1,5- | +++ |
| | a]pyrimidin-7-yl]phenylcarbamate | |
| 19 | prop-2-ynyl 3-[3-(thien-2-ylcarbonyl)pyrazolo[1,5- | ++ |
| | a]pyrimidin-7-yl]phenylcarbamate | |
| 22 | 2-chloroethyl 3-[3-(thien-2-ylcarbonyl)pyrazolo[1,5- | +++ |
| | a]pyrimidin-7- | |
| | yl]phenylcarbamate | |
| 25 | ethyl 3-[3-(thien-2-ylcarbonyl)pyrazolo[1,5-a]pyrimidin- | +++ |
| | 7-yl]phenylcarbamate | |
| 26 | 4-chlorobutyl 3-[3-(thien-2-ylcarbonyl)pyrazolo[1,5- | +++ |
| | a]pyrimidin-7-yl]phenylcarbamate | |
| 44 | 2-methyl-N-{3-[3-(thien-2-ylcarbonyl)pyrazolo[1,5- | +++ |
| | a]pyrimidin-7-yl]phenyl}acrylamide | |
| 46 | N,N-dimethyl-N′-{3-[3-(thien-2-ylcarbonyl)pyrazolo[1,5- | + |
| | a]pyrimidin-7- | |
| | yl]phenyl}urea | |
| 51 | N-{3-[3-(thien-2-ylcarbonyl)pyrazolo[1,5-a]pyrimidin-7- | +++ |
| | yl]phenyl}thiophene-2-carboxamide | |
| 54 | 4,7,7-trimethyl-3-oxo-N-{3-[3-(thien-2- | +++ |
| | ylcarbonyl)pyrazolo[1,5-a]pyrimidin-7- | |
| | yl]phenyl}-2-oxabicyclo[2.2.1]heptane-1-carboxamide | |
| 55 | 3,3-dimethyl-N-{3-[3-(thien-2-ylcarbonyl)pyrazolo[1,5- | +++ |
| | a]pyrimidin-7-yl]phenyl}butanamide | |
| 56 | 4-methyl-N-{3-[3-(thien-2-ylcarbonyl)pyrazolo[1,5- | +++ |
| | a]pyrimidin-7-yl]phenyl}pentanamide | |
| 72 | 2-cyclopropyl-N-{3-[3-(thien-2-ylcarbonyl)pyrazolo[1,5- | ++ |
| | a]pyrimidin-7-yl]phenyl}acetamide | |
| 74 | 2-cyclohexyl-N-{3-[3-(thien-2-ylcarbonyl)pyrazolo[1,5- | +++ |
| | a]pyrimidin-7-yl]phenyl}acetamide | |
| 75 | 2-cycloheptyl-N-{3-[3-(thien-2-ylcarbonyl)pyrazolo[1,5- | +++ |
| | a]pyrimidin-7-yl]phenyl}acetamide | |
| 76 | 2-bicyclo[2.2.1]hept-2-yl-N-{3-[3-(thien-2- | +++ |
| | ylcarbonyl)pyrazolo[1,5-a]pyrimidin- | |
| | 7-yl]phenyl}acetamide | |
| 93 | 2-methoxy-N-{3-[3-(thien-2-ylcarbonyl)pyrazolo[1,5- | +++ |
| | a]pyrimidin-7-yl]phenyl}benzamide | |
| 94 | 2-methyl-N-{3-[3-(thien-2-ylcarbonyl)pyrazolo[1,5- | +++ |
| | a]pyrimidin-7-yl]phenyl}benzamide | |
| 95 | 2-chloro-N-{3-[3-(thien-2-ylcarbonyl)pyrazolo[1,5- | +++ |
| | a]pyrimidin-7-yl]phenyl}benzamide | |
| 96 | N-{3-[3-(thien-2-ylcarbonyl)pyrazolo[1,5-a]pyrimidin-7- | +++ |
| | yl]phenyl}-2-(trifluoromethyl)benzamide | |
| 148 | N-{3-[3-(thien-2-ylcarbonyl)pyrazolo[1,5-a]pyrimidin-7- | +++ |
| | yl]phenyl}butanamide | |
| 152 | N-{3-[3-(thien-2-ylcarbonyl)pyrazolo[1,5-a]pyrimidin-7- | +++ |
| | yl]phenyl}pentanamide | |
| 155 | N-{3-[3-(thien-2-ylcarbonyl)pyrazolo[1,5-a]pyrimidin-7- | +++ |
| | yl]phenyl}piperidine-1-carboxamide | |
| 156 | N-butyl-N-methyl-N′-13-[3-(thien-2- | ++ |
| | ylcarbonyl)pyrazolo[1,5-a]pyrimidin-7- | |
| | yl]phenyl}urea | |
| 157 | N-{3-[3-(thien-2-ylcarbonyl)pyrazolo[1,5-a]pyrimidin-7- | ++ |
| | yl]phenyl}morpholine-4-carboxamide | |
| 158 | N-(sec-butyl)-N′-{3-[3-(thien-2-ylcarbonyl)pyrazolo[1,5- | +++ |
| | a]pyrimidin-7-yl]phenyl}urea | |
| 159 | N,N-diethyl-N′-{3-[3-(thien-2-ylcarbonyl)pyrazolo[1,5- | ++ |
| | a]pyrimidin-7-yl]phenyl}urea | |
| 160 | N-(cyclohexylmethyl)-N′-{3-[3-(thien-2- | ++ |
| | ylcarbonyl)pyrazolo[1,5-a]pyrimidin-7- | |
| | yl]phenyl}urea | |
| 162 | N-cyclopentyl-N′-{3-[3-(thien-2- | +++ |
| | ylcarbonyl)pyrazolo[1,5-a]pyrimidin-7- | |
| | yl]phenyl}urea | |
| 163 | N-(tetrahydrofuran-2-ylmethyl)-N′-{3-[3-(thien-2- | ++ |
| | ylcarbonyl)pyrazolo[1,5- | |
| | a]pyrimidin-7-yl]phenyl}urea | |
| 164 | N-neopentyl-N′-{3-[3-(thien-2-ylcarbonyl)pyrazolo[1,5- | +++ |
| | a] pyrimidin-7-yl]phenyl}urea | |
| 165 | N-pentyl-N′-{3-[3-(thien-2-ylcarbonyl)pyrazolo[1,5- | ++ |
| | a]pyrimidin-7-yl]phenyl}urea | |
| 168 | N,N-bis(2-methoxyethyl)-N′-{3-[3-(thien-2- | + |
| | ylcarbonyl)pyrazolo[1,5-a]pyrimidin-7-yl]phenyl}urea | |
| 169 | N-cyclopropyl-N′-{3-[3-(thien-2- | ++ |
| | ylcarbonyl)pyrazolo[1,5-a]pyrimidin-7-yl]phenyl}urea | |
| 170 | N-{3-[3-(thien-2-ylcarbonyl)pyrazolo[1,5-a]pyrimidin-7- | ++ |
| | yl]phenyl}pyrrolidine-1- | |
| | carboxamide | |
| 171 | cyclopentyl 3-[3-(thien-2-ylcarbonyl)pyrazolo[1,5- | +++ |
| | a]pyrimidin-7-yl]phenylcarbamate | |
| 172 | cyclohexyl 3-[3-(thien-2-ylcarbonyl)pyrazolo[1,5- | +++ |
| | a]pyrimidin-7-yl]phenylcarbamate | |
| 173 | cyclohex-2-en-1-yl 3-[3-(thien-2- | +++ |
| | ylcarbonyl)pyrazolo[1,5-a]pyrimidin-7- | |
| | yl]phenylcarbamate | |
| 174 | cyclobutylmethyl 3-[3-(thien-2-ylcarbonyl)pyrazolo[1,5- | +++ |
| | a]pyrimidin-7-yl]phenylcarbamate | |
| 175 | cyclohexylmethyl 3-[3-(thien-2-ylcarbonyl)pyrazolo[1,5- | +++ |
| | a]pyrimidin-7-yl]phenylcarbamate | |
| 176 | tetrahydro-2H-pyran-4-yl 3-[3-(thien-2- | +++ |
| | ylcarbonyl)pyrazolo[1,5-a]pyrimidin-7- | |
| | yl]phenylcarbamate | |
| 177 | 3-methylbut-2-enyl 3-[3-(thien-2- | +++ |
| | ylcarbonyl)pyrazolo[1,5-a]pyrimidin-7- | |
| | yl]phenylcarbamate | |
| 178 | 2-(methylthio)ethyl 3-[3-(thien-2- | +++ |
| | ylcarbonyl)pyrazolo[1,5-a]pyrimidin-7- | |
| | yl]phenylcarbamate | |
| 194 | propyl 3-[5-methyl-3-(thien-2-ylcarbonyl)pyrazolo[1,5- | +++ |
| | a]pyrimidin-7-yl]phenylcarbamate | |
| 195 | butyl 3-[5-methyl-3-(thien-2-ylcarbonyl)pyrazolo[1,5- | +++ |
| | a]pyrimidin-7-yl]phenylcarbamate | |
| 196 | allyl 3-[5-methyl-3-(thien-2-ylcarbonyl)pyrazolo[1,5- | ++ |
| | a]pyrimidin-7-yl]phenylcarbamate | |
| 197 | isopropyl 3-[5-methyl-3-(thien-2- | +++ |
| | ylcarbonyl)pyrazolo[1,5-a]pyrimidin-7- | |
| | yl]phenylcarbamate | |
| 198 | 2-chloroethyl 3-[5-methyl-3-(thien-2- | +++ |
| | ylcarbonyl)pyrazolo[1,5-a]pyrimidin-7- | |
| | yl]phenylcarbamate | |
| 199 | but-3-enyl 3-[5-methyl-3-(thien-2- | +++ |
| | ylcarbonyl)pyrazolo[1,5-a]pyrimidin-7- | |
| | yl]phenylcarbamate | |
| 200 | neopentyl 3-[5-methyl-3-(thien-2- | ++ |
| | ylcarbonyl)pyrazolo[1,5-a]pyrimidin-7- | |
| | yl]phenylcarbamate | |
| 201 | 3-methyl-N-{3-[5-methyl-3-(thien-2- | +++ |
| | ylcarbonyl)pyrazolo[1,5-a]pyrimidin-7- | |
| | yl]phenyl}butanamide | |
| 202 | N-{3-[5-methyl-3-(thien-2-ylcarbonyl)pyrazolo[1,5- | ++ |
| | a]pyrimidin-7-yl]phenyl}benzamide | |
| 204 | N-{3-[5-methyl-3-(thien-2-ylcarbonyl)pyrazolo[1,5- | ++ |
| | a]pyrimidin-7-yl]phenyl}cyclopentanecarboxamide | |
| 205 | N-{3-[5-methyl-3-(thien-2-ylcarbonyl)pyrazolo[1,5- | ++ |
| | a]pyrimidin-7-yl]phenyl}-2-thien-2-ylacetamide | |
| 206 | N-butyl-N′-{3-[5-methyl-3-(thien-2- | ++ |
| | ylcarbonyl)pyrazolo[1,5-a]pyrimidin-7- | |
| | yl]phenyl}urea | |
| 207 | N-isopropyl-N′-{3-[5-methyl-3-(thien-2- | ++ |
| | ylcarbonyl)pyrazolo[1,5-a]pyrimidin-7- | |
| | yl]phenyl}urea | |
| 208 | propyl 3-[6-methyl-3-(thien-2-ylcarbonyl)pyrazolo[1,5- | ++ |
| | a]pyrimidin-7-yl]phenylcarbamate | |
| 211 | isopropyl 3-[6-methyl-3-(thien-2- | + |
| | ylcarbonyl)pyrazolo[1,5-a]pyrimidin-7- | |
| | yl]phenylcarbamate | |
| 214 | neopentyl 3-[6-methyl-3-(thien-2- | + |
| | ylcarbonyl)pyrazolo[1,5-a]pyrimidin-7- | |
| | yl]phenylcarbamate | |
| 217 | N-{3-[6-methyl-3-(thien-2-ylcarbonyl)pyrazolo[1,5- | +++ |
| | a]pyrimidin-7- | |
| | yl]phenyl}cyclopropanecarboxamide | |

+++, <0.1 μM;
++, 0.1-1.0 μM;
+; 1.0-2.0 μM

TABLE 3
Cellular activity in 80s14

| | | IC₅₀ for | |
|---|---|---|---|
| Example | | 80S14 | |
| Number | Chemical Name | (uM) | MP (° C.) |
| 223 | [7-(3-trifluoromethylphenyl)pyrazolo[1,5- | ++ | NA |
| | a]-pyrimidin-3-yl]-2-thienyl-methanone | | |
| 224 | 2-thienyl[7-(2-thienyl)pyrazolo[1,5-a]- | ++ | 193-195 |
| | pyrimidin-3-yl]-methanone | | |
| 225 | [7-(3-fluorophenyl)pyrazolo[1,5-a]- | + | 235-237 |
| | pyrimidin-3-yl]-2-thienyl-methanone | | |
| 226 | 1-[3-[3-(2-furanylcarbonyl)pyrazolo- | + | 159-160 |
| | [1,5-a]pyrimidin-7-yl]phenyl]-2-piperidinone | | |
| 227 | 2-Piperidinone, 1-[3-(3-benzoylpyrazolo[1,5-a]- | + | 160-162 |
| | pyrimidin-7-yl)phenyl]- | | |
| 228 | 1-[3-[3-(2-furanylcarbonyl)pyrazolo- | + | 210-211 |
| | [1,5-a]pyrimidin-7-yl]phenyl]-2-pyrrolidinone | | |
| 229 | 1-[3-(3-benzoylpyrazolo[1,5-a]- | + | 173-174 |
| | pyrimidin-7-yl)phenyl]-2-pyrrolidinone | | |
| 230 | N-[3-(3-benzoylpyrazolo-[1, | ++ | 210-212 |
| | 5-a]pyrimidin-7-yl)phenyl]-cyclopropanecarboxamide | | |
| 231 | [7-[3-(methylamino)phenyl]pyrazolo[1,5-a]- | + | 134-136 |
| | pyrimidin-3-yl]phenyl-methanone | | |
| 232 | N-[3-(3-benzoylpyrazolo- | + | 168-170 |
| | [1,5-a]pyrimidin-7-yl)phenyl]-N-methyl- | | |
| | cyclobutanecarboxamide | | |
| 233 | N-[3-[3-(2-furanylcarbonyl)- | + | 166-167 |
| | pyrazolo[1,5-a]pyrimidin-7-yl]phenyl]-N- | | |
| | methylcyclobutanecarboxamide | | |
| 234 | N-[3-(3-cyanopyrazolo[1,5-a]- | ++ | 157-158 |
| | pyrimidin-7-yl)phenyl]-N-methyl- | | |
| | cyclobutanecarboxamide | | |
| 235 | N-[3-[3-(2-furanylcarbonyl)- | +++ | 227-230 |
| | pyrazolo[1,5-a]pyrimidin.-7-yl]phenyl]- | | |
| | cyclopropanecarboxamide | | |
| 236 | N-[3-(3-benzoylpyrazolo[1,5-a]-pyrimidin-7-yl)phenyl]- | + | 168-170 |
| | N-methyl-cyclopropanecarboxamide | | |
| 237 | N-methyl-N-[3-[3-(2-thienylcarbonyl)- | ++ | 186-187 |
| | pyrazolo[1,5-a]pyrimidin-7-yl]phenyl]-propanamide | | |
| 238 | N-methyl-N-[3-[3-(2-thienylcarbonyl)- | + | 190-192 |
| | pyrazolo[1,5-a]pyrimidin-7-yl]phenyl]-acetamide | | |
| 239 | [3-[3-(2-thienylcarbonyl)pyrazolo-[1, | ++ | 215-217 |
| | 5-a]pyrimidin-7-yl]phenyl]-methyl ester carbamic acid | | |
| 240 | Pyrazolo[1,5-a]pyrimidine-3-carboxylic acid,7-[3- | + | 123-125 |
| | [(cyclobutylcarbonyl)amino]phenyl]-ethyl ester | | |
| 241 | N-[3-[3-(2-furanylcarbonyl)pyrazolo-[1, | ++ | 189-191 |
| | 5-a]pyrimidin-7-yl]phenyl]-2-methyl-propanamide | | |
| 242 | N-[3-(3-benzoylpyrazolo[1,5-a]- | ++ | 160-161 |
| | pyrimidin-7-yl)phenyl]-2,2-dimethyl-propanamide | | |
| 243 | N-[3-[3-(2-furanylcarbonyl)pyrazolo-[1, | ++ | 203-204 |
| | 5-a]pyrimidin-7-yl]phenyl]-2,2-dimethyl-propanamide | | |
| 244 | [7-[3-(1H-imidazol-1-yl)phenyl]pyrazolo- | ++ | 242-244 |
| | [1,5-a]pyrimidin-3-yl]-2-thienyl-methanone | | |
| 245 | [7-[3-(2-methyl-1H-inidazol-1-yl)phenyl]- | + | 172-174 |
| | pyrazolo[1,5-a]pyrimidin-3-yl]-2-thienyl-methanone | | |
| 246 | [7-(2,4-dimethoxyphenyl)pyrazolo[1,5-a]- | + | 146-148 |
| | pyrimidin-3-yl]-2-thienyl-methanone | | |
| 247 | [7-[3-(4-methyl-1H-imidazol-1-yl)phenyl]- | ++ | 232-234 |
| | pyrazolo[1,5-a]pyrimidin-3-yl]-2-thienyl-methanone | | |
| 248 | N,N-diethyl-3-[3-(2-thienylcarbonyl)- | + | 165-167 |
| | pyrazolo[1,5-a]pyrimidin-7-yl]-benzamide | | |
| 249 | N-[3-[3-(2-thienylcarbonyl)-pyrazolo[1,5-a]pyrimidin-7- | +++ | 192-194 |
| | yl] phenyl]cyclopropanecarboxamide | | |
| 250 | N-[3-[3-(2-thienylcarbonyl)pyrazolo[1,5-a] | ++ | 245-247 |
| | pyrimidin-7-yl]phenyl]-acetamide | | |
| 251 | N-[3-[3-(2-thienylcarbonyl)pyrazolo[1,5- | +++ | 208-210 |
| | a]pyrimidin-7-yl]phenyl]-propanamide | | |
| 252 | N-[3-[3-[(5-methyl-2- | ++ | 243-245 |
| | thienyl)carbonyl]pyrazolo[1,5-a]pyrimidin-7-yl]phenyl]- | | |
| | cyclopropanecarboxamide | | |
| 253 | N-[3-(3-benzoylpyrazolo[1,5-a]pyrimidin-7-yl)phenyl]- | + | 153-155 |
| | N-ethyl-cyclopropanecarboxamide | | |
| 254 | N-ethyl-N-(3-pyrazolo[1,5- | + | 127-129 |
| | a]pyrimidin-7-ylphenyl)-cyclopropanecarboxamide | | |
| 255 | N-[3-(3-chloropyrazolo[1,5- | + | 150-151 |
| | a]pyrimidin-7-yl)phenyl]-N-ethyl- | | |
| | cyclopropanecarboxamide | | |
| 256 | N-[3-(3-cyanopyrazolo[1,5- | + | 166-168 |
| | a]pyrimidin-7-yl)phenyl]-N-ethyl- | | |
| | cyclopropanecarboxamide | | |
| 257 | N-ethyl-N-[3-[3-(2-furanylcarbonyl)pyrazolo[1,5- | + | 182-184 |
| | a]pyrimidin-7-yl]phenyl]-cyclopropanecarboxamide | | |
| 258 | Pyrazolo[1,5-a]pyrimidine-3-carboxylic acid, 7-[3- | + | 163-164 |
| | [((cyclopropylcarbonyl)-2-propynylamino]phenyl]- | | |
| | ethyl ester | | |
| 259 | N-2-propynyl-N-[3-[3-(2- | ++ | 221-223 |
| | thienylcarbonyl)pyrazolo[1,5-a]pyrimidin-7-yl]phenyl]- | | |
| | cyclopropanecarboxamide | | |
| 260 | N-[3-[3-(2-thienylcarbonyl)pyrazolo[1,5-a] | ++ | 233-236 |
| | pyrimidin-7-yl]phenyl]-formamide | | |
| 261 | N-methyl-N-[3-[3-(2-thienylcarbonyl) | ++ | 171-173 |
| | pyrazolo [1,5-a]pyrimidin-7-yl]phenyl]-formamide | | |
| 262 | [7-(3-aminophenyl)pyrazolo[1,5-a]pyrimidin- | + | 183-185 |
| | 3-yl]-2-thienyl-methanone | | |
| 263 | [7-(3-ethoxyphenyl)pyrazolo[1,5-a]pyrimidin- | ++ | 146-148 |
| | 3-yl]-2-thienyl-methanone | | |
| 264 | [7-[3-(2-methoxyethoxy)phenyl]pyrazolo[1, | ++ | 112-116 |
| | 5-a]pyrimidin-3-yl]-2-thienyl-methanone | | |
| 265 | [7-[3-(cyclopentyloxy)phenyl]pyrazolo[1, | ++ | 135-138 |
| | 5-a]pyrimidin-3-yl]-2-thienyl-methanone | | |
| 266 | [7-[3-(methylamino)phenyl]pyrazolo[1,5-a] | ++ | 188-190 |
| | pyrimidin-3-yl]-2-thienyl-methanone | | |
| 267 | [7-[3-(propylamino)phenyl]pyrazolo[1,5-a] | ++ | 128-130 |
| | pyrimidin-3-yl]-2-thienyl-methanone | | |
| 268 | N-methyl-N-[3-[3-(4-methyl | + | 189-190 |
| | benzoyl)pyrazolo[1,5-a]pyrimidin-7-yl]phenyl]- | | |
| | cyclopropanecarboxamide | | |
| 269 | 2-furanyl[7-[3-(1H-pyrrol-1-yl)phenyl] | ++ | 204-206 |
| | pyrazolo [1,5-a]pyrimidin-3-yl]-methanone | | |
| 270 | [7-[3-(1H-pyrrol-1-yl)phenyl]pyrazolo[1,5- | ++ | 211-213 |
| | a]pyrimidin-3-yl]-2-thienyl-methanone | | |
| 271 | [7-[4-(ethylamino)phenyl]pyrazolo[1,5-a] | + | 130-132 |
| | pyrimidin-3-yl]-2-thienyl-methanone | | |
| 272 | N-{3-[3-(2-thienylcarbonyl)pyrazolo[1,5-a]pyrimidin-7- | +++ | 192-194 |
| | yl]phenyl}acrylamide | | |
| 273 | 2-methyl-N-{3-[3-(2-thienylcarbonyl)pyrazolo[1,5- | +++ | 180-181 |
| | a]pyrimidin-7-yl]phenyl}propanamide | | |
| 274 | N-{3-[3-(2-thienylcarbonyl)pyrazolo[1,5-a]pyrimidin-7- | +++ | 202-203 |
| | yl]phenyl}-2-butynamide | | |
| 275 | N-{3-[3-(2-thienylcarbonyl)pyrazolo[1,5-a]pyrimidin-7- | +++ | 202-203 |
| | yl]phenyl}cyclobutanecarboxamide | | |
| 276 | N-{3-[5-methyl-3-(2-thienylcarbonyl)pyrazolo[1,5- | +++ | 184-186 |
| | a]pyrimidin-7-yl]phenyl}cyclopropanecarboxamide | | |
| 277 | N-{3-[3-(2-thienylcarbonyl)pyrazolo[1,5-a]pyrimidin-7- | +++ | 70 |
| | yl]phenyl}-2-oxiranecarboxamide | | (decomp.) |
| 278 | N-{3-[5-methyl-3-(2-thienylcarbonyl)pyrazolo[1,5- | +++ | 70 |
| | a]pyrimidin-7-yl]phenyl}-2-oxiranecarboxamide | | (decomp.) |
| 279 | N-{3-[5-methyl-3-(2-thienylcarbonyl)pyrazolo[1,5- | +++ | 121-123 |
| | a]pyrimidin-7-yl]phenyl}cyclobutanecarboxamide | | |
| 280 | N-{3-[3-(2-thienylcarbonyl)pyrazolo[1,5-a]pyrimidin-7- | + | 208-210 |
| | yl]phenyl}-2,5-bis(trifluoromethyl)benzamide | | |
| 281 | N-{3-[3-(2-thienylcarbonyl)pyrazolo[1,5-a]pyrimidin-7- | +++ | 135-140 |
| | yl]phenyl}-3-(trifluoromethyl)benzamide | | |
| 282 | N-{3-[3-(2-thienylcarbonyl)pyrazolo[1,5-a]pyrimidin-7- | +++ | 239-240 |
| | yl]phenyl}cyclohexanecarboxamide | | |
| 283 | N-{3-[3-(2-thienylcarbonyl)pyrazolo[1,5-a]pyrimidin-7- | +++ | 209-210 |
| | yl]phenyl}cyclopentanecarboxamide | | |
| 284 | N-{3-[3-(2-thienylcarbonyl)pyrazolo[1,5-a]pyrimidin-7- | +++ | 211-213 |
| | yl]phenyl}benzamide | | |
| 285 | N-{3-[3-(2-thienylcarbonyl)pyrazolo[1,5-a]pyrimidin-7- | +++ | >240 |
| | Yl]phenyl}-2-furamide | | |
| 286 | 3-bromo-N-{3-[3-(2-thienylcarbonyl)pyrazolo[1,5- | +++ | >300 |
| | a]pyrimidin-7-yl]phenyl}benzamide | | |
| 287 | 4-(tert-butyl)-N-{3-[3-(2-thienylcarbonyl)pyrazolo[1,5- | + | Foam, |
| | a]pyrimidin-7-yl]phenyl}benzamide | | M + H, 491 |
| 288 | 3,5-dinitro-N-{3-[3-(2-thienylcarbonyl)pyrazolo[1,5- | + | dec >145 |
| | a]pyrimidin-7-yl]phenyl}benzamide | | |
| 289 | 2,4-dichloro-N-{3-[3-(2-thienylcarbonyl)pyrazolo[1,5- | ++ | 132-134 |
| | a]pyrimidin-7-yl]phenyl}benzamide | | |
| 290 | N-{3-[3-(2-thienylcarbonyl)pyrazolo[1,5-a]pyrimidin-7- | +++ | 191-194 |
| | Yl]phenyl}nicotinamide | | |
| 291 | N-{3-[3-(2-thienylcarbonyl)pyrazolo[1,5-a]pyrimidin-7- | +++ | 220-222 |
| | yl]phenyl}isonicotinamide | | |
| 292 | N,N′-dibutyl-N-{3-[3-(2-thienylcarbonyl)pyrazolo[1,5- | 4.5 | 180-181 |
| | a]pyrimidin-7-yl]phenyl)dicarbonimidic diamide | | |
| 293 | isopropyl 3-[3-(3-thienylcarbonyl)pyrazolo[1,5- | +++ | 122-124 |
| | a]pyrimidin-7-yl]phenylcarbamate | | |
| 294 | isopropyl 3-(3-benzoylpyrazolo[1,5-aJpyrimidin-7- | +++ | 116-118 |
| | yl)phenylcarbamate | | |
| 295 | isopropyl 3-[3-(cyclopentylcarbonyl)pyrazolo[1,5- | ++ | 57-59 |
| | a]pyrimidin-7-yl)phenylcarbamate | | |
| 296 | N-{3-[3-(cyclopentylcarbonyl)pyrazolo[1,5-a]pyrimidin- | ++ | 69-71 |
| | 7-yl]phenyl}-3-methylbutanamide | | |
| 297 | 3-methyl-N-{3-[3-(3-thienylcarbonyl)pyrazolo[1,5- | +++ | 137-139 |
| | a]pyrimidin-7-yl]phenyl}butanamide | | |
| 298 | N-{3-[3-(2-furoyl)pyrazolo[1,5-a]pyrimidin-7- | +++ | 143 (dec) |
| | yl]phenyl}-N′-isopropylurea | | |
| 299 | isopropyl 3-[3-(2-furoyl)pyrazolo[1,5-a]pyrimidin-7- | +++ | 140-143 |
| | yl]phenylcarbamate | | |
| 300 | N-{3-[3-(2-furoyl)pyrazolo[1,5-a]pyrimidin-7- | ++ | 220-222 |
| | yl]phenyl}benzamide | | |
| 401 | N-{3-[3-(2-furoyl)pyrazolo[1,5-a]pyrimidin-7- | +++ | 134 (dec) |
| | yl]phenyl}-3-methylbutanamide | | |
| 302 | N-{3-[3-(2-furoyl)pyrazolo[1,5-a]pyrimidin-7- | +++ | 143 (dec.) |
| | yl]phenyl}cyclopentanecarboxamide | | |
| 303 | 2-cyclopentyl-N-{3-[3-(2-furoyl)pyrazolo[1.5- | +++ | 142 (dec.) |
| | a]pyrimidin-7-yl]phenyl}acetamide | | |
| 304 | N-{3-[3-(3-furoyl)pyrazolo[1,5-a]pyrimidin-7- | ++ | 144-146 |
| | yl]phenyl}-3-methylbutanamide | | |
| 305 | isopropyl 3-[3-(3-furoyl)pyrazolo[1,5-a]pyrimidin-7- | ++ | 127-129 |
| | yl]phenylcarbamate | | |
| 306 | methyl 3-(3-(2-thienylcarbonyl)pyrazolo[1,5- | + | 180-184 |
| | a]pyrimidin-7-yl]benzoate | | |
| 307 | 3-methyl-N-{3-[2-methyl-3-(2- | + | 137-139 |
| | thienylcarbonyl)pyrazolo[1,5-a]pyrimidin-7- | | |
| | yl]phenyl}butanamide | | |
| 308 | isopropyl 3-[2-methyl-3-(2- | + | 186-188 |
| | thienylcarbonyl)pyrazolo[1,5- | | |
| | a]pyrimidin-7-yl]phenylcarbamate | | |
| 309 | N-{2-methyl-5-[3-(2-thienylcarbonyl)pyrazolo[1,5- | ++ | 216-218 |
| | a]pyrimidin-7-yl]phenyl}benzamide | | |
| 310 | 3-methyl-N-{2-methyl-5-[3-(2- | ++ | 185-187 |
| | thienylcarbonyl)pyrazolo[1,5-a]pyrimidin-7- | | |
| | yl]phenyl}butanamide | | |
| 311 | N-isopropyl-N′-{2-methyl-5-[3-(2- | +++ | 225-227 |
| | thienylcarbonyl)pyrazolo[1,5-a]pyrimidin-7- | | |
| | yl]phenyl}urea | | |
| 312 | N-{2-chloro-5-[3-(2-thienylcarbonyl)pyrazolo[1,5- | +++ | 217-218 |
| | a]pyrimidin-7-yl]phenyl}benzamide | | |
| 313 | N-{2-chloro-5-[3-(2-thienylcarbonyl)pyrazolo [1,5- | +++ | 172-175 |
| | a]pyrimidin-7-yl]phenyl}-3-methylbutanamide | | |
| 314 | isopropyl 2-methyl-5-[3-(2- | +++ | 198-200 |
| | thienylcarbonyl)pyrazolo[1,5- | | |
| | a]pyrimidin-7-yl]phenylcarbamate | | |
| 315 | N-{2-methoxy-5-[3-(2-thienylcarbonyl)pyrazolo[1,5- | ++ | 201-203 |
| | a]pyrimidin-7-yl]phenyl}benzamide | | |
| 316 | isopropyl 2-methoxy-5-[3-(2- | ++ | 193-195 |
| | thienylcarbonyl)pyrazolo[1,5-a]pyrimidin-7- | | |
| | yl]phenylcarbamate | | |
| 317 | N-{2-methoxy-5-[3-(2-thienylcarbonyl)pyrazolo[1,5- | ++ | 192-194 |
| | a]pyrimidin-7-yl]phenyl}-3-methylbutanamide | | |
| 318 | N-isobutyl-3-[3-(2-thienylcarbonyl)pyrazolo[1,5- | +++ | 174-177 |
| | a]pyrimidin-7-yl]benzamide | | |
| 319 | N-butyl-3-{3-(2-thienylcarbonyl)pyrazolo[1,5- | +++ | 172-174 |
| | a]pyrimidin-7-yl]benzamide | | |
| 320 | N-cyclopentyl-3-[3-(2-thienylcarbonyl)pyrazolo[1,5- | +++ | 207-209 |
| | a]pyrimidin-7-yl]benzamide | | |
| 321 | N-phenyl-3-[3-(2-thienylcarbonyl)pyrazolo[1,5- | +++ | 211-212 |
| | a]pyrimidin-7-yl]benzamide | | |
| 322 | N-isopropyl-3-[3-(2-thienylcarbonyl)pyrazolo[1,5- | +++ | 218-220 |
| | a]pyrimidin-7-yl]benzamide | | |
| 323 | N-isopropyl-N′-{2-methoxy-5-[3-(2- | ++ | 202-204 |
| | thienylcarbonyl)pyrazolo[1,5-a]pyrimidin-7- | | |
| | yl]phenyl}urea | | |
| 324 | 3-methyl-N-{5-[3-(2-thienylcarbonyl)pyrazolo[1,5- | ++ | 217-219 |
| | a]pyrimidin-7-yl]-3-pyridinyl}butanamide | | |
| 325 | 7-(3-nitrophenyl)-3-(3-thienyl)pyrazolo[1,5- | + | 148-150 |
| | a]pyrimidine | | |
| 326 | 3-(3-bromopyrazolo[1,5-a]pyrimidin-7-yl)aniline | + | 120-121 |
| 327 | isobutyl 3-(3-bromopyrazolo[1,5-a]pyrimidin-7- | + | 90-92 |
| | yl)phenylcarbamate | | |
| 328 | [7-(5-nitro-3-thienyl)pyrazolo[1,5-a]pyrimidin-3-yl](2- | ++ | 225-226 |
| | thienyl)methanone | | |
| 329 | N-isopropyl-N′-{5-[3-(thien-2-ylcarbonyl)pyrazolo[1,5- | ++ | 162-165 |
| | a]pyrimidin-7-yl]thien-2-yl}urea | | |
| 330 | N-[3-(3-bromopyrazolo[1,5-a]pyrimidin-7-yl)phenyl]-3- | + | 132-134 |
| | methylbutanamide | | |
| 331 | [7-(5-aminothien-3-yl)pyrazolo[1,5-a]pyrimidin-3- | ++ | 174-176 |
| | yl](thien-2-yl)methanone | | |
| 332 | O-ethyl 3-[3-(thien-2-ylcarbonyl)pyrazolo[1,5- | +++ | 209-211 |
| | a]pyrimidin-7-yl]phenylthiocarbamate | | |
| 333 | 3-methyl-N-[3-(3-thien-2-ylpyrazolo[1,5-a]pyrimidin-7- | + | 148-150 |
| | yl)phenyl]butanamide | | |
| 334 | N-phenyl-N′-{3-[3-(thien-2-ylcarbonyl)pyrazolo[1,5- | +++ | 124-126 |
| | a]pyrimidin-7-yl]phenyl)thiourea | | |
| 335 | isopropyl 4-chloro-3-[3-(thien-2- | +++ | 234-236 |
| | ylcarbonyl)pyrazolo[1,5-a.]pyrimidin-7- | | |
| | yl]phenylcarbamate | | |
| 336 | [7-(2-chloro-5-nitrophenyl)pyrazolo[1,5-a]pyrimidin-3- | + | 162-166 |
| | yl](thien-2-yl)methanone | | |
| 337 | N-{3-[3-(pyridin-2-ylcarbonyl)pyrazolo[1,5- | + | 214-215 |
| | a]pyrimidin- | | |
| | 7-yl]phenyl}cyclopropanecarboxamide | | |
| 338 | N-{3-[3-(1,3-thiazol-2-ylcarbonyl)pyrazolo[1,5- | ++ | 226-229 |
| | a]pyrimidin-7-yl]phenyl}cyclopropanecarboxamide | | |
| 339 | N-{4-chloro-3-[3-(thien-2-ylcarbonyl)pyrazolo[1,5- | ++ | 162-165 |
| | a]pyrimidin-7-yl]phenyl}-3-methylbutanamide | | |
| 340 | N-{4-chloro-3-[3-(thien-2-ylcarbonyl)pyrazolo[1,5- | ++ | 194-195 |
| | a]pyrimidin-7-yl]phenyl}-N′-isopropylthiourea | | |
| 341 | N-{4-chloro-3-[3-(thien-2-ylcarbonyl)pyrazolo[1,5- | ++ | 194-195 |
| | a]pyrimidin-7-yl]phenyl}benzamide | | |
| 342 | N-{2-fluoro-5-[3-(thien-2-ylcarbonyl)pyrazolo[1,5- | +++ | 192-194 |
| | a]pyrimidin-7-yl]phenyl}-3-methylbutanamide | | |
| 343 | 3-methyl-N-{5-[3-(thien-2-ylcarbonyl)pyrazolo[1,5- | ++ | 268-270 |
| | a]pyrimidin-7-yl]thien-2-yl}butanamide | | |
| 344 | isopropyl 5-[3-(thien-2-ylcarbonyl)pyrazolo[1,5- | ++ | 210-215 |
| | a]pyrimidin-7-yl]thien-2-ylcarbamate | | |
| 345 | [7-(5-aminothien-2-yl)pyrazolo[1,5-a]pyrimidin-3- | + | 232-235 |
| | yl] (thien-2-yl)methanone | | (dec.) |
| 346 | 4-chloro-N-{3-[3-(thien-2-ylcarbonyl)pyrazolo[1,5- | ++ | 97-100 |
| | a]pyrimidin-7-yl]phenyl}butanamide | | |
| 347 | isopropyl 2-fluoro-5-[3-(thien-2- | +++ | >200 |
| | ylcarbonyl)pyrazolo[1,5- | | |
| | a]pyrimidin-7-yl]phenylcarbamate | | |
| 348 | isopropyl 2-(dimethylamino)-5-[3-(thien-2- | ++ | 164-165 |
| | ylcarbonyl)pyrazolo[1,5-a]pyrimidin-7- | | |
| | yl]phenylcarbamate | | |
| 349 | {7-[3-(benzylamino)phenyl]pyrazolo[1,5-a]pyrimidin-3- | ++ | 90-92 |
| | yl}(thien-2-yl)methanone | | |
| 350 | {7-[3-(isopentylamino)phenyl]pyrazolo[1,5-a]pyrimidin- | +++ | 75-77 |
| | 3-yl}(thien-2-yl)methanone | | |
| 351 | 3-methyl-N-{4-[3-(thien-2-ylcarbonyl)pyrazolo[1,5- | ++ | 234-235 |
| | a]pyrimidin-7-yl]thien-2-yl}butanamide | | |
| 352 | [7-(3-amino-4-fluorophenyl)pyrazolo[1,5-a]pyrimidin-3- | ++ | 241-242 |
| | yl](thien-2-yl)methanone | | |
| 353 | N-cyclohexyl-N′-{3-[3-(thien-2- | +++ | 233-236 |
| | ylcarbonyl)pyrazolo[1,5- | | |
| | a]pyrimidin-7-yl]phenyl}urea | | |
| 354 | 4,4,4-trichloro-N-{3-[3-(thien-2- | +++ | 189-191 |
| | ylcarbonyl)pyrazolo[1,5- | | |
| | a]pyrimidin-7-yl]phenyl}butanamide | | |
| 355 | N-{2-fluoro-5-[3-(thien-2-ylcarbonyl)pyrazolo[1,5- | +++ | 180-181 |
| | a]pyrimidin-7-yl]phenyl}-3,3-dimethylbutanamide | | |
| 356 | 4,4,4-trifluoro-N-{2-fluoro-5-[3-(thien-2- | +++ | 185-186 |
| | ylcarbonyl)pyrazolo[1,5-a]pyrimidin-7- | | |
| | yl]phenyl}-3-methylbutanamide | | |
| 357 | bis(2,2,2-trichloroethyl)2-fluoro-5-[3-(thien-2- | +++ | 94-97 |
| | ylcarbonyl)pyrazolo[1,5- | | |
| | a]pyrimidin-7-yl]phenylimidodicarbonate | | |
| 358 | 2-fluoro-N-{3-[3-(thien-2-ylcarbonyl)pyrazolo[1,5- | +++ | 190-191 |
| | a]pyrimidin-7- | | |
| | yl]phenyl}benzamide | | |
| 359 | 2,6-difluoro-N-{3-[3-(thien-2-ylcarbonyl)pyrazolo[1,5- | +++ | 159-160 |
| | a]pyrimidin-7-yl]phenyl}benzamide | | |
| 360 | N-{3-[3-(thien-2-ylcarbonyl)pyrazolo[1,5-a]pyrimidin- | +++ | 198-200 |
| | 7-yl]phenyl}pyridine-2-carboxamide | | |
| 361 | N-{5-[3-(thien-2-ylcarbonyl)pyrazolo[1,5-a]pyrimidin- | + | 162-165 |
| | 7-yl]thien-2-yl}benzamide | | |
| 362 | (7-{3-[(2-phenylethyl)amino]phenyl}pyrazolo[1,5- | + | 118-120 |
| | a]pyrimidin-3-yl)(thien-2-yl)methanone | | |
| 363 | (7-{3-[(2-methoxybenzyl)amino]phenyl}pyrazolo[1,5- | + | 138-140 |
| | a]pyrimidin-3-yl)(thien-2-yl)methanone | | |
| 364 | N-butyl-N′-{3-[3-(thien-2-ylcarbonyl)pyrazolo[1,5- | +++ | 155-157 |
| | a]pyrimidin-7-yl]phenyl}thiourea | | |
| 365 | (7-{3-[(3-chlorobenzyl)amino]phenyl}pyrazolo[1,5- | + | 133-135 |
| | a]pyrimidin-3-yl)(thien-2-yl)methanone | | |
| 366 | (7-{3-[(4-chlorobenzyl)amino]phenyl}pyrazolo[1,5- | + | 149-152 |
| | a]pyrimidin-3-yl)(thien-2-yl)methanone | | |
| 367 | (7-{3-[(2-furylmethyl)amino]phenyl}pyrazolo[1,5- | ++ | M + H 401 |
| | a]pyrimidin-3-yl)(thien-2-yl)methanone | | |
| 368 | (7-{3-[(2-chlorobenzyl)amino]phenyl}pyrazolo[1,5- | ++ | M + H 445 |
| | a]pyrimidin-3-yl)(thien-2-yl)methanone | | |
| 369 | (7-{3-[(3-furylmethyl)amino]phenyl}pyrazolo[1,5- | ++ | 140-144 |
| | a]pyrimidin-3-yl)(thien-2-yl)methanone | | |
| 370 | (7-{3-[(2E)-but-2-enylamino]phenyl}pyrazolo[1,5- | +++ | 110-113 |
| | a]pyrimidin-3-yl)(thien-2-yl)methanone | | |
| 371 | (7-{3-[(3-methylbut-2-enyl)amino]phenyl}pyrazolo[1,5- | +++ | 116-118 |
| | a]pyrimidin-3-yl)(thien-2-yl)methanone | | |
| 372 | (7-{3-[(3,3-dimethylbutyl)amino]phenyl}pyrazolo[1,5- | +++ | 156-158 |
| | a]pyrimidin-3-yl)(thien-2-yl)methanone | | |
| 373 | [7-(6-chloropyridin-3-yl)pyrazolo[1,5-a]pyrimidin-3- | + | 145-148 |
| | yl] (thien-2-yl)methanone | | |
| 374 | 2,6-difluoro-N-{2-fluoro-5-[3-(thien-2- | +++ | 198-200 |
| | ylcarbonyl)pyrazolo[1,5-a]pyrimidin-7- | | |
| | yl]phenyl}benzamide | | |
| 375 | 2-fluoro-N-{2-fluoro-5-[3-(thien-2- | +++ | 214-216 |
| | ylcarbonyl)pyrazolo[1,5-a]pyrimidin-7- | | |
| | yl]phenyl}benzamide | | |
| 376 | 2-chloro-N-{2-fluoro-5-[3-(thien-2- | +++ | 166-168 |
| | ylcarbonyl)pyrazolo[1,5-a]pyrimidin-7- | | |
| | yl]phenyl}benzamide | | |
| 377 | (7-{3-[(2-methylbenzyl)amino]phenyl}pyrazolo[1,5- | ++ | 177-179 |
| | a]pyrimidin-3-yl)(thien-2-yl)methanone | | |
| 378 | (7-{3-[(2-phenylpropyl)amino]phenyl}pyrazolo[1,5- | ++ | 108-110 |
| | a]pyrimidin-3-yl)(thien-2-yl)methanone | | |
| 379 | (7-{3-[(cyclohexylmethyl)amino]phenyl}pyrazolo[1,5- | +++ | 101-104 |
| | a]pyrimidin-3-yl)(thien-2- | | |
| | yl)methanone | | |
| 380 | {7-[3-(butylamino)phenyl]pyrazolo[1,5-a]pyrimidin-3- | +++ | 87-91 |
| | yl}(thien-2-yl)methanone | | |
| 381 | 2-chloroethyl 2-fluoro-5-[3-(thien-2- | +++ | 194-196 |
| | ylcarbonyl)pyrazolo[1,5-a]pyrimidin-7- | | |
| | yl]phenylcarbamate | | |
| 382 | N-{2-fluoro-5-[3-(thien-2-ylcarbonyl)pyrazolo[1,5- | +++ | >200 |
| | a]pyrimidin-7-yl]phenyl}-N′-isopropylurea | | |
| 383 | 4-(1H-pyrrol-1-yl)-N-{3-[3-(thien-2- | ++ | 224-226 |
| | ylcarbonyl)pyrazolo[1,5-a]pyrimidin-7- | | |
| | yl]phenyl}benzamide | | |
| 384 | 2,6-dichloro-N-{3-[3-(thien-2-ylcarbonyl)pyrazolo[1,5- | ++ | 245-246 |
| | a]pyrimidin-7-yl]phenyl}benzamide | | |
| 385 | N-{3-[3-(thien-2-ylcarbonyl)pyrazolo[1,5-a]pyrimidin- | +++ | 222-224 |
| | 7-yl]phenyl}pyrazine-2-carboxamide | | |
| 386 | phenyl N′-cyano-N-{3-[3-(thien-2- | ++ | 220 (dec) |
| | ylcarbonyl)pyrazolo[1,5-a]pyrimidin-7- | | |
| | yl]phenyl}imidocarbamate | | |
| 387 | 1-methyl-N-{3-[3-(thien-2-ylcarbonyl)pyrazolo[1,5- | +++ | 120(dec) |
| | a]pyrimidin-7-yl]phenyl}-1H-pyrrole-2-carboxamide | | |
| 388 | N-{2-fluoro-5-[3-(thien-2-ylcarbonyl)pyrazolo[1,5- | +++ | 175-179 |
| | a]pyrimidin-7-yl]phenyl}isonicotinamide | | |
| 389 | N-isopropyl-N′-{3-[3-(thien-2-ylcarbonyl)pyrazolo[1,5- | +++ | 208-210 |
| | a]pyrimidin-7-yl]phenyl}thiourea | | |
| 390 | (7-chloropyrazolo[1,5-a]pyrimidin-3-yl)(thien-2- | ++ | 148-150 |
| | yl)methanone | | |
| 391 | 2,6-dichloro-N-(2,6-dichlorobenzoyl)-N-{3-[3-(thien-2- | + | 238-240 |
| | ylcarbonyl)pyrazolo[1,5-a]pyrimidin-7- | | |
| | yl]phenyl}benzamide | | |
| 392 | tert-butyl 4-[2-oxo-2-({3-[3-(thien-2- | +++ | 173-175 |
| | ylcarbonyl)pyrazolo[1,5-a]pyrimidin-7- | | |
| | yl]phenyl}amino)ethyl]piperidine-1-carboxylate | | |
| 393 | 2-piperidin-4-yl-N-{3-[3-(thien-2- | ++ | 216-220 |
| | ylcarbonyl)pyrazolo[1,5-a]pyrimidin-7- | | |
| | yl]phenyl}acetamide | | |
| 394 | {7-[3-(diethylamino)phenyl]pyrazolo[1,5-a]pyrimidin-3- | ++ | 102-104 |
| | yl}(thien-2-yl)methanone | | |
| 395 | [7-(3-{[(2E)-3-(2-methoxyphenyl)prop-2- | + | 128-131 |
| | enyl]amino}phenyl)pyrazolo[1,5- | | |
| | a]pyrimidin-3-yl](thien-2-yl)methanone | | |
| 396 | [7-(3-{[(2E)-3-(4-methoxyphenyl)prop-2- | + | 176-179 |
| | enyl]amino}phenyl)pyrazolo[1,5- | | |
| | a]pyrimidin-3-yl](thien-2-yl)methanone | | |
| 397 | [7-(3-{[(2E)-2-methyl-3-phenylprop-2- | + | 74-76 |
| | enyl]amino}phenyl)pyrazolo[1,5-a]pyrimidin- | | |
| | 3-yl](thien-2-yl)methanone | | |
| 398 | [7-(3-{[(2E)-3-(4-nitrophenyl)prop-2- | + | 163-166 |
| | enyl]amino}phenyl)pyrazolo[1,5-a]pyrimidin- | | |
| | 3-yl](thien-2-yl)methanone | | |
| 399 | (7-{3-[(3,3-diphenylprop-2- | + | 156-158 |
| | enyl)amino]phenyl pyrazolo[1,5-a]pyrimidin-3- | | |
| | yl)(thien- | | |
| | 2-yl)methanone | | |
| 400 | (7-{3-[(3-phenylbutyl)amino]phenyl}pyrazolo[1,5- | ++ | 64-68 |
| | a]pyrimidin-3-yl)(thien-2-yl)methanone | | |
| 401 | [7-(3-{[(2E)-3-(4-hydroxy-3-methoxyphenyl)prop-2- | + | 180-184 |
| | enyl]amino}phenyl)pyrazolo[1,5- | | |
| | a]pyrimidin-3-yl](thien-2-yl)methanone | | |
| 402 | (7-{3-[(2-methylbutyl)amino]phenyl}pyrazolo[1,5- | ++ | 210-212 |
| | a]pyrimidin-3-yl)(thien-2-yl)methanone | | |
| 403 | (7-{3-[(2-ethylbutyl)amino]phenyl}pyrazolo[1,5- | ++ | 192-194 |
| | a]pyrimidin-3-yl)(thien-2-yl)methanone | | |
| 404 | {7-[3-(isobutylamino)phenyl]pyrazolo[1,5-a]pyrimidin- | ++ | 186-188 |
| | 3-yl}(thien-2-yl)methanone | | |
| 405 | tert-butyl 4-[({3-[3-(thien-2-ylcarbonyl)pyrazolo[1,5- | ++ | 148-150 |
| | a]pyrimidin-7- | | |
| | yl]phenyl}amino)carbonyl]piperidine-1-carboxylate | | |
| 406 | N,N′-bis{3-[3-(thien-2-ylcarbonyl)pyrazolo[1,5- | + | 151-153 |
| | a]pyrimidin-7-yl]phenyl}thiourea | | |
| 407 | isobutyl 3-(3-thien-2-ylpyrazolo[1,5-a]pyrimidin-7- | ++ | 140-141 |
| | yl)phenylcarbamate | | |
| 408 | N-{3-[3-(thien-2-ylcarbonyl)pyrazolo[1,5-a]pyrimidin- | ++ | 159-161 |
| | 7-yl]phenyl}piperidine-4-carboxamide | | |
| 409 | [7-(3-{[(2E)-2-hexyl-3-phenylprop-2- | + | 110-115 |
| | enyl]amino}phenyl)pyrazolo[1,5-a]pyrimidin- | | |
| | 3-yl](thien-2-yl)methanone | | |
| 410 | [7-(3-{[(2E)-2-pentyl-3-phenylprop-2- | + | 110-112 |
| | enyl]amino}phenyl)pyrazolo[1,5-a]pyrimidin- | | |
| | 3-yl](thien-2-yl)methanone | | |
| 411 | N-isopropyl-N′-{3-[3-(thien-2-ylcarbonyl)pyrazolo[1,5- | + | >200 |
| | A]pyrimidin-7-yl]phenyl}guanidine | | |
| 412 | [7-(3-{[(2E)-3-phenylprop-2- | + | 123-125 |
| | enyl]amino}phenyl)pyrazolo[1,5-a]pyrimidin-3- | | |
| | yl](thien-2-yl)methanone | | |
| 413 | [7-(3-{[(2E)-3-(4-tert-butylphenyl)-2-methylprop-2- | + | 135-137 |
| | enyl]amino}phenyl)pyrazolo[1,5-a]pyrimidin-3- | | |
| | yl](thien-2-yl)methanone | | |
| 414 | N″-cyano-N-isopropyl-N′-{3-[3-(thien-2- | +++ | 240-241 |
| | ylcarbonyl)pyrazolo[1,5-a]pyrimidin-7- | | |
| | yl]phenyl}guanidine | | |
| 415 | N-{3-[3-(thien-2-ylcarbonyl)pyrazolo[1,5-a]pyrimidin- | +++ | 280-282 |
| | 7-yl]phenyl}-1H-imidazole-5-carboxamide | | |
| 416 | pyridin-4-ylmethyl 3-[3-(thien-2- | +++ | 185(dec) |
| | ylcarbonyl)pyrazolo[1,5-a]pyrimidin-7- | | |
| | yl]phenylcarbamate | | |
| 417 | 1-methyl-N-{3-[3-(thien-2-ylcarbonyl)pyrazolo[1,5- | + | 236 |
| | a]pyrimidin-7-yl]phenyl}piperidine-4-carboxamide | | |
| 418 | (7-{3-[(2-methylpentyl)amino]phenyl}pyrazolo[1,5- | ++ | 133-135 |
| | a]pyrimidin-3-yl)(thien-2-yl)methanone | | |
| 419 | (7-{3-[(2-ethylhexyl)amino]phenyl}pyrazolo[1,5- | ++ | 134-138 |
| | a]pyrimidin-3-yl)(thien-2-yl)methanone | | |
| 420 | 3-(1,1-dioxidothiomorpholin-4-yl)propyl 3-[3-(thien-2- | +++ | 148-150 |
| | ylcarbonyl)pyrazolo[1,5- | | |
| | a]pyrimidin-7-yl]phenylcarbamate | | |
| 421 | 2-chloro-N-{2-(dimethylamino)-5-[3-(thien-2- | +++ | 220-222 |
| | ylcarbonyl)pyrazolo[1,5-a] pyrimidin- | | |
| | 7-yl]phenyl}benzamide | | |
| 422 | {7-[3-(ethylamino)phenyl]pyrazolo[1,5-a]pyrimidin-3- | ++ | 154-155 |
| | yl}(thien-2-yl)methanone | | |
| 423 | 4-morpholin-4-yl-N-{3-[3-(thien-2- | ++ | 127-133 |
| | ylcarbonyl)pyrazolo[1,5-a]pyrimidin-7- | | |
| | yl]phenyl}butanamide | | |
| 424 | pyridin-3-ylmethyl 3-[3-(thien-2- | +++ | >200 |
| | ylcarbonyl)pyrazolo[1,5-a]pyrimidin-7- | | |
| | yl]phenylcarbamate | | |
| 425 | 3-morpholin-4-ylpropyl 3-[3-(thien-2- | ++ | 198-199 |
| | ylcarbonyl)pyrazolo[1,5-a]pyrimidin-7- | | |
| | yl]phenylcarbamate | | |
| 426 | [7-(3-{[(2E)-2-methylpent-2- | ++ | 132-133 |
| | enyl]amino}phenyl)pyrazolo[1,5-a]pyrimidin-3-yl] | | |
| | (thien-2-yl)methanone | | |
| 427 | 2-pyridin-4-yl-N-{3-[3-(thien-2- | +++ | 130-131 |
| | ylcarbonyl)pyrazolo[1,5- | | |
| | a]pyrimidin-7-yl]phenyl}acetamide | | |
| 428 | N-{2-(dimethylamino)-5-[3-(thien-2- | ++ | 151-153 |
| | ylcarbonyl)pyrazolo[1,5-a]pyrimidin-7- | | |
| | yl]phenyl}isonicotinamide | | |
| 429 | pyridin-2-ylmethyl 3-[3-(thien-2- | ++ | 121-125 |
| | ylcarbonyl)pyrazolo[1,5-a]pyrimidin-7- | | |
| | yl]phenylcarbamate | | |
| 430 | (7-{3-[(2,3-dimethylpentyl)amino]phenyl}pyrazolo[1,5- | ++ | 84-86 |
| | a]pyrimidin-3-yl)(thien-2- | | |
| | yl)methanone | | |
| 431 | [7-(3-{[(2E)-2-methylbut-2- | ++ | 118-120 |
| | enyl]amino}phenyl)pyrazolo [1,5-a]pyrimidin-3- | | |
| | yl](thien-2-yl)methanone | | |
| 432 | (7-{3-[(2E)-pent-2-enylamino]phenyl}pyrazolo[1,5- | +++ | 72-75 |
| | a]pyrimidin-3-yl)(thien-2-yl)methanone | | |
| 433 | thien-2-yl(7-{3-[(3,5,5- | ++ | 81-85 |
| | trimethylhexyl)amino]phenyl}pyrazolo[1,5-a]pyrimidin- | | |
| | 3-yl)methanone | | |
| 434 | (7-{3-[(2-methylprop-2-enyl) | ++ | 104-106 |
| | amino]phenyl}pyrazolo[1,5-a]pyrimidin-3-yl)(thien- | | |
| | 2-yl)methanone | | |
| 435 | (7-{3-[(2-methylprop-2-enyl) | ++ | 79-82 |
| | amino]phenyl}pyrazolo[1,5-a]pyrimidin-3-yl)(thien- | | |
| | 2-yl)methanone | | |
| 436 | (7-{3-[(2-ethylprop-2-enyl)amino]phenyl}pyrazolo[1,5- | +++ | 78-81 |
| | a]pyrimidin-3-yl)(thien-2-yl)methanone | | |
| 437 | 1-{3-[3-(thien-2-ylcarbonyl)pyrazolo[1,5-a]pyrimidin-7- | +++ | >200 |
| | yl]phenyl}pyrrolidin-2-one | | |
| 438 | 2-chloro-N-(2-chlorobenzoyl)-N-{2-fluoro-5-[3-(thien- | +++ | 150-151 |
| | 2-ylcarbonyl)pyrazolo[1,5-a]pyrimidin-7- | | |
| | yl]phenyl}benzamide | | |
| 439 | 2-pyridin-3-yl-N-{3-[3-(thien-2- | ++ | >200 |
| | ylcarbonyl)pyrazolo[1,5- | | |
| | a]pyrimidin-7-yl]phenyl}acetamide | | |
| 440 | 3-pyridin-4-yl-N-{3-[3-(thien-2- | ++ | 176-181 |
| | ylcarbonyl)pyrazolo[1,5- | | |
| | a]pyrimidin-7-yl]phenyl}propanamide | | |
| 441 | N-{3-[3-(thien-2-ylcarbonyl)pyrazolo[1,5-a]pyrimidin- | + | 165-170 |
| | 7-yl]phenyl}-1H-pyrazole-4-carboxamide | | |
| 442 | 4-oxo-4-({3-[3-(thien-2-ylcarbonyl)pyrazolo[1,5- | + | 240-241 |
| | a]pyrimidin-7-yl]phenyl}amino)butanoic acid | | |
| 443 | 2-[({3-[3-(thien-2-ylcarbonyl)pyrazolo[1,5-a]pyrimidin- | + | 215-216 |
| | 7-yl]phenyl}amino)carbonyl]benzoic acid | | |
| 444 | 5-oxo-5-({3-[3-(thien-2-ylcarbonyl)pyrazolo[1,5- | + | 189-190 |
| | a]pyrimidin-7-yl]phenyl}amino)pentanoic acid | | |
| 445 | 3-methyl-5-oxo-5-({3-[3-(thien-2- | + | 135-136 |
| | ylcarbonyl)pyrazolo[1,5-a]pyrimidin-7- | | |
| | yl]phenyl}amino)pentanoic acid | | |
| 446 | 4,5-dichloro-2-[({3-[3-(thien-2-ylcarbonyl)pyrazolo[1,5- | + | >270 |
| | a]pyrimidin-7-yl]phenyl}amino)carbonyl]benzoic acid | | |
| 447 | (7-{3-[(2-methylundecyl)amino]phenyl}pyrazolo[1,5- | + | 108-110 |
| | a]pyrimidin-3-yl)(thien-2-yl)methanone | | |
| 448 | 3-pyridin-3-yl-N-{3-[3-(thien-2- | + | >200 |
| | ylcarbonyl)pyrazolo[1,5- | | |
| | a]pyrimidin-7-yl]phenyl}propanamide | | |
| 449 | 2-(pyridin-4-ylthio)-N-{3-[3-(thien-2- | + | 138-144 |
| | ylcarbonyl)pyrazolo[1,5-a]pyrimidin-7- | | |
| | yl]phenyl}acetamide | | |
| 450 | {7-[4′-(dimethylamino)-1,1′-biphenyl-3-yl]pyrazolo[1,5- | +++ | 210-202 |
| | a]pyrimidin-3-yl}(thien-2-yl)methanone | | |
| 451 | [7-(3′-amino-1,1′-biphenyl-3-yl)pyrazolo[1,5- | +++ | 117-121 |
| | a]pyrimidin-3-yl](thien-2-yl)methanone | | |
| 452 | {7-[4′-(hydroxymethyl)-1,1′-biphenyl-3-yl]pyrazolo[1,5- | +++ | 106-110 |
| | a]pyrimidin-3-yl}(thien-2-yl)methanone | | |
| 453 | {7-[3′-(dimethylamino)-1,1′-biphenyl-3-yl]pyrazolo[1,5- | ++ | 167-169 |
| | a]pyrimidin-3-yl}(thien-2-yl)methanone | | |
| 454 | {7-[4′-(dimethylamino)-6-fluoro-1,1′-biphenyl-3- | ++ | 219-221 |
| | yl]pyrazolo[1,5-a]pyrimidin-3-yl}(thien-2-yl)methanone | | |
| 455 | {7-[6-fluoro-4′-(hydroxymethyl)-1,1′-biphenyl-3- | ++ | 140-145 |
| | yl]pyrazolo[1,5-a]pyrimidin-3-yl}(thien-2-yl)methanone | | |
| 456 | [7-(4′-ethyl-1,1′-biphenyl-3-yl)pyrazolo[1,5-a]pyrimidin- | +++ | 117-119 |
| | 3-yl](thien-2-yl)methanone | | |
| 457 | 3-{3′-[3-(thien-2-ylcarbonyl)pyrazolo[1,5-a]pyrimidin- | + | 117-119 |
| | 7-yl]-1,1′-biphenyl-4-yl}propanoic acid | | |
| 458 | 3′-[3-(thien-2-ylcarbonyl)pyrazolo[1,5-a]pyrimidin-7- | ++ | 156-158 |
| | yl]-1,1′-biphenyl-4-carbaldehyde | | |
| 459 | {7-[4′-(morpholin-4-ylmethyl)-1,1′-biphenyl-3- | +++ | 175-178 |
| | yl]pyrazolo[1,5-a]pyrimidin-3-yl}(thien-2-yl)methanone | | |
| 460 | (7-{4′-[(4-methylpiperazin-1-yl)methyl]-1,1′-biphenyl-3- | +++ | 199-202 |
| | yl}pyrazolo[1,5-a]pyrimidin-3-yl)(thien-2-yl)methanone | | |
| 461 | [7-(4′-{[(2-morpholin-4-ylethyl)amino]methyl}-1,1′- | ++ | 100-106 |
| | biphenyl-3-yl)pyrazolo[1,5-a]pyrimidin-3-yl](thien-2- | | |
| | yl)methanone | | |
| 462 | (7-{4′-[(diethylamino)methyl]-1,1′-biphenyl-3- | ++ | 144-146 |
| | yl}pyrazolo[1,5-a]pyrimidin-3-yl)(thien-2-yl)methanone | | |
| 463 | (7-{4′-[(dimethylamino)methyl]-1,1′-biphenyl-3- | +++ | 192-195 |
| | yl}pyrazolo[1,5-a]pyrimidin-3-yl)(thien-2-yl)methanone | | |
| 464 | N-{3-[3-(3-aminophenyl)pyrazolo[1,5-a]pyrimidin-7- | + | 173-175 |
| | yl]phenyl}-3-methylbutanamide | | |
| 465 | N-(3-{3-[3-(dimethylamino)phenyl]pyrazolo[1,5- | +++ | 182-184 |
| | a]pyrimidin-7-yl}phenyl)-3-methylbutanamide | | |
| 466 | N-{3-[3-(1H-indol-5-yl)pyrazolo[1,5-a]pyrimidin-7- | ++ | 167-169 |
| | yl]phenyl}-3-methylbutanamide | | |
| 467 | 3-methyl-N-{3-[3-(3,4,5- | +++ | 86-90 |
| | trimethoxyphenyl)pyrazolo[1,5-a]pyrimidin-7- | | |
| | yl]phenyl}butanamide | | |
| 468 | 3-methyl-N-[3-(3-quinolin-8-ylpyrazolo[1,5- | ++ | 96-99 |
| | a]pyrimidin-7-yl)phenyl]butanamide | | |
| 469 | N-(3-{3-[4-(hydroxymethyl)phenyl]pyrazolo[1,5- | + | 170-172 |
| | a]pyrimidin-7-yl}phenyl)-3-methylbutanamide | | |
| 470 | N-{3-[3-(4-methoxyphenyl)pyrazolo[1,5-a]pyrimidin-7- | + | 225-227 |
| | yl]phenyl}-3-methylbutanamide | | |
| 471 | N-{3-[3-(3-chlorophenyl)pyrazolo[1,5-a]pyrimidin-7- | + | 169-171 |
| | yl]phenyl}-3-methylbutanamide | | |
| 472 | N-{3-[3-(4-chlorophenyl)pyrazolo[1,5-a]pyrimidin-7- | + | 215-217 |
| | yl]phenyl}-3-methylbutanamide | | |
| 473 | N-{3-[3-(2,3-dichlorophenyl)pyrazolo[1,5-a]pyrimidin- | ++ | 154-156 |
| | 7-yl]phenyl}-3-methylbutanamide | | |
| 474 | N-{3-[3-(3,4-dichlorophenyl)pyrazolo[1,5-a]pyrimidin- | + | 215-217 |
| | 7-yl]phenyl}-3-methylbutanamide | | |
| 475 | N-{3-[3-(3,5-dichlorophenyl)pyrazolo[1,5-a]pyrimidin- | ++ | 223-225 |
| | 7-yl]phenyl}-3-methylbutanamide | | |
| 476 | N-{3-[3-(3,5-dimethylphenyl)pyrazolo[1,5-a]pyrimidin- | +++ | 178-180 |
| | 7-yl]phenyl}-3-methylbutanamide | | |
| 477 | 3-methyl-N-(3-{3-[3- | + | 170-173 |
| | (trifluoromethyl)phenyl]pyrazolo[1,5-a]pyrimidin-7- | | |
| | yl}phenyl)butanamide | | |
| 478 | N-{3-[3-(3-methoxyphenyl)pyrazolo[1,5-a]pyrimidin-7- | + | 153-154 |
| | yl]phenyl}-3-methylbutanamide | | |
| 479 | N-{3-[3-(3-ethoxyphenyl)pyrazolo[1,5-a]pyrimidin-7- | + | 156-158 |
| | yl]phenyl}-3-methylbutanamide | | |
| 480 | 3-methyl-N-{3-[3-(4-phenoxyphenyl)pyrazolo[1,5- | + | 175-178 |
| | a]pyrimidin-7-yl]phenyl}butanamide | | |
| 481 | N-{3-[3-(1,3-benzodioxol-5-yl)pyrazolo[1,5- | ++ | 172-173 |
| | a]pyrimidin-7-yl]phenyl}-3-methylbutanamide | | |
| 482 | N-{3-[3-(3-cyanophenyl)pyrazolo[1,5-a]pyrimidin-7- | + | 166-167 |
| | yl]phenyl}-3-methylbutanamide | | |
| 483 | N-{3-[3-(3-acetylphenyl)pyrazolo[1,5-a]pyrimidin-7- | + | 190-192 |
| | yl]phenyl}-3-methylbutanamide | | |
| 484 | N-{3-[3-(3-formyl-4-methoxyphenyl)pyrazolo[1,5- | ++ | 187-189 |
| | a]pyrimidin-7-yl]phenyl}-3-methylbutanamide | | |
| 485 | N-{3-[3-(1,1′-biphenyl-3-yl)pyrazolo[1,5-a]pyrimidin-7- | + | 182-185 |
| | yl]phenyl}-3-methylbutanamide | | |
| 486 | N-{3-[3-(1,1′-biphenyl-4-yl)pyrazolo[1,5-a]pyrimidin-7- | + | 157-160 |
| | yl]phenyl}-3-methylbutanamide | | |
| 487 | N-{3-[3-(3-formylphenyl)pyrazolo[1,5-a]pyrimidin-7- | + | 163-165 |
| | yl]phenyl}-3-methylbutanamide | | |
| 488 | 3-methyl-N-(3-{3-[3-(morpholin-4- | + | 170-173 |
| | ylmethyl)phenyl]pyrazolo[1,5-a]pyrimidin-7- | | |
| | yl}phenyl)butanamide | | |
| 489 | 3-methyl-N-[3-(3-{3-[(4-methylpiperazin-1- | ++ | 135-138 |
| | yl)methyl]phenyl}pyrazolo[1,5-a]pyrimidin-7- | | |
| | yl)phenyl]butanamide | | |
| 490 | 3-[3-(3,5-dimethylphenyl)pyrazolo[1,5-a]pyrimidin-7- | + | 147 |
| | yl]benzonitrile | | |
| 491 | 3-(3,5-dimethylphenyl)-7-[3-(1H-tetraazol-5- | ++ | 250(dec) |
| | yl)phenyl]pyrazolo[1,5-a]pyrimidine | | |
| 492 | 7-{3-[2-(cyclobutylmethyl)-2H-tetraazol-5-yl]phenyl}- | ++ | 64 |
| | 3-(3,5-dimethylphenyl)pyrazolo[1,5-a]pyrimidine | | |
| 493 | 3-(3,5-dimethylphenyl)-7-{3-[2-(2-morpholin-4- | ++ | 68 |
| | ylethyl)-2H-tetraazol-5-yl]phenyl}pyrazolo[1,5- | | |
| | a]pyrimidine | | |
| 494 | 3-(3,5-dimethylphenyl)-7-{3-[2-(pyridin-3-ylmethyl)- | ++ | 73 |
| | 2H-tetrazol-5-yl]phenyl}pyrazolo[1,5-a]pyrimidine | | |
| 495 | 3-(3,5-dimethylphenyl)-7-{3-[2-(pyridin-2-ylmethyl)- | + | 77 |
| | 2H-tetrazol-5-yl]phenyl}pyrazolo[1,5-a]pyrimidine | | |
| 496 | 3-(3,5-dimethylphenyl)-7-[3-(2-ethyl-2H-tetrazol-5- | ++ | 65 |
| | yl)phenyl]pyrazolo[1,5-a]pyrimidine | | |
| 497 | N-[3-(3-{3- | + | 179-182 |
| | [(dimethylamino)methyl]phenyl}pyrazolo[1,5- | | |
| | a]pyrimidin-7-yl)phenyl]-3-methylbutanamide | | |
| 498 | 3-(3,5-dimethylphenyl)-7-{3-[2-(2-pyrrolidin-1-ylethyl)- | ++ | 60 |
| | 2H-tetrazol-5-yl]phenyl}pyrazolo[1,5-a]pyrimidine | | |
| 499 | 3-(3,5-dimethylphenyl)-7-{3-[2-(2-piperidin-1-ylethyl)- | ++ | 66 |
| | 2H-tetrazol-5-yl]phenyl}pyrazolo[1,5-a]pyrimidine | | |
| 500 | 3-(3,5-dimethylphenyl)-7-{3-[2-(pyridin-4-ylmethyl)- | ++ | 172 |
| | 2H-tetrazol-5-yl]phenyl}pyrazolo[1,5-a]pyrimidine | | |
| 501 | {7-[3-(1H-tetraazol-5-yl)phenyl]pyrazolo[1,5- | + | 290 |
| | a]pyrimidin-3-yl}(thien-2-yl)methanone | | |
| 502 | {7-[3-(2-ethyl-2H-tetraazol-5-yl)phenyl]pyrazolo[1,5- | ++ | 174 |
| | a]pyrimidin-3-yl}(thien-2-yl)methanone | | |
| 503 | (7-{3-[2-(pyridin-3-ylmethyl)-2H-tetraazol-5- | +++ | 95 |
| | yl]phenyl}pyrazolo[1,5-a]pyrimidin-3-yl)(thien-2- | | |
| | yl)methanone | | |
| 504 | {7-[3-(2-isobutyl-2H-tetraazol-5- | +++ | 141 |
| | yl)phenyl]pyrazolo[1,5-a]pyrimidin-3-yl}(thien-2- | | |
| | yl)methanone | | |
| 505 | (7-{3-[2-(cyclobutylmethyl)-2H-tetraazol-5- | +++ | 170 |
| | yl]phenyl}pyrazolo[1,5-a]pyrimidin-3-yl)(thien-2- | | |
| | yl)methanone | | |
| 506 | {7-[3-(2-butyl-2H-tetraazol-5-yl)phenyl]pyrazolo[1,5- | +++ | 90 |
| | a]pyrimidin-3-yl}(thien-2-yl)methanone | | |
| 507 | (7-{3-[2-(2-morpholin-4-ylethyl)-2H-tetraazol-5- | ++ | 103 |
| | yl]phenyl}pyrazolo[1,5-a]pyrimidin-3-yl)(thien-2- | | |
| | yl)methanone | | |
| 508 | (7-{3-[1-(cyclobutylmethyl)-1H-tetraazol-5- | + | 204 |
| | yl]phenyl}pyrazolo[1,5-a]pyrimidin-3-yl)(thien-2- | | |
| | yl)methanone | | |
| 509 | (7-{3-[2-(pyridin-2-ylmethyl)-2H-tetraazol-5- | +++ | 184 |
| | yl]phenyl}pyrazolo[1,5-a]pyrimidin-3-yl)(thien-2- | | |
| | yl)methanone | | |
| 510 | (7-{3-[2-(pyridin-4-ylmethyl)-2H-tetraazol-5- | +++ | 170 |
| | yl]phenyl}pyrazolo[1,5-a]pyrimidin-3-yl)(thien-2- | | |
| | yl)methanone | | |
| 511 | (7-{3-[2-(2-pyrrolidin-1-ylethyl)-2H-tetraazol-5- | +++ | 98 |
| | yl]phenyl}pyrazolo[1,5-a]pyrimidin-3-yl)(thien-2- | | |
| | yl)methanone | | |
| 512 | (7-{3-[2-(2-piperidin-1-ylethyl)-2H-tetraazol-5- | ++ | 117 |
| | yl]phenyl}pyrazolo[1,5-a]pyrimidin-3-yl)(thien-2- | | |
| | yl)methanone | | |
| 513 | (7-{3-[2-(2-hydroxyethyl)-2H-tetraazol-5- | ++ | 134 |
| | yl]phenyl}pyrazolo[1,5-a]pyrimidin-3-yl)(thien-2- | | |
| | yl)methanone | | |
| 514 | {7-[3-(1H-pyrazol-3-yl)phenyl]pyrazolo[1,5- | +++ | 129 |
| | a]pyrimidin-3-yl}(thien-2-yl)methanone | | |
| 515 | (7-{3-[1-(cyclobutylmethyl)-1H-pyrazol-3- | +++ | 60 |
| | yl]phenyl}pyrazolo[1,5-a]pyrimidin-3-yl)(thien-2- | | |
| | yl)methanone | | |
| 516 | (7-{3-[1-(2-morpholin-4-ylethyl)-1H-pyrazol-3- | +++ | 73 |
| | yl]phenyl}pyrazolo[1,5-a]pyrimidin-3-yl)(thien-2- | | |
| | yl)methanone | | |
| 517 | (7-{3-[1-(pyridin-2-ylmethyl)-1H-pyrazol-3- | +++ | 66 |
| | yl]phenyl}pyrazolo[1,5-a]pyrimidin-3-yl)(thien-2- | | |
| | yl)methanone | | |
| 518 | {7-[3-(1-ethyl-1H-pyrazol-3-yl)phenyl]pyrazolo[1,5- | +++ | 61 |
| | a]pyrimidin-3-yl}(thien-2-yl)methanone | | |
| 519 | (7-{3-[1-(2-piperidin-1-ylethyl)-1H-pyrazol-3- | ++ | 62 |
| | yl]phenyl}pyrazolo[1,5-a]pyrimidin-3-yl)(thien-2- | | |
| | yl)methanone | | |
| 520 | (7-{3-[1-(2-pyrrolidin-1-ylethyl)-1H-pyrazol-3- | ++ | 63 |
| | yl]phenyl}pyrazolo[1,5-a]pyrimidin-3-yl)(thien-2- | | |
| | yl)methanone | | |

+++, <1 μM;
++, 1-10 μM;
+; 10-50 μM

TABLE 4
Cellular activity in LoVo cell

| Example | | Inhibition |
|---|---|---|
| number | Chemical Name | of LoVo |
| 245 | [7-[3-(2-methyl-1H-imidazol-1-yl)phenyl]- | + |
| | pyrazolo[1,5-a]pyrimidin-3-yl]-2-thienyl-methanone | |
| 247 | [7-[3-(4-methyl-1H-imidazol-1-yl)phenyl]- | ++ |
| | pyrazolo[1,5-a]pyrimidin-3-yl]-2-thienyl-methanone | |
| 267 | [7-[3-propylamino)phenyl]pyrazolo[1,5- | ++ |
| | a]pyrimidin-3-yl]-2-thienyl-methanone | |
| 273 | 2-methyl-N-{3-[3-(2-thienylcarbonyl)pyrazolo[1,5- | ++ |
| | a]pyrimidin-7-yl]phenyl}propanamide | |
| 274 | N-{3-[3-(2-thienylcarbonyl)pyrazolo[1,5-a]pyrimidin-7- | +++ |
| | yl]phenyl}-2-butynamide | |
| 275 | N-{3-[3-(2-thienylcarbonyl)pyrazolo[1,5-a]pyrimidin-7- | +++ |
| | yl]phenyl}cyclobutanecarboxamide | |
| 276 | N-{3-[5-methyl-3-(2-thienylcarbonyl)pyrazolo[1,5- | +++ |
| | a]pyrimidin-7-yl]phenyl}cyclopropanecarboxamide | |
| 282 | N-{3-[3-(2-thienylcarbonyl)pyrazolo[1,5-a]pyrimidin-7- | ++ |
| | yl]phenyl}cyclohexanecarboxamide | |
| 283 | N-{3-[3-(2-thienylcarbonyl)pyrazolo[1,5-a]pyrimidin-7- | +++ |
| | yl]phenyl}cyclopentanecarboxamide | |
| 284 | N-{3-[3-(2-thienylcarbonyl)pyrazolo[1,5-a]pyrimidin-7- | +++ |
| | yl]phenyl}benzamide | |
| 285 | N-{3-[3-(2-thienylcarbonyl)pyrazolo[1,5-a]pyrimidin-7- | +++ |
| | yl]phenyl}-2-furamide | |
| 290 | N-{3-[3-(2-thienylcarbonyl)pyrazolo[1,5-a]pyrimidin-7- | ++ |
| | yl]phenyl}nicotinamide | |
| 291 | N-{3-[3-(2-thienylcarbonyl)pyrazolo[1,5-a]pyrimidin-7- | +++ |
| | yl]phenyl}isonicotinamide | |
| 293 | Isopropyl-3-[3-(3-thienylcarbonyl)pyrazolo[1,5- | +++ |
| | a]pyrimidin-7-yl]phenylcarbamate | |
| 297 | 3-methyl-N-{3-[3-(3-thienylcarbonyl)pyrazolo[1,5- | +++ |
| | a]pyrimidin-7-yl]phenyl}butanamide | |
| 303 | 2-cyclopentyl-N-{3-[3-(2-furoyl)pyrazolo[1,5- | +++ |
| | a]pyrimidin-7-yl]phenyl}acetamide | |
| 311 | N-isopropyl-N′-{2-methyl-5-[3-(2- | +++ |
| | thienylcarbonyl)pyrazolo[1,5-a]pyrimidin-7- | |
| | yl]phenyl}urea | |
| 312 | N-{2-chloro-5-[3-(2-thienylcarbonyl)pyrazolo[1,5- | +++ |
| | a]pyrimidin-7-yl]phenyl}benzamide | |
| 313 | N-{2-chloro-5-[3-(2-thienylcarbonyl)pyrazolo[1,5- | +++ |
| | a]pyrimidin-7-yl]phenyl}-3-methylbutanamide | |
| 314 | Isopropyl-2-methyl-5-[3-(2-thienylcarbonyl)pyrazolo[1,5- | +++ |
| | a]pyrimidin-7-yl]phenylcarbamate | |
| 324 | 3-methyl-N-{5-[3-(2-thienylcarbonyl)pyrazolo[1,5- | ++ |
| | a]pyrimidin-7-yl]-3-pyridinyl}butanamide | |
| 328 | [7-(5-nitro-3-thienyl)pyrazolo[1,5-a]pyrimidin-3-yl](2- | ++ |
| | thienyl)methanone | |
| 329 | N-isopropyl-N′-{5-[3-(thien-2-ylcarbonyl)pyrazolo[1,5- | ++ |
| | a]pyrimidin-7-yl]thien-2-yl}urea | |
| 331 | [7-(5-aminothien-3-yl)pyrazolo[1,5-a]pyrimidin-3- | + |
| | yl](thien-2-yl)methanone | |
| 332 | O-ethyl 3-[3-(thien-2-ylcarbonyl)pyrazolo[1,5- | +++ |
| | a]pyrimidin-7-yl]phenylthiocarbamate | |
| 334 | N-phenyl-N′-{3-[3-(thien-2-ylcarbonyl)pyrazolo[1,5- | ++ |
| | a]pyrimidin-7-yl]phenyl}thiourea | |
| 335 | isopropyl 4-chloro-3-[3-(thien-2- | ++ |
| | ylcarbonyl)pyrazolo[1,5-a]pyrimidin-7- | |
| | yl]phenylcarbamate | |
| 338 | N-{3-[3-(1,3-thiazol-2-ylcarbonyl)pyrazolo[1,5- | ++ |
| | a]pyrimidin-7-yl]phenyl}cyclopropanecarboxamide | |
| 339 | N-{4-chloro-3-[3-(thien-2-ylcarbonyl)pyrazolo[1,5- | ++ |
| | a]pyrimidin-7-yl]phenyl}-3-methylbutanamide | |
| 340 | N-(4-chloro-3-[3-(thien-2-ylcarbonyl)pyrazolo[1,5- | ++ |
| | a]pyrimidin-7-yl]phenyl}-N′-isopropylthiourea | |
| 341 | N-{4-chloro-3-[3-(thien-2-ylcarbonyl)pyrazolo[1,5- | ++ |
| | a]pyrimidin-7-yl]phenyl}benzamide | |
| 342 | N-{2-fluoro-5-[3-(thien-2-ylcarbonyl)pyrazolo[1,5- | +++ |
| | a]pyrimidin-7-yl]phenyl}-3-methylbutanamide | |
| 343 | 3-methyl-N-{5-[3-(thien-2-ylcarbonyl)pyrazolo[1,5- | + |
| | a]pyrimidin-7-yl]thien-2-yl}butanamide | |
| 344 | isopropyl 5-[3-(thien-2-ylcarbonyl)pyrazolo[1,5- | + |
| | a]pyrimidin-7-yl]thien-2-ylcarbamate | |
| 345 | [7-(5-aminothien-2-yl)pyrazolo[1,5-a]pyrimidin-3- | + |
| | yl](thien-2-yl)methanone | |
| 346 | 4-chloro-N-{3-[3-(thien-2-ylcarbonyl)pyrazolo[1,5- | ++ |
| | a]pyrimidin-7-yl]phenyl}butanamide | |
| 347 | isopropyl 2-fluoro-5-[3-(thien-2-ylcarbonyl)pyrazolo[1,5- | +++ |
| | a]pyrimidin-7-yl]phenylcarbamate | |
| 348 | isopropyl 2-(dimethylamino)-5-[3-(thien-2- | ++ |
| | ylcarbonyl)pyrazolo[1,5-a]pyrimidin-7- | |
| | yl]phenylcarbamate | |
| 349 | {7-[3-(benzylamino)phenyl]pyrazolo[1,5-a]pyrimidin-3- | ++ |
| | yl}(thien-2-yl)methanone | |
| 350 | {7-[3-(isopentylamino)phenyl]pyrazolo[1,5-a]pyrimidin- | +++ |
| | 3-yl}(thien-2-yl)methanone | |
| 351 | 3-methyl-N-{4-[3-(thien-2-ylcarbonyl)pyrazolo[1,5- | + |
| | a]pyrimidin-7-yl]thien-2-yl}butanamide | |
| 352 | [7-(3-amino-4-fluorophenyl)pyrazolo[1,5-a]pyrimidin-3- | + |
| | yl](thien-2-yl)methanone | |
| 353 | N-cyclohexyl-N′-{3-[3-(thien-2-ylcarbonyl)pyrazolo[1,5- | +++ |
| | a]pyrimidin-7-yl]phenyl}urea | |
| 354 | 4,4,4-trichloro-N-{3-[3-(thien-2-ylcarbonyl)pyrazolo[1,5- | +++ |
| | a]pyrimidin-7-yl]phenyl}butanamide | |
| 355 | N-{2-fluoro-5-[3-(thien-2-ylcarbonyl)pyrazolo[1,5- | +++ |
| | a]pyrimidin-7-yl]phenyl}-3,3-dimethylbutanamide | |
| 356 | 4,4,4-trifluoro-N-{2-fluoro-5-[3-(thien-2- | +++ |
| | ylcarbonyl)pyrazolo[1,5-a]pyrimidin-7-yl]phenyl}-3- | |
| | methylbutanamide | |
| 357 | bis(2,2,2-trichloroethyl)2-fluoro-5-[3-(thien-2- | +++ |
| | ylcarbonyl)pyrazolo[1,5- | |
| | a]pyrimidin-7-yl]phenylimidodicarbonate | |
| 358 | 2-fluoro-N-{3-[3-(thien-2-ylcarbonyl)pyrazolo[1,5- | +++ |
| | a]pyrimidin-7-yl]phenyl}benzamide | |
| 359 | 2,6-difluoro-N-{3-[3-(thien-2-ylcarbonyl)pyrazolo[1,5- | +++ |
| | a]pyrimidin-7-yl]phenyl}benzamide | |
| 360 | N-{3-[3-(thien-2-ylcarbonyl)pyrazolo[1,5-a]pyrimidin-7- | +++ |
| | yl]phenyl}pyridine-2-carboxamide | |
| 361 | N-{5-[3-(thien-2-ylcarbonyl)pyrazolo[1,5-a]pyrimidin-7- | + |
| | yl]thien-2-yl}benzamide | |
| 362 | (7-{3-[(2-phenylethyl)amino]phenyl}pyrazolo[1,5-a]pyrimidin-3- | + |
| | yl)(thien-2-yl)methanone | |
| 363 | (7-{3-[(2-methoxybenzyl)amino]phenyl}pyrazolo[1,5-a]pyrimidin- | ++ |
| | 3-yl)(thien-2-yl)methanone | |
| 364 | N-butyl-N′-{3-[3-(thien-2-ylcarbonyl)pyrazolo[1,5- | +++ |
| | a]pyrimidin-7-yl]phenyl}thiourea | |
| 365 | (7-{3-[(3-chlorobenzyl)amino]phenyl}pyrazolo[1,5- | + |
| | a]pyrimidin-3-yl)(thien-2-yl)methanone | |
| 366 | (7-{3-[(4-chlorobenzyl)amino]phenyl}pyrazolo[1,5- | + |
| | a]pyrimidin-3-yl)(thien-2-yl)methanone | |
| 367 | (7-{3-[(2-furylmethyl)amino]phenyl}pyrazolo[1,5- | ++ |
| | a]pyrimidin-3-yl)(thien-2-yl)methanone | |
| 368 | (7-{3-[(2-chlorobenzyl)amino]phenyl}pyrazolo[1,5- | ++ |
| | a]pyrimidin-3-yl)(thien-2-yl)methanone | |
| 369 | (7-{3-[(3-furylmethyl)amino]phenyl}pyrazolo[1,5- | ++ |
| | a]pyrimidin-3-yl)(thien-2-yl)methanone | |
| 370 | (7-{3-[(2E)-but-2-enylamino]phenyl}pyrazolo[1,5- | +++ |
| | a]pyrimidin-3-yl)(thien-2-yl)methanone | |
| 371 | (7-{3-[(3-methylbut-2-enyl)amino]phenyl)pyrazolo[1,5- | +++ |
| | a]pyrimidin-3-yl)(thien-2-yl)methanone | |
| 372 | (7-{3-[(3,3-dimethylbutyl)amino]phenyl}pyrazolo[1,5- | +++ |
| | a]pyrimidin-3-yl)(thien-2-yl)methanone | |
| 373 | [7-(6-chloropyridin-3-yl)pyrazolo[1,5-a]pyrimidin-3- | + |
| | yl](thien-2-yl)methanone | |
| 374 | 3,6-difluoro-N-{2-fluoro-5-[3-(thien-2- | +++ |
| | ylcarbonyl)pyrazolo[1,5-a]pyrimidin-7- | |
| | yl]phenyl}benzamide | |
| 375 | 2-fluoro-N-{2-fluoro-5-[3-(thien-2- | +++ |
| | ylcarbonyl)pyrazolo[1,5-a]pyrimidin-7- | |
| | yl]phenyl}benzamide | |
| 376 | 2-chloro-N-{2-fluoro-5-[3-(thien-2- | +++ |
| | ylcarbonyl)pyrazolo[1,5-a]pyrimidin-7- | |
| | yl]phenyl}benzamide | |
| 377 | (7-{3-[(2-methylbenzyl)amino]phenyl}pyrazolo[1,5- | ++ |
| | a]pyrimidin-3-yl)(thien-2-yl)methanone | |
| 378 | (7-{3-[(2-phenylpropyl)amino]phenyl}pyrazolo[1,5- | + |
| | a]pyrimidin-3-yl)(thien-2-yl)methanone | |
| 379 | (7-{3-[(cyclohexylmethyl)amino]phenyl}pyrazolo[1,5- | +++ |
| | a]pyrimidin-3-yl)(thien-2-yl)methanone | |
| 380 | {7-[3-(butylamino)phenyl]pyrazolo[1,5-a]pyrimidin-3- | +++ |
| | yl}(thien-2-yl)methanone | |
| 381 | 2-chloroethyl 2-fluoro-5-[3-(thien-2- | +++ |
| | ylcarbonyl)pyrazolo[1,5-a]pyrimidin-7- | |
| | yl]phenylcarbamate | |
| 382 | N-{2-fluoro-5-[3-(thien-2-ylcarbonyl)pyrazolo[1,5- | +++ |
| | a]pyrimidin-7-yl]phenyl}-N′-isopropylurea | |
| 383 | 4-(1H-pyrrol-1-yl)-N-{3-[3-(thien-2- | ++ |
| | ylcarbonyl)pyrazolo[1,5-a]pyrimidin-7- | |
| | yl]phenyl}benzamide | |
| 384 | 2,6-dichloro-N-{3-[3-(thien-2-ylcarbonyl)pyrazolo[1,5- | ++ |
| | a]pyrimidin-7-yl]phenyl}benzamide | |
| 385 | N-{3-[3-(thien-2-ylcarbonyl)pyrazolo[1,5-a]pyrimidin-7- | +++ |
| | yl]phenyl}pyrazine-2-carboxamide | |
| 386 | phenyl N′-cyano-N-{3-[3-(thien-2- | + |
| | ylcarbonyl)pyrazolo[1,5-a]pyrimidin-7- | |
| | yl]phenyl}imidocarbamate | |
| 387 | 1-methyl-N-{3-[3-(thien-2-ylcarbonyl)pyrazolo[1,5- | +++ |
| | a]pyrimidin-7-yl]phenyl}-1 H-pyrrole-2-carboxamide | |
| 388 | N-{2-fluoro-5-[3-(thien-2-ylcarbonyl)pyrazolo[1,5- | +++ |
| | a]pyrimidin-7-yl]phenyl}isonicotinamide | |
| 389 | N-isopropyl-N′-{3-[3-(thien-2-ylcarbonyl)pyrazolo[1,5- | +++ |
| | a]pyrimidin-7-yl]phenyl}thiourea | |
| 390 | (7-chloropyrazolo[1,5-a]pyrimidin-3-yl)(thien-2- | + |
| | yl)methanone | |
| 391 | 2,6-dichloro-N-(2,6-dichlorobenzoyl)-N-{3-[3-(thien-2- | + |
| | ylcarbonyl)pyrazolo[1,5-a]pyrimidin-7- | |
| | yl]phenyl}benzamide | |
| 392 | tert-butyl 4-[2-oxo-2-({3-[3-(thien-2- | +++ |
| | ylcarbonyl)pyrazolo[1,5-a]pyrimidin-7- | |
| | yl]phenyl}amino)ethyl]piperidine-1-carboxylate | |
| 393 | 2-piperidin-4-yl-N-{3-[3-(thien-2- | ++ |
| | ylcarbonyl)pyrazolo[1,5-a]pyrimidin-7- | |
| | yl]phenyl}acetamide | |
| 394 | {7-[3-(diethylamino)phenyl]pyrazolo[1,5-a]pyrimidin-3- | ++ |
| | yl}(thien-2-yl)methanone | |
| 395 | [7-(3-{[(2E)-3-(2-methoxyphenyl)prop-2- | + |
| | enyl]amino}phenyl)pyrazolo[1,5-a]pyrimidin-3-yl](thien- | |
| | 2-yl)methanone | |
| 396 | [7-(3-{[(2E)-3-(4-methoxyphenyl)prop-2- | + |
| | enyl]amino}phenyl)pyrazolo[1,5-a]pyrimidin-3-yl](thien- | |
| | 2-yl)methanone | |
| 398 | [7-(3-{[(2E)-3-(4-nitrophenyl)prop-2- | + |
| | enyl]amino}phenyl)pyrazolo[1,5-a]pyrimidin- | |
| | 3-yl](thien-2-yl)methanone | |
| 399 | (7-{3-[(3,3-diphenylprop-2- | + |
| | enyl)amino]phenyl}pyrazolo[1,5-a]pyrimidin-3-yl)(thien- | |
| | 2-yl)methanone | |
| 419 | (7-{3-[(2-ethylhexyl)amino]phenyl}pyrazolo[1,5- | + |
| | a]pyrimidin-3-yl)(thien-2-yl)methanone | |
| 420 | 3-(1,1-dioxidothiomorpholin-4-yl)propyl3-[3-(thien-2- | ++ |
| | ylcarbonyl)pyrazolo[1,5-a]pyrimidin-7- | |
| | yl]phenylcarbamate | |
| 421 | 2-chloro-N-{2-(dimethylamino)-5-[3-(thien-2- | ++ |
| | ylcarbonyl)pyrazolo[1,5-a]pyrimidin- | |
| | 7-yl]phenyl}benzamide | |
| 422 | {7-[3-(ethylamino)phenyl]pyrazolo[1,5-a]pyrimidin-3- | ++ |
| | yl}(thien-2-yl)methanone | |
| 423 | 4-morpholin-4-yl-N-{3-[3-(thien-2- | ++ |
| | ylcarbonyl)pyrazolo[1,5-a]pyrimidin-7- | |
| | yl]phenyl}butanamide | |
| 424 | pyridine-3-ylmethyl 3-[3-(thien-2- | + |
| | ylcarbonyl)pyrazolo[1,5-a]pyrimidin-7- | |
| | y]phenylcarbamate | |
| 425 | 3-morpholin-4-ylpropyl 3-[3-(thien-2- | ++ |
| | ylcarbonyl)pyrazolo[1,5-a]pyrimidin-7- | |
| | yl]phenylcarbamate | |
| 426 | [7-(3-{[(2E)-2-methylpent-2- | ++ |
| | enyl]amino}phenyl)pyrazolo[1,5-a]pyrimidin-3-yl](thien- | |
| | 2-yl)methanone | |
| 427 | 2-pyridin-4-yl-N-{3-[3-(thien-2-ylcarbonyl)pyrazolo[1,5- | + |
| | a]pyrimidin-7-yl]phenyl}acetamide | |
| 429 | pyridin-2-ylmethyl 3-[3-(thien-2- | ++ |
| | ylcarbonyl)pyrazolo[1,5-a]pyrimidin-7- | |
| | yl]phenylcarbamate | |
| 430 | (7-{3-[(2,3-dimethylpentyl)amino]phenyl}pyrazolo[1,5- | ++ |
| | a]pyrimidin-3-yl)(thien-2-yl)methanone | |
| 431 | [7-(3-{[(2E)-2-methylbut-2- | ++ |
| | enyl]amino}phenyl)pyrazolo[1,5-a]pyrimidin- | |
| | 3-yl](thien-2-yl)methanone | |
| 432 | (7-{3-[(2E)-pent-2- | +++ |
| | enylamino]phenyl}pyrazolo[1,5-a]pyrimidin-3- | |
| | yl)(thien-2-yl)methanone | |
| 433 | thien-2-yl(7-{3-[(3,5,5- | ++ |
| | trimethylhexyl)amino]phenyl}pyrazolo[1,5-a]pyrimidin- | |
| | 3-yl)methanone | |
| 434 | (7-{3-[(2-methylprop-2- | ++ |
| | enyl)amino]phenyl}pyrazolo[1,5-a]pyrimidin-3-yl)(thien- | |
| | 2-yl)methanone | |
| 435 | (7-{3-[(2-methylprop-2- | ++ |
| | enyl)amino]phenyl}pyrazolo[1,5-a]pyrimidin-3-yl)(thien- | |
| | 2-yl)methanone | |
| 436 | (7-{3-[(2-ethylprop-2-enyl)amino]phenyl}pyrazolo[1,5- | +++ |
| | a]pyrimidin-3-yl)(thien-2-yl)methanone | |
| 437 | 1-{3-[3-(thien-2-ylcarbonyl)pyrazolo[1,5-a]pyrimidin-7- | +++ |
| | yl]phenyl}pyrrolidin-2-one | |
| 438 | 2-chloro-N-(2-chlorobenzoyl)-N-{2-fluoro-5-[3-(thien-2- | +++ |
| | ylcarbonyl)pyrazolo[1,5-a]pyrimidin-7- | |
| | yl]phenyl}benzamide | |
| 439 | 2-pyridin-3-yl-N-{3-[3-(thien-2-ylcarbonyl)pyrazolo[1,5- | +++ |
| | a]pyrimidin-7-yl]phenyl}acetamide | |
| 440 | 3-pyridin-4-yl-N-{3-[3-(thien-2-ylcarbonyl)pyrazolo[1,5- | ++ |
| | a]pyrimidin-7-yl]phenyl}propanamide | |
| 441 | N-{3-[3-(thien-2-ylcarbonyl)pyrazolo[1,5-a]pyrimidin-7- | + |
| | Yl]phenyl}-1H-pyrazole-4-carboxamide | |
| 442 | 4-oxo-4-({3-[3-(thien-2-ylcarbonyl)pyrazolo[1,5- | + |
| | a]pyrimidin-7-yl]phenyl}amino)butanoic acid | |
| 443 | 2-[({3-[3-(thien-2-ylcarbonyl)pyrazolo[1,5-a]pyrimidin- | + |
| | 7-yl]phenyl}amino)carbonyl]benzoic acid | |
| 444 | 5-oxo-5-({3-[3-(thien-2-ylcarbonyl)pyrazolo[1,5- | + |
| | a]pyrimidin-7-yl]phenyl}amino)pentanoic acid | |
| 445 | 3-methyl-5-oxo-5-({3-[3-(thien-2- | + |
| | ylcarbonyl)pyrazolo[1,5-a]pyrimidin-7- | |
| | yl]phenyl}amino)pentanoic acid | |
| 446 | 4,5-dichloro-2-[({3-[3-(thien-2-ylcarbonyl)pyrazolo[1,5- | + |
| | a]pyrimidin-7-yl]phenyl}amino)carbonyl]benzoic acid | |
| 447 | (7-{3-[(2-methylundecyl)amino]phenyl}pyrazolo[1,5- | + |
| | a]pyrimidin-3-yl)(thien-2-yl)methanone | |
| 448 | 3-pyridin-3-yl-N-{3-[3-(thien-2-ylcarbonyl)pyrazolo[1,5- | ++ |
| | a]pyrimidin-7-yl]phenyl}propanamide | |
| 449 | 2-(pyridin-4-ylthio)-N-{3-[3-(thien-2- | ++ |
| | ylcarbonyl)pyrazolo[1,5-a]pyrimidin-7- | |
| | yl]phenyl}acetamide | |
| 450 | {7-[4′-(dimethylamino)-1,1′-biphenyl-3-yl]pyrazolo[1,5- | +++ |
| | a]pyrimidin-3-yl}(thien-2-yl)methanone | |
| 451 | [7-(3′-amino-1,1′-biphenyl-3-yl)pyrazolo[1,5-a]pyrimidin- | ++ |
| | 3-yl](thien-2-yl)methanone | |
| 452 | {7-[4′-(hydroxymethyl)-1,1′-biphenyl-3-yl]pyrazolo[1,5- | +++ |
| | a]pyrimidin-3-yl}(thien-2-yl)methanone | |
| 453 | {7-[3′-(dimethylamino)-1,1′-biphenyl-3-yl]pyrazolo[1,5- | ++ |
| | a]pyrimidin-3-yl}(thien-2-yl)methanone | |
| 454 | {7-[4′-(dimethylamino)-6-fluoro-1,1′-biphenyl-3- | ++ |
| | yl]pyrazolo[1,5-a]pyrimidin-3-yl}(thien-2-yl)methanone | |
| 455 | {7-[6-fluoro-4′-(hydroxymethyl)-1,1′-biphenyl-3- | ++ |
| | yl]pyrazolo[1,5-a]pyrimidin-3-yl}(thien-2-yl)methanone | |
| 456 | [7-(4′-ethyl-1,1′-biphenyl-3-yl)pyrazolo[1,5-a]pyrimidin-3- | +++ |
| | yl](thien-2-yl)methanone | |
| 457 | 3-{3′-[3-(thien-2-ylcarbonyl)pyrazolo[1,5-a]pyrimidin-7- | ++ |
| | yl]-1,1′-biphenyl-4-yl}propanoic acid | |
| 458 | 3′-[3-(thien-2-ylcarbonyl)pyrazolo[1,5-a]pyrimidin-7-yl]- | ++ |
| | 1,1′-biphenyl-4-carbaldehyde | |
| 459 | {7-[4′-(morpholin-4-ylmethyl)-1,1′-biphenyl-3- | NT |
| | yl]pyrazolo[1,5-a]pyrimidin-3-yl}(thien-2-yl)methanone | |
| 460 | (7-{4′-[(4-methylpiperazin-1-yl)methyl]-1,1′-biphenyl-3- | NT |
| | yl}pyrazolo[1,5-a]pyrimidin-3-yl)(thien-2-yl)methanone | |
| 461 | [7-(4′-{[(2-morpholin-4-ylethyl)amino]methyl}-1,1′- | ++ |
| | biphenyl-3-yl)pyrazolo[1,5-a]pyrimidin-3-yl](thien-2- | |
| | yl)methanone | |
| 462 | (7-{4′-[(diethylamino)methyl]-1,1′-biphenyl-3- | ++ |
| | yl}pyrazolo[1,5-a]pyrimidin-3-yl)(thien-2-yl)methanone | |
| 463 | (7-{4′-[(dimethylamino)methyl]-1,1′-biphenyl-3- | +++ |
| | yl}pyrazolo[1,5-a]pyrimidin-3-yl)(thien-2-yl)methanone | |
| 464 | N-{3-[3-(3-aminophenyl)pyrazolo[1,5-a]pyrimidin-7- | + |
| | yl]phenyl}-3-methylbutanamide | |
| 465 | N-(3-{3-[3-(dimethylamino)phenyl]pyrazolo[1,5- | +++ |
| | a]pyrimidin-7-yl}phenyl)-3-methylbutanamide | |
| 466 | N-{3-[3-(1H-indol-5-yl)pyrazolo[1,5-a]pyrimidin-7- | +++ |
| | yl]phenyl}-3-methylbutanamide | |
| 467 | 3-methyl-N-{3-[3-(3,4,5-trimethoxyphenyl)pyrazolo[1,5- | +++ |
| | a]pyrimidin-7-yl]phenyl}butanamide | |
| 468 | 3-methyl-N-[3-(3-quinolin-8-ylpyrazolo[1,5-a]pyrimidin-7- | ++ |
| | yl)phenyl]butanamide | |
| 469 | N-(3-{3-[4-(hydroxymethyl)phenyl]pyrazolo[1,5- | + |
| | a]pyrimidin-7-yl}phenyl)-3-methylbutanamide | |
| 470 | N-{3-[3-(4-methoxyphenyl)pyrazolo[1,5-a]pyrimidin-7- | + |
| | yl]phenyl}-3-methylbutanamide | |
| 471 | N-{3-[3-(3-chlorophenyl)pyrazolo[1,5-a]pyrimidin-7- | + |
| | yl]phenyl}-3-methylbutanamide | |
| 472 | N-{3-[3-(4-chlorophenyl)pyrazolo[1,5-a]pyrimidin-7- | + |
| | yl]phenyl}-3-methylbutanamide | |
| 473 | N-{3-[3-(2,3-dichlorophenyl)pyrazolo[1,5-a]pyrimidin-7- | ++ |
| | yl]phenyl}-3-methylbutanamide | |
| 474 | N-{3-[3-(3,4-dichlorophenyl)pyrazolo[1,5-a]pyrimidin-7- | + |
| | yl]phenyl}-3-methylbutanamide | |
| 475 | N-{3-[3-(3,5-dichlorophenyl)pyrazolo[1,5-a]pyrimidin-7- | + |
| | yl]phenyl}-3-methylbutanamide | |
| 476 | N-{3-[3-(3,5-dimethylphenyl)pyrazolo[1,5-a]pyrimidin-7- | +++ |
| | yl]phenyl}-3-methylbutanamide | |
| 477 | 3-methyl-N-(3-{3-[3-(trifluoromethyl)phenyl]pyrazolo[1,5- | + |
| | a]pyrimidin-7-yl}phenyl)butanamide | |
| 478 | N-{3-[3-(3-methoxyphenyl)pyrazolo[1,5-a]pyrimidin-7- | + |
| | yl]phenyl}-3-methylbutanamide | |
| 479 | N-{3-[3-(3-ethoxyphenyl)pyrazolo[1,5-a]pyrimidin-7- | + |
| | yl]phenyl}-3-methylbutanamide | |
| 480 | 3-methyl-N-{3-[3-(4-phenoxyphenyl)pyrazolo[1,5- | + |
| | a]pyrimidin-7-yl]phenyl}butanamide | |
| 481 | N-{3-[3-(1,3-benzodioxol-5-yl)pyrazolo[1,5-a]pyrimidin-7- | ++ |
| | yl]phenyl}-3-methylbutanamide | |
| 482 | N-{3-[3-(3-cyanophenyl)pyrazolo[1,5-a]pyrimidin-7- | + |
| | yl]phenyl}-3-methylbutanamide | |
| 483 | N-{3-[3-(3-acetylphenyl)pyrazolo[1,5-a]pyrimidin-7- | + |
| | yl]phenyl}-3-methylbutanamide | |
| 484 | N-{3-[3-(3-formyl-4-methoxyphenyl)pyrazolo[1,5- | + |
| | a]pyrimidin-7-yl]phenyl}-3-methylbutanamide | |
| 485 | N-{3-[3-(1,1′-biphenyl-3-yl)pyrazolo[1,5-a]pyrimidin-7- | + |
| | yl]phenyl}-3-methylbutanamide | |
| 486 | N-{3-[3-(1,1′-biphenyl-4-yl)pyrazolo[1,5-a]pyrimidin-7- | + |
| | yl]phenyl}-3-methylbutanamide | |
| 487 | N-{3-[3-(3-formylphenyl)pyrazolo[1,5-a]pyrimidin-7- | + |
| | yl]phenyl}-3-methylbutanamide | |
| 488 | 3-methyl-N-(3-{3-[3-(morpholin-4- | + |
| | ylmethyl)phenyl]pyrazolo[1,5-a]pyrimidin-7- | |
| | yl}phenyl)butanamide | |
| 489 | 3-methyl-N-[3-(3-{3-[(4-methylpiperazin-1- | + |
| | yl)methyl]phenyl}pyrazolo[1,5-a]pyrimidin-7- | |
| | yl)phenyl]butanamide | |
| 490 | 3-[3-(3,5-dimethylphenyl)pyrazolo[1,5-a]pyrimidin-7- | + |
| | yl]benzonitrile | |
| 491 | 3-(3,5-dimethylphenyl)-7-[3-(1H-tetraazol-5- | + |
| | yl)phenyl]pyrazolo[1,5-a]pyrimidine | |
| 492 | 7-{3-[2-(cyclobutylmethyl)-2H-tetraazol-5-yl]phenyl}-3- | ++ |
| | (3,5-dimethylphenyl)pyrazolo[1,5-a]pyrimidine | |
| 493 | 3-(3,5-dimethylphenyl)-7-{3-[2-(2-morpholin-4-ylethyl)- | ++ |
| | 2H-tetraazol-5-yl]phenyl}pyrazolo[1,5-a]pyrimidine | |
| 494 | 3-(3,5-dimethylphenyl)-7-{3-[2-(pyridin-3-ylmethyl)-2H- | ++ |
| | tetrazol-5-yl]phenyl}pyrazolo[1,5-a]pyrimidine | |
| 495 | 3-(3,5-dimethylphenyl)-7-{3-[2-(pyridin-2-ylmethyl)-2H- | ++ |
| | tetrazol-5-yl]phenyl}pyrazolo[1,5-a]pyrimidine | |
| 496 | 3-(3,5-dimethylphenyl)-7-[3-(2-ethyl-2H-tetrazol-5- | ++ |
| | yl)phenyl]pyrazolo[1,5-a]pyrimidine | |
| 497 | N-[3-(3-{3-[(dimethylamino)methyl]phenyl}pyrazolo[1,5- | NT |
| | a]pyrimidin-7-yl)phenyl]-3-methylbutanamide | |
| 498 | 3-(3,5-dimethylphenyl)-7-{3-[2-(2-pyrrolidin-1-ylethyl)- | NT |
| | 2H-tetrazol-5-yl]phenyl}pyrazolo[1,5-a]pyrimidine | |
| 499 | 3-(3,5-dimethylphenyl)-7-{3-[2-(2-piperidin-1-ylethyl)-2H- | NT |
| | tetrazol-5-yl]phenyl}pyrazolo[1,5-a]pyrimidine | |
| 500 | 3-(3,5-dimethylphenyl)-7-{3-[2-(pyridin-4-ylmethyl)-2H- | NT |
| | tetrazol-5-yl]phenyl}pyrazolo[1,5-a]pyrimidine | |
| 501 | {7-[3-(1H-tetraazol-5-yl)phenyl]pyrazolo[1,5-a]pyrimidin- | + |
| | 3-yl}(thien-2-yl)methanone | |
| 502 | {7-[3-(2-ethyl-2H-tetraazol-5-yl)phenyl]pyrazolo[1,5- | ++ |
| | a]pyrimidin-3-yl}(thien-2-yl)methanone | |
| 503 | (7-{3-[2-(pyridin-3-ylmethyl)-2H-tetraazol-5- | ++ |
| | yl]phenyl}pyrazolo[1,5-a]pyrimidin-3-yl)(thien-2- | |
| | yl)methanone | |
| 504 | {7-[3-(2-isobutyl-2H-tetraazol-5-yl)phenyl]pyrazolo[1,5- | ++ |
| | a]pyrimidin-3-yl}(thien-2-yl)methanone | |
| 505 | (7-{3-[2-(cyclobutylmethyl)-2H-tetraazol-5- | +++ |
| | yl]phenyl}pyrazolo[1,5-a]pyrimidin-3-yl)(thien-2- | |
| | yl)methanone | |
| 506 | {7-[3-(2-butyl-2H-tetraazol-5-yl)phenyl]pyrazolo[1,5- | +++ |
| | a]pyrimidin-3-yl}(thien-2-yl)methanone | |
| 507 | (7-{3-[2-(2-morpholin-4-ylethyl)-2H-tetraazol-5- | ++ |
| | yl]phenyl}pyrazolo[1,5-a]pyrimidin-3-yl)(thien-2- | |
| | yl)methanone | |
| 508 | (7-{3-[1-(cyclobutylmethyl)-1H-tetraazol-5- | + |
| | yl]phenyl}pyrazolo[1,5-a]pyrimidin-3-yl)(thien-2- | |
| | yl)methanone | |
| 509 | (7-{3-[2-(pyridin-2-ylmethyl)-2H-tetraazol-5- | ++ |
| | yl]phenyl}pyrazolo[1,5-a]pyrimidin-3-yl)(thien-2- | |
| | yl)methanone | |
| 510 | (7-{3-[2-(pyridin-4-ylmethyl)-2H-tetraazol-5- | +++ |
| | yl]phenyl}pyrazolo[1,5-a]pyrimidin-3-yl)(thien-2- | |
| | yl)methanone | |
| 511 | (7-{3-[2-(2-pyrrolidin-1-ylethyl)-2H-tetraazol-5- | ++ |
| | yl]phenyl}pyrazolo[1,5-a]pyrimidin-3-yl)(thien-2- | |
| | yl)methanone | |
| 512 | (7-{3-[2-(2-piperidin-1-ylethyl)-2H-tetraazol-5- | ++ |
| | yl]phenyl}pyrazolo[1,5-a]pyrimidin-3-yl)(thien-2- | |
| | yl)methanone | |
| 513 | (7-{3-[2-(2-hydroxyethyl)-2H-tetraazol-5- | ++ |
| | yl]phenyl}pyrazolo[1,5-a]pyrimidin-3-yl)(thien-2- | |
| | yl)methanone | |
| 514 | {7-[3-(1H-pyrazol-3-yl)phenyl]pyrazolo[1,5-a]pyrimidin-3- | ++ |
| | yl}(thien-2-yl)methanone | |
| 515 | (7-{3-[1-(cyclobutylmethyl)-1H-pyrazol-3- | +++ |
| | yl]phenyl}pyrazolo[1,5-a]pyrimidin-3-yl)(thien-2- | |
| | yl)methanone | |
| 516 | (7-{3-[1-(2-morpholin-4-ylethyl)-1H-pyrazol-3- | +++ |
| | yl]phenyl}pyrazolo[1,5-a]pyrimidin-3-yl)(thien-2- | |
| | yl)methanone | |
| 517 | (7-{3-[1-(pyridin-2-ylmethyl)-1H-pyrazol-3- | ++ |
| | yl]phenyl}pyrazolo[1,5-a]pyrimidin-3-yl)(thien-2- | |
| | yl)methanone | |
| 518 | {7-[3-(1-ethyl-1H-pyrazol-3-yl)phenyl]pyrazolo[1,5- | +++ |
| | a]pyrimidin-3-yl}(thien-2-yl)methanone | |
| 519 | (7-{3-[1-(2-piperidin-1-ylethyl)-1H-pyrazol-3- | ++ |
| | yl]phenyl}pyrazolo[1,5-a]pyrimidin-3-yl)(thien-2- | |
| | yl)methanone | |
| 520 | (7-{3-[1-(2-pyrrolidin-1-ylethyl)-1H-pyrazol-3- | ++ |
| | yl]phenyl}pyrazolo[1,5-a]pyrimidin-3-yl)(thien-2- | |
| | yl)methanone | |

+++, <0.1 μM;
++, 0.1-1.0 μM;
+; 1.0-20 μM

Experimental
General Procedures

General Procedures 1-8 were used for compounds 1-222. The specifically named product for compounds prepared via these procedures can be found in Table 1. The procedures below are not intended to limit the scope of the invention in any way.

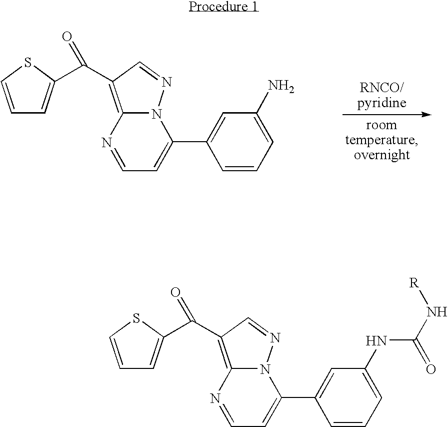

Examples 1-12 were synthesized in parallel as described below: [7-(3—aminophenyl)pyrazolo[1,5-a]pyrimidin-3-yl]-2-thienyl-methanone (0.1 mmole, 32 mg) was dissolved in 2 mL of pyridine and treated with appropriate isocyanate (0.12 mmole) at room temperature. The reaction was stirred at room temperature overnight and the volatiles were removed under reduced pressure. The resulting residue was dissolved in a 1:1 mixture of dimethyl sulfoxide/acetonitrile and purified by preparative HPLC. The fractions were analyzed by LC/MS to identify the product. The mass spectrum data of the isolated products are provided in Table 1.

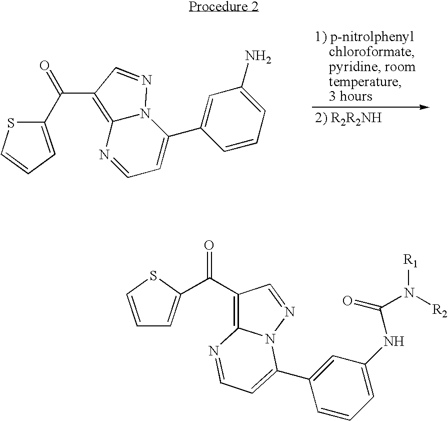

Examples 46, 155-170 were prepared in parallel as described below: [7-(3-aminophenyl)pyrazolo[1,5-a]pyrimidin-3-yl]-2-thienyl-methanone (0.1 mmole, 32 mg) was dissolved in 2 mL of pyridine and treated with p-nitrophenyl chloroformate (0.15 mmole). The mixture was stirred at room temperature for 3 h and treated with appropriate amine (0.2 mmole) and stirring was continued at room temperature overnight. The volatiles were removed under reduced pressure. The resulting residue was dissolved in a 1:1 mixture of dimethyl sulfoxide/acetonitrile and purified by preparative HPLC. The fractions were analyzed by LC/MS to identify the product. The mass spectrum data of the isolated products are provided in Table 1.

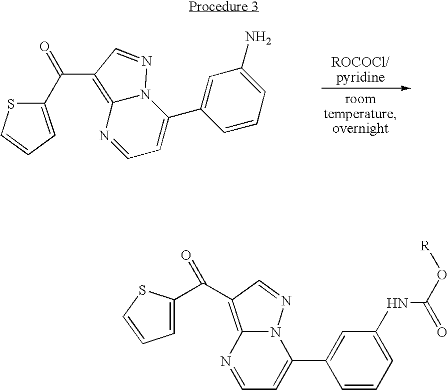

Examples 13-26 and 194-200 were prepared in parallel as described below: [7-(3-aminophenyl)pyrazolo[1,5-a]pyrimidin-3-yl]-2-thienyl-methanone (0.1 mmole, 32 mg) was dissolved in 2 mL of pyridine and was treated with appropriate chloroformates (0.12 mmole) at room temperature. The reaction was stirred at room temperature overnight and the volatiles were removed under reduced pressure. The resulting residue was dissolved in a 1:1 mixture of dimethylsulfoxide/acetonitrile and purified by preparative HPLC. The fractions were analyzed by LC/MS to identify the product. The mass spectrum data of the isolated products are provided in Table 1.

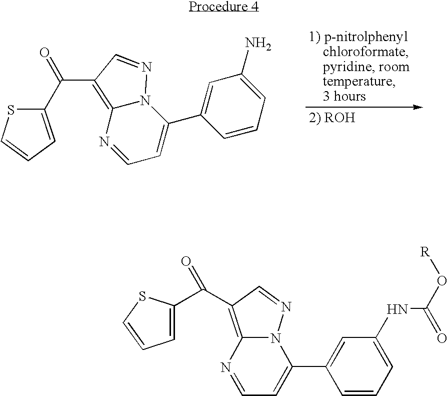

Examples 171-193 and 208-214 were prepared in parallel using the following procedure: [7-(3-aminophenyl)pyrazolo[1,5-a]pyrimidin-3-yl]-2-thienyl-methanone (0.1 mmole, 32 mg) was dissolved in 2 mL of pyridine and treated with p-nitrophenyl chloroformate (0.15 mmole). The mixture was stirred at room temperature for 3 h and treated with appropriate alcohol (0.2 mmole) and stirring was continued overnight. Volatiles were removed under reduced pressure. The resulting residue was dissolved in a 1:1 mixture of dimethyl sulfoxide/acetonitrile and purified by preparative HPLC. The fractions were analyzed by LC/MS to identify the product. The mass spectrum data of the isolated products are provided in Table 1.

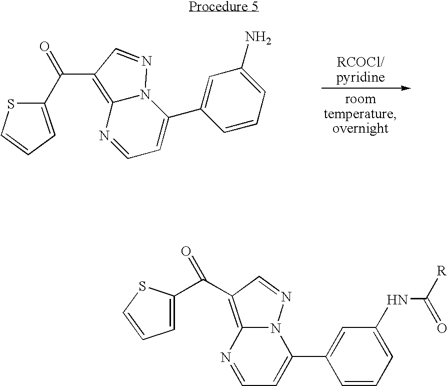

Examples 41-54, 87-153, 201-205, and 215-220 were synthesized in parallel by the procedure described below: [7-(3-aminophenyl)pyrazolo[1,5-a]pyrimidin-3-yl]-2-thienyl-methanone (0.1 mmole, 32 mg) was dissolved in 2 mL of pyridine and was treated with appropriate carboxylic acid chlorides (0.12 mmole) at room temperature. The reaction mixture was stirred at room temperature overnight and the volatiles were removed under reduced pressure. The resulting residue was dissolved in a 1:1 mixture of dimethylsulfoxide/acetonitrile and purified by preparative HPLC. The fractions were analyzed by LC/MS to identify the product. The mass spectrum data of isolated products are provided in Table 1.

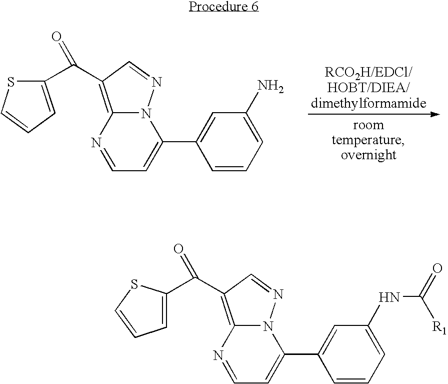

Examples 77-86 were also prepared in parallel by following procedure: [7-(3-aminophenyl)pyrazolo[1,5-a]pyrimidin-3-yl]-2-thienyl-methanone (0.1 mmole, 32 mg) was dissolved in 2 mL of DMF and was treated with carboxylic acids (0.15 mmole) in the presence of EDCI (0.15 mmol), HOBT (0.15 mmol) and DIEA (0.3 mmol). The reaction mixture was stirred at room temperature overnight and the volatiles were removed under reduced pressure. The resulting residue was dissolved in a 1:1 mixture of dimethylsulfoxide and acetonitrile, and was purified by preparative HPLC. The fractions were analyzed by LC/MS to identify the product. The mass spectrum data of the isolated products are provided in Table 1.

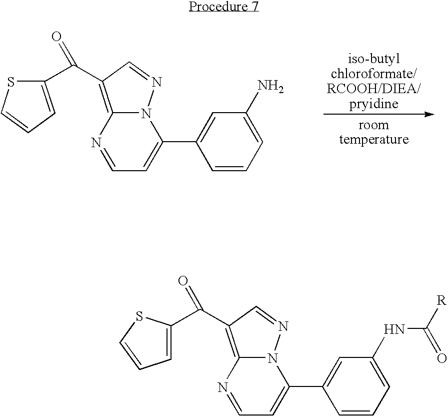

Examples 55-76, 206-7, 221-222 were prepared alternatively in parallel by the following procedure: [7-(3-aminophenyl)pyrazolo[1,5-a]pyrimidin-3-yl]-2-thienyl-methanone (0.1 mmole) was dissolved in 2 mL of pyridine and was treated with anhydrides formed from carboxylic acids (0.15 mmole), isobutyryl chloroformate (0.15) and DIEA (0.3 mmol) using tetrahydrofuran as the solvent. The reaction mixture was stirred at room temperature overnight and the volatiles were removed under reduced pressure. The resulting residue was dissolved in a 1:1 mixture of dimethylsulfoxide and acetonitrile, and was purified by preparative HPLC. The fractions were analyzed by LC/MS to identify the product. The mass spectrum data of the isolated products are provided in Table 1.

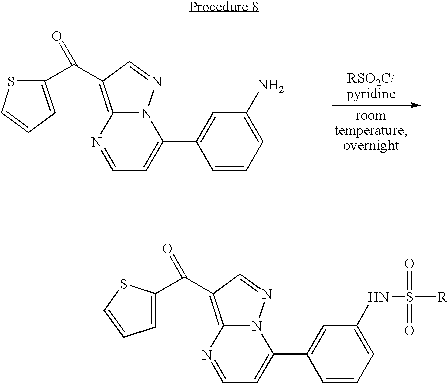

Examples 27-40 were synthesized in parallel by the following procedure: [7-(3-aminophenyl)pyrazolo[1,5-a]pyrimidin-3-yl]-2-thienyl-methanone (0.1 mmole) was dissolved in 2 mL of pyridine and was treated with appropriate sulfonyl chlorides (0.12 mmole) at room temperature. The reaction was stirred at room temperature overnight and the volatiles were removed under reduced pressure. The resulting residue was dissolved in a 1:1 mixture of dimethyl sulfoxide and acetonitrile and purified by preparative HPLC. The fractions were analyzed by LC/MS to identify the product. The mass spectrum data of the isolated products are provided in Table 1.

### DETAILED PROCEDURES

Specific compounds are identified by both an example number and their IUPAC name. Exemplary methods are labeled A-P. The structures are set forth in the table following the examples. In the event the IUPAC name is unclear or inconsistent, the structure prevails over name.

Reference Method A

### [7-(3-Nitrophenyl)pyrazolo[1,5-a]pyrimidin-3-yl]-2-thienyl-methanone

A mixture of (3-amino-1H-pyrazol-4-yl)-2-thienyl-methanone (2.0 g, 10.4 mmol) and 3-(dimethylamino)-1-(3-nitrophenyl)-2-propen-1-one (2.24 g, 10.4 mmol) in acetic acid (10 mL) was heated at reflux for 3 hours. The reaction mixture was cooled to room temperature and slurried with water. The thick suspension was filtered, washed thoroughly with water and dried to give 3.38 g (93%) of [7-(3-nitrophenyl)pyrazolo[1,5-a]pyrimidin-3-yl]-2-thienyl-methanone as a white solid, mp 199-201° C.

The preparation of (3-amino-1H-pyrazol-4-yl)-2-thienyl-methanone is described in DE patent 3422876.

Reference Method B

### 3-(Dimethylamino)-1-(3-nitrophenyl)-2-propen-1-one

3-Nitroacetophenone (5.0 g, 30.3 mmol) in dimethylformamide-dimethylacetal (10 mL) was heated at reflux overnight. The reaction mixture was cooled to room temperature and evaporated to remove the volatiles. The residue was slurried in ethyl ether and the suspension was filtered and washed with ether to give 10.5 g (79%) of 3-(dimethylamino)-1-(3-nitrophenyl)-2-propen-1-one, 104-105° C.

Reference Method C

[7-(3-Aminophenyl)pyrazolo[1,5-a]pyrimidin-3-yl]-2-thienyl-methanone A 2.0 L three neck flask equipped with mechanical stirrer was charged [7-(3-nitrophenyl)pyrazolo[1,5-a]pyrimidin-3-yl]-2-thienyl-methanone (30 g, 86 mmol), and ammonium chloride (23 g, 428 mmol) in methanol (200 mL) and water (200 mL). The mixture was stirred for 5 minutes. Iron powder (19.1 g, 343 mmol) was added slowly with stirring followed by an additional 200 mL of methanol and 200 mL of water. The reaction mixture was heated gradually to reflux and maintained at reflux overnight, cooled to room temperature and filtered. The red solid cake was washed thoroughly with hot methanol and hot ethyl acetate. The combined filtrates were evaporated to give 20.1 g (74%) of [7-(3-aminophenyl)pyrazolo[1,5-a]pyrimidin-3-yl]-2-thienyl-methanone as a light brown solid, mp 183-184° C. The crude product was used directly for the next step without further purification.

Reference Method D

### 2-Fluoro-N-{3-[3-(thien-2-ylcarbonyl)pyrazolo[1,5-a]pyrimidin-7-yl]phenyl}benzamide

To a mixture of [7-(3-aminophenyl)pyrazolo[1,5-a]pyrimidin-3-yl]-2-thienyl-methanone (100 mg, 0.31 mmol), 4-dimethylaminopyridine (4 mg, 0.03 mmol) and triethylamine (47 mg, 0.47 mmol) in methylene chloride (2 mL) was added o-fluorobenzoyl chloride (60 mg, 0.38 mmol) in methylene chloride (1 mL) via syringe. The resulting mixture was stirred at room temperature for 2 hours, diluted with methylene chloride (100 mL) and washed with saturated sodium bicarbonate and water. After drying over sodium sulfate, the methylene chloride solution was concentrated and the residue was column chromatographed using ethyl acetate/hexanes (1:2) as the eluting solvent to give 93 mg (67%) of 2-Fluoro-N-{3-[3-(thien-2-ylcarbonyl)pyrazolo[1,5-a]pyrimidin-7-yl]phenyl}benzamide as an off-white solid, mp, 190-191° C.

Reference Method E

### N-{3-[3-(2-Thienylcarbonyl)pyrazolo[1,5-a]pyrimidin-7yl]phenyl}isonicotinamide

The mixture of isonicotinic acid (15 mg, 0.94 mmol) and N-methylmorpholine (108 mg, 1.1 mmol) in methylene chloride (2 mL) was cooled with an ice-water bath. A solution of isobutyl chloroformate (128 mg, 0.94 mmol) in methylene chloride (1 mL) was added via syringe. The resulting mixture was stirred at 0-5° C. for 2 hours before adding [7-(3-aminophenyl)pyrazolo[1,5-a]pyrimidin-3-yl]-2-thienyl-methanone (100 mg, 0.31 mmol), triethylamine (47 mg, 0.47 mmol) and 4-dimethylaminopyridine (catalytic amount). The resulting mixture was stirred at room temperature overnight, diluted with methylene chloride (100 mL) and washed with saturated sodium bicarbonate and water. After drying over sodium sulfate, the methylene chloride solution was concentrated and the residue was column chromatographed eluting with a mixture of methanol and methylene chloride to give 85 mg (64%) of N-{3-[3-(2-thienylcarbonyl)pyrazolo[1,5-a]pyrimidin-7-yl]phenyl}isonicotinamide as an off-white solid, mp, 212-215° C.

Reference Method F

### {7-[3-(Benzylamino)phenyl]pyrazolo[1,5-a]pyrimidin-3-yl}(thien-2-yl)methanone;

[7-(3-Aminophenyl)pyrazolo[1,5-a]pyrimidin-3-yl]-2-thienyl-methanone (160 mg, 0.5 mmol) and benzaldehyde (56 mg, 0.53 mmol) were mixed in methylene chloride (5.0 mL) at room temperature. Sodium triacetoxyborohydride (530 mg, 2.5 mmol) was added in portions over 30 minutes. The resulting mixture was stirred at room temperature overnight and partitioned between water and ethyl acetate. The combined organics were dried over sodium sulfate and concentrated to give a syrupy residue which was column chromatographed eluting with 1:1 ethyl acetate/hexanes to give 117 mg (73%) of the title compound as a light yellow solid, mp 90-92° C.

Reference Method G

### N-Butyl-N′-{3-[3-(thien-2-ylcarbonyl)pyrazolo[1,5-a]pyrimidin-7-yl)phenyl}urea

[7-(3-Aminophenyl)pyrazolo[1,5-a]pyrimidin-3-yl]-2-thienyl-methanone (1.2 g, 3.75 mmol) was suspended in tetrahydrofuran (100 mL). Butyl isocyanate (1.5 g, 15.0 mmol) was added. Triethylamine (0.76 g, 7.5 mmol) was added with stirring. The resulting mixture was heated to reflex overnight and evaporated. The residue was dissolved in methylene chloride and washed with saturated aqueous sodium bicarbonate. The organic layer was seperated, dried over magnesium sulfate, concentrated and column chromatographed eluting with a gradient mixture of methanol and methylene chloride to give 1.11 g (69%) of the title compound as an off-white solid, mp 158-160° C.

Reference Method H

### 2,2,2-Trichloroethyl 3-[3-(thien-2-ylcarbonyl)pyrazolo[1,5-a]pyrimidin-7-yl]phenylcarbamate

[7-(3-Aminophenyl)pyrazolo[1,5-a]pyrimidin-3-yl]-2-thienyl-methanone (410 mg, 1.28 mmol) was suspended in dry tetrahydrofuran (10 mL) followed by addition of triethylamine (142 mg, 1.40 mmol). The mixture was stirred at room temperature for 30 minutes. 2,2,2-Trichloroethyl chloroformate (271 mg; 1.28 mmol) was added slowly and the reaction mixture was stirred overnight at room temperature. The volatiles were removed by evaporation under reduced pressure and the residue was partitioned between water and ether/ethyl acetate. The combined organics were dried over magnesium sulfate and concentrated to give a yellowish semi-solid. The crude solid was recrystalized from ethyl acetate to give 478 mg (75%) of the title compound as an off-white solid, mp 197-200° C.

Reference Method I

### 3-(1,1-Dioxidothiomorpholin-4-yl)propyl 3-[3-(thien-2-ylcarbonyl)pyrazolo[1,5-a]pyrimidin-7-yl]phenylcarbamate

p-Nitrophenyl chloroformate (314 mg, 1.57 mmol) was dissolved in methylene chloride (2.5 mL) and cooled to 0-5° C. 3-(1,1-Dioxido-4-thiomorpholinyl)-1-propanol (GB patent 0000373) (302 mg, 1.57 mmol) and 4-methylmorpholine (237 mg, 2.35 mmol) were added and the reaction mixture was stirred with cooling for 2 hours. [7-(3-Aminophenyl)pyrazolo[1,5-a]pyrimidin-3-yl]-2-thienyl-methanone (100 mg, 0.31 mmol) was added, followed by addition of pyridine (2.5 mL) and 4-dimethylaminopyridine (catalytic amount). The resultant mixture was heated at 100-105° C. for 2 hours and cooled to room temperature, and diluted with methylene chloride. The organic solution was washed with saturated aqueous sodium carbonate and water, dried over sodium sulfate and concentrated on silica gel. Flash column chromatography afforded 99 mg (59%) of the title compound as an off-white solid, mp 148-159° C.

Reference Method J

### N-Isobutyl-3-[3-(2-thienylcarbonyl)pyrazolo[1,5-a]pyrimidin-7-yl]benzamide

A mixture of 3-[3-(2-thienylcarbonyl)pyrazolo[1,5-a]pyrimidin-7-yl]benzoic acid (150 mg, 0.43 mmol), diisopropylethylamine (167 mg, 1.3 mmol), benzotriazole-1-yloxy-tripyrrolidinophosphonium hexafluorophosphate (290 mg, 0.56 mmol) and isobutylamine (31 mg, 0.43 mmol) in methylene chloride (4 mL) was stirred at room temperature for 5 hours. The reaction mixture was partitioned between saturated aqueous sodium bicarbonate and methylene chloride. The combined organics were dried over sodium sulfate, concentrated and purified by flash column chromatography eluting with a gradient mixture of methanol and methylene chloride to give 170 mg (98%) of the title compound as a white solid.

Preparation of 3-[3-(2-thienylcarbonyl)pyrazolo[1,5-a]pyrimidin-7-yl]benzoic acid: a mixture of methyl 3-[3-(2-thienylcarbonyl)pyrazolo[1,5-a]pyrimidin-7-yl]benzoate (177 mg, 0.49 mmol), potassium hydroxide (1.0 M, 5.0 mL) and methanol (0.3 mL) was stirred at room temperature overnight and then heated at 50° C. for 15 minutes. The reaction mixture was cooled to room temperature and solid precipitated. The resulting suspension was diluted with water until a solution and hydrochloric acid (conc.) was added until acidic pH. The precipitate was collected, washed with water and dried to give 154 mg (90%) of the title compound as a light yellow solid, mp 174-177° C.

Reference Method K

### (5-Amino-1H-pyrazol-4-yl)(thien-2-yl)methanone

A mixture of 2-acetylthiophene (30.2 g, 239 mmol) and dimethyl formamide-dimethyl acetal 990 mL) was heated at reflux for 15 hours. The reaction mixture was cooled and the volatiles were removed under reduced pressure. The residue was slurried in ether, filtered and washed with ether to give 39.35 g of 3-(dimethylamino)-1-(2-thienyl)-2-propen-1-one as a light orange solid, which was then treated with hydroxylamine hydrochloride (18.1 g, 260 mmol) in methanol (200 mL). The above mixture was heated at reflux for 3 hours and at room temperature overnight to result in a clear yellow solution. Dimethyl formamide-dimethyl acetal (90 mL) was added and the resultant mixture was stirred at reflux for 3 hours. After cooling to room temperature, the precipitate was collected by filtration and washed with ether to give 36.4 g (74%) of α-[(dimethylamino)methylene]-β-oxo-2-thiophenyl-propanenitrile as an orange crystal.

A mixture of α-[(dimethylamino)methylene]-β-oxo-2-thiophenyl-propanenitrile (30.9 g, 150 mmol), aminoguanidine nitrate (24.7 g, 180 mmol) and 10 N sodium hydroxide (18 mL) in ethanol (450 mL) was heated at reflux for 3.5 hours. The reaction mixture was cooled to room temperature and the volatiles were evaporated under reduced pressure. The residue was diluted with water and cooled with an ice-water bath. The precipitate was collected by filtration and washed with ethanol. More solids were obtained from the filtrate after reducing the volume and diluting with water to give a total of 22.2 g (76%) of the title compound as an off-white solid, mp 114-115° C.

Reference Method L

### α-[(Dimethylamino)methylene]-β-oxo-2-furane-propanenitrile

A 50 mL portion of dimethylformamide-dimethylacetal was added to p-oxo-2-furanepropanenitrile (25 g, 198 mmol) slowly. The reaction mixture was stirred at room temperature for 2 hours and the volatiles were removed under reduced pressure. The residue was dissolved in methylene chloride and the solution was passed through a short pad of hydrous magnesium silicate. The eluate was refluxed with the gradual addition of hexanes to the point of turbidity. Cooling and filtration gave 35.2 g of the title compound, mp 117-125° C.

Reference Method M

### {7-[4′-(Dimethylamino)-1,1′-biphenyl-3-yl]pyrazolo[1,5-a]pyrimidin-3-yl}(thien-2-yl)methanone

A mixture of 4-N,N-dimethyaminophenyl boronic acid (0.22 g, 1.3 mmol), [7-(3-bromophenyl)pyrazolo[1,5-a]pyrimidin-3-yl]-2-thienyl-methanone (0.25 g, 0.65 mmol), Pd(PPh₃)₄ (40 mg, 0.03 mmol) and Na₂CO₃ (aq) (2.0 M, 0.33 mL) in EtOH (0.5 mL)/H₂O (0.7 mL)/DME (2 mL) was irradiated in a microwave at 100° C. for 4 minutes. After cooling to room temperature, the reaction mixture was diluted with CHCl₃ (200 mL), washed with water and saturated aqueous NaHCO₃, dried over Na₂SO₄ and concentrated. The residue was purifed by silica gel flash column chromatography to give 598 mg (72%) of the desired product as a yellow solid, mp 200-202° C.

[7-(3-Bromophenyl)pyrazolo[1,5-a]pyrimidin-3-yl]-2-thienyl-methanone is prepared using a procedure similar to Method A from (5-amino-1H-pyrazol-4-yl)(thien-2-yl)methanone and 1-(3-bromo-phenyl)-3-dimethylamino-propenone, mp 193-195° C.

Reference Method N

### {7-[4′-(Morpholin-4-ylmethyl)-1,1′-biphenyl-3-yl]pyrazolo[1,5-a]pyrimidin-3-yl}(thien-2-yl)methanone

A mixture of 3′-[3-(thien-2-ylcarbonyl)pyrazolo[1,5-a]pyrimidin-7-yl]-1,1′-biphenyl-4-carbaldehyde (100 mg, 0.24 mmol) and morpholine (106 mg, 1.22 mmol) was dissolved in CH₂Cl₂ (15 mL)/DMF (0.2 mL) and stirred for 10 minutes, with the cooling of ice water bath and. Sodium triacetoxyborohydride (311 mg, 1.5 mmol) was added and the mixture was stirred from 30 minutes. Acetic acid (0.2 mL) was added, followed by another 2.5 hours of stirring. The resulting mixture was quenched with water and the organic phase was washed with saturated aqueous NaHCO₃ and water and evaporated. The residue was purifed with silica gel flash column chromatography to give 88 mg (75%) of the desired product as a gray crystal, mp 175-178° C.

Reference Method O

### 3-(3,5-Dimethylphenyl)-7-[3-(1H-tetraazol-5-yl)phenyl]pyrazolo[1,5-a]pyrimidine (WAY-189354)

A mixture of 3-[3-(3,5-dimethylphenyl)pyrazolo[1,5-a]pyrimidin-7-yl]benzonitrile (2.51 g, 7.75 mmol), sodium azide (2.76 g, 42.5 mmol) and ammonium chloride (2.3 g, 5.55 mmol) in DMF (17 mL) was heated at 100° C. under nitrogen overnight. The reaction mixture was cooled to room temperature and was diluted with water. The precipitation was collected by filtration, washed with water and ether to give 2.4 g (84%) of desired product as a red solid, mp 250° C. (dec.).

Reference Method P

### 7-{3-[2-(Cyclobutylmethyl)-2H-tetraazol-5-yl]phenyl}-3-(3,5-dimethylphenyl)pyrazolo[1,5-a]pyrimidine

3-(3,5-Dimethylphenyl)-7-[3-(1H-tetraazol-5-yl)phenyl]pyrazolo[1,5-a]pyrimidine (150 mg, 0.41 mmol) was dissolved in acetonitrile (6 mL) an DMF (6 mL). Cs₂CO₃ (0.2 g, 0.61 mmol) was added followed by cyclobutylmethyl bromide (0.064 mL, 0.57 mmol). The resulting mixture was heated at reflux for 48 hours and partitioned between water and ethyl acetate. The combined organics were dried over Na₂SO₄ and concentrated. The residue was purified by silica gel flash column chromatograph to give 120 mg (67%) of the desired product as a yellow foam, mp 64° C.

### EXAMPLE 223

### [7-(3-Trifluoromethylphenyl)pyrazolo[1,5-a]-pyrimidin-3-yl}-2-thienyl-methanone

The preparation of example 223 was described in EP patent 129847.

### EXAMPLE 224

### 2-Thienyl[7-(2-thienyl)pyrazolo[1,5-a]-pyrimidin-3-yl}-methanone

2-Thienyl[7-(2-thienyl)pyrazolo[1,5-a]-pyrimidin-3-yl}-methanone is prepared using a procedure similar to Method A from 3-(dimethylamino)-1-(2-thienyl)-2-propen-1-one and (3-amino-1H-pyrazol-4-yl)-2-thienyl-methanone, mp 193-195° C. The preparation of 3-(dimethylamino)-1-(2-thienyl)-2-propen-1-one is described in U.S. Pat. No. 4,374,988.

### EXAMPLE 225

### [7-(3-Fluorophenyl)pyrazolo[1,5-a]-pyrimidin-3-yl}-2-thienyl-methanone

The title compound was prepared using a procedure similar to Method A from 3-(dimethylamino)-1-(3-fluorophenyl)-2-propen-1-one and (3-amino-1H-pyrazol-4-yl)-2-thienyl-methanone, mp 235-237° C.

3-(Dimethylamino)-1-(3-fluorophenyl)-2-propen-1-one was prepared using a procedure similar to Method B by treating 3-fluoroacetophenone with dimethyl formamide-dimethyl acetal.

### EXAMPLE 226

### 1-[3-[3-(2-Furanylcarbonyl)pyrazolo-[1,5-a]pyrimidin-7-yl]phenyl}-2-piperidinone

The title compound was prepared using a procedure similar to Method A from 1-[3-[3-(dimethylamino)-1-oxo-2-propenyl]phenyl-2-piperidinone and (3-amino-1H-pyrazol-4-yl)-2-furanyl-methanone, mp 159-160° C.

1-[3-[3-(Dimethylamino)-1-oxo-2-propenyl]phenyl-2-piperidino was prepared using a procedure similar to Method B by treating 1-(3-acetylphenyl)tetrahydro-2(1H)-pyridinone with dimethyl formamide-dimethyl acetal.

### EXAMPLE 227

### 1-[3-(3-Benzoylpyrazolo[1,5-a]-pyrimidin-7-yl)phenyl]-2-piperidinone

The title compound was prepared using a procedure similar to Method A from 1-[3-[3-(dimethylamino)-1-oxo-2-propenyl]phenyl-2-piperidinone and (3-amino-1H-pyrazol-4-yl)phenyl-methanone, mp 160-162° C.

### EXAMPLE 228

### 1-[3-[3-(2-Furanylcarbonyl)pyrazolo-[1,5-a]pyrimidin-7-yl]phenyl]-2-pyrrolidinone

The title compound was prepared using a procedure similar to Method A from 1-[3-[3-(dimethylamino)-1-oxo-2-propenyl]phenyl-2-pyrrolidinone and (3-amino-1H-pyrazol-4-yl)-2-furanyl-methanone, mp 210-211° C.

1-[3-[3-(Dimethylamino)-1-oxo-2-propenyl]phenyl-2 pyrrolidinone was prepared using a procedure similar to Method B by treating 1-(3-acetylphenyl)tetrahydro-2(1H)-pyrrolidinone with dimethyl formamide-dimethyl acetal.

### EXAMPLE 229

### 1-[3-(3-Benzoylpyrazolo[1,5-a]-pyrimidin-7-yl)phenyl]-2-pyrrolidinone

The title compound was prepared using a procedure similar to Method A from 1-[3-[3-(dimethyl amino)-1-oxo-2-propenyl] phenyl-2-pyrrolidinone and (3-amino-1H-pyrazol-4-yl)phenyl-methanone, mp 173-174° C.

### EXAMPLE 230

### N-[3-(3-Benzoylpyrazolo-[1,5-a]pyrimidin-7-yl)phenyl]-cyclopropanecarboxamide

The title compound was prepared using a procedure similar to Method A from N-[4-[3-(dimethylamino)-1-oxo-2-propenyl]phenyl}-cyclopropanecarboxamide and (3-amino-1H-pyrazol-4-yl)phenyl-methanone, mp 210-212° C.

N-[4-[3-(Dimethylamino)-1-oxo-2-propenyl]phenyl}-cyclopropanecarboxamide was prepared from N-(3-acetylphenyl)-cyclopropanecarboxamide and dimethyl formamide-dimethyl acetal.

### EXAMPLE 231

### [7-[3-(Methylamino)phenyl]pyrazolo[1,5-a]-pyrimidin-3-yl}phenyl-methanone

The title compound was prepared using a procedure similar to Method A from N-[3-(3-dimethylamino-1-oxo-2-propenyl)phenyl]-methylamine and (3-amino-1H-pyrazol-4-yl)phenyl-methanone, mp 134-136° C.

### EXAMPLE 232

### N-[3-(3-Benzoylpyrazolo-[1,5-a]pyrimidin-7-yl)phenyl]-N-methylcyclobutanecarboxamide

The title compound was prepared using a procedure similar to Method A from N-[3-(3-dimethylamino-1-oxo-2-propenyl)phenyl]-N-methyl-cyclobutanecarboxamide and (3-amino-1H-pyrazol-4-yl)phenyl-methanone, mp 168-170° C.

N-[3-(3-Dimethylamino-1-oxo-2-propenyl)phenyl]-N-methyl.-cyclobutanecarboxamide was prepared using an analogous procedure to Method B from N-(3-acetylphenyl)-N-methyl-cyclobutanecarboxamide and dimethyl formamide-dimethyl acetal.

### EXAMPLE 233

### N-[3-[3-(2-Furanylcarbonyl)-pyrazolo[1,5-a]pyrimidin-7-yl]phenyl}-N-methyl-cyclobutanecarboxamide

The title compound was prepared using a procedure similar to Method A from N-[3-(3-dimethylamino-1-oxo-2-propenyl)phenyl]-N-methyl-cyclobutanecarboxamide and (3-amino-1H-pyrazol-4-yl)furanyl-methanone, mp 166-167° C.

### EXAMPLE 234

### N-[3-(3-Cyanopyrazolo[1,5-a]-pyrimidin-7-yl)phenyl]-N-methyl-cyclobutanecarboxamide

The title compound was prepared using a procedure similar to Method A from N-[3-(3-dimethylamino-1-oxo-2-propenyl)phenyl]-N-methyl-cyclobutanecarboxamide and 3-amino-4-cyano-1H-pyrazol mp 157-158° C.

### EXAMPLE 235

### N-[3-[3-(2-Furanylcarbonyl)-pyrazolo[1,5-a]pyrimidin-7-yl]phenyl}-cyclopropanecarboxamide

The title compound was prepared using a procedure similar to Method A.

### EXAMPLE 236

### N-[3-(3-benzoylpyrazolo[1,5-a]-pyrimidin-7-yl)phenyl]-N-methyl-cyclopropanecarboxamide

The title compound was prepared using a procedure similar to Method A from N-[3-(3-dimethylamino-1-oxo-2-propenyl)phenyl]-N-methyl-cyclopropanecarboxamide and (3-amino-1H-pyrazol-4-yl)phenyl-methanone, mp 168-170° C.

### EXAMPLE 237

### N-Methyl-N-[3-[3-(2-thienylcarbonyl)-pyrazolo[1,5-a]pyrimidin-7-yl]phenyl}-propanamide

The title compound was prepared using a procedure similar to Method A from N-[4-[3-(dimethylamino)-1-oxo-2-propenyl]phenyl}-N-methyl-propanamide and (3-amino-1H-pyrazol-4-yl)-2-thienyl-methanone, mp 186-187° C.

N-[4-[3-(Dimethylamino)-1-oxo-2-propenyl]phenyl}-N-methyl-propanamide was prepared using an analogous procedure to Method B from N-(3-acetylphenyl)-N-methyl-propanamide and dimethyl formamide-dimethyl acetal.

### EXAMPLE 238

### N-Methyl-N-[3-[3-(2-thienylcarbonyl)-pyrazolo[1,5-a]pyrimidin-7-yl]phenyl}-acetamide

The title compound was prepared using a procedure similar to Method A from N-[4-[3-(dimethylamino)-1-oxo-2-propenyl]phenyl}-N-methyl-acetamide and (3-amino-1H-pyrazol-4-yl)-2-thienyl-methanone, mp 190-192° C.

N-[4-[3-(Dimethylamino)-1-oxo-2-propenyl]phenyl}-N-methyl-acetamide was prepared using an analogous procedure to Method B from N-(3-acetylphenyl)-N-methyl-acetamide and dimethyl formamide-dimethyl acetal.

### EXAMPLE 239

### Methyl [3-[3-(2-thienylcarbonyl)pyrazolo-[1,5-a]pyrimidin-7-yl]phenyl}-carbamic acid

The title compound was prepared using a procedure similar to Method A from methyl [3-[3-(dimethylamino)-1-oxo-2-propenyl]-phenyl]-carbamic acid and (3-amino-1H-pyrazol-4-yl)-2-thienyl-methanone, mp 215-217° C.

Methyl [3-[3-(dimethyl amino)-1-oxo-2-propenyl]-phenyl]-carbamic acid was prepared using an analogous procedure to Method B from methyl (3-acetylphenyl)-carbamic acid and dimethyl formamide-dimethyl acetal.

### EXAMPLE 240

### Ethyl 7-[3-[(cyclobutylcarbonyl)amino]phenyl]-pyrazolo[1,5-a]pyrimidine-3-carboxylic acid

The title compound was prepared using a procedure similar to Method A from N-[3-(3-dimethylamino-1-oxo-2-propenyl)phenyl]-cyclobutanecarboxamide and ethyl 3-amino-1H-pyrazole-4-carboxylic acid, mp 123-25° C.

### EXAMPLE 241

### N-[3-[3-(2-Furanylcarbonyl)pyrazolo-[1,5-a]pyrimidin-7-yl]phenyl}-2-methyl-propanamide

The title compound was prepared using a procedure similar to Method A from N-[4-[3-(dimethylamino)-1-oxo-2-propenyl]phenyl}-2-methyl-propanamide and (3-amino-1H-pyrazol-4-yl)-2-furanyl-methanone, mp 189-191° C.

### EXAMPLE 242

### N-[3-(3-Benzoylpyrazolo[1,5-a]-pyrimidin-7-yl)phenyl]-2,2-dimethyl-propanamide

The title compound was prepared using a procedure similar to Method A from N-[4-[3-(dimethylamino)-1-oxo-2-propenyl]phenyl}-2,2-dimethyl-propanamide and (3-amino-1H-pyrazol-4-yl)phenyl-methanone, mp 160-161° C.

### EXAMPLE 243

### N-[3-[3-(2-Furanylcarbonyl)pyrazolo-[1,5-a]pyrimidin-7-yl]phenyl}-2,2-dimethyl-propanamide

The title compound was prepared using a procedure similar to Method A from N-[4-[3-(dimethylamino)-1-oxo-2-propenyl]phenyl}-2,2-dimethyl-propanamide and (3-amino-1H-pyrazol-4-yl)-2-furanyl-methanone, mp 203-204° C.

### EXAMPLE 244

### [7-[3-(1H-imidazol-1-yl)phenyl]pyrazolo-[1,5-a]pyrimidin-3-yl]-2-thienyl-methanone

The preparation of [7-[3-(1H-imidazol-1-yl)phenyl]pyrazolo-[1,5-a]pyrimidin-3-yl]-2-thienyl-methanone is described in U.S. Pat. No. 5,538,977.

### EXAMPLE 245

### [7-[3-(2-Methyl-1H-imidazol-1-yl)phenyl]-pyrazolo[1,5-a]pyrimidin-3-vy]-2-thienyl-methanone

The preparation of [7-[3-(2-methyl-1H-imidazol-1-yl)phenyl]-pyrazolo[1,5-a]pyrimidin-3-yl]-2-thienyl-methanone is described in U.S. Pat. No. 5,538,977.

### EXAMPLE 246

### [7-(2,4-dimethoxyphenyl)pyrazolo[1,5-a]-pyrimidin-3-yl}-2-thienyl-methanone

The title compound was prepared using a procedure analogous to Method A from (3-amino-1H-pyrazol-4-yl)-2-thienyl-methanone (2.0 g, 10.4 mmol) and 3-(dimethylamino)-1-(2,4-dimethoxyphenyl)-2-propen-1-one, mp 146-148° C.

3-(Dimethylamino)-1-(2,4-dimethoxyphenyl)-2-propen-1-one was prepared from 2,4-dimethoxyacetophenone and dimethyl formamide-dimethyl acetal using a procedure analogous to Method B.

### EXAMPLE 247

### [7-[3-(4-Methyl-1H-imidazol-1-yl)phenyl]-pyrazolo[1,5-a]pyrimidin-3-yl]-2-thienyl-methanone

The preparation of the title compound is described in U.S. Pat. No. 5,538,977.

### EXAMPLE 248

### N,N′-diethyl-3-[3-(2-thienylcarbonyl)-pyrazolo[1,5-a]pyrimidin-7-yl]-benzamide

The title compound was prepared using a procedure similar to Method A from 3-[3-(dimethylamino)-1-oxo-2-propenyl]-N,N-diethyl-benzamide and (3-amino-1H-pyrazol-4-yl)-2-thienyl-methanone, mp 165-167° C.

3-[3-(Dimethylamino)-1-oxo-2-propenyl]-N,N-diethyl-benzamide was prepared using an analogous procedure to Method B from methyl (3-acetylphenyl)-N,N-diethyl-benzamide and dimethyl formamide-dimethyl acetal.

### EXAMPLE 249

### N-[3-[3-(2-Thienylcarbonyl)pyrazolo[1,5-a]pyrimidin-7-yl]phenyl}-cyclopropanecarboxamide

The title compound was prepared using a procedure similar to Method A from N-[3-(3-dimethylamino-1-oxo-2-propenyl)phenyl]-cyclopropanecarboxamide and (3-amino-1H-pyrazol-4-yl)-2-thienyl-methanone, mp 191-192° C.

### EXAMPLE 250

### N-[3-[3-(2-Thienylcarbonyl)pyrazolo[1,5-a]pyrimidin-7-yl]phenyl}-acetamide

The title compound was prepared using a procedure similar to Method A from N-[4-[3-(dimethylamino)-1-oxo-2-propenyl]phenyl}-acetamide and (3-amino-1H-pyrazol-4-yl)-2-thienyl-methanone, mp 245-247° C.

N-[4-[3-(Dimethylamino)-1-oxo-2-propenyl]phenyl}-N-acetamide was prepared using an analogous procedure to Method B from N-(3-acetylphenyl)-acetamide and dimethyl formamide-dimethyl acetal.

### EXAMPLE 251

### N-[3-[3-(2-Thienylcarbonyl)pyrazolo[1,5-a]pyrimidin-7-yl]phenyl}-propanamide

The title compound was prepared using a procedure similar to Method A from N-[4-[3-(dimethylamino)-1-oxo-2-propenyl]phenyl}-propanamide and (3-amino-1H-pyrazol-4-yl)-2-thienyl-methanone, mp 208-210° C.

N-[4-[3-(Dimethylamino)-1-oxo-2-propenyl]phenyl}-N-propanamide was prepared using an analogous procedure to Method B from N-(3-acetylphenyl)-propanamide and dimethyl formamide-dimethyl acetal.

### EXAMPLE 252

### N-[3-[3-[(5-Methyl-2-thienyl)carbonyl]pyrazolo[1,5-a]pyrimidin-7-yl]phenyl}-cyclopropanecarboxamide

The title compound was prepared using a procedure similar to Method A from N-[3-(3-dimethylamino-1-oxo-2-propenyl)phenyl]-cyclopropanecarboxamide and (3-amino-1H-pyrazol-4-yl)-2-(5-methylthienyl)-methanone, mp 243-245° C.

The preparation of (3-amino-1H-pyrazol-4-yl)(5-methyl-2-thienyl)-methanone was described in DE patent 3422876.

### EXAMPLE 253

### N-[3-(3-Benzoylpyrazolo[1,5-a]pyrimidin-7-yl)phenyl]-N-ethyl-cyclopropanecarboxamide

The title compound was prepared using a procedure similar to Method A from N-[3-(3-dimethylamino-1-oxo-2-propenyl)phenyl]-N-ethyl-cyclopropanecarboxamide and (3-amino-1H-pyrazol-4-yl)phenyl-methanone, mp 153-155° C.

### EXAMPLE 254

### N-Ethyl-N-(3-pyrazolo[1,5-a]pyrimidin-7-ylphenyl)-cyclopropanecarboxamide

The title compound was prepared using a procedure similar to Method A from N-[3-(3-dimethylamino-1-oxo-2-propenyl)phenyl]-N-ethyl-cyclopropanecarboxamide and 3-amino-1H-pyrazol, mp 127-129° C.

### EXAMPLE 255

### N-[3-(3-Chloropyrazolo[1,5-a]pyrimidin-7-yl)phenyl]-N-ethyl-cyclopropanecarboxamide

The title compound was prepared using a procedure similar to Method A from N-[3-(3-dimethylamino-1-oxo-2-propenyl)phenyl]-N-ethyl-cyclopropanecarboxamide and 3-amino-4-chloro-1H-pyrazol, mp 150-151° C.

### EXAMPLE 256

### N-[3-(3-Cyanopyrazolo[1,5-a]pyrimidin-7-yl)phenyl]-N-ethyl-cyclopropanecarboxamide

The title compound was prepared using a procedure similar to Method A from N-[3-(3-dimethylamino-1-oxo-2-propenyl)phenyl]-N-ethyl-cyclopropanecarboxamide and 3-amino-4-cyano-1H-pyrazol, mp 166-168° C.

### EXAMPLE 257

### N-Ethyl-N-[3-[3-(2-furanylcarbonyl)pyrazolo[1,5-a]pyrimidin-7-yl]phenyl}-cyclopropanecarboxamide

The title compound was prepared using a procedure similar to Method A from N-[3-(3-dimethylamino-1-oxo-2-propenyl)phenyl]-N-ethyl-cyclopropanecarboxamide and (3-amino-1H-pyrazol-4-yl)-2-furanyl-methanone, mp 182-184° C.

### EXAMPLE 258

### Ethyl 7-[3-[(cyclopropylcarbonyl)-2-propynylamino]phenyl]-pyrazolo[1,5-a]pyrimidine-3-carboxylic acid

The title compound was prepared using a procedure similar to Method A from N-[3-[3-(dimethylamino)-1-oxo-2-propenyl]phenyl}-N-2-propynyl-cyclopropanecarboxamide and ethyl 3-amino-1H-pyrazole-4-carboxylic acid, mp 163-164° C.

N-[3-[3-(Dimethylamino)-1-oxo-2-propenyl]phenyl}-N-2-propynyl-cyclopropanecarboxamide was prepared using an analogous procedure to Method B from N-(3-acetylphenyl)-N-propynyl-cyclopropanecarboxamide and dimethyl formamide-dimethyl acetal.

### EXAMPLE 259

### N-2-Propynyl-N-[3-[3-(2-thienylcarbonyl)pyrazolo[1,5-a]pyrimidin-7-yl]phenyl}-cyclopropanecarboxamide

The title compound was prepared using a procedure similar to Method A from N-[3-[3-(dimethylamino)-1-oxo-2-propenyl]phenyl}-N-2-propynyl-cyclopropanecarboxamide and (3-amino-1H-pyrazol-4-yl)-2-thienyl-methanone, mp 221-223° C.

### EXAMPLE 260

### N-[3-[3-(2-Thienylcarbonyl)pyrazolo[1,5-a]pyrimidin-7-yl]phenyl}-formamide

The title compound was prepared using a procedure similar to Method A from N-[4-[3-(dimethylamino)-1-oxo-2-propenyl]phenyl}-formamide and (3-amino-1H-pyrazol-4-yl)-2-thienyl-methanone, mp 233-236° C.

N-[4-[3-(Dimethylamino)-1-oxo-2-propenyl]phenyl}-formamide was prepared using an analogous procedure to Method B from N-(3-acetylphenyl)-formamide and dimethyl formamide-dimethyl acetal.

### EXAMPLE 261

### Formamide, N-methyl-N-[3-[3-(2-thienylcarbonyl) pyrazolo[1,5-a]pyrimidin-7-yl]phenyl}

The title compound was prepared using a procedure similar to Method A from N-[4-[3-(dimethylamino)-1-oxo-2-propenyl]phenyl}-N-methylformamide and (3-amino-1H-pyrazol-4-yl)-2-thienyl-methanone, mp 171-173° C.

N-[4-[3-(Dimethylamino)-1-oxo-2-propenyl]phenyl}-N-methylformamide was prepared using an analogous procedure to Method B from N-(3-acetylphenyl)-N-methylformamide and dimethyl formamide-dimethyl acetal.

### EXAMPLE 262

### [7-(3-Aminophenyl)pyrazolo[1,5-a]pyrimidin-3-yl]-2-thienyl-methanone

The preparation of the title compound was described in Method C.

### EXAMPLE 263

### [7-(3-Ethoxyphenyl)pyrazolo[1,5-a]pyrimidin-3-yl]-2-thienyl-methanone

The title compound was prepared using a procedure analogous to Method A from (3-amino-1H-pyrazol-4-yl)-2-thienyl-methanone and 3-(dimethylamino)-1-(3-ethoxyphenyl)-2-propen-1-one, mp 146-148° C.

3-(Dimethylamino)-1-(3-ethoxyphenyl)-2-propen-1-one was prepared from 3-thoxyacetophenone and dimethyl formamide-dimethyl acetal using an analogous procedure to Method B.

### EXAMPLE 264

### [7-[3-(2-Methoxyethoxy)phenyl]pyrazolo[1,5-a]pyrimidin-3-yl]-2-thienyl-methanone

The title compound was prepared using a procedure analogous to Method A from (3-amino-1H-pyrazol-4-yl)-2-thienyl-methanone and 3-(dimethylamino)-1-[3-(2-methoxy)ethoxyphenyl]-2-propen-1-one, mp 112-116° C.

3-(Dimethylamino)-1—[3-(2-methoxy)ethoxyphenyl]-2-propen-1-one was prepared from 3-ethoxyacetophenone and dimethyl formamide-dimethyl acetal using an analogous procedure to Method B.

### EXAMPLE 265

### [7-[3-(Cyclopentyloxy)phenyl]pyrazolo[1,5-a]pyrimidin-3-yl]-2-thienyl-methanone

The title compound was prepared using a procedure analogous to Method A from (3-amino-1H-pyrazol-4-yl)-2-thienyl-methanone and 3-(dimethylamino)-1-[3-(cyclopentyloxy)phenyl]-2-propen-1-one, mp 135-138° C.

3-(Dimethylamino)-1-[3-(cyclopentyloxy)phenyl]-2-propen-1-one was prepared from 3-cyclopentyloxyacetophenone and dimethyl formamide-dimethyl acetal using an analogous procedure to Method B.

### EXAMPLE 266

### [7-[3-(Methylamino)phenyl]pyrazolo[1,5-a]pyrimidin-3-yl]-2-thienyl-methanone

The title compound was prepared using an analogous procedure to Method G from [7-(3-aminophenyl)pyrazolo[1,5-a]pyrimidin-3-yl]-2-thienyl-methanone and formaldehyde, mp 188-190° C.

### EXAMPLE 267

### [7-[3-(Propylamino)phenyl]pyrazolo[1,5-a]pyrimidin-3-yl]-2-thienyl-methanone

The title compound was prepared using an analogous procedure to Method G from [7-(3-aminophenyl)pyrazolo[1,5-a]pyrimidin-3-yl]-2-thienyl-methanone and propionaldehyde, mp 128-130° C.

### EXAMPLE 268

### N-Methyl-N-[3-[3-(4-methylbenzoyl)pyrazolo[1,5-a]pyrimidin-7-yl]phenyl}-cyclopropanecarboxamide

The title compound was prepared using a procedure similar to Method A from N-[3-(3-dimethylamino-1-oxo-2-propenyl)(4-methylphenyl)]-N-methyl-cyclopropanecarboxamide and (3-amino-1H-pyrazol-4-yl)phenyl-methanone, mp 189-190° C.

### EXAMPLE

### 2-Furanyl[7-[3-(1H-pyrrol-1-yl)phenyl]pyrazolo[1,5-a]pyrimidin-3-yl]-methanone

The title compound was prepared using a procedure analogous to Method A from (3-amino-1H-pyrazol-4-yl)-2-furanyl-methanone and 3-(dimethylamino)-1-[3-(1H-pyrrol-1-yl)phenyl]-2-propen-1-one, mp 204-106° C.

3-(Dimethylamino)-1-[3-(1H-pyrrol-1-yl)phenyl]-2-propen-1-one was prepared from 3-(1H-pyrrol-1-yl)acetophenone and dimethyl formamide-dimethyl acetal using an analogous procedure to Method B.

### EXAMPLE 270

### [7-[3-(1H-Pyrrol-1-yl)phenyl]pyrazolo[1,5-a]pyrimidin-3-yl]-2-thienyl-methanone

The title compound was prepared using a procedure analogous to Method A from (3-amino-1H-pyrazol-4-yl)-2-thienyl-methanone and 3-(dimethylamino)-1-[3-(1H-pyrrol-1-yl)phenyl]-2-propen-1-one, mp 211-213° C.

### EXAMPLE 271

### [7-[4-(Ethylamino)phenyl]pyrazolo[1,5-a]pyrimidin-3-yl]-2-thienyl-methanone

The title compound was prepared using an analogous procedure to Method G from [7-(3-aminophenyl)pyrazolo[1,5-a]pyrimidin-3-yl]-2-thienyl-methanone and acetaldehyde, mp 130-132° C.

### EXAMPLE 272

### N-{3-[3-(2-Thienylcarbonyl)pyrazolo[1,5-a]pyrimidin-7-yl]phenyl}acrylamide

The title compound was prepared using a procedure analogous to Method D from [7-(3-aminophenyl)pyrazolo[1,5-a]pyrimidin-3-yl]-2-thienyl-methanone and acryloyl chloride, mp 192-194° C.

### EXAMPLE 273

### 2-Methyl-N-{3-[3-(2-thienylcarbonyl)pyrazolo[1,5-a]pyrimidin-7-yl]phenyl}propanamide

The title compound was prepared using a procedure analogous to Method D from [7-(3-aminophenyl)pyrazolo[1,5-a]pyrimidin-3-yl]-2-thienyl-methanone and isobutyryl chloride, mp 180-181° C.

### EXAMPLE 274

### N-{3-[3-(2-Thienylcarbonyl)pyrazolo[1,5-a]pyrimidin-7-yl]phenyl}-2-butynamide

The title compound was prepared using an analogous procedure to Method E from [7-(3-aminophenyl)-5-methyl-pyrazolo[1,5-a]pyrimidin-3-yl]-2-thienyl-methanone and 2-butynoic acid, mp 202-203° C.

### EXAMPLE 275

### Thienylcarbonyl)pyrazolo[1,5-a]pyrimidin-7-yl]phenyl}cyclobutanecarboxamide

The title compound was prepared using a procedure analogous to Method D from [7-(3-aminophenyl)pyrazolo[1,5-a]pyrimidin-3-yl]-2-thienyl-methanone and cyclobutanecarbonyl chloride, mp 202-203° C.

### EXAMPLE 276

### N-{3-[5-Methyl-3-(2-thienylcarbonyl)pyrazolo[1,5-a]pyrimidin-7-yl]phenyl}cyclopropanecarboxamide

The title compound was prepared using a procedure analogous to Method D from [7-(3-aminophenyl-5-methyl)pyrazolo[1,5-a]pyrimidin-3-yl]-2-thienyl-methanone and cyclopropanecarbonyl chloride, mp 184-186° C.

[7-(3-Aminophenyl-5-methyl)pyrazolo[1,5-a]pyrimidin-3-yl]-2-thienyl-methanone is prepared using an analogous procedure to Method A and C from (3-amino-1H-pyrazol-4-yl)-(2-thienyl)-methanone and 3-(dimethylamino)-1-(3-nitrophenyl)-2-butene-1-one. 3-(Dimethylamino)-1-(3-nitrophenyl)-2-butene-1-one is prepared using an analogous procedure to method B from 3-nitroacetophenone and dimethyl acetamide-dimethyl acetal.

### EXAMPLE 277

### N-{3-[3-(2-Thienylcarbonyl)pyrazolo[1,5-a]pyrimidin-7-yl]phenyl}-2-oxiranecarboxamide

The title compound was prepared using an analogous procedure to Method E from [7-(3-aminophenyl)-5-methyl-pyrazolo[1,5-a]pyrimidin-3-yl]-2-thienyl-methanone and glycidic acid, mp 70° C. (decomposition).

### EXAMPLE 278

### {3-[5-Methyl-3-(2-thienylcarbonyl)pyrazolo[1,5-a]pyrimidin-7-yl]phenyl}-2-oxiranecarboxamide

The title compound was prepared using an analogous procedure to Method D from [7-(3-aminophenyl)-5-methyl-pyrazolo[1,5-a]pyrimidin-3-yl]-2-thienyl-methanone and cyclopropanecarbonyl chloride, mp 70° C. (decomposition).

### EXAMPLE 279

### N-{3-[5-Methyl-3-(2-thienylcarbonyl)pyrazolo[1,5-a]pyrimidin-7-yl]phenyl}cyclobutanecarboxamide

The title compound was prepared using a procedure analogous to Method D from [7-(3-aminophenyl)pyrazolo[1,5-a]pyrimidin-3-yl]-2-thienyl-methanone and cyclobutanecarbonyl chloride, mp 121-123° C.

### EXAMPLE 280

### N-{3-[3-(2-Thienylcarbonyl)pyrazolo[1,5-a]pyrimidin-7-yl]phenyl}-2,5-bis(trifluoromethyl)benzamide

The title compound was prepared using a procedure analogous to Method D from [7-(3-aminophenyl)pyrazolo[1,5-a]pyrimidin-3-yl]-2-thienyl-methanone and 2,5-bis(trifluoromethyl)benzoyl chloride, mp 208-210° C.

### EXAMPLE 281

### N-{3-[3-(2-Thienylcarbonyl)pyrazolo[1,5-a]pyrimidin-7-yl]phenyl}-3-trifluoromethyl)benzamide

The title compound was prepared using a procedure analogous to Method D from [7-(3-aminophenyl)pyrazolo[1,5-a]pyrimidin-3-yl]-2-thienyl-methanone and 3-(trifluoromethyl)benzoyl chloride, mp 135-140° C.

### EXAMPLE 282

### N-{3-[3-(2-Thienylcarbonyl)pyrazolo[1,5-a]pyrimidin-7-yl]phenyl}cyclohexanecarboxamide

The title compound was prepared using a procedure analogous to Method D from [7-(3-aminophenyl)pyrazolo[1,5-a]pyrimidin-3-yl]-2-thienyl-methanone and cyclohexanecarbonyl chloride, mp 239-240° C.

### EXAMPLE 283

### N-{3-[3-(2-Thienylcarbonyl)pyrazolo[1,5-a]pyrimidin-7-yl]phenyl}cyclopentanecarboxamide

The title compound was prepared using a procedure analogous to Method D from [7-(3-aminophenyl)pyrazolo[1,5-a]pyrimidin-3-yl]-2-thienyl-methanone and cyclopentnecarbonyl chloride, mp 209-210° C.

### EXAMPLE 284

### N-{3-[3-(2-Thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and benzoyl chloride, mp 211-213° C.

### EXAMPLE 285

### N-{3-[3-(2-Thienylcarbonyl)pyrazolo[1,5-a]pyrimidin-7-yl]phenyl}-2-furamide

The title compound was prepared using a procedure analogous to Method D from [7-(3-aminophenyl)pyrazolo[1,5-a]pyrimidin-3-yl]-2-thienyl-methanone and furoyl chloride, mp>240° C.

### EXAMPLE 286

### 3-Bromo-N-{3-[3-(2-thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and 3-bromobenzoyl chloride, mp>300° C.

### EXAMPLE 287

### 4-(tert-butyl)-N-{3-[3-(2-thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and 4-tert-butylbenzoyl chloride as a yellow foam, M+H 491.

### EXAMPLE 288

### 3.5-Dinitro-N-{3-[3-(2-thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and 3,5-dinitrobenzoyl chloride, mp 145° C. (dec.).

### EXAMPLE 289

### 2,4-Dichloro-N-{3-[3-(2-thienylcarbonyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-thienyl-methanone and 2,4-dichlorobenzoyl chloride, mp 132-134° C.

### EXAMPLE 290

### N-{3-[3-(2-Thienylcarbonyl)pyrazolo[1,5-a]pyrimidin-7-yl]phenyl}nicotinamide

The title compound was prepared using a procedure analogous to Method D from [7-(3-aminophenyl)pyrazolo[1,5-a]pyrimidin-3-yl]-2-thienyl-methanone and nicotinoyl chloride hydrochloride, mp 191-194° C.

### EXAMPLE 291

### N-{3-[3-(2-Thienylcarbonyl)pyrazolo[1,5-a]pyrimidin-7-yl]phenyl}isonicotinamide

The title compound was prepared using a procedure analogous to Method D from [7-(3-aminophenyl)pyrazolo[1,5-a]pyrimidin-3-yl]-2-thienyl-methanone and isonicotinoyl chloride hydrochloride or prepared from [7-(3-aminophenyl-5-methyl)pyrazolo[1,5-a]pyrimidin-3-yl]-2-thienyl-methanone and isonicotinic acid as described in Method E, mp 220-222° C.

### EXAMPLE 292

### N,N′-Dibutyl-N-{3-[3-(2-thienylcarbonyl)pyrazolo[1,5-a]pyrimidin-7-yl]phenyl}dicarbonimidic diamide

The title compound was prepared using a procedure analogous to Method G from [7-(3-aminophenyl)pyrazolo[1,5-a]pyrimidin-3-yl]-2-thienyl-methanone and excess of butyl isocyanate, mp 180-181° C.

### EXAMPLE 293

### Isopropyl 3-[3-(3-thienylcarbonyl)pyrazolo[1,5-a]pyrimidin-7-yl]phenylcarbamate

The title compound was prepared using a procedure analogous to Method H from [7-(3-aminophenyl)pyrazolo[1,5-a]pyrimidin-3-yl]-3-thienyl-methanone and isopropyl chloroformate, mp 122-124° C.

### EXAMPLE 294

### Isopropyl 3-(3-benzoylpyrazolo[1,5-a]pyrimidin-7-yl)phenylcarbamate

The title compound was prepared using a procedure analogous to Method H from [7-(3-aminophenyl)pyrazolo[1,5-a]pyrimidin-3-yl]phenyl-methanone and isopropyl chloroformate, mp 116-118° C.

### EXAMPLE 295

### Isopropyl 3-[3-(cyclopentylcarbonyl)pyrazolo[1,5-a]pyrimidin-7-yl]phenylcarbamate

The title compound was prepared using a procedure analogous to Method H from [7-(3-aminophenyl)pyrazolo[1,5-a]pyrimidin-3-yl]cyclopentyl-methanone and isopropyl chloroformate, mp 57-59° C.

[7-(3-Aminophenyl)pyrazolo[1,5-a]pyrimidin-3-yl]cyclopentyl ûmethanone was prepared from [7-(3-nitrophenyl)pyrazolo[1,5-a]pyrimidin-3-yl]cyclopentylumethanone by an analogous procedure to Method C.

[7-(3-Nitrophenyl)pyrazolo[1,5-a]pyrimidin-3-yl]cyclopentylumethanone was prepared from 3-(dimethylamino)-1-(3-nitrophenyl)-2-propen-1-one and (5-amino-1H-pyrazol-4-yl)(cyclopentyl)methanone by an analogous procedure to Method A.

(5-Amino-1H-pyrazol-4-yl)(cyclopentyl)methanone was prepared using an analogous procedure described in Method K from (2E)-2-(cyclopentylcarbonyl)-3-dimethylamino-2-propenenitrile.

(2E)-2-(Cyclopentylcarbonyl)-3-dimethylamino-2-propenenitrile was prepared from (β-oxo-cyclopentanepropanenitrile (EP patent 157260) by an analogous procedure to Method L.

### EXAMPLE 296

### N-{3-[3-(Cyclopentylcarbonyl)pyrazolo[1,5-a]pyrimidin-7-yl]phenyl}-3-methylbutanamide

The title compound was prepared using a procedure analogous to Method H from [7-(3-aminophenyl)pyrazolo[1,5-a]pyrimidin-3-yl]cyclopentyl-methanone (Example 295) and isobutyl chloroformate, mp 69-71° C.

### EXAMPLE 297

### 3-Methyl-N-{3-[3-(3-thienylcarbonyl)pyrazolo[1,5-a]pyrimidin-7-yl]phenyl}butanamide

The title compound was prepared using a procedure analogous to Method D from [7-(3-aminophenyl)pyrazolo[1,5-a]pyrimidin-3-yl]-3-thienyl-methanone and isovaleryl chloride, mp 137-139° C.

### EXAMPLE 298

### N-{3-[3-(2-Furoyl)pyrazolo[1,5-a]pyrimidin-7-yl]phenyl}-N′-isopropylurea

The title compound was prepared using a procedure analogous to Method G from [7-(3-aminophenyl)pyrazolo[1,5-a]pyrimidin-3-yl]-2-furanyl-methanone and isopropyl isocyanate, mp 143° C. (dec.).

### EXAMPLE 299

### Isopropyl 3-[3-(2-furoyl)pyrazolo[1,5-a]pyrimidin-7-yl]phenylcarbamate

The title compound was prepared using a procedure analogous to Method H from [7-(3-aminophenyl)pyrazolo[1,5-a]pyrimidin-3-yl]-2-furanyl-methanone and isopropyl chloroformate, mp 140-143° C.

### EXAMPLE 300

### N-{3-[3-(2-Furoyl)pyrazolo[1,5-a]pyrimidin-7-yl]phenyl}benzamide

The title compound was prepared using a procedure analogous to Method D from [7-(3-aminophenyl)pyrazolo[1,5-a]pyrimidin-3-yl]-2-furanyl-methanone and benzoyl chloride, mp 220-222° C.

### EXAMPLE 301

### N-{3-[3-(2-Furoyl)pyrazolo[1,5-a]pyrimidin-7-yl]phenyl}-3-methylbutanamide

The title compound was prepared using a procedure analogous to Method D from [7-(3-aminophenyl)pyrazolo[1,5-a]pyrimidin-3-yl]-2-furanyl-methanone and isovaleryl chloride, mp 134° C. (dec.).

### EXAMPLE 302

### N-{3-[3-(2-Furoyl)pyrazolo[1,5-a]pyrimidin-7-yl]phenyl}cyclopentanecarboxamide

The title compound was prepared using a procedure analogous to Method D from [7-(3-aminophenyl)pyrazolo[1,5-a]pyrimidin-3-yl]-2-furanyl-methanone and cyclopentanecarbonyl chloride, mp 143° C. (dec.).

### EXAMPLE 303

### 2-Cyclopentyl-N-{3-[3-(2-furoyl)pyrazolo[1,5-a]pyrimidin-7-yl]phenyl}acetamide

The title compound was prepared using a procedure analogous to Method E from [7-(3-aminophenyl)pyrazolo[1,5-a]pyrimidin-3-yl]-2-furanyl-methanone and cyclopentanacetic acid, mp 142° C. (dec.).

### EXAMPLE 304

### N-{3-[3-(3-Furoyl)pyrazolo[1,5-a]pyrimidin-7-yl]phenyl}-3-methylbutanamide

The title compound was prepared using a procedure analogous to Method D from [7-(3-aminophenyl)pyrazolo[1,5-a]pyrimidin-3-yl]-3-furanyl-methanone and isovelaryl chloride, mp 144-146° C.

[7-(3-Aminophenyl)pyrazolo[1,5-a]pyrimidin-3-yl]-3-furanyl-methanone was prepared using an analogous procedure to Method C from [7-(3-nitrophenyl)pyrazolo[1,5-a]pyrimidin-3-yl]-3-furanyl-methanone.

[7-(3-Nitrophenyl)pyrazolo[1,5-a]pyrimidin-3-yl]-3-furanyl-methanone was prepared using an analogous procedure to Method A from (3-amino-1H-pyrazol-4-yl)-3-furanyl-methanone and 3-(dimethylamino)-1-(3-nitrophenyl)-2-propen-1-one.

(3-Amino-1H-pyrazol-4-yl)-3-furanyl-methanone was prepared using an analogous procedure to Method K from 3-acetylfuran.

### EXAMPLE 305

### Isopropyl 3-[3-(3-furoyl)pyrazolo[1,5-a]pyrimidin-7-yl]phenylcarbamate

The title compound was prepared using a procedure analogous to Method H from [7-(3-aminophenylpyrazolo[1,5-a]pyrimidin-3-yl]-3-furanyl-methanone (Example 304) isopropyl chloroformate, mp 127-129° C.

### EXAMPLE 306

### Methyl 3-[3-(2-thienylcarbonyl)pyrazolo[1,5-a]pyrimidin-7-yl]benzoate

The title compound was prepared using a procedure analogous to Method A from (3-amino-1H-pyrazol-4-yl)-2-thienyl-methanone and methyl 3-[3-(dimethylamino)-1-oxo-2-propenyl]benzoate, mp 180-184° C.

Methyl 3-[3-(imethylamino)-1-oxo-2-propenyl]benzoate was prepared from methyl 3-acetylbenzoate and dimethyl formamide-dimethyl acetal using an analogous procedure to Method B.

### EXAMPLE 307

### 3-Methyl-N-{3-[2-methyl-3-(2-thienylcarbonyl)pyrazolo[1,5-a]pyrimidin-7-yl]phenyl}butanamide

The title compound was prepared using a procedure analogous to Method D from [7-(3-aminophenyl-2-methyl)pyrazolo[1,5-a]pyrimidin-3-yl]-2-thienyl-methanone and isovaleryl chloride, mp 137-139° C.

### EXAMPLE 308

### Isopropyl 3-[2-methyl-3-(2-thienylcarbonyl)pyrazolo[1,5-a]pyrimidin-7-yl]phenylcarbamate

The title compound was prepared using a procedure analogous to Method H from [7-(3-aminophenyl-2-methyl)pyrazolo[1,5-a]pyrimidin-3-yl]-2-thienyl-methanone and isopropyl chloroformate, mp 186-188° C.

### EXAMPLE 309

### N-{2-Methyl-5-[3-(2-thienylcarbonyl)pyrazolo[1,5-a]pyrimidin-7-yl]phenyl}benzamide

The title compound was prepared using a procedure analogous to Method D from {[7-(3-amino-4-methyl)phenyll]pyrazolo[1,5-a]pyrimidin-3-yl}-2-thienyl-methanone and benzoyl chloride, mp 216-218° C.

### EXAMPLE 310

### 3-Methyl-N-{2-methyl-5-[3-(2-thienylcarbonyl)pyrazolo[1,5-a]pyrimidin-7-yl]phenyl}butanamide

The title compound was prepared using a procedure analogous to Method D from {[7-(3-amino-4-methyl)phenyl]pyrazolo[1,5-a]pyrimidin-3-yl}-2-thienyl-methanone and isovaleryl chloride, mp 185-187° C.

### EXAMPLE 311

### N-Isopropyl-N′-{2-methyl-5-[3-(2-thienylcarbonyl)pyrazolo[1,5-a]pyrimidin-7-yl]phenyl}urea

The title compound was prepared using a procedure analogous to Method G from {[7-(3-amino-4-methyl)phenyl]pyrazolo[1,5-a]pyrimidin-3-yl}-2-thienyl-methanone and isopropyl isocyanate, mp 225-227° C.

### EXAMPLE 312

### N-{2-Chloro-5-[3-(2-thienylcarbonyl)pyrazolo[1,5-a]pyrimidin-7-yl]phenyl}benzamide

The title compound was prepared using a procedure analogous to Method D from {[7-(3-amino-4-chloro)phenyl]pyrazolo[1,5-a]pyrimidin-3-yl}-2-thienyl-methanone and benzoyl chloride, mp 217-218° C.

### EXAMPLE 313

### N-{2-Chloro-5-[3-(2-thienylcarbonyl)pyrazolo[1,5-a]pyrimidin-7-yl]phenyl}-3-Methylbutanamide

The title compound was prepared using a procedure analogous to Method D from {[7-(3-amino-4-chloro)phenyl]pyrazolo[1,5-a]pyrimidin-3-yl}-2-thienyl-methanone and isovaleryl chloride, mp 172-175° C.

### EXAMPLE 314

### Isopropyl 2-methyl-5-[3-(2-thienylcarbonyl)pyrazolo[1,5-a]pyrimidin-7-yl]phenylcarbamate

The title compound was prepared using a procedure analogous to Method H from {[7-(3-amino-4-methyl)phenyl]pyrazolo[1,5-a]pyrimidin-3-yl}-2-thienyl-methanone and isopropyl chloroformate, mp 198-200° C.

### EXAMPLE 315

### N-{2-Methoxy-5-[3-(2-thienylcarbonyl)pyrazolo[1,5-a]pyrimidin-7-yl]phenyl}benzamide

The title compound was prepared using a procedure analogous to Method D from {[7-(3-amino-4-methoxy)phenyl]pyrazolo[1,5-a]pyrimidin-3-yl}-2-thienyl-methanone and benzoyl chloride, mp 201-203° C.

### EXAMPLE 316

### Isopropyl 2-methoxy-5-[3-(2-thienylcarbonyl)pyrazolo[1,5-a]pyrimidin-7-yl]phenylcarbamate

The title compound was prepared using a procedure analogous to Method H from {[7-(3-amino-4-methoxy)phenyl]pyrazolo[1,5-a]pyrimidin-3-yl}-2-thienyl-methanone and isopropyl chloroformate, mp 193-195° C.

### EXAMPLE 317

### N-{2-Methoxy-5-[3-(2-thienylcarbonyl)pyrazolo[1,5-a]pyrimidin-7-yl]phenyl}-3-methylbutanamide

The title compound was prepared using a procedure analogous to Method D from {[7-(3-amino-4-methoxy)phenyl]pyrazolo[1,5-a]pyrimidin-3-yl}-2-thienyl-methanone and isovaleryl chloride, mp 192-194° C.

### EXAMPLE 318

### N-Isobutyl-3-[3-(2-thienylcarbonyl)pyrazolo[1,5-a]pyrimidin-7-yl]benzamide

The title compound was prepared as described in Method J from 3-[3-(2-thienylcarbonyl)pyrazolo[1,5-a]pyrimidin-7-yl]benzoic acid and isobutyl amine, mp 174-177° C.

### EXAMPLE 319

### N-Butyl-3-[3-(2-thienylcarbonyl)pyrazolo[1,5-a]pyrimidin-7-yl]benzamide

The title compound was prepared using an analogous procedure to Method J from 3-[3-(2-thienylcarbonyl)pyrazolo[1,5-a]pyrimidin-7-yl]benzoic acid and n-butyl amine, mp 172-174° C.

### EXAMPLE 320

### N-Cyclopentyl-3-[3-(2-thienylcarbonyl)pyrazolo[1,5-a]pyrimidin-7-yl]benzamide

The title compound was prepared using an analogous procedure to Method J from 3-[3-(2-thienylcarbonyl)pyrazolo[1,5-a]pyrimidin-7-yl]benzoic acid and cyclopentylamine, mp 207-209° C.

### EXAMPLE 321

### N-Phenyl-3-[3-(2-thienylcarbonyl)pyrazolo[1,5-a]pyrimidin-7-yl]benzamide

The title compound was prepared using an analogous procedure to Method J from 3-[3-(2-thienylcarbonyl)pyrazolo[1,5-a]pyrimidin-7-yl]benzoic acid and aniline, mp 211-212° C.

### EXAMPLE 322

### N-Isopropyl-3-[3-(2-thienylcarbonyl)pyrazolo[1,5-a]pyrimidin-7-yl]benzamide

The title compound was prepared using an analogous procedure to Method J from 3-[3-(2-thienylcarbonyl)pyrazolo[1,5-a]pyrimidin-7-yl]benzoic acid and isopropylamine, mp 218-220° C.

### EXAMPLE 323

### N-Isopropyl-N′-{2-methoxy-5-[3-(2-thienylcarbonyl)pyrazolo[1,5-a]pyrimidin-7-yl]phenyl}urea

The title compound was prepared using a procedure analogous to Method G from {[7-(3-amino-4-methoxy)phenyl]pyrazolo[1,5-a]pyrimidin-3-yl}-2-thienyl-methanone and isopropyl isocyanate, mp 202-204° C.

### EXAMPLE 324

### 3-Methyl-N-{5-[3-(2-thienylcarbonyl)pyrazolo[1,5-a]pyrimidin-7-yl]-3-pyridinyl}butanamide

The title compound was prepared using a procedure analogous to Method D from [7-(5-amino-3-pyridinyl)pyrazolo[1,5-1]pyrimidin-3-yl](2-thienyl)methanone and isovaleryl isocyanate, mp 217-219° C.

### EXAMPLE 325

### 7-(3-Nitrophenyl)-3-(3-thienyl)pyrazolo[1,5-a]pyrimidine

A mixture of 3-bromo-7-(3-nitrophenylpyrazolo[1,5-a]pyrimidine (240 mg, 0.75 mmol), 3-thiopheneboronic acid (192 mg, 1.5 mmol) and palladium tetrakis(triphenylphosphine) (58 mg, 0.05 mmol) in dimethoxy ethane (6 mL) and saturated aqueous sodium bicarbonate (3 mL) was heated at 100-105° C. under nitrogen for 20 hours. The reaction mixture was partitioned between water and ethyl acetate. The combined organics were dried, concentrated and purified by flash column chromatography eluting with a gradient mixture of ethyl acetate/hexanes to give 246 mg (76%) of the title compound as an orange solid, mp 148-150° C.

### EXAMPLE 326

### 3-(3-Bromopyrazolo[1,5-a]pyrimidin-7-yl)aniline

The title compound was prepared using an analogous procedure to Method C from 3-bromo-7-(3-nitrophenylpyrazolo[1,5-a]pyrimidine, mp 120-121° C.

### EXAMPLE 327

### Isobutyl 3-(3-bromopyrazolo[1,5-a]pyrimidin-7-yl)phenylcarbamate

The title compound was prepared using an analogous procedure to Method H from 3-(3-bromopyrazolo[1,5-a]pyrimidin-7-yl)aniline and isobutyl chlorofomate, mp 90-92° C.

### EXAMPLE 328

### [7-(5-Nitro-3-thienyl)pyrazolo[1,5-a]pyrimidin-3-yl](2-thienyl)methanone

The title compound was prepared using a procedure analogous to Method A from (3-amino-1H-pyrazol-4-yl)-2-thienyl-methanone and 3-(dimethylamino)-1-[5-nitro-3-thienyl]-2-propen-1-one, mp 225-226° C. 3-(Dimethylamino)-1-[5-nitril-3-thienyl]-2-propen-1-one was prepared using an analogous procedure to Method B from 2-acetyl-4-nitrothiophene and dimethyl formamide-dimethyl acetal.

### EXAMPLE 329

### N-Isopropyl-N′-{5-[3-(thien-2-ylcarbonyl)pyrazolo[1,5-a]pyrimidin-7-yl]thien-2-yl}urea

The title compound was prepared using a procedure analogous to Method G from and [7-(5-amino-2-thienyl)pyrazolo[1,5-a]pyrimidin-3-yl](thien-2-yl)methanone and isopropyl isocyanate, mp 162-165° C.

### EXAMPLE 330

### N-[3-(3-Bromopyrazolo[1,5-a]pyrimidin-7-yl)phenyl]-3-methylbutanamide

The title compound was prepared using an analogous procedure to Method D from 3-(3-bromopyrazolo[1,5-a]pyrimidin-7-yl)aniline and isovaleryl chloride, mp 132-134° C.

### EXAMPLE 331

### [7-(5-Aminothien-3-yl)pyrazolo[1,5-a]pyrimidin-3-yl](thien-2-yl)methanone

The title compound was prepared using an analogous procedure to Method C from [7-(5-nitro-3-thienyl)pyrazolo[1,5-a]pyrimidin-3-yl](2-thienyl)methanone, mp 174-176° C.

### EXAMPLE 332

### O-Ethyl 3-[3-(thien-2-ylcarbonyl)pyrazolo[1,5-a]pyrimidin-7-yl]phenylthiocarbamate

A mixture of [7-(3-aminophenyl)pyrazolo[1,5-a]pyrimidin-3-yl]-2-thienyl-methanone (320 mg, 1.0 mmol) and phenyl thioisocyanate (135 mg, 1.0 mmol) in ethanol (5 mL)and tetrahydrofuran (10 mL) was heated at reflux overnight. Additional phenyl thioisocyanate (135 mG, 1.0 mmol) was added and the reflux was continued for another 24 hours. The reaction mixture was diluted with methylene chloride, concentrated on silica gel and purified by flash column chromatography eluting with 1:6 ethyl acetate/hexanes to give 48 mg (12%) of the title compound as an off-white solid, mp 209-211° C.

### EXAMPLE 333

### 3-Methyl-N-[3-(3-thien-2-ylpyrazolo[1,5-a]pyrimidin-7-yl)phenyl]butanamide

The title compound was prepared using a procedure analogous to Example 325 from N-[3-(3-bromopyrazolo[1,5-a]pyrimidin-7-yl)phenyl]-3-methylbutanamide and 2-thiopheneboronic acid, mp 148-150° C.

### EXAMPLE 334

### N-Phenyl-N′-{3-[3-(thien-2-ylcarbonyl)pyrazolo[1,5-a]pyrimidin-7-yl]phenyl}thiourea

A mixture of [7-(3-aminophenyl)pyrazolo[1,5-a]pyrimidin-3-yl]-2-thienyl-methanone (320 mg, 1.0 mmol) and phenyl thioisocyanate (135 mg, 1.0 mmol) in tetrahydrofuran (5 mL) and pyridine (1 mL) was heated for 2 hours. The reaction mixture was diluted with saturated aqueous sodium bicarbonate. The precipitated solid was collected by filtration and washed with water. The crude solid was purified by purified by flash column chromatography eluting with 1:1 ethyl acetate/hexanes to give 181 mg (40%) of the title compound as an off-white solid, mp 124-126° C.

### EXAMPLE 335

### Isopropyl 4-chloro-3-[3-(thien-2-ylcarbonyl)pyrazolo[1,5-a]pyrimidin-7-yl]phenylcarbamate

The title compound was prepared using an analogous procedure to Method H from {[7-(5-amino-2-chloro)phenyl]pyrazolo[1,5-a]pyrimidin-3-yl}-2-thienyl-methanone and isobutyl chlorofomate, mp 234-236° C.

### EXAMPLE 336

### [7-(2-Chloro-5-nitrophenyl)pyrazolo[1,5-a]pyrimidin-3-yl](thien-2-yl)methanone

The title compound was prepared using an analogous procedure to Method A from (3-amino-1H-pyrazol-4-yl)-2-thienyl-methanone and 3-(dimethylamino)-1-(2-chloro-5-nitrophenyl)-2-propen-1-one, mp 162-166° C.

3-(Dimethylamino)-1-(2-chloro-5-nitrophenyl)-2-propen-1-one was prepared using an analogous procedure to Method B from 2-chloro-5-nitroacetophenone and dimethyl formamide-dimethyl acetal.

### EXAMPLE 337

### N-{3-[3-(Pyridin-2-ylcarbonyl)pyrazolo[1,5-a]pyrimidin-7-yl]phenyl}cyclopropanecarboxamide

The title compound was prepared using an analogous procedure to Method D from {[7-(3-aminophenyl)-2-methyl]pyrazolo[1,5-a]pyrimidin-3-yl}-2-pyridinyl-methanone and cyclopropanecarbonyl chloride, mp 214-215° C.

{[7-(3-Aminophenyl)-2-methyl]pyrazolo[1,5-a]pyrimidin-3-yl}-2-pyridinyl-methanone was prepared from (3-amino-1H-pyrazol-4-yl)-2-pyridinyl-methanone and 3-(dimethylamino)-1-(3-nitrophenyl)-2-propen-1-one using procedures analogous to Method A and C.

### EXAMPLE 338

### N-{3-[3-(1,3-Thiazol-2-ylcarbonyl)pyrazolo[1,5-a]pyrimidin-7-yl]phenyl}cyclopropanecarboxamide

The title compound was prepared using an analogous procedure to Method D from {[7-(3-aminophenyl)]pyrazolo[1,5-a]pyrimidin-3-yl}-2-(1,3-thiazol)-methanone and cyclopropanecarbonyl chloride, mp 226-229° C.

{[7-(3-Aminophenyl)-2-methyl]pyrazolo[1,5-a]pyrimidin-3-yl}-2-(1,3-thiazol)—methanone was prepared from (3-amino-1H-pyrazol-4-yl)-2-(1,3-thiazol)-methanone and 3-(dimethylamino)-1-(3-nitrophenyl)-2-propen-1-one using procedures analogous to Method A and C.

### EXAMPLE 339

### N-{4-Chloro-3-[3-(thien-2-ylcarbonyl)pyrazolo[1,5-a]pyrimidin-7-yl]phenyl}-3-methylbutanamide

The title compound was prepared using an analogous procedure to Method D from {[7-(5-amino-2-chloro)phenyl]pyrazolo[1,5-a]pyrimidin-3-yl}-2-thienyl-methanone and isovaleryl chloride, mp 162-165° C.

### EXAMPLE 340

### N-{4-Chloro-3-[3-(thien-2-ylcarbonyl)pyrazolo[1,5-a]yrimidin-7-yl]phenyl}-N′-isopropylthiourea

The title compound was prepared using an analogous procedure to Example 334 from {[7-(5-amino-2-chloro)phenyl]pyrazolo[1,5-a]pyrimidin-3-yl}-2-thienyl-methanone and isopropyl thioisocyanate, mp 194-195° C.

### EXAMPLE 341

### N-{4-Chloro-3-[3-(thien-2-ylcarbonyl)pyrazolo[1,5-a]pyrimidin-7-yl]phenyl}benzamide

The title compound was prepared using an analoous procedure to Method D from {[7-(5-amino-2-chloro)phenyl]pyrazolo[1,5-a]pyrimidin-3-yl}-2-thienyl-methanone and benzoyl chloride, mp 194-195° C.

### EXAMPLE 342

### N-{2-Fluoro-5-[3-(thien-2-ylcarbonyl)pyrazolo[1,5-a]pyrimidin-7-yl]phenyl}-3-methylbutanamide

The title compound was prepared using a procedure analogous to Method D from {[7-(3-amino-4-fluoro)phenyl]pyrazolo[1,5-a]pyrimidin-3-yl}-2-thienyl-methanone and isovaleryl chloride, mp 192-194° C.

### EXAMPLE 343

### 3-Methyl-N-{5-[3-(thien-2-ylcarbonyl)pyrazolo[1,5-a]pyrimidin-7-yl]thien-2-yl}butanamide

The title compound was prepared using a procedure analogous to Method D from and [7-(5-amino-2-thienyl)pyrazolo[1,5-a]pyrimidin-3-yl](thien-2-yl)methanone and isovaleryl chloride, mp 268-270° C.

### EXAMPLE 344

### Isopropyl 5-[3-(thien-2-ylcarbonyl)pyrazolo[1,5-a]pyrimidin-7-yl]thien-2-ylcarbamate

The title compound was prepared using a procedure analogous to Method H from and [7-(5-amino-2-thienyl)pyrazolo[1,5-a]pyrimidin-3-yl](thien-2-yl)methanone and isopropyl chloroformate, mp 210-215° C.

### EXAMPLE 345

### [7-(5-Aminothien-2-yl)pyrazolo[1,5-a]pyrimidin-3-yl](thien-2-yl)methanone

The title compound was prepared using an analogous procedure to Method C from [7-(5-nitro-2-thienyl)pyrazolo[1,5-a]pyrimidin-3-yl](2-thienyl)methanone, mp 232-235° C. (dec.).

### EXAMPLE 346

### 4-Chloro-N-{3-[3-(thien-2-ylcarbonyl)pyrazolo[1,5-a]pyrimidin-7-yl]phenyl}butanamide

The title compound was prepared using a procedure analogous to Method D from [7-(3-aminophenyl)pyrazolo[1,5-a]pyrimidin-3-yl]-2-thienyl-methanone and 4-chlorobutyryl chloride, mp 97-100° C.

### EXAMPLE 347

### Isopropyl 2-fluoro-5-[3-(thien-2-ylcarbonyl)pyrazolo[1,5-a]pyrimidin-7-yl]phenylcarbamate

The title compound was prepared using a procedure analogous to Method H from {[7-(3-amino-4-fluoro)phenyl]pyrazolo[1,5-a]pyrimidin-3-yl}-2-thienyl-methanone and isopropyl chloroformate, mp>200° C. (dec).

### EXAMPLE 348

### Isopropyl 2-(dimethylamino)-5-[3-(thien-2-ylcarbonyl)pyrazolo[1,5-a]pyrimidin-7-yl]phenylcarbamate

The title compound was prepared using a procedure analogous to Method H from ([7-(3-amino-4-dimethylamino)phenyl]pyrazolo[1,5-a]pyrimidin-3-yl}-2-thienyl-methanone and isopropyl chloroformate, mp 164-165° C.

{[7-(3-Amino-4-dimethylamino)phenyl]pyrazolo[1,5-a]pyrimidin-3-yl}-2-thienyl-methanone was prepared using an analogous procedure to Method C from {[7-(4-dimethylamino-3-nitro)phenyl]pyrazolo[1,5-a]pyrimidin-3-yl}-2-thienyl-methanone.

{[7-(4-Dimethylamino-3-nitro)phenyl]pyrazolo[1,5-a]pyrimidin-3-yl}-2-thienyl-methanone was obtained as an additional product in the reaction of (3-amino-1H-pyrazol-4-yl)-2-thienyl-methanone and 3-(dimethylamino)-1-(4-fluoro-3-nitrophenyl)-2-propen-1-one in acetic acid.

### EXAMPLE 349 (184858)

### {7-[3-(Benzylamino)phenyl]pyrazolo[1,5-a]pyrimidin-3-yl}(thien-2-yl)methanone

The title compound was prepared as described in Method F, mp 90-92° C.

### EXAMPLE 350

### {7-[3-(Isopentylamino)phenyl]pyrazolo[1,5-a]pyrimidin-3-yl}(thien-2-yl)methanone

The title compound was prepared using an analogous procedure to Method F from [7-(3-Aminophenyl)pyrazolo[1,5-a]pyrimidin-3-yl]-2-thienyl-methanone and isobutyraldehyde, mp 75-55° C.

### EXAMPLE 351

### 3-Methyl-N-{4-[3-(thien-2-ylcarbonyl)pyrazolo[1,5-a]pyrimidin-7-yl]thien-2-yl}butanamide

The title compound was prepared using a procedure analogous to Method D from and [7-(5-amino-3-thienyl)pyrazolo[1,5-a]pyrimidin-3-yl](thien-2-yl)methanone and isovaleryl chloride, mp 234-235° C.

### EXAMPLE 352

### [7-(3-Amino-4-fluorophenyl)pyrazolo[1,5-a]pyrimidin-3-yl](thien-2-yl)methanone

The title compound was prepared using an analogous procedure to Method C from {[7-(4-fluoro-3-nitro)phenyl]pyrazolo[1,5-a]pyrimidin-3-yl}-2-thienyl-methanone, mp 241-242° C.

### EXAMPLE 353

### N-Cyclohexyl-N′-{3-[3-(thien-2-ylcarbonyl)pyrazolo[1,5-a]pyrimidin-7-yl]phenyl}urea

The title compound was prepared using a procedure analogous to Method D from {[7-(3-amino-4-fluoro)phenyl]pyrazolo[1,5-a]pyrimidin-3-yl}-2-thienyl-methanone and tert-butylacetyl chloride, mp 233-236° C.

### EXAMPLE 354

### 4,4,4-Trichloro-N-{3-[3-(thien-2-ylcarbonyl)pyrazolo[1,5-a]pyrimidin-7-yl]phenyl}butanamide

The title compound was prepared using a procedure analogous to Method D from [7-(3-aminophenyl)pyrazolo[1,5-a]pyrimidin-3-yl]-2-thienyl-methanone and 4,4,4-trichlorobutyryl chloride, mp 189-191° C.

### EXAMPLE 355

### N-{2-Fluoro-5-[3-(thien-2-ylcarbonyl)pyrazolo[1,5-a]pyrimidin-7-yl]phenyl}-3,3-dimethylbutanamide

The title compound was prepared using a procedure analogous to Method D from {[7-(3-amino-4-fluoro)phenyl]pyrazolo[1,5-a]pyrimidin-3-yl}-2-thienyl-methanone and tert-butylacetyl chloride, mp 180-181° C.

### EXAMPLE 356

### 4,4,4-Trifluoro-N-{2-fluoro-5-[3-(thien-2-ylcarbonyl)pyrazolo[1,5-a]pyrimidin-7-yl]phenyl}-3-methylbutanamide

The title compound was prepared using a procedure analoous to Method E from {[7-(3-amino-4-fluoro)phenyl]pyrazolo[1,5-a]pyrimidin-3-yl}-2-thienyl-methanone and 3-methyl-4,4,4-trifluorobutyric acid, mp 185-186° C.

### EXAMPLE 357

### bis(2,2,2-Trichloroethyl) 2-fluoro-5-[3-(thien-2-ylcarbonyl)pyrazolo[1,5-a]pyrimidin-7-yl]phenylimidodicarbonate

The title compound was prepared using a procedure analogous to Method H from {[7-(3-amino-4-fluoro)phenyl]pyrazolo[1,5-a]pyrimidin-3-yl}-2-thienyl-methanone and excess of 2,2,2-trichloroethyl chloroformate, mp 94-97° C.

### EXAMPLE 358

### 2-Fluoro-N-{3-[3-(thien-2-ylcarbonyl)pyrazolo[1,5-a]pyrimidin-7-yl]phenyl}benzamide

The title compound was prepared as described in Method D, mp 190-191° C.

### EXAMPLE 359

### 2,6-Difluoro-N-{3-[3-(thien-2-ylcarbonyl)pyrazolo[1,5-a]pyrimidin-7-yl]phenyl}benzamide

The title compound was prepared using a procedure analogous to Method D from {[7-(3-amino-4-fluoro)phenyl]pyrazolo[1,5-a]pyrimidin-3-yl}-2-thienyl-methanone and 2,6-difluorobenzoyl chloride, mp 159-160° C.

### EXAMPLE 360

### N-{3-[3-(Thien-2-ylcarbonyl)pyrazolo[1,5-a]pyrimidin-7-yl]phenyl}pyridine-2-carboxamide

The title compound was prepared using a procedure analogous to Method E from {[7-(3-amino-4-fluoro)phenyl]pyrazolo[1,5-a]pyrimidin-3-yl}-2-thienyl-methanone and picolinic acid, mp 198-200° C.

### EXAMPLE 361

### N-{5-[3-(Thien-2-ylcarbonyl)pyrazolo[1,5-a]pyrimidin-7-yl]thien-2-yl}benzamide

The title compound was prepared using a procedure analogous to Method D from and [7-(5-amino-2-thienyl)pyrazolo[1,5-a]pyrimidin-3-yl](thien-2-yl)methanone and benzoyl chloride, mp 162-165° C.

### EXAMPLE 362

### (7-{3-[(2-Phenylethyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and phenyl acetaldehyde, mp 118-120° C.

### EXAMPLE 363

### (7-{3-[(2-Methoxybenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-methoxybenzaldehyde, mp 138-140° C.

### EXAMPLE 364

### N-Butyl-N′-{3-[3-(thien-2-ylcarbonyl)pyrazolo[1,5-a]pyrimidin-7-yl]phenyl}thiourea

The title compound was prepared using an analogous procedure to Example 334 from [7-(3-aminophenyl)pyrazolo[1,5-a]pyrimidin-3-yl]-2-thienyl-methanone and butyl thioisocyanate, mp 155-157° C.

### EXAMPLE 365

### (7-{3-[(3-Chlorobenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 3-chlorobenzaldehyde, mp 133-135° C.

### EXAMPLE 366

### (7-{3-[(4-Chlorobenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 4-chlorobenzaldehyde, mp 149-152° C.

### EXAMPLE 367

### (7-{3-[(2-Furylmethyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-furylaldehyde, as an off-white semi-solid, mp 124-128° C.

### EXAMPLE 368

### (7-{3-[(2-Chlorobenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-chlorobenzaldehyde, as an off-white semi-solid (decomp).

### EXAMPLE 369

### (7-{3-[(3-Furylmethyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 3-furylaldehyde, mp 140-144° C.

### EXAMPLE 370

### (7-{3-[(2E)-But-2-enylamino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and crotonaldehyde, mp 110-113° C.

### EXAMPLE 371

### (7-{3-[(3-Methylbut-2-enyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 3-methyl-3-butenal, mp 116-118° C.

### EXAMPLE 372

### (7-{3-[(3,3-Dimethylbutyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 3,3-dimethylbutyraldehyde, mp 156-158° C.

### EXAMPLE 373

### [7-(6-Chloropyridin-3-yl)pyrazolo[1,5-a]pyrimidin-3-yl](thien-2-yl)methanone

The title compound was prepared from (3-amino-1H-pyrazol-4-yl)-2-thienyl-methanone and (2E)-1-(6-chloropyridine-3-yl)-3-(dimethylamino)prop-2-en-1-one by an analogous procedure to Method A, mp 145-148° C.

(2E)-1-(6-Chloropyridine-3-yl)-3-(dimethylamino)prop-2-en-1-one was prepared from 6-chloro-3-acetyl-pyridine and dimethylformamide-dimethylacetal using an analogous to Method B.

### EXAMPLE 374

### 2,6-Difluoro-N-{2-fluoro-5-[3-(thien-2-ylcarbonyl)pyrazolo[1,5-a]pyrimidin-7-yl]phenyl}benzamide

The title compound was prepared using a procedure analogous to Method D from {[7-(3-amino-4-fluoro)phenyl]pyrazolo[1,5-a]pyrimidin-3-yl}-2-thienyl-methanone and 2,6-difluorobenzoyl chloride, mp 198-200° C.

### EXAMPLE 375

### 2-fluoro-N-{2-fluoro-5-[3-(thien-2-ylcarbonyl)pyrazolo[1,5-a]pyrimidin-7-yl]phenyl}benzamide

The title compound was prepared using a procedure analogous to Method D from {[7-(3-amino-4-fluoro)phenyl]pyrazolo[1,5-a]pyrimidin-3-yl}-2-thienyl-methanone and 2-fluorobenzoyl chloride, mp 214-216° C.

### EXAMPLE 376

### 2-Chloro-N-{2-fluoro-5-[3-(thien-2-ylcarbonyl)pyrazolo[1,5-a]pyrimidin-7-yl]phenyl}benzamide

The title compound was prepared using a procedure analogous to Method D from {[7-(3-amino-4-fluoro)phenyl]pyrazolo[1,5-a]pyrimidin-3-yl}-2-thienyl-methanone and 2-chlorobenzoyl chloride, mp 166-168° C.

### EXAMPLE 377

### (7-{3-[(2-Methylbenzyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-methylbenzaldehyde, mp 177-179° C.

### EXAMPLE 378

### (7-{3-[(2-Phenylpropyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-phenylpropinonaldehyde, mp 108-110° C.

### EXAMPLE 379

### (7-{3-[(Cyclohexylmethyl)amino]phenyl)pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and cyclohexanecarboxaldehyde, mp 101-104° C.

### EXAMPLE 380

### {7-[3-(Butylamino)phenyl]pyrazolo[1,5-a]pyrimidin-3-yl}(thien-2-yl)methanone

The title compound was prepared using an analogous procedure to Method F from [7-(3-aminophenyl)pyrazolo[1,5-a]pyrimidin-3-yl]-2-thienyl-methanone and butyraldehyde, mp 87-91° C.

### EXAMPLE 381

### 2-Chloroethyl 2-fluoro-5-[3-(thien-2-ylcarbonyl)pyrazolo[1,5-a]pyrimidin-7-yl]phenylcarbamate

The title compound was prepared using a procedure analogous to Method H from {[7-(3-amino-4-fluoro)phenyl]pyrazolo[1,5-a]pyrimidin-3-yl}-2-thienyl-methanone and 2-chloroethyl chloroformate, mp 194-196° C.

### EXAMPLE 382

### WAY0185720

### N-{2-Fluoro-5-[3-(thien-2-ylcarbonyl)pyrazolo[1,5-a]pyrimidin-7-yl]phenyl}-N′-isopropylurea

The title compound was prepared using a procedure analogous to Method G from {[7-(3-amino-4-fluoro)phenyl]pyrazolo[1,5-a]pyrimidin-3-yl}-2-thienyl-methanone and isopropyl isocyanate, mp>200° C.

### EXAMPLE 383

### 4-(1H-pyrrol-1-yl)-N-{3-[3-(thien-2-ylcarbonyl)pyrazolo[1,5-a]pyrimidin-7-yl]phenyl}benzamide

The title compound was prepared using an analogous procedure to Method E from [7-(3-aminophenyl)pyrazolo[1,5-a]pyrimidin-3-yl]-2-thienyl-methanone and 4-(1H-pyrrol-1-yl)benzoic acid, mp 224-226° C.

### EXAMPLE 384

### 2,6-Dichloro-N-{3-[3-(thien-2-ylcarbonyl)pyrazolo[1,5-a]pyrimidin-7-yl]phenyl}benzamide

The title compound was prepared using an analogous procedure to Method D from [7-(3-aminophenyl)pyrazolo[1,5-a]pyrimidin-3-yl]-2-thienyl-methanone and 2,6-dichlorobenzoyl chloride, mp 245-246° C.

### EXAMPLE 385

### N-{3-[3-(Thien-2-ylcarbonyl)pyrazolo[1,5-a]pyrimidin-7-yl]phenyl}pyrazine-2-carboxamide

The title compound was prepared using an analogous procedure to Method E from [7-(3-aminophenyl)pyrazolo[1,5-a]pyrimidin-3-yl]-2-thienyl-methanone and 2-pyrazinecarboxylic acid, mp 222-224° C.

### EXAMPLE 386

### Phenyl N′-cyano-N-{3-[3-(thien-2-ylcarbonyl)pyrazolo[1,5-a]pyrimidin-7-yl]phenyl}imidocarbamate

To a mixture of [7-(3-aminophenyl)pyrazolo[1,5-a]pyrimidin-3-yl]-2-thienyl-methanone (200 mg, 0.63 mmol) and diphenylcyanocarbonimidate (149 mg, 0.63 mmol) was added acetonitrile (3 mL). The reaction mixture was heated at reflux for 3 hours, then stirred at room temperature overnight. The resultant suspension was slurried with acetonitrile, filtered and washed with acetonitrile. The crude solid was further purified with flash column chromatography eluting with 0.5% methanol/methylene chloride to give 160 mg (55%) of phenyl N′-cyano-N′-{3-[3-(thien-2-ylcarbonyl)pyrazolo[1,5-a]pyrimidin-7-yl]phenyl}imidocarbamate as an off-white solid, mp 220° C. (dec.).

### EXAMPLE 387

### 1-Methyl-N-{3-[3-(thien-2-ylcarbonyl)pyrazolo[1,5-a]pyrimidin-7-yl]phenyl}-1H-pyrrole-2-carboxamide

The title compound was prepared using an analogous procedure to Method E from [7-(3-aminophenyl)pyrazolo[1,5-a]pyrimidin-3-yl]-2-thienyl-methanone and N-methyl-2-pyrrolcarboxylic acid, mp 120° C. (dec.).

### EXAMPLE 388

### N-{2-Fluoro-5-[3-(thien-2-ylcarbonyl)pyrazolo[1,5-a]pyrimidin-7-yl]phenyl}isonicotinamide

The title compound was prepared using an analogous procedure to Method D from {[7-(3-amino-4-fluoro)phenyl]pyrazolo[1,5-a]pyrimidin-3-yl}-2-thienyl-methanone and isonicotinyl chloride, mp 175-179° C.

### EXAMPLE 389

### N-Isopropyl-N′-{3-[3-(thien-2-ylcarbonyl)pyrazolo[1,5-a]pyrimidin-7-yl]phenyl}thiourea

The title compound was prepared using an analogous procedure to Example 334 from [7-(3-aminophenyl)pyrazolo[1,5-a]pyrimidin-3-yl]-2-thienyl-methanone and isopropyl thioisocyanate, mp 208-210° C.

### EXAMPLE 390

### (7-Chloropyrazolo[1,5-a]pyrimidin-3-yl)(thien-2-yl)methanone

A mixture of (4-oxo-pyrazolo[1,5-a]pyrimidin-3-yl)(thien-2-yl)methanone (100 mg, 0.41 mmol), phosphorous oxychloride (313 mg, 2.0 mmol) and N,N-diethylaniline (73 mg, 0.49 mmol) in toluene (4 mL) was heated at reflux for 6 hours followed by stirring at room temperature overnight. The resultant suspension was filtered and the filtrate was partitioned between water and ethyl acetate. The combined organics were dried over sodium sulfate, concentrated and purified by flash column chromatography to give 27 mg (25%) of the title compound as a light yellow solid, mp 148-150° C.

Preparation of (4-oxo-pyrazolo[1,5-a]pyrimidin-3-yl)(thien-2-yl)methanone: Sodium suspension (30% in toluene) (794 mg, 10.4 mmol) was diluted with toluene (50 mL). Ethyl acetate was added via syringe dropwise, followed by the addition of ethyl formate (766 mg, 10.4 mmol) over 5 minutes. The reaction mixture was stirred at room temperature overnight. (3-Amino-1H-pyrazol-4-yl)-2-thienyl-methanone (1.0 g, 5.2 mmol) in ethanol (40 mL) was added via syringe and the reaction mixture was heated at reflux for 24 hours. Volatiles were evaporated under reduced pressure and the residue was slurried in water. The mixture was acidified with acetic acid to pH 4-5. The precipitate was collected by filtration, washed with diluted acetic acid, water and ether and dried to give 464 mg (37%) of (4-oxo-pyrazolo[1,5-a]pyrimidin-3-yl)(thien-2-yl)methanone as a light pink solid.

### EXAMPLE 391

### 2,6-Dichloro-N-(2,6-dichlorobenzoyl)-N-{3-[3-(thien-2-ylcarbonyl)pyrazolo[1,5-a]pyrimidin-7-yl]phenyl}benzamide

The title compound was prepared using an analogous procedure to Method D from [7-(3-aminophenyl)pyrazolo[1,5-a]pyrimidin-3-yl]-2-thienyl-methanone and excess of 2,6-dichlorobenzoyl chloride, mp 238-240° C.

### EXAMPLE 392

### tert-Butyl 4-[2-oxo-2-({3-[3-(thien-2-ylcarbonyl)pyrazolo[1.5-a]pyrimidin-7-yl]phenyl}amino)ethyl]piperidine-1-carboxylate

The title compound was prepared using an analogous procedure to Method E from [7-(3-aminophenyl)pyrazolo[1,5-a]pyrimidin-3-yl]-2-thienyl-methanone and 1-t-Boc-piperidineacetic acid, mp 173-175° C.

Please Advise, What Literature?).

### EXAMPLE 393

### 2-Piperidin-4-yl-N-{3-[3-(thien-2-ylcarbonyl)pyrazolo[1,5-a]pyrimidin-7-yl]phenyl}acetamide

A mixture of tert-butyl 4-[2-oxo-2-({3-[3-(thien-2-ylcarbonyl)pyrazolo[1,5-a]pyrimidin-7-yl]phenyl}amino)ethyl]piperidine-1-carboxylate (Example 286) (280 mg, 0.48 mmol) and trifluoroacetic acid (0.37 mL) in methylene chloride (6 ml) was stirred at room temperature overnight. The reaction mixture was evaporated under reduced pressure and the residue was slurried in saturated aqueous sodium bicarbonate. The solid was collected by filtration and recrystalized with ethyl acetate/hexanes to give 210 mg (91%) of the title compound as an off-white solid, mp 216-220° C.

### EXAMPLE 394

### {7-[3-(Diethylamino)phenyl]pyrazolo[1,5-a]pyrimidin-3-yl}(thien-2-yl)methanone

The title compound was prepared using an analogous procedure to Method F from [7-(3-aminophenyl)pyrazolo[1,5-a]pyrimidin-3-yl]-2-thienyl-methanone and excess of acetaldehyde, mp 102-104° C.

### EXAMPLE 395

### [7-(3-{[(2E)-3-(2-Methoxyphenyl)prop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 2-methoxycinnamaldehyde, mp 228-131° C.

### EXAMPLE 396

### [7-(3-{[(2E)-3-(4-Methoxyphenyl)prop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 4-methoxycinnamaldehyde, mp 176-179° C.

### EXAMPLE 397

### [7-(3-{[(2E)-2-Methyl-3-phenylprop-2-enyl]amino}phenyl)pyrazolo[1,5-a]pyrimidin-3-yl] (thien-2-yl)methanone

The title compound was prepared using an analogous procedure to Method F from [7-(3-aminophenyl)pyrazolo[1,5-a]pyrimidin-3-yl]-2-thienyl-methanone and α-methyl-trans-cinnamaldehyde, mp 74-76° C.

### EXAMPLE 398

### [7-(3-{[(2E)-3-(4-Nitrophenyl)prop-2-enyl]amino}phenyl)pyrazolo[1,5-a]pyrimidin-3-yl] (thien-2-yl)methanone

The title compound was prepared using an analogous procedure to Method F from [7-(3-aminophenyl)pyrazolo[1,5-a]pyrimidin-3-yl]-2-thienyl-methanone and 4-nitrocinnamaldehyde, mp 163-166° C.

### EXAMPLE 399

### (7-{3-[(3,3-Diphenylprop-2-enyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and β-phenylcinnamaldehyde, mp 156-158° C.

### EXAMPLE 400

### (7-{3-[(3-Phenylbutyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 3-phenylbutyraldehyde, mp 64-68° C.

### EXAMPLE 401

### [7-(3-{[(2E)-3-(4-Hydroxy-3-methoxyphenyl)prop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 3,4-dimethoxycinnamaldehyde, mp 180-184° C.

### EXAMPLE 402

### (7-{3-[(2-Methylbutyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-methylbutyraldehyde, mp 210-212° C.

### EXAMPLE 403

### (7-{3-[(2-Ethylbutyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-ethylbutyraldehyde, mp 192-194° C.

### EXAMPLE 404

### {7-[3-(Isobutylamino)phenyl]pyrazolo[1,5-a]pyrimidin-3-yl}(thien-2-yl)methanone

The title compound was prepared using an analogous procedure to Method F from [7-(3-aminophenyl)pyrazolo[1,5-a]pyrimidin-3-yl]-2-thienyl-methanone and isobutyraldehyde, mp 186-188° C.

### EXAMPLE 405

### tert-Butyl 4-[({3-[3-[3-(thien-2-ylcarbonyl)pyrazolo[1,5-a]pryimidin-7-yl]phenyl}amino)carbonyl]piperidine-1-carboxylate

The title compound was prepared using an analogous procedure to Method E from [7-(3-aminophenyl)pyrazolo[1,5-a]pyrimidin-3-yl]-2-thienyl-methanone and 1-t-Boc-carboxylic acid, mp 148-150° C.

### EXAMPLE 406

### N,N′-bis {3-[3-(Thien-2-ylcarbonyl)pyrazolo[1,5-a]pyrimidin-7-yl]phenyl}thiourea

The title compound was obtained as an additional product in the preparation of Example 334, mp 151-153° C.

### EXAMPLE 407

### Isobutyl 3-(3-thien-2-ylpyrazolo[1,5-a]pyrimidin-7-yl)phenylcarbamate

The title compound was prepared using a procedure analogous to Example 325 from isobutyl 3-(3-bromopyrazolo[1,5-a]pyrimidin-7-yl)phenylcarbamate and 2-thiopheneboronic acid, mp 140-141° C.

### EXAMPLE 408

### N-{3-[3-(Thien-2-ylcarbonyl)pyrazolo[1,5-a]pyrimidin-7-yl]phenyl}piperidine-4-carboxamide

The title compound was prepared using an analogous procedure to Example 393 from tert-butyl 4-[({3-[3-(thien-2-ylcarbonyl)pyrazolo[1,5-a]pyrimidin-7-yl]phenyl}amino)carbonyl]-piperidine-1-carboxylate, mp 159-161° C.

### EXAMPLE 409

### [7-(3-{[(2E)-2-Hexyl-3-phenylprop-2-enyl]amino}phenyl)pyrazolo[1,5-a]pyrimidin-3-yl] (thien-2-yl)methanone

The title compound was prepared using an analogous procedure to Method F from [7-(3-aminophenyl)pyrazolo[1,5-a]pyrimidin-3-yl]-2-thienyl-methanone and 2-hexylcinnamaldehyde, mp 110-115° C.

### EXAMPLE 410

### [7-(3-{[(2E)-2-Pentyl-3-phenylprop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 2-pentylcinnamaldehyde, mp 110-112° C.

### EXAMPLE 411

### N-Isopropyl-N′-{3-[3-(thien-2-ylcarbonyl)pyrazolo[1,5-a]pyrimidin-7-yl]phenyl}guanidine

[7-(3-Aminophenyl)pyrazolo[1,5-a]pyrimidin-3-yl]-2-thienyl-methanone (100 mg, 0.31 mmol) was suspended in methanol (10 mL) and cooled to 0° C. with an ice bath. Hydrogen chloride gas was bubbled into the suspension for 30 minutes. The volatiles were evaporated under reduced pressure and the residue was dried to give an orange solid which was used for the next step without further purification, mp>200° C.

[7-(3-Aminophenyl)pyrazolo[1,5-a]pyrimidin-3-yl]-2-thienyl-methanone hydrochloride salt (100 mg, 0.31 mmol) and isopropyl cyanamide (35 mg, 0.41 mmol) in methylene chloride (5.0 mL) were heated at reflux overnight. The reaction mixture was cooled, concentrated on silica gel and purified by flash column chromatography eluting with a gradient mixture of methanol/methylene chloride to give 84 mg (55%) of the title compound as an off-white solid.

Preparation of isopropyl cyanamide: Cyanogen bromide (1.13 g, 10.7 mmol) in ether (5 mL) was added slowly to a solution of isopropylamine (1 g, 16.9 mmol) in ether (10 mL) with cooling of an ice bath. The reaction mixture was stirred overnight at room temperature, filtered and washed with ether. The combined organics were concentrated to give a yellow liquid which was used for the next step directly.

### EXAMPLE 412

### [7-(3-{[(2E)-3-Phenylprop-2-enyl]amino}phenyl)pyrazolo[1,5-a]pyrimidin-3-yl] (thien-2-yl)methanone

The title compound was prepared using an analogous procedure to Method F from [7-(3-aminophenyl)pyrazolo[1,5-a]pyrimidin-3-yl]-2-thienyl-methanone and cinnamaldehyde, mp 123-125° C.

### EXAMPLE 413

### [7-(3-{[(2E)-3-(4-tert-Butylphenyl)-2-methylprop-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and α-methyl-4-tert-butylcinnamaldehyde, mp 135-137° C.

### EXAMPLE 414

### N″-Cyano-N-isopropyl-N′-{3-[3-(thien-2-ylcarbonyl)pyrazolo[1,5-a]pyrimidin-7-yl]phenyl}guanidine

A mixture of phenyl N′-cyano-N′-{3-[3-(thien-2-ylcarbonyl)pyrazolo [1,5-a]pyrimidin-7-yl]phenyl}imidocarbamate (Example 386) (120 mg, 0.26 mmol) and isopropylamine (30 mg, 0.52 mmol) in 2-propanol (3 mL) were heated at reflux for 72 hours. The resultant mixture was cooled and filtered. The crude solid was further purified by flash column chromatography eluting with a gradient mixture of methanol and methylene chloride to give 45 mg (41%) of the title compound as a white solid.

### EXAMPLE 415

### N-{3-[3-(Thien-2-ylcarbonyl)pyrazolo[1,5-a]pyrimidin-7-yl]phenyl}-1H-imidazole-5-carboxamide

A mixture of [7-(3-aminophenyl)pyrazolo[1,5-a]pyrimidin-3-yl]-2-thienyl-methanone (100 mg, 0.31 mmol) and 4-imidazolecarbonyl chloride-HCl (157 mg, 0.94 mmol) (J. Org. Chem. 1998:8084) was suspended in acetic acid (30 mL). Sodium acetate (256 mg, 3.13 mmol) in acetic acid (10 mL) was added dropwise via a dropping funnel over 60 minutes. The resultant mixture was stirred at room temperature for 3 days and evaporated under reduced pressure to remove the volatiles. The residue was purified by flash column chromatography eluting with 2% methanol/methylene chloride to give 63 mg (49%) of the title compound as a white solid, mp 280-282° C.

### EXAMPLE 416

### Pyridin-4-ylmethyl 3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and 4-pyridylcarbinol, mp 185° C. (dec.).

### 417

### 1-Methyl-N-{3-[3-(thien-2-ylcarbonyl)pyrazolo[1,5-a]pyrimidin-7-yl]phenyl}piperidine-4-carboxaamide Example

The title compound was prepared using an analogous procedure to Method E from [7-(3-aminophenyl)pyrazolo[1,5-a]pyrimidin-3-yl]-2-thienyl-methanone and N-methylpiperazinecarboxylic acid, mp 236° C.

### EXAMPLE 418

### (7-{3-[(2-Methylpentyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-methylvaleraldehyde, mp 133-135° C.

### EXAMPLE 419

### (7-{3-[(2-Ethylhexyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-ethylhexanal, mp 134-138° C.

### EXAMPLE 420

### 3-(1,1-Dioxidothiomorpholin-4-yl)propyl 3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and (1,1-dioxidothiomorpholin-4-yl)propanol (International publication WO 98/13354), mp 148-150° C.

### EXAMPLE 421

### 2-Chloro-N-{2-(dimethylamino)-5-[3-(thien-2-ylcarbonyl)pyrazolo[1,5-a]pyrimidin-7-yl]phenyl}benzamide

The title compound was prepared using an analogous procedure to Method D from [7-(3-amino-4-dimethylaminophenyl)pyrazolo[1,5-a]pyrimidin-3-yl]-2-thienyl-methanone and 2-chlorobenzoyl chloride, mp 220-222° C.

### EXAMPLE 422

### {7-[3-(Ethylamino)phenyl]pyrazolo[1,5-a]pyrimidin-3-yl}(thien-2-yl)methanone

The title compound was prepared using an analogous procedure to Method F from [7-(3-aminophenyl)pyrazolo[1,5-a]pyrimidin-3-yl]-2-thienyl-methanone and acetaldehyde, mp 154-155° C.

### EXAMPLE 423

### 4-Morpholin-4-yl-N-{3-[3-(thien-2-ylcarbonyl)pyrazolo[1,5-a]pyrimidin-7-yl]phenyl}butanamide

The title compound was prepared using an analogous procedure to Method E from [7-(3-aminophenyl)pyrazolo[1,5-a]pyrimidin-3-yl]-2-thienyl-methanone and 3-(1-morpholine)butyric acid, mp 127-133° C.

### EXAMPLE 424

### Pyridin-3-ylmethyl 3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and 3-pyridylcarbinol, mp>200° C.

### EXAMPLE 425

### 3-Morpholin-4-ylpropyl 3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and 3-morpholinepropanol, mp 198-199° C.

### EXAMPLE 426

### [7-(3-{[(2E)-2-Methylpent-2-enyl]amino}phenyl)pyrazolo[1,5-a]pyrimidin-3-yl](thien-2-yl)methanone

The title compound was prepared using an analogous procedure to Method F from [7-(3-aminophenyl)pyrazolo[1,5-a]pyrimidin-3-yl]-2-thienyl-methanone and 2-methyl-2-pentenal, mp 132-133° C.

### EXAMPLE 427

### 2-Pyridin-4-yl-N-{3-[3-(thien-2-ylcarbonyl)pyrazolo[1,5-a]pyrimidin-7-yl]phenyl}acetamide

The title compound was prepared using an analogous procedure to Method E from [7-(3-aminophenyl)pyrazolo[1,5-a]pyrimidin-3-yl]-2-thienyl-methanone and 4-pyridineacetic acid, mp 130-131° C.

### EXAMPLE 428

### N-{2-(Dimethylamino)-5-[3-(thien-2-ylcarbonyl)pyrazolo[1,5-a]pyrimidin-7-yl]phenyl}isonicotinamide

The title compound was prepared using an analogous procedure to Method D from [7-(3-amino-4-dimethylaminophenyl)pyrazolo[1,5-a]pyrimidin-3-yl]-2-thienyl-methanone and isonicotinyl chloride, mp 151-153° C.

### EXAMPLE 429

### Pyridin-2-ylmethyl 3-[3-(thien-2-ylcarbonyl)pyrazolo[1,5-a]pyrimidin-7-yl]phenylcarbamate

The title compound was prepared using an analogous procedure to Method I from [7-(3-aminophenyl)pyrazolo[1,5-a]pyrimidin-3-yl]-2-thienyl-methanone and 2-pyridylcarbinol, mp 121-125° C.

### EXAMPLE 430

### (7-{3-[(2,3-Dimethylpentyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2,3-dimethylvaleraldehyde, mp 84-86° C.

### EXAMPLE 431

### [7-(3-{[(2E)-2-Methylbut-2-enyl]amino}phenyl)pyrazolo[1,5-a]pyrimidin-3-yl] (thien-2-yl)methanone

The title compound was prepared using an analogous procedure to Method F from [7-(3-aminophenyl)pyrazolo[1,5-a]pyrimidin-3-yl]-2-thienyl-methanone and trans-2-methyl-2-butenal, mp 118-120° C.

### EXAMPLE 432

### (7-{3-[(2E)-Pent-2-enylamino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-pentenal, mp 72-75° C.

### EXAMPLE 433

### Thien-2-yl(7-{3-[(3,5,5-trimethylhexyl)amino]phenyl}pyrazolo[1,5-a]pyrimidin-3-yl)methanone

The title compound was prepared using an analogous procedure to Method F from [7-(3-aminophenyl)pyrazolo[1,5-a]pyrimidin-3-yl]-2-thienyl-methanone and 3,5,5-trimethylhexanal, mp 81-85° C.

### EXAMPLE 434

### (7-{3-[(2-Methylprop-2-enyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-methyl-2-propenal, mp 104-106° C.

### EXAMPLE 435

### (7-{3-[(2-Ethylprop-2-enyl)amino]phenyl}pyrazolo[1,5-a]pyrimidin-3-yl)(thien-2-yl)methanone

The title compound was prepared using an analogous procedure to Method F from [7-(3-aminophenyl)pyrazolo[1,5-a]pyrimidin-3-yl]-2-thienyl-methanone and 2-ethyl-2-propenal, mp 79-82° C.

### EXAMPLE 436

### (7-{3-[(cyclohex-3-en-1-ylmethyl)amino]phenyl}pyrazolo[1,5-a]pyrimidin-3-yl)-(thien-2-yl)-methanone

The title compound was prepared using an analogous procedure to Method F from [7-(3-aminophenyl)pyrazolo[1,5-a]pyrimidin-3-yl]-2-thienyl-methanone and 3-cyclohexenecarboxyaldehyde, mp 78-81° C.

### EXAMPLE 437

### 1-{3-[3-(Thien-2-ylcarbonyl)pyrazolo[1,5-a]pyrimidin-7-yl]phenyl}pyrrolidin-2-one

The title compound was prepared from [7-(3-aminophenyl)pyrazolo[1,5-a]pyrimidin-3-yl]-2-thienyl-methanone and 4-bromobutyryl chloride by an analogous procedure to Method D, mp>200° C.

### EXAMPLE 438

### 2-Chloro-N-(2-chlorobenzoyl)-N-{2-fluoro-5-[3-(thien-2-ylcarbonyl)pyrazolo[1,5-a]pyrimidin-7-yl]phenyl}benzamide

The title compound was prepared from [7-(3-aminophenyl)pyrazolo[1,5-a]pyrimidin-3-yl]-2-thienyl-methanone and excess amount of 2-chlorobenzoyl chloride by an analogous procedure to Method D, mp 150-151° C.

### EXAMPLE 439

### 2-Pyridin-3-yl-N-{3-[3-(thien-2-ylcarbonyl)pyrazolo[1,5-a]pyrimidin-7-yl]phenyl}acetamide

The title compound was prepared using an analogous procedure to Method E from [7-(3-aminophenyl)pyrazolo[1,5-a]pyrimidin-3-yl]-2-thienyl-methanone and 3-pyridineacetic acid, mp>200° C.

### EXAMPLE 440

### 3-Pyridin-4-yl-N-{3-[3-(thien-2-ylcarbonyl)pyrazolo[1,5-a]pyrimidin-7-yl]phenyl}propanamide

The title compound was prepared using an analogous procedure to Method E from [7-(3-aminophenyl)pyrazolo[1,5-a]pyrimidin-3-yl]-2-thienyl-methanone and 4-pyridinepropionic acid, mp 176-181° C.

### EXAMPLE 441

### N-{3-[3-(Thien-2-ylcarbonyl)pyrazolo[1,5-a]pyrimidin-7-yl]phenyl}-1H-pyrazole-4-carboxamide

The title compound was prepared using an analogous procedure to Method E from [7-(3-aminophenyl)pyrazolo[1,5-a]pyrimidin-3-yl]-2-thienyl-methanone and 3-pyrazolecarboxylic acid, mp 165-170° C.

### EXAMPLE 442

### 4-Oxo-4-({3-[3-(thien-2-ylcarbonyl)pyrazolo[1,5-a]pyrimidin-7-yl]phenyl}amino)butanoic acid

To a suspension of [7-(3-aminophenyl)pyrazolo[1,5-a]pyrimidin-3-yl]-2-thienyl-methanone (100 mg, 0.31 mmol) in dioxane (6 mL) was added succinic anhydride (47 mg, 0.47 mmol). The reaction mixture was stirred at room temperature overnight followed by stirring at 60° C. for 12 hours, cooled and diluted with ether. The precipitate was collected by filtration and washed with ether to afford 106 mg (82%) of the title compound as an off-white solid, mp 240-241° C.

### EXAMPLE 443

### 2-[({3-[3-(Thien-2-ylcarbonyl)pyrazolo[1,5-a]pyrimidin-7-yl]phenyl}amino)carbonyl]benzoic acid

The title compound was prepared from [7-(3-aminophenyl)pyrazolo[1,5-a]pyrimidin-3-yl]-2-thienyl-methanone and phthalic anhydride by an analogous procedure described in Example 442, mp 215-216° C.

### EXAMPLE 444

### 5-Oxo-5-({3-[3-(thien-2-ylcarbonyl)pyrazolo[1,5-a]pyrimidin-7-yl]phenyl}amino)pentanoic acid

The title compound was prepared from [7-(3-aminophenyl)pyrazolo[1,5-a]pyrimidin-3-yl]-2-thienyl-methanone and glutaric anhydride by an analogous procedure described in Example 442, mp 189-190° C.

### EXAMPLE 445

### 3-Methyl-5-oxo-5-({3-[3-(thien-2-ylcarbonyl)pyrazolo[1,5-a]pyrimidin-7-yl]phenyl}amino)pentanoic acid

The title compound was prepared from [7-(3-aminophenyl)pyrazolo[1,5-a]pyrimidin-3-yl]-2-thienyl-methanone and 3-methylglutaric anhydride by an analogous procedure described in Example 442, mp 135-136° C.

### EXAMPLE 446

### 4,5-Dichloro-2-[({3-[3-(thien-2-ylcarbonyl)pyrazolo[1,5-a]pyrimidin-7-yl]phenyl}amino)carbonyl]benzoic acid

The title compound was prepared from [7-(3-aminophenyl)pyrazolo[1,5-a]pyrimidin-3-yl]-2-thienyl-methanone and 4,5-dichlorophthalic anhydride by an analogous procedure described in Example 442, mp>270° C.

### EXAMPLE 447

### (7-{3-[(2-Methylundecyl)amino]phenyl}pyrazolo[1,5-a]pyrimidin-3-yl)(thien-2-yl)methanone

The title compound was prepared from [7-(3-aminophenyl)pyrazolo[1,5-a]pyrimidin-3-yl]-2-thienyl-methanone and 2-methylundecanal by an analogous procedure described in Method F, mp 108-110° C.

### EXAMPLE 448

### 3-Pyridin-3-yl-N-{3-[3-(thien-2-ylcarbonyl)pyrazolo[1,5-a]pyrimidin-7-yl]phenyl}propanamide

The title compound was prepared using an analogous procedure to Method E from [7-(3-aminophenyl)pyrazolo[1,5-a]pyrimidin-3-yl]-2-thienyl-methanone and 3-pyridinepropionic acid, mp>200° C.

### EXAMPLE 449

### 2-(Pyridin-4-ylthio)-N-{3-[3-(thien-2-ylcarbonyl)pyrazolo[1,5-a]pyrimidin-7-yl]phenyl}acetamide

The title compound was prepared using an analogous procedure to Method E from [7-(3-aminophenyl)pyrazolo[1,5-a]pyrimidin-3-yl]-2-thienyl-methanone and (4-pyridylthio)acetic acid, mp 138-144° C.

### EXAMPLE 450

### {7-[4′-(Dimethylamino)-1,1′-biphenyl-3-yl]pyrazolo[1,5-a]pyrimidin-3-yl}(thien-2-yl)methanone

The title compound was prepared as described Method M from 4-N,N-dimethyaminophenyl boronic acid and [7-(3-bromophenyl)pyrazolo[1,5-a]pyrimidin-3-yl]-2-thienyl-methanone, mp 200-202° C.

### EXAMPLE 451

### [7-(3′-Amino-1,1′-biphenyl-3-yl)pyrazolo[1,5-a]pyrimidin-3-yl](thien-2-yl)methanone

The title compound was prepared using an analogous procedure to Method M from 3-aminophenyl boronic acid and [7-(3-bromophenyl)pyrazolo[1,5-a]pyrimidin-3-yl]-2-thienyl-methanone, mp 117-121° C.

### EXAMPLE 452

### {7-[4′-(Hydroxymethyl)-1,1′-biphenyl-3-yl]pyrazolo[1,5-a]pyrimidin-3-yl}(thien-2-yl)methanone

The title compound was prepared using an analogous procedure to Method M from 4-(hydroxymethyl)phenyl boronic acid and [7-(3-bromophenyl)pyrazolo[1,5-a]pyrimidin-3-yl]-2-thienyl-methanone, mp 106-110° C.

### EXAMPLE 453

### {7-[3′-(Dimethylamino)-1,1′-biphenyl-3-yl]pyrazolo[1,5-a]pyrimidin-3-yl}(thien-2-yl)methanone

The title compound was prepared using an analogous procedure to Method M from 4-(N,N-dimethylamino)phenyl boronic acid and [7-(3-bromophenyl)pyrazolo[1,5-a]pyrimidin-3-yl]-2-thienyl-methanone, mp 167-169° C.

### EXAMPLE 454

### {7-[4′-(Dimethylamino)-6-fluoro-1,1′-biphenyl-3-yl]pyrazolo[1,5-a]pyrimidin-3-yl}(thien-2-yl)methanone

The title compound was prepared using an analogous procedure to Method M from 4-(N,N-dimethylamino)phenyl boronic acid and [7-(3-bromo-4-fluorophenyl)pyrazolo[1,5-a]pyrimidin-3-yl]-2-thienyl-methanone, mp 219-221° C.

[7-(3-bromo-4-fluorophenyl)pyrazolo[1,5-a]pyrimidin-3-yl]-2-thienyl-methanone was prepared using an analogous procedure to Method A from (5-amino-1H-pyrazol-4-yl)(thien-2-yl)methanone and 1-(3-bromo-4-fluoro-phenyl)-3-dimethylamino-propenone, mp 74-76° C.

### EXAMPLE 455

### {7-[6-Fluoro-4′-(hydroxymethyl)-1,1′-biphenyl-3-yl]pyrazolo[1,5-a]pyrimidin-3-yl}(thien-2-yl)methanone

The title compound was prepared using an analogous procedure to Method M from 4-(hydroxymethyl)phenyl boronic acid and [7-(3-bromo-4-fluorophenyl)pyrazolo[1,5-a]pyrimidin-3-yl]-2-thienyl-methanone, mp 140-145° C.

### EXAMPLE 456

### [7-(4′-Ethyl-1,1′-biphenyl-3-yl)pyrazolo[1,5-a]pyrimidin-3-yl](thien-2-yl)methanone

The title compound was prepared using an analogous procedure to Method M from 4-ethylphenyl boronic acid and [7-(3-bromo-4-fluorophenyl)pyrazolo[1,5-a]pyrimidin-3-yl]-2-thienyl-methanone, mp 117-119° C.

### EXAMPLE 457

### 3-{3′-[3-(Thien-2-ylcarbonyl)pyrazolo[1,5-a]pyrimidin-7-yl]-1,1′-biphenyl-4-yl}propanoic acid

The title compound was prepared using an analogous procedure to Method M from 4-(2-carboxyethyl)phenyl boronic acid and [7-(3-bromo-4-fluorophenyl)pyrazolo[1,5-a]pyrimidin-3-yl]-2-thienyl-methanone, mp 117-119° C.

### EXAMPLE 458

### 3′-[3-(Thien-2-ylcarbonyl)pyrazolo[1,5-a]pyrimidin-7-yl]-1,1′-biphenyl-4-carbaldehyde

The title compound was prepared using an analogous procedure to Method M from 4-formylphenyl boronic acid and [7-(3-bromo-4-fluorophenyl)pyrazolo[1,5-a]pyrimidin-3-yl]-2-thienyl-methanone, mp 156-158° C.

### EXAMPLE 459

### {7-[4′-(Morpholin-4-ylmethyl)-1,1′-biphenyl-3-yl]pyrazolo[1,5-a]pyrimidin-3-yl}(thien-2-yl)methanone

The title compound was prepared as described Method N from 3′-[3-(thien-2-ylcarbonyl)pyrazolo[1,5-a]pyrimidin-7-yl]-1,1′-biphenyl-4-carbaldehyde and morpholine, mp 175-178° C.

### EXAMPLE 460

### (7-{4′-[(4-Methylpiperazin-1-yl)methyl]-1,1′-biphenyl-3-yl}pyrazolo[1,5-a]pyrimidin-3-yl)(thien-2-yl)methanone

The title compound was prepared using an analogous procedure to Method N from 3′-[3-(thien-2-ylcarbonyl)pyrazolo[1,5-a]pyrimidin-7-yl]-1,1′-biphenyl-4-carbaldehyde and N-methylpiperazine, mp 199-202° C.

### EXAMPLE 461

### [7-(4′-{[(2-Morpholin-4-ylethyl)amino]methyl}-1,1′-biphenyl-3-yl)pyrazolo[1,5-a]pyrimidin-3-yl](thien-2-yl)methanone

The title compound was prepared using an analogous procedure to Method N from 3′-[3-(thien-2-ylcarbonyl)pyrazolo[1,5-a]pyrimidin-7-yl]-1,1′-biphenyl-4-carbaldehyde and 4-(2-aminoethyl)morpholine, mp 100-106° C.

### EXAMPLE 462

### (7-{4′-[(Diethylamino)methyl]-1,1′-biphenyl-3-yl}pyrazolo[1,5-a]pyrimidin-3-yl)(thien-2-yl)methanone

The title compound was prepared using an analogous procedure to Method N from 3′-[3-(thien-2-ylcarbonyl)pyrazolo[1,5-a]pyrimidin-7-yl]-1,1′-biphenyl-4-carbaldehyde and diethylamine, mp 144-146° C.

### EXAMPLE 463

### (7-{4′-[(Dimethylamino)methyl]-1,1′-biphenyl-3-yl}pyrazolo[1,5-a]pyrimidin-3-yl)(thien-2-yl)methanone

The title compound was prepared using an analogous procedure to Method N from 3′-[3-(thien-2-ylcarbonyl)pyrazolo[1,5-a]pyrimidin-7-yl]-1,1′-biphenyl-4-carbaldehyde and dimethylamine, mp 192-195° C.

### EXAMPLE 464

### N-{3-[3-(3-Aminophenyl)pyrazolo[1,5-a]pyrimidin-7-yl]phenyl}-3-methylbutanamide

The title compound was prepared as described Method M from N-[3-(3-bromopyrazolo[1,5-a]pyrimidin-7-yl)phenyl]-3-methylbutanamide and 3-aminophenyl boronic acid, mp 173-175° C.

N-[3-(3-Bromopyrazolo[1,5-a]pyrimidin-7-yl)phenyl]-3-methylbutanamide was prepared using an analogous procedure to Method E from 3-(3-bromopyrazolo[1,5-a]pyrimidin-7-yl)aniline and isobutyl chloroformate, mp 132-134° C.

3-(3-Bromopyrazolo[1,5-a]pyrimidin-7-yl)aniline was prepared using an analogous procedure to Method C from 3-bromo-7-(3-nitrophenyl)pyrazolo[1,5-a]pyrimidine, mp 120-121° C.

3-Bromo-7-(3-nitrophenyl)pyrazolo[1,5-a]pyrimidine was prepared as follows: To a solution of 7-(3-nitrophenyl)pyrazolo[1,5-a]pyrimidine (2.0 g, 8.3 mmol) in chloroform (20 mL) was added N-bromosuccinimide (1.56 g, 8.75 mmol) in portions and thre reaction mixture was heated at reflux for 20 minutes to resulted in a thick suspension. The solid was collected via filtration and was further purifed by silica gel flash column chromatography to give 1.98 g (75%) of the desired product as a yellow solid, mp 237-238° C.

7-(3-Nitrophenyl)pyrazolo[1,5-a]pyrimidine was prepared using an analogous procedure to Method A from 1-(3-nitro-phenyl)-3-dimethylamino-propenone and 3-aminopyrazole, mp 204-206° C.

### EXAMPLE 465

### N-(3-{3-[3-(Dimethylamino)phenyl]pyrazolo[1,5-a]pyrimidin-7-yl}phenyl)-3-methylbutanamide

The title compound was prepared using an analogous procedure to Method M from N-[3-(3-bromopyrazolo[1,5-a]pyrimidin-7-yl)phenyl]-3-methylbutanamide and 3-N,N-dimethylaminophenyl boronic acid, mp 182-184° C.

### EXAMPLE 466

### N-{3-[3-(1H-Indol-5-yl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 5-Indolylboronic acid, mp 167-169° C.

### EXAMPLE 467

### 3-Methyl-N-{3-[3-(3,4,5-trimethoxyphenyl)pyrazolo[1,5-a]pyrimidin-7-yl]phenyl}butanamide

The title compound was prepared using an analogous procedure to Method M from N-[3-(3-bromopyrazolo[1,5-a]pyrimidin-7-yl)phenyl]-3-methylbutanamide and 3,4,5-trimethoxyphenyl boronic acid, mp 86-90° C.

### EXAMPLE 468

### 3-Methyl-N-[3-(3-quinolin-8-ylpyrazolo[1,5-a]pyrimidin-7-yl)phenyl]butanamide

The title compound was prepared using an analogous procedure to Method M from N-[3-(3-bromopyrazolo[1,5-a]pyrimidin-7-yl)phenyl]-3-methylbutanamide and 8-quinolineboronic acid, mp 96-99° C.

### EXAMPLE 469

### N-(3-{3-[4-(Hydroxymethyl)phenyl]pyrazolo[1,5-a]pyrimidin-7-yl}phenyl)-3-methylbutanamide

The title compound was prepared using an analogous procedure to Method M from N-[3-(3-bromopyrazolo[1,5-a]pyrimidin-7-yl)phenyl]-3-methylbutanamide and 4-hydroxymethylboronic acid, mp 170-172° C.

### EXAMPLE 470

### N-{3-[3-(4-Methoxyphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 4-methoxyphenyl boronic acid, mp 225-227° C.

### EXAMPLE 471

### N-{3-[3-(3-Chlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-chlorophenyl boronic acid, mp 169-171° C.

### EXAMPLE 472

### N-{3-[3-(4-Chlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 4-chlorophenyl boronic acid, mp 215-217° C.

### EXAMPLE 473

### N-{3-[3-(2,3-Dichlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 2,3-dichlorophenyl boronic acid, mp 154-156° C.

### EXAMPLE 474

### N-{3-[3-(3,4-Dichlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3,4-dichlorophenyl boronic acid, mp 215-217° C.

### EXAMPLE 475

### N-{3-[3-(3,5-Dichlorophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3,5-dichlorophenyl boronic acid, mp 223-225° C.

### EXAMPLE 476

### N-{3-[3-(3,5-Dimethylphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3,5-dimethylphenyl boronic acid, mp 178-180° C.

### EXAMPLE 477

### 3-Methyl-N-(3-{3-[3-(trifluoromethyl)phenyl]pyrazolo[1,5-a]pyrimidin-7-yl}phenyl)butanamide

The title compound was prepared using an analogous procedure to Method M from N-[3-(3-bromopyrazolo[1,5-a]pyrimidin-7-yl)phenyl]-3-methylbutanamide and 3-trifluoromethylphenyl boronic acid, mp 170-173° C.

### EXAMPLE 478

### N-{3-[3-(3-Methoxyphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-methoxyphenyl boronic acid, mp 153-154° C.

### EXAMPLE 479

### N-{3-[3-(3-Ethoxyphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-ethoxyphenyl boronic acid, mp 156-158° C.

### EXAMPLE 480

### 3-Methyl-N-{3-[3-(4-phenoxyphenyl)pyrazolo[1,5-a]pyrimidin-7-yl]phenyl}butanamide

The title compound was prepared using an analogous procedure to Method M from N-[3-(3-bromopyrazolo[1,5-a]pyrimidin-7-yl)phenyl]-3-methylbutanamide and 4-phenoxyphenyl boronic acid, mp 175-178° C.

### EXAMPLE 481

### N-{3-[3-(1,3-Benzodioxol-5-yl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3,4-methylenedioxyphenyl boronic acid, mp 172-173° C.

### EXAMPLE 482

### N-{3-[3-(3-Cyanophenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-cyanophenyl boronic acid, mp 166-167° C.

### EXAMPLE 483

### N-{3-[3-(3-Acetylphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-acetylphenyl boronic acid, mp 190-192° C.

### EXAMPLE 484

### N-{3-[3-(3-Formyl-4-methoxyphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-formyl-4-methoxyphenyl boronic acid, mp 187-189° C.

### EXAMPLE 485

### N-{3-[3-(1,1′-Biphenyl-3-yl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-biphenyl boronic acid, mp 182-185° C.

### EXAMPLE 486

### N-{3-[3-(1,1′-Biphenyl-4-yl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 4-biphenyl boronic acid, mp 157-160° C.

### EXAMPLE 487

### N-{3-[3-(3-Formylphenyl)pyrazolo[1,5-a]pyrimidin-7-yl]phenyl}-3-methylbutanamide

The title compound was prepared using an analogous procedure to Method M from N-[3-(3-bromopyrazolo[1,5-a]pyrimidin-7-yl)phenyl]-3-methylbutanamide and 3-formylphenyl boronic acid, mp 163-165° C.

### EXAMPLE 488

### 3-Methyl-N-(3-{3-[3-(morpholin-4-ylmethyl)phenyl]pyrazolo[1,5-a]pyrimidin-7-yl}phenyl)butanamide

The title compound was prepared using an analogous procedure to Method N from N-{3-[3-(3-formylphenyl)pyrazolo[1,5-a]pyrimidin-7-yl]phenyl}-3-methylbutanamide and morpholine, mp 170-173° C.

### EXAMPLE 489

### 3-Methyl-N-[3-(3-{3-[(4-methylpiperazin-1-yl)methyl]phenyl}pyrazolo[1,5-a]pyrimidin-7-yl)phenyl]butanamide

The title compound was prepared using an analogous procedure to Method N from N-{3-[3-(3-formylphenyl)pyrazolo[1,5-a]pyrimidin-7-yl]phenyl}-3-methylbutanamide and N-methylpipeprazine, mp 135-138° C.

### EXAMPLE 490

### 3-[3-(3,5-Dimethylphenyl)pyrazolo[1,5-a]pyrimidin-7-yl]benzonitrile

The title compound was prepared using an analogous procedure to Method M from 3-(3-bromopyrazolo[1,5-a]pyrimidin-7-yl)benzonitrile and 3,5-dimethylphenylboronic acid, mp 147° C.

### EXAMPLE 491

### 3-(3,5-Dimethylphenyl)-7-[3-(1H-tetraazol-5-yl)phenyl]pyrazolo[1,5-a]pyrimidine

The title compound was prepared as described in Method O from 3-[3-(3,5-dimethylphenyl)pyrazolo[1,5-a]pyrimidin-7-yl]benzonitrile and sodium azide, mp 250° C. (dec.).

### EXAMPLE 492

### 7-{3-[2-(Cyclobutylmethyl)-2H-tetraazol-5-yl]phenyl}-3-(3,5-dimethylphenyl)pyrazolo[1,5-a]pyrimidine

The title compound was prepared as described in Method P from 3-(3,5-dimethylphenyl)-7-[3-(1H-tetraazol-5-yl)phenyl]pyrazolo[1,5-a]pyrimidine and cyclobutylmethyl bromide, mp 64° C.

### EXAMPLE 493

### 3-(3,5-Dimethylphenyl)-7-{3-[2-(2-morpholin-4-ylethyl)₇₂H-tetraazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 4-(2-chloroethyl)morpholine, mp 68° C.

### EXAMPLE 494

### 3-(3,5-Dimethylphenyl)-7-{3-[2-(pyridin-3-ylmethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 3-bromomethylpyridine, mp 73° C.

### EXAMPLE 495

### 3-(3,5-Dimethylphenyl)-7-{3-[2-(pyridin-2-ylmethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 2-bromomethylpyridine, mp 77° C.

### EXAMPLE 496

### 3-(3,5-Dimethylphenyl)-7-[3-(2-ethyl-2H-tetrazol-5-yl)phenyl]pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and bromoethane, mp 65° C.

### EXAMPLE 497

### N-[3-(3-{3-[(Dimethylamino)methyl]phenyl}pyrazolo[1,5-a]pyrimidin-7-yl)phenyl]-3-methylbutanamide

The title compound was prepared using an analogous procedure to Method N from N-{3-[3-(3-formylphenyl)pyrazolo[1,5-a]pyrimidin-7-yl]phenyl}-3-methylbutanamide and dimethylamine, mp 179-182° C.

### EXAMPLE 498

### 3-(3,5-Dimethylphenyl)-7-{3-[2-(2-pyrrolidin-1-ylethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 1-(2-chloroethyl)pyrrolidine, mp 60° C.

### EXAMPLE 499

### 3-(3,5-Dimethylphenyl)-7-{3-[2-(2-piperidin-1-ylethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 1-(2-chloroethyl)piperidine, mp 66° C.

### EXAMPLE 500

### 3-(3,5-Dimethylphenyl)-7-{3-[2-(2-piperidin-1-ylethyl)-2H-tetrazol-5-yl]phenyl}pyrazolo[1,5-a]pyrimidine

The title compound was prepared using an analogous procedure to Method P from 3-(3,5-dimethylphenyl)-7-[3-(1H-tetraazol-5-yl)phenyl]pyrazolo[1,5-a]pyrimidine and 4-bromoethylpyridine, mp 172° C.

### EXAMPLE 501

### {7-[3-(1H-Tetraazol-5-yl)phenyl]pyrazolo[1,5-a]pyrimidin-3-yl}(thien-2-yl)methanone

The title compound was prepared using an analogous procedure to Method O from 3-[3-(2-thienylcarbonyl)pyrazolo[1,5-a]-pyrimidin-7-yl}-benzonitrile and sodium azide, mp 290° C.

### EXAMPLE 502

### {7-[3-(2-Ethyl-2H-tetraazol-5-yl)phenyl]pyrazolo[1,5-a]pyrimidin-3-yl}(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and ethyl iodide, mp 174° C.

### EXAMPLE 503

### (7-{3-[2-(Pyridin-3-ylmethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 3-bromomethylpyridine, mp 95° C.

### EXAMPLE 504

### {7-[3-(2-Isobutyl-2H-tetraazol-5-yl)phenyl]pyrazolo[1,5-a]pyrimidin-3-yl}(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and isobutyl bromide, mp 141° C.

### EXAMPLE 505

### (7-{3-[2-(Cyclobutylmethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and cyclobutylmethyl bromide, mp 170° C.

### EXAMPLE 506

### {7-[3-(2-Butyl-2H-tetraazol-5-yl)phenyl]pyrazolo[1,5-a]pyrimidin-3-yl) (thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and butyl iodide, mp 90° C.

### EXAMPLE 507

### (7-{3-[2-(2-Morpholin-4-ylethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 4-(2-chloroethyl)morpholine, mp 103° C.

### EXAMPLE 508

### (7-{3-[1-(Cyclobutylmethyl)-1H-tetraazol-5-yl]phenyl}pyrazolo[1,5-a]pyrimidin-3-yl)(thien-2-yl)methanone

The title compound was prepared as a minor product using an analogous procedure to Method P from {7-[3-(1H-tetraazol-5-yl)phenyl]pyrazolo[1,5-a]pyrimidin-3-yl}(thien-2-yl)methanone and cyclobutylmethyl bromide, mp 204° C.

### EXAMPLE 509

### (7-{3-[2-(Pyridin-2-ylmethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 2-bromomethylpyridine, mp 184° C.

### EXAMPLE 510

### (7-{3-[2-(Pyridin-4-ylmethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 4-bromomethylpyridine, mp 170° C.

### EXAMPLE 511

### (7-{3-[2-(2-Pyrrolidin-1-ylethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 1-(2-chloroethyl)pyrrolidine, mp 98° C.

### EXAMPLE 512

### (7-{3-[2-(2-Piperidin-1-ylethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 1-(2-chloroethyl)piperidine, mp 117° C.

### EXAMPLE 513

### (7-{3-[2-(2-Hydroxyethyl)-2H-tetraazol-5-yl]phenyl}pyrazolo[1,5-a]pyrimidin-3-yl)(thien-2-yl)methanone

The title compound was prepared using an analogous procedure to Method P from {7-[3-(1H-tetraazol-5-yl)phenyl]pyrazolo[1,5-a]pyrimidin-3-yl}(thien-2-yl)methanone and 2-bromoethanol, mp 134° C.

### EXAMPLE 513

### (7-{3-[2-(2-Hydroxyethyl)-2H-tetraazol-5-yl]phenyl}pyrazolo[1,5-a]pyrimidin-3-yl)(thien-2-yl)methanone

The title compound was prepared using an analogous procedure to Method P from (7-{3-[2-(2-{[tert-butyl(dimethyl)silyl]oxy}ethyl)-2H-tetraazol-5-yl]phenyl}pyrazolo[1,5-a]pyrimidin-3-yl)(thien-2-yl)methanone and 2-bromoethanol, mp 134° C.

### EXAMPLE 514

### {7-[3-(1H-Pyrazol-3-yl)phenyl]pyrazolo[1,5-a]pyrimidin-3-yl}(thien-2-yl)methanone

The title compound was prepared using an analogous procedure to Method A from (2E)-3-(dimethylamino)-1-[3-(1H-pyrazol-3-yl)phenyl]prop-2-en-1-one and (5-amino-1H-pyrazol-4-yl)(thien-2-yl)methanone, mp 129° C.

(2E)-3-(Dimethylamino)-1-[3-(1H-pyrazol-3-yl)phenyl]prop-2-en-1-one was prepared as described below: 3-dimethylamino-1-[3-(3-dimethylamino-acryloyl)-phenyl]-propenone (prepared from 1,3-diacetylbenzene and DMF-DMA) (2.0 g, 7.35 mmol) was dissolved in ethanol (100 mL) and hydrazine (0.50 mL, 10.28 mmol) wad added. The resulting mixture was heated at 60° C. for 6 hours and evaporated to remove ethanol. The residue was purifed by silica gel flash column chromatograph to give 830 mg (47%) of the desired product as a yellow solid, mp 155° C.

### EXAMPLE 515

### (7-{3-[1-(Cyclobutylmethyl)-1H-pyrazol-3-yl]phenyl}pyrazolo[1,5-a]pyrimidin-3-yl)(thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and cyclobutylmethyl bromide, mp 60° C.

### EXAMPLE 516

### (7-{3-[1-(Pyridin-2-ylmethyl)-1H-pyrazol-3-yl]phenyl}pyrazolo[1,5-a]pyrimidin-3-yl)(thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and 2-bromomethylpyridine, mp 66° C.

### EXAMPLE 517

### (7-{3-[1-(Pyridin-2-ylmethyl)-1H-pyrazol-3-yl]phenyl}pyrazolo[1,5-a]pyrimidin-3-yl)(thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and 2-bromomethylpyridine, mp 66° C.

### EXAMPLE 518

### {7-[3-(1-Ethyl-1H-pyrazol-3-yl)phenyl]pyrazolo[1,5-a]pyrimidin-3-yl (thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and bromoethane, mp 61° C.

### EXAMPLE 520

### (7-{3-[1-(2-Pyrrolidin-1-ylethyl)-1H-pyrazol-3-yl]phenyl}pyrazolo[1,5-a]pyrimidin-3-yl)(thien-2-yl)methanone

The title compound was prepared using an analogous procedure to Method P from {7-[3-(1H-Pyrazol-3-yl)phenyl]pyrazolo[1,5-a]pyrimidin-3-yl}(thien-2-yl)methanone and 1-(2-chloroethyl)pyrrolidine, mp 63° C.

What is claimed is:

1. A method of inhibiting abnormal cell growth in a mammal which comprises administering to said mammal a therapeutically effective amount of at least one compound of formula 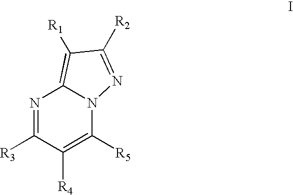
or a pharmaceutically acceptable salt or prodrug thereof,
wherein
R₁ is selected from the groups consisting of hydrogen, cyano, halogen, carbamoyl, formyl, carboxy, C(O)O-alkyl, C(O)O-cycloalkyl, C(O)cycloalkyl, R₆, C(O)R₆, and C(S)R₆;
R₆ is unsubstituted, monosubstituted or disubstituted aryl or heteroaryl wherein aryl or heteroaryl is phenyl, naphthalenyl, thiazolyl, biphenyl, thienyl, furanyl, or pyridinyl; and the substituents are selected from the groups consisting of halogen, nitro, cyano, CF₃, OCF₃, alkyl, alkoxy, trifluoromethyl, alkanol, alkylamino, alkylthio, dialkylamino, methylenedioxy, alkylsulfonyl and alkanoylamino;
R₂, R₃, and R₄ are hydrogen, CF₃, or alkyl;
R₅ is unsubstituted aryl or heteroaryl, or aryl or heteroaryl substituted by R₇, R₈, R₉, or R₁₀ with the proviso that R₁₀ must be present if the aryl or heteroaryl is substituted;
R₇, R₅, and R₉ are independently selected from the groups consisting of hydrogen, nitro, cyano, carbamoyl, halogen, N(CH₃)₂, CF₃, OCF₃, alkyl, alkoxy, and carboxy;
R₁₀ is selected from nitro, cyano, carboxy, carbamoyl, halogen, CF₃, OCF₃, alkyl, alkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl or R₁₀ is selected from unsubstituted, monosubstituted, or disubstituted aryl or heteroaryl which is a 5- or 6-membered aromatic ring moiety containing at least 1-4 heteroatoms selected from O, S, and N;
R₁₁ and R₁₂ are independently selected from the groups consisting of hydrogen, alkyl, alkenyl, alkynyl, cycloalkyl, Q₁, Q₂, -L-Q₁ and -L-Q₂; wherein Q₁ is unsubstituted aryl or heteroaryl, monosubstituted or disubstituted aryl or heteroaryl; wherein Q₁ comprises phenyl, naphthalenyl, biphenyl, thiazolyl, oxazolyl, pyrrolyl, pyrrazolyl, thienyl, furanyl, 1,2,3-triazolyl, 1,2,4-triazolyl, tetrazolyl, pyrrolidinyl, imidazolyl, and pyridinyl; and the substituents are selected from halogen, alkyl, CF₃, OCF₃, cyano, nitro, carboxy, hydroxy, alkoxy; wherein Q₂ is alkyl or heterocycle containing at least one and up to 4 heteroatoms selected from O, S, and N, optionally including their common protecting groups; Q₂ further comprises alkyl or cycloalkyl containing or substituted by functional groups selected from halogen, carboxy, carbamoyl, hydroxy, carbonyl, and cycloalkyl with a site of unsaturation;
R₁₁ and R₁₂ together with the N to which they are attached may join to form a 3 to 8 membered ring;
R₁₃ is hydrogen, alkyl, alkenyl, alkynyl or cycloalkyl; and R₁₁ and R₁₃ together with the N to which they are attached may join to form a 3 to 8 membered ring.

2. The method according to claim 1, wherein the unsubstituted or substituted aryl or heteroaryl of R₅ is selected from the group consisting of phenyl, naphthalenyl, thiazolyl, biphenyl, thienyl, furanyl and pyridinyl.

3. The method according to claim 1, wherein the unsubstituted, monosubstituted, or disubstituted aryl or heteroaryl of R₁₀ is selected from the group consisting of phenyl, naphthalenyl, thiazolyl, biphenyl, thienyl, furanyl and pyridinyl.

4. The method according to claim 3, wherein the one or two substituents of the aryl or heteroaryl of R₁₀ are independently selected from the group consisting of halogen, nitro, cyano, CF₃, OCF₃, carboxy, carbamoyl, alkyl, alkoxy, alkanol, trifluoromethyl, alkyl amino, alkyl thio, dialkylamino, methylenedioxy, alkylsulfonyl and alkanoylamino.

5. The method according to claim 1, wherein R₁ is C(O)R₆, or C(S)R₆; and R₂, R₃, and R₄ are hydrogen.

6. The method according to claim 5, wherein R₁ is C(O)R₆.

7. A method of treating, inhibiting the progression of, or eradicating a neoplasm which comprises administering to a mammal in need thereof a therapeutically effective amount of at least one compound of Formula I 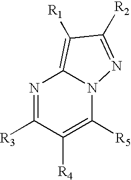
or a pharmaceutically acceptable salt or prodrug thereof
wherein
R₁ is selected from the groups consisting of hydrogen, cyano, halogen, carbamoyl, formyl, carboxy, C(O)O-alkyl, C(O)O-cycloalkyl, C(O)cycloalkyl, R₆, C(O)R₆, and C(S)R₆;
R₆ is unsubstituted, monosubstituted or disubstituted aryl or heteroaryl wherein aryl or heteroaryl is phenyl, naphthalenyl, thiazolyl, biphenyl, thienyl, furanyl, or pyridinyl; and the substituents are selected from the groups consisting of halogen, nitro, cyano, CF₃, OCF₃, alkyl, alkanol, alkoxy, trifluoromethyl, alkylamino, alkylthio, dialkylamino, methylenedioxy, alkylsulfonyl and alkanoylamino;
R₂, R₃, and R₄ are hydrogen, CF₃, or alkyl;
R₅ is unsubstituted aryl or heteroaryl, or aryl or heteroaryl substituted by R₇, R₈, R₉, or R₁₀ with the proviso that R₁₀ must be present if the aryl or heteroaryl is substituted;
R₇, R₈, and R₉ are independently selected from the groups consisting of hydrogen, nitro, cyano, carboxy, carbamoyl, halogen, N(CH₃)₂, CF₃, OCF₃, alkyl, and alkoxy;
R₁₀ is selected from nitro, cyano, carboxy, carbamoyl, halogen, CF3, OCF3, alkyl, alkoxy, alkoxyalkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₁₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl or R₁₀ is selected from unsubstituted, monosubstituted, or disubstituted aryl or heteroaryl which is a 5- or 6-membered aromatic ring moiety containing at least 1-4 heteroatoms selected from O, S, and N;
R₁₁ and R₁₂ are independently selected from the groups consisting of hydrogen, alkyl, alkenyl, alkynyl, cycloalkyl, Q₁, Q₂, -L-Q₁ and -L-Q₂; wherein Q₁ is unsubstituted aryl or heteroaryl, monosubstituted or disubstituted aryl or heteroaryl; wherein Q₁ comprises phenyl, naphthalenyl, biphenyl, thiazolyl, oxazolyl, pyrrolyl, pyrrazolyl, thienyl, furanyl, 1,2,3-triazolyl, 1,2,4-triazolyl, tetrazolyl, pyrrolidinyl, imidazolyl, and pyridinyl; and the substituents are selected from halogen, alkyl, CF₃, OCF₃, cyano, nitro, carboxy, hydroxy, alkoxy; wherein Q₂ is alkyl or heterocycle containing at least one and up to 4 heteroatoms selected from O, S, and N, optionally including their common protecting groups; Q₂ further comprises alkyl or cycloalkyl containing or substituted by functional groups selected from halogen, carboxy, carbamoyl, hydroxy, carbonyl, and cycloalkyl with a site of unsaturation;
R₁₁ and R₁₂ together with the N to which they are attached may join to form a 3 to 8 membered ring;
R₁₃ is hydrogen, alkyl, alkenyl, alkynyl or cycloalkyl; and R₁₁ and R₁₃ together with the N to which they are attached may join to form a 3 to 8 membered ring.

8. The method according to claim 7, wherein the unsubstituted or substituted aryl or heteroaryl of R₅ is selected from the group consisting of phenyl, naphthalenyl, thiazolyl, biphenyl, thienyl, furanyl and pyridinyl.

9. The method according to claim 7, wherein the unsubstituted, monosubstituted, or disubstituted aryl or heteroaryl of R₁₀ is selected from the group consisting of phenyl, naphthalenyl, thiazolyl, biphenyl, thienyl, furanyl and pyridinyl.

10. The method according to claim 9, wherein the one or two substituents of the aryl or heteroaryl of R₁₀ are independently selected from the group consisting of halogen, nitro, cyano, CF₃, OCF₃, carboxy, carbamoyl, alkyl, alkoxy, alkanol, trifluoromethyl, alkyl amino, alkyl thio, dialkylamino, methylenedioxy, alkylsulfonyl and alkanoylamino.

11. The method according to claim 7, wherein R₁ is C(O)R₆, or C(S)R₆; and R₂, R₃, and R₄ are hydrogen.

12. The method according to claim 11, wherein R₁ is C(O)R₆.

13. The method according to claim 7, wherein the neoplasm is selected from the group consisting of colon, breast, kidney, bladder, mouth, larynx, esophagus, stomach, colon, ovary, and lung.

14. A method of treating, inhibiting the progression of, or eradicating colorectal neoplasm in a mammal which comprises administering to said mammal a therapeutically effective amount of at least one compound of formula I 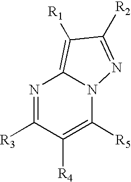
or a pharmaceutically acceptable salt or prodrug thereof,
wherein
R₁ is selected from the groups consisting of hydrogen, cyano, halogen, carbamoyl, formyl, carboxy, C(O)O-alkyl, C(O)O-cycloalkyl, C(O)cycloalkyl, R₆, C(O)R₆, and C(S)R₆;
R₆ is unsubstituted, monosubstituted or disubstituted aryl or heteroaryl wherein aryl or heteroaryl is phenyl, naphthalenyl, thiazolyl, biphenyl, thienyl, furanyl, or pyridinyl; and the substituents are selected from the groups consisting of halogen, nitro, cyano, CF₃, OCF₃, alkyl, alkanol, alkoxy, trifluoromethyl, alkylamino, alkylthio, dialkylamino, methylenedioxy, alkylsulfonyl and alkanoylamino;
R₂, R₃, and R₄ are hydrogen, CF₃, or alkyl;
R₅ is unsubstituted aryl or heteroaryl, or aryl or heteroaryl substituted by R₇, R₈, R₉, or R₁₀ with the proviso that R₁₀ must be present if the aryl or heteroaryl is substituted;
R₇, R₈, and R₉ are independently selected from the groups consisting of hydrogen, nitro, cyano, carboxy, carbamoyl, halogen, N(CH₃)₂, CF₃, OCF₃, alkyl, and alkoxy;
R₁₀ is selected from nitro, cyano, carboxy, carbamoyl, halogen, CF₃, OCF₃, alkyl, alkoxy, alkanol, NR₁₁R₁₂, N(R₁₃)COR₁₁, N(R₁₃)CONR₁₁R₁₂, OCONR₁₁R₁₂, N(R₁₃)CO₂R₁₁, N(R₁₃)CSR₁₁, N(R₁₃)CS(NR₁₁R₁₂), N(R₁₃)CS(OR₁₁), N(R₁₃)SO₂R₁₁, N(CONR₁₃R₁₁)₂, N(CO₂R₁₁)₂, N(COR₁₁)₂, N(CONR₁₃R₁₁)₂, CONR₁₁R₁₂, CO₂R₁₁, NHC(═NH)NHR₁₁, NHC(═NH—CN)NR₁₁R₁₂, NHC(═NH—CN)OR₁₁, C(═NH)NHR₁₁, C(═NH)NR₁₁R₁₂, LCONH₂, LCONR₁₁R₁₂, or LCO₂R₁₁ wherein L is alkyl, alkenyl or alkynyl or R₁₀ is selected from unsubstituted, monosubstituted, or disubstituted aryl or heteroaryl which is a 5- or 6-membered aromatic ring moiety containing at least 1-4 heteroatoms selected from O, S, and N;
R₁₁ and R₁₂ are independently selected from the groups consisting of hydrogen, alkyl, alkenyl, alkynyl, cycloalkyl, Q₁, Q₂, -L-Q₁ and -L-Q₂; wherein Q₁ is unsubstituted aryl or heteroaryl, monosubstituted or disubstituted aryl or heteroaryl; wherein Q₁ comprises phenyl, naphthalenyl, biphenyl, thiazolyl, oxazolyl, pyrrolyl, pyrrazolyl, thienyl, furanyl, 1,2,3-triazolyl, 1,2,4-triazolyl, tetrazolyl, pyrrolidinyl, imidazolyl, and pyridinyl; and the substituents are selected from halogen, alkyl, CF₃, OCF₃, cyano, nitro, carboxy, hydroxy, alkoxy; wherein Q₂ is alkyl or heterocycle containing at least one and up to 4 heteroatoms selected from O, S, and N, optionally including their common protecting groups; Q₂ further comprises alkyl or cycloalkyl containing or substituted by functional groups selected from halogen, carboxy, carbamoyl, hydroxy, carbonyl, and cycloalkyl with a site of unsaturation;
R₁₁ and R₁₂ together with the N to which they are attached may join to form a 3 to 8 membered ring;
R₁₃ is hydrogen, alkyl, alkenyl, alkynyl or cycloalkyl; and R₁₁ and R₁₃ together with the N to which they are attached may join to form a 3 to 8 membered ring.

15. The method according to claim 14, wherein the unsubstituted or substituted aryl or heteroaryl of R₅ is selected from the group consisting of phenyl, naphthalenyl, thiazolyl, biphenyl, thienyl, furanyl and pyridinyl.

16. The method according to claim 14, wherein the unsubstituted, monosubstituted, or disubstituted aryl or heteroaryl of R₁₀ is selected from the group consisting of phenyl, naphthalenyl, thiazolyl, biphenyl, thienyl, furanyl and pyridinyl.

17. The method according to claim 16, wherein the one or two substituents of the aryl or heteroaryl of R₁₀ are independently selected from the group consisting of halogen, nitro, cyano, CF₃, OCF₃, carboxy, carbamoyl, alkyl, alkoxy, alkanol, trifluoromethyl, alkyl amino, alkyl thio, dialkylamino, methylenedioxy, alkylsulfonyl and alkanoylamino.

18. The method according to claim 14, wherein R₁ is C(O)R₆, or C(S)R₆; and R₂, R₃, and R₄ are hydrogen.

19. The method according to claim 18, wherein R₁ is C(O)R₆.

20. The method of claim 1 wherein the compound is:
N-Phenyl-N′-{3-[3-(thien-2-ylcarbonyl)pyrazolo[1,5-a]pyrimidin-7-yl]phenyl}urea
N-(2-Phenylethyl)-N′-{3-[3-(thien-2-ylcarbonyl)pyrazolo[1,5-a]pyrimidin-7-yl]phenyl}urea
N-Cyclohexyl-N′-{3-[3-(thien-2-ylcarbonyl)pyrazolo[1,5-a]pyrimidin-7-yl]phenyl}urea
Methyl N-[l {3-[3-(thien-2-ylcarbonyl)pyrazolo[1,5-a]pyrimidin-7-yl]phenyl}amino)-carbonyl]-L-valinate
N-Ethyl-N′-{3-[3-(thien-2-ylcarbonyl)pyrazolo[1,5-a]pyrimidin-7-yl]phenyl}urea
N-Isopropyl-N′-(3-[3-(thien-2-ylcarbonyl)pyrazolo[1,5-a]pyrimidin-7-yl]phenyl}urea
N-Allyl-N′-{3-[3-(thien-2-ylcarbonyl)pyrazolo[1,5-a]pyrimidin-7-yl]phenyl}urea
N-Butyl-N′-{3-[3-(thien-2-ylcarbonyl)pyrazolo[1,5-a]pyrimidin-7-yl]phenyl}urea
N-{3-[3-(Thien-2-ylcarbonyl)pyrazolo[1,5-a]pyrimidin-7-yl]phenyl}-N′-(trichloroacetyl)urea
Ethyl N-[({3-[3-(thien-2-ylcarbonyl)pyrazolo[1,5-a]pyrimidin-7-yl]phenyl}amino)-carbonyl]glycinate
N-[(1S)-1-Phenylethyl]-N′-{3-[3-(thien-2-ylcarbonyl)pyrazolo[1,5-a]pyrimidin-7-yl]phenyl}urea
N-[(1S,2R)-2-Phenylcyclopropyl]-N′-{3-[3-(thien-2-ylcarbonyl)pyrazolo[1,5-a]pyrimidin-7-yl]phenyl}urea
Butyl 3-[3-(thien-2-ylcarbonyl)pyrazolo[1,5-a]pyrimidin-7-yl]phenylcarbamate
Allyl 3-[3-(thien-2-ylcarbonyl)pyrazolo[1,5-a]pyrimidin-7-yl]phenylcarbamate
Isopropyl 3-[3-(thien-2-ylcarbonyl)pyrazolo[1,5-a]pyrimidin-7-yl]phenylcarbamate
Isobutyl 3-[3-(thien-2-ylcarbonyl)pyrazolo[1,5-a]pyrimidin-7-yl]phenylcarbamate
Neopentyl 3-[3-(thien-2-ylcarbonyl)pyrazolo[1,5-a]pyrimidin-7-yl]phenylcarbamate 2,2,2-trichloroethyl 3-[3-(thien-2-ylcarbonyl)pyrazolo[1,5-a]pyrimidin-7-yl]phenylcarbamate
Prop-2-ynyl 3-[3-(thien-2-ylcarbonyl)pyrazolo[1,5-a]pyrimidin-7-yl]phenylcarbamate
3-Butenyl 3-[3-(2-thienylcarbonyl)pyrazolo[1,5-a]pyrimidin-7-yl]phenylcarbamate
Isopropenyl 3-[3-(thien-2-ylcarbonyl)pyrazolo[1,5-a]pyrimidin-7-yl]phenylcarbamate
2-Chloroethyl 3-[3-(thien-2-ylcarbonyl)pyrazolo[1,5-a]pyrimidin-7-yl]phenylcarbamate
4-Methylphenyl3-[3-(thien-2-ylcarbonyl)pyrazolo[1,5-a]pyrimidin-7-yl]phenylcarbamate
Benzyl 3-[3-(thien-2-ylcarbonyl)pyrazolo[1,5-a]pyrimidin-7-yl]phenylcarbamate
Ethyl 3-[3-(thien-2-ylcarbonyl)pyrazolo[1,5-a]pyrimidin-7-yl]phenylcarbamate
4-Chlorobutyl 3-[3-(thien-2-ylcarbonyl)pyrazolo[1,5-a]pyrimidin-7-yl]phenylcarbamate
N-{3-[3-(Thien-2-ylcarbonyl)pyrazolo[1,5-a]pyrimidin-7-yl]phenyl}methanesulfonamide
N-{3-[3-(Thien-2-ylcarbonyl)pyrazolo[1,5-a]pyrimidin-7-yl]phenyl}ethanesulfonamide
N-{3-[3-(Thien-2-ylcarbonyl)pyrazolo[1,5-a]pyrimidin-7-yl]phenyl}butane-1-sulfonamide
N-{3-[3-(Thien-2-ylcarbonyl)pyrazolo[1,5-a]pyrimidin-7-yl]phenyl}propane-2-sulfonamide
N-{3-[3-(Thien-2-ylcarbonyl)pyrazolo[1,5-a]pyrimidin-7-yl]phenyl}benzene-sulfonamide
4-Methyl-N-{3-[3-(thien-2-ylcarbonyl)pyrazolo[1,5-a]pyrimidin-7-yl]phenyl}benzenesulfonamide
2-Methyl-N-{3-[3-(thien-2-ylcarbonyl)pyrazolo[1,5-a]pyrimidin-7-yl]phenyl}benzenesulfonamide
4-Fluoro-N-{3-[3-(thien-2-ylcarbonyl)pyrazolo[1,5-a]pyrimidin-7-yl]phenyl}benzenesulfonamide
4-Bromo-N-{3-[3-(thien-2-ylcarbonyl)pyrazolo[1,5-a]pyrimidin-7-yl]phenyl}benzenesulfonamide
4-Methoxy-N-{3-[3-(thien-2-ylcarbonyl)pyrazolo[1,5-a]pyrimidin-7-yl]phenyl benzenesulfonamide
(E)-2-Phenyl-N-{3-[3-(thien-2-ylcarbonyl)pyrazolo[1,5-a]pyrimidin-7-yl]phenyl}ethene-sulfonamide
N-{3-[3-(Thien-2-ylcarbonyl)pyrazolo[1,5-a]pyrimidin-7-yl]phenyl}thiophene-2-sulfonamide
1-Methyl-N-{3-[3-(thien-2-ylcarbonyl)pyrazolo[1,5-a]pyrimidin-7-yl]phenyl}-1H-imidazole-4-sulfonamide
N,N-Dimethyl-N′-{3-[3-(thien-2-ylcarbonyl)pyrazolo[1,5-a]pyrimidin-7-yl]phenyl}-sulfamide
Ethyl oxo({3-[3-(thien-2-ylcarbonyl)pyrazolo[1,5-a]pyrimidin-7-yl]phenyl}amino)acetate
Methyl 4-oxo-4-({13-[3-(thien-2-ylcarbonyl)pyrazolo[1,5-a]pyrimidin-7-yl]phenyl}amino)-butanoate
2-Methyl-N-{3-[3-(thien-2-ylcarbonyl)pyrazolo[1,5-a]pyrimidin-7-yl]phenyl}acrylamide
3-Methyl-N-{3-[3-(thien-2-ylcarbonyl)pyrazolo[1,5-a]pyrimidin-7-yl]phenyl}but-2-enamide
N,N-Eimethyl-N′-{3-[3-(thien-2-ylcarbonyl)pyrazolo[1,5-a]pyrimidin-7-yl]phenyl}urea 3-Methyl-N-{3-[3-(thien-2-ylcarbonyl)pyrazolo[1,5-a]pyrimidin-7-yl]phenyl}butanamide
2,2-Dimethyl-N-{3-[3-(thien-2-ylcarbonyl)pyrazolo[1,5-a]pyrimidin-7-yl]phenyl}-propanamide
2,2,2-Trichloro-N-{3-[3-(thien-2-ylcarbonyl)pyrazolo[1,5-a]pyrimidin-7-yl]phenyl}-acetamide
3-(Methylthio)-N-{3-[3-(thien-2-ylcarbonyl)pyrazolo[1,5-a]pyrimidin-7-yl]phenyl}-propanamide
N-{3-[3-(Thien-2-ylcarbonyl)pyrazolo[1,5-a]pyrimidin-7-yl]phenyl}thiophene-2-carboxamide
N-{3-[3-(Thien-2-ylcarbonyl)pyrazolo[1,5-a]pyrimidin-7-yl]phenyl}-1-(trifluoroacetyl)-L-prolinamide
N-{3-[3-(Thien-2-ylcarbonyl)pyrazolo[1,5-a]pyrimidin-7-yl]phenyl}adamantane-1-carboxamide
4,7,7-Trimethyl-3-oxo-N-{3-[3-(thien-2-ylcarbonyl)pyrazolo[1,5-a]pyrimidin-7-yl]phenyl}-2-oxabicyclo[2.2.1]heptane-1-carboxamide
3,3-Dimethyl-N-{3-[3-(thien-2-ylcarbonyl)pyrazolo[1,5-a]pyrimidin-7-yl]phenyl}-butanamide
4-Methyl-N-{3-[3-(thien-2-ylcarbonyl)pyrazolo[1,5-a]pyrimidin-7-yl]phenyl}-pentanamide
N-{3-[3-(Thien-2-ylcarbonyl)pyrazolo[1,5-a]pyrimidin-7-yl]phenyl}pent-4-enamide
N-{3-[3-(Thien-2-ylcarbonyl)pyrazolo[1,5-a]pyrimidin-7-yl]phenyl}pent-4-ynamide
4,4,4-Trifluoro-3-methyl-N-{3-[3-(thien-2-ylcarbonyl)pyrazolo[1,5-a]pyrimidin-7-yl]phenyl}butanamide
2-Cyano-N-{3-[3-(thien-2-ylcarbonyl)pyrazolo[1,5-a]pyrimidin-7-yl]phenyl}acetamide
2-Methoxy-N-{3-[3-(thien-2-ylcarbonyl)pyrazolo[1,5-a]pyrimidin-7-yl]phenyl}acetamide
2-Oxo-2-({3-[3-(thien-2-ylcarbonyl)pyrazolo[1,5-a]pyrimidin-7-yl]phenyl}amino)ethyl acetate
2-(2-Methoxyethoxy)-N-{3-[3-(thien-2-ylcarbonyl)pyrazolo[1,5-a]pyrimidin-7-yl]phenyl}acetamide
N-2-Dimethyl-N-1-{3-[3-(thien-2-ylcarbonyl)pyrazolo[1,5-a]pyrimidin-7-yl]phenyl}-glycinamide
N-3-Diethyl-N-1-{3-[3-(thien-2-ylcarbonyl)pyrazolo[1,5-a]pyrimidin-7-yl]phenyl}-beta-alaninamide
2-Thien-2-yl-N-{3-[3-(thien-2-ylcarbonyl)pyrazolo[1,5-a]pyrimidin-7-yl]phenyl}acetamide
2-(2,3-Dihydro-1H-inden-2-yl)-N-{3-[3-(thien-2-ylcarbonyl)pyrazolo[1,5-a]pyrimidin-7-yl]phenyl}acetamide
2-(2-Methylphenyl)-N-{3-[3-(thien-2-ylcarbonyl)pyrazolo[1,5-a]pyrimidin-7-yl]phenyl}-acetamide
2-(1,3-Benzodioxol-5-yl)-N-{3-[3-(thien-2-ylcarbonyl)pyrazolo[1,5-a]pyrimidin-7-yl]phenyl}acetamide
N-{3-[3-(Thien-2-ylcarbonyl)pyrazolo[1,5-a]pyrimidin-7-yl]phenyl {quinoline-3-carboxamide
(2E)-3-Phenyl-N-{3-[3-(thien-2-ylcarbonyl)pyrazolo[1,5-a]pyrimidin-7-yl]phenyl}prop-2-enamide
2-Cyclopropyl-N-{-[3-(thien-2-ylcarbonyl)pyrazolo[1,5-a]pyrimidin-7-yl]phenyl}-acetamide
2-Cyclopentyl-N-{3-[3-(thien-2-ylcarbonyl)pyrazolo[1,5-a]pyrimidin-7-yl]phenyl}-acetamide
2-Cyclohexyl-N-{3-[3-(thien-2-ylcarbonyl)pyrazolo[1,5-a]pyrimidin-7-yl]phenyl}acetamide
2-Cycloheptyl-N-{3-[3-(thien-2-ylcarbonyl)pyrazolo[1,5-a]pyrimidin-7-yl]phenyl}acetamide
2-Bicyclo[2.2.1]hept-2-yl-N-{3-[3-(thien-2-ylcarbonyl)pyrazolo[1,5-a]pyrimidin-7-yl]phenyl}acetamide
N-1-{3-[3-(Thien-2-ylcarbonyl)pyrazolo[1,5-a]pyrimidin-7-yl]phenyl}glycinamide
N-1-{3-[3-(Thien-2-ylcarbonyl)pyrazolo[1,5-a]pyrimidin-7-yl]phenyl}-L-alaninamide
N-1-{3-[3-(Thien-2-ylcarbonyl)pyrazolo[1,5-a]pyrimidin-7-yl]phenyl}-L-leucinamide
N-1-{3-[3-(Thien-2-ylcarbonyl)pyrazolo[1,5-a]pyrimidin-7-yl]phenyl}-L-isoleucinamide
N-{3-[3-(Thien-2-ylcarbonyl)pyrazolo[1,5-a]pyrimidin-7-yl]phenyl}-L-phenylalaninamide
3-Cyclohexyl-N-1-{3-[3-(thien-2-ylcarbonyl)pyrazolo[1,5-a]pyrimidin-7-yl]phenyl}-L-alaninamide
N-1-{3-[3-(Thien-2-ylcarbonyl)pyrazolo[1,5-a]pyrimidin-7-yl]phenyl}-L-valinamide
4-Amino-N-{3-[3-(thien-2-ylcarbonyl)pyrazolo[1,5-a]pyrimidin-7-yl]phenyl}butanamide
N-{3-[3-(Thien-2-ylcarbonyl)pyrazolo[1,5-a]pyrimidin-7-yl]phenyl}-L-prolinamide
(2S)-2-Amino-2-cyclohexyl-N-{3-[3-(thien-2-ylcarbonyl)pyrazolo[1,5-a]pyrimidin-7-yl]phenyl}ethanamide
4-Methyl-N-{3-[3-(thien-2-ylcarbonyl)pyrazolo[1,5-a]pyrimidin-7-yl]phenyl}benzamide
4-Methoxy-N-{3-[3-(thien-2-ylcarbonyl)pyrazolo[1,5-a]pyrimidin-7-yl]phenyl}benzamide
4-Chloro-N-{3-[3-(thien-2-ylcarbonyl)pyrazolo[1,5-a]pyrimidin-7-yl]phenyl}benzamide
4-Bomo-N-{3-[3-(thien-2-ylcarbonyl)pyrazolo[1,5-a]pyrimidin-7-yl]phenyl}benzamide
4-(Heptyloxy)-N-{3-[3-(thien-2-ylcarbonyl)pyrazolo[1,5-a]pyrimidin-7-yl]phenyl}-benzamide
4-Pentyl-N-{3-[3-(thien-2-ylcarbonyl)pyrazolo[1,5-a]pyrimidin-7-yl]phenyl}benzamide
2-Methoxy-N-{3-[3-(thien-2-ylcarbonyl)pyrazolo[1,5-a]pyrimidin-7-yl]phenyl}benzamide
2-Methyl-N-{3-[3-(thien-2-ylcarbonyl)pyrazolo[1,5-a]pyrimidin-7-ylphenyl}benzamde
2-Chloro-N-{3-[3-(thien-2-ylcarbonyl)pyrazolo[1,5-a]pyrimidin-7-yl]phenyl}benzamide
N-{3-[3-(Thien-2-ylcarbonyl)pyrazolo[1,5-a]pyrimidin-7-yl]phenyl}-2-(trifluoromethyl)-benzamide
3-Methoxy-N-{3-[3-(thien-2-ylcarbonyl)pyrazolo[1,5-a]pyrimidin-7-yl]phenyl}benzamide
3-Fluoro-N-(3-[3-(thien-2-ylcarbonyl)pyrazolo[1,5-a]pyrimidin-7-yl]phenyl}benzamide
3,4-Dimethoxy-N-{3-[3-(thien-2-ylcarbonyl)pyrazolo[1,5-a]pyrimidin-7-yl]phenyl}-benzamide
3,4-Dichloro-N-{3-[3-(thien-2-ylcarbonyl)pyrazolo[1,5-a]pyrimidin-7-yl]phenyl}benzamide
2,6-Dimethoxy-N-{3-[3-(thien-2-ylcarbonyl)pyrazolo[1,5-a]pyrimidin-7-yl]phenyl}-benzamide
3,4-Difluoro-N-{3-[3-(thien-2-ylcarbonyl)pyrazolo[1,5-a]pyrimidin-7-yl]phenyl}benzamide
N-{3-[3-(Thien-2-ylcarbonyl)pyrazolo[1,5-a]pyrimidin-7-yl]phenyl}-1-naphthamide
N-{3-[3-(Thien-2-ylcarbonyl)pyrazolo[1,5-a]pyrimidin-7-yl]phenyl}-2-naphthamide
2-Phenyl-N-{3-[3-(thien-2-ylcarbonyl)pyrazolo[1,5-a]pyrimidin-7-yl]phenyl}acetamide
2-(4-Methoxyphenyl)-N-{3-[3-(thien-2-ylcarbonyl)pyrazolo[1,5-a]pyrimidin-7-yl]phenyl}-acetamide
2-(3-Methoxyphenyl)-N-{3-[3-(thien-2-ylcarbonyl)pyrazolo[1,5-a]pyrimidin-7-yl]phenyl}-acetamide
2-(4-Chlorophenyl)-N-{3-[3-(thien-2-ylcarbonyl)pyrazolo[1,5-a]pyrimidin-7-yl]phenyl}-acetamide
2-(4-Fluorophenyl)-N-{3-[3-(thien-2-ylcarbonyl)pyrazolo[1,5-a]pyrimidin-7-yl]phenyl}-acetamide
2-(2,5-Dimethoxyphenyl)-N-{3-[3-(thien-2-ylcarbonyl)pyrazolo[1,5-a]pyrimidin-7-yl]phenyl}acetamide
2-Phenoxy-N-{3-[3-(thien-2-ylcarbonyl)pyrazolo[1,5-a]pyrimidin-7-yl]phenyl}acetamide
4-Fluoro-N-{3-[3-(thien-2-ylcarbonyl)pyrazolo[1,5-a]pyrimidin-7-yl]phenyl}benzamide
Ethyl 2-oxo-2-({3-[3-(thien-2-ylcarbonyl)pyrazolo[1,5-a]pyrimidin-yl]phenyl}amino)-ethylcarbamate
Propyl 2-oxo-2-({3-[3-(thien-2-ylcarbonyl)pyrazolo[1,5-a]pyrimidin-7-yl]phenyl}amino)-ethylcarbamate
Isopropyl 2-oxo-2-({3-[3-(thien-2-ylcarbonyl)pyrazolo[1,5-a]pyrimidin-7yl]phenyl}amino)-ethylcarbamate
Butyl 2-oxo-2-({3-[3-(thien-2-ylcarbonyl)pyrazolo[1,5-a]pyrimidin-7-yl]phenyl}amino)-ethylcarbamate
Isobutyl 2-oxo-2-({3-[3-(thien-2-ylcarbonyl)pyrazolo[1,5-a]pyrimidin-7-yl]phenyl}amino)-ethylcarbamate
But-3-enyl 2-oxo-2-({3-[3-(thien-2-ylcarbonyl)pyrazolo[1,5-a]pyrimidin-7-yl]phenyl}amino)-ethylcarbamate
4-Chlorobutyl 2-oxo-2-({3-[3-(thien-2-ylcarbonyl)pyrazolo[1,5-a]pyrimidin-7-yl]phenyl}amino)ethylcarbamate
2-Chloroethyl 2-oxo-2-({3-[3-(thien-2-ylcarbonyl)pyrazolo[1,5-a]pyrimidin-7-yl]phenyl}amino)ethylcarbamate
Neopentyl 2-oxo-2-({3-[3-(thien-2-ylcarbonyl)pyrazolo[1,5-a]pyrimidin-7-yl]phenyl}amino)ethylcarbamnate
Hexyl 2-oxo-2-({3-[3-(thien-2-ylcarbonyl)pyrazolo[1,5-a]pyrimidin-7-yl]phenyl}amino)ethylcarbamate
Prop-2-ynyl 2-oxo-2-({3-[3-(thien-2-ylcarbonyl)pyrazolo[1,5-a]pyrimidin-7-yl]phenyl}amino)ethylcarbamate
Allyl 2-oxo-2-({3-[3-(thien-2-ylcarbonyl)pyrazolo[1,5-a]pyrimidin-7-yl]phenyl}amino)ethylcarbamate
Benzyl 2-oxo-2-({3-[3-(thien-2-ylcarbonyl)pyrazolo[1,5-a]pyrimidin-7-yl]phenyl}amino)ethylcarbaamate
N-[2-Oxo-2-({3-[3-(thien-2-ylcarbonyl)pyrazolo[1,5-a]pyrimidin-7-yl]phenyl}amino)ethyl]cyclopropanecarboxamide
N-[2-Oxo-2-({3-[3-(thien-2-ylcarbonyl)pyrazolo[1,5-a]pyrimidin-7-yl]phenyl}amino)ethyl]cyclobutanecarboxamide
N-[2-Oxo-2-({3-[3-(thien-2-ylcarbonyl)pyrazolo[1,5-a]pyrimidin-7-yl]phenyl}amino)ethyl]cyclopentanecarboxamide
N-[2-Oxo-2-({3-[3-(thien-2-ylcarbonyl)pyrazolo[1,5-a]pyrimidin-7-yl]phenyl}amino)ethyl]cyclohexanecarboxamide
2-Methyl-N-[2-oxo-2-({3-[3-(thien-2-ylcarbonyl)pyrazolo[1,5-a]pyrimidin-7-yl]phenyl}amino)ethyl]butanamide
2-Ethyl-N-[2-oxo-2-({3-[3-(thien-2-ylcarbonyl)pyrazolo[1,5-a]pyrimidin-7-yl]phenyl}amino)ethyl]butanamide
2,2-Dimethyl-N-[2-oxo-2-({3-[3-(thien-2-ylcarbonyl)pyrazolo[1,5-a]pyrimidin-7-yl]phenyl}amino) ethyl]propanamide
3-Methyl-N-[2-oxo-2-({3-[3-(thien-2-ylcarbonyl)pyrazolo[1,5-a]pyrimidin-7-yl]phenyl}amino)ethyl]butanamide
N-[2-Oxo-2-({3-[3-(thien-2-ylcarbonyl)pyrazolo[1,5-a]pyrimidin-7-yl]phenyl}amino)ethyl]adamantane-1-carboxamide
N-[2-Oxo-2-({3-[3-(thien-2-ylcarbonyl)pyrazolo[1,5-a]pyrimidin-7-yl]phenyl}amino)ethyl]benzamide
N-2-[(Pentylamino)carbonyl]-N-1-{3-[3-(thien-2-ylcarbonyl)pyrazolo[1,5-a]pyrimidin-7-yl]phenyl}glycinamide
N-2—[(Ethylamino)carbonyl]-N-1-{3-[3-(thien-2-ylcarbonyl)pyrazolo[1,5-a]pyrimidin-7-yl]phenyl}glycinamide
N-2—[(Cyclohexylamino)carbonyl]-N-1-{3-[3-(thien-2-ylcarbonyl)pyrazolo[1,5-a]pyrimidin-7-yl]phenyl}glycinamide
3-Chloro-N-{3-[3-(thien-2-ylcarbonyl)pyrazolo[1,5-a]pyrimidin-7-yl]phenyl}propanamide
3,5,5-Trimethyl-N-{3-[3-(thien-2-ylcarbonyl)pyrazolo[1,5-a]pyrimidin-7-yl]phenyl}hexanamide
3-Chloro-2-(chloromethyl)-2-methyl-N-{3-[3-(thien-2-ylcarbonyl)pyrazolo[1,5-a]pyrimidin-7-yl]phenyl}propanamide
N-{3-[3-(Thien-2-ylcarbonyl)pyrazolo[1,5-a]pyrimidin-7-yl]phenyl}undecanamide
1-Methyl-N-{3-[3-(thien-2-ylcarbonyl)pyrazolo[1,5-a]pyrimidin-7-yl]phenyl}cyclohexanecarboxamide
2,3,3-Trichloro-N-{3-[3-(thien-2-ylcarbonyl)pyrazolo[1,5-a]pyrimidin-7-yl]phenyl}acrylamide
2-Methyl-N-{3-[3-(thien-2-ylcarbonyl)pyrazolo[1,5-a]pyrimidin-7-yl]phenyl}butanamide
3-Cyclopentyl-N-{3-[3-(thien-2-ylcarbonyl)pyrazolo[1,5-a]pyrimidin-7-yl]phenyl}propanamide
2-Ethyl-N-{3-[3-(thien-2-ylcarbonyl)pyrazolo[1,5-a]pyrimidin-7-yl]phenyl}hexanamide
N-{3-[3-(Thien-2-ylcarbonyl)pyrazolo[1,5-a]pyrimidin-7-yl]phenyl}butanamide
(2E)-N-{3-[3-(Thien-2-ylcarbonyl)pyrazolo[1,5-a]pyrimidin-7-yl]phenyl}but-2-enamide
2-Ethyl-N-{3-[3-(thien-2-ylcarbonyl)pyrazolo[1,5-a]pyrimidin-7-yl]phenyl}butanamide
N-{3-[3-(Thien-2-ylcarbonyl)pyrazolo[1,5-a]pyrimidin-7-yl]phenyl}heptanamide
N-{3-[3-(Thien-2-ylcarbonyl)pyrazolo[1,5-a]pyrimidin-7-yl]phenyl}pentanamide
N-{3-[3-(Thien-2-ylcarbonyl)pyrazolo[1,5-a]pyrimidin-7-yl]phenyl}hexanamide
2-Methyl-N-{3-[3-(thien-2-ylcarbonyl)pyrazolo[1,5-a]pyrimidin-7-yl]phenyl}pentanamide
N-{3-[3-(Thien-2-ylcarbonyl)pyrazolo[1,5-a]pyrimidin-7-yl]phenyl}piperidine-1-carboxamide
N-Butyl-N-methyl-N′-{3-[3-(thien-2-ylcarbonyl)pyrazolo[1,5-a]pyrimidin-7-yl]phenyl}urea
N-{3-[3-(Thien-2-ylcarbonyl)pyrazolo[1,5-a]pyrimidin-7-yl]phenyl}morpholine-4-carboxaamide
N-(sec-Butyl)-N′-{3-[3-(thien-2-ylcarbonyl)pyrazolo[1,5-a]pyrimidin-7-yl]phenyl}urea
N,N-Diethyl-N′-{3-[3-(thien-2-ylcarbonyl)pyrazolo[1,5-a]pyrimidin-7-yl]phenyl}urea
N-(Cyclohexylmethyl)-N′-{3-[3-(thien-2-ylcarbonyl)pyrazolo[1,5-a]pyrimidin-7-yl]phenyl}urea
N-[2-(Dimethylamino)ethyl]-N′-{3-[3-(thien-2-ylcarbonyl)pyrazolo[1,5-a]pyrimidin-7-yl]phenyl}urea
N-Cyclopentyl-N′-{3-[3-(thien-2-ylcarbonyl)pyrazolo[1,5-a]pyrimidin-7-yl]phenyl}urea
N-(Tetrahydrofuran-2-ylmethyl)-N′-{3-[3-(thien-2-ylcarbonyl)pyrazolo[1,5-a]pyrimidin-7-yl]phenyl}urea
N-Neopentyl-N′-{3-[3-(thien-2-ylcarbonyl)pyrazolo[1,5-a]pyrimidin-7-yl]phenyl}urea
N-Pentyl-N′-{3-[3-(thien-2-ylcarbonyl)pyrazolo[1,5-a]pyrimidin-7-yl]phenyl}urea
4-Methyl-N-{3-[3-(thien-2-ylcarbonyl)pyrazolo[1,5-a]pyrimidin-7-yl]phenyl}piperazine-1-carboxamide
4-Methyl-N-{3-[3-(thien-2-ylcarbonyl)pyrazolo[1,5-a]pyrimidin-7-yl]phenyl}-1,4-diazepane-1-carboxamide
N,N-Bis(2-methoxyethyl)-N′-{3-[3-(thien-2-ylcarbonyl)pyrazolo[1,5-a]pyrimidin-7-yl]phenyl}urea
N-Cyclopropyl-N′-{3-[3-(thien-2-ylcarbonyl)pyrazolo[1,5-a]pyrimidin-7-yl]phenyl}urea
N-{3-[3-(Thien-2-ylcarbonyl)pyrazolo[1,5-a]pyrimidin-7-yl]phenyl}pyrrolidine-1-carboxamide
Cyclopentyl 3-[3-(thien-2-ylcarbonyl)pyrazolo[1,5-a]pyrimidin-7-yl]phenylcarbamate
Cyclohexyl 3-[3-(thien-2-ylcarbonyl)pyrazolo[1,5-a]pyrimidin-7-yl]phenylcarbamate
Cyclohex-2-en-1-yl 3-[3-(thien-2-ylcarbonyl)pyrazolo[1,5-a]pyrimidin-7-yl]phenylcarbamate
Cyclobutylmethyl 3-[3-(thien-2-ylcarbonyl)pyrazolo[1,5-a]pyrimidin-7-yl]phenylcarbamate
Cyclohexylmethyl 3-[3-(thien-2-ylcarbonyl)pyrazolo[1,5-a]pyrimidin-7-yl]phenylcarbamate
Tetrahydro-2H-pyran-4-yl 3-[3-(thien-2-ylcarbonyl)pyrazolo[1,5-a]pyrimidin-7-yl]phenylcarbamate
3-Methylbut-2-enyl 3-[3-(thien-2-ylcarbonyl)pyrazolo[1,5-a]pyrimidin-7-yl]phenylcarbamate
2-(Methylthio)ethyl 3-[3-(thien-2-ylcarbonyl)pyrazolo[1,5-a]pyrimidin-7-yl]phenylcarbamate
2-Thien-3-ylethyl 3-[3-(thien-2-ylcarbonyl)pyrazolo[1,5-a]pyrimidin-7-yl]phenylcarbamate
2-Piperidin-1-ylethyl 3-[3-(thien-2-ylcarbonyl)pyrazolo[1,5-a]pyrimidin-7-yl]phenylcarbamate
2-Morpholin-4-ylethyl 3-[3-(thien-2-ylcarbonyl)pyrazolo[1,5-a]pyrimidin-7-yl]phenylcarbamate
1-Methylprop-2-enyl 3-[3-(thien-2-ylcarbonyl)pyrazolo[1,5-a]pyrimidin-7-yl]phenylcarbamate
1-Methylbut-3-ynyl 3-[3-(thien-2-ylcarbonyl)pyrazolo[1,5-a]pyrimidin-7-yl]phenylcarbamate
1-Ethyl-2-methylpropyl 3-[3-(thien-2-ylcarbonyl)pyrazolo[1,5-a]pyrimidin-7-yl]phenylcarbamate
1,3-Dimethylbutyl 3-[3-(thien-2-ylcarbonyl)pyrazolo[1,5-a]pyrimidin-7-yl]phenylcarbamate
1-Ethylpropyl 3-[3-(thien-2-ylcarbonyl)pyrazolo[1,5-a]pyrimidin-7-yl]phenylcarbamate
(2-Methylcyclopropyl)methyl 3-[3-(thien-2-ylcarbonyl)pyrazolo[1,5-a]pyrimidin-7-yl]phenylcarbamate
3-Methylcyclopentyl 3-[3-(thien-2-ylcarbonyl)pyrazolo[1,5-a]pyrimidin-7-yl]phenylcarbamate
1-Methylbut-3-enyl 3-[3-(thien-2-ylcarbonyl)pyrazolo[1,5-a]pyrimidin-7-yl]phenylcarbamate
1-Cyclopropylethyl 3-[3-(thien-2-ylcarbonyl)pyrazolo[1,5-a]pyrimidin-7-yl]phenylcarbamate
1,2-Dimethylpropyl 3-[3-(thien-2-ylcarbonyl)pyrazolo[1,5-a]pyrimidin-7-yl]phenylcarbamate
1-Isopropylbutyl 3-[3-(thien-2-ylcarbonyl)pyrazolo[1,5-a]pyrimidin-7-yl]phenylcarbamate
But-3-ynyl 3-[3-(thien-2-ylcarbonyl)pyrazolo[1,5-a]pyrimidin-7-yl]phenylcarbamate
Propyl 3-[5-methyl-3-(thien-2-ylcarbonyl)pyrazolo[1,5-a]pyrimidin-7-yl]phenylcarbamate
Butyl 3-[5-methyl-3-(thien-2-ylcarbonyl)pyrazolo[1,5-a]pyrimidin-7-yl]phenylcarbamate
Allyl 3-[5-methyl-3-(thien-2-ylcarbonyl)pyrazolo[1,5-a]pyrimidin-7-yl]phenylcarbamate
Isopropyl 3-[5-methyl-3-(thien-2-ylcarbonyl)pyrazolo[1,5-a]pyrimidin-7-yl]phenylcarbamate
2-Chloroethyl 3-[5-methyl-3-(thien-2-ylcarbonyl)pyrazolo[1,5-a]pyrimidin-7-yl]phenylcarbamate
But-3-enyl 3-[5-methyl-3-(thien-2-ylcarbonyl)pyrazolo[1,5-a]pyrimidin-7-yl]phenylcarbamate
Neopentyl 3-[5-methyl-3-(thien-2-ylcarbonyl)pyrazolo[1,5-a]pyrimidin-7-yl]phenylcarbamate
3-Methyl-N-{3-[5-methyl-3-(thien-2-ylcarbonyl)pyrazolo[1,5-a]pyrimidin-7-yl]phenyl}butanamide
N-{3-[5-Methyl-3-(thien-2-ylcarbonyl)pyrazolo[1,5-a]pyrimidin-7-yl]phenyl}benzamide
N-{3-[5-Methyl-3-(thien-2-ylcarbonyl)pyrazolo[1,5-a]pyrimdin-7-yl]phenyl}cyclopentanecarboxamide
N-{3-[5-Methyl-3-(thien-2-ylcarbonyl)pyrazolo[1,5-a]pyrimidin-7-yl]phenyl}2-thien-2-ylacetamide
N-Butyl-N′-{3-[5-methyl-3-(thien-2-ylcarbonyl)pyrazolo[1,5-a]pyrimidin-7-yl]phenyl}urea
N-Isopropyl-N′-{3-[5-methyl-3-(thien-2-ylcarbonyl)pyrazolo[1,5-a]pyrimidin-7-yl]phenyl}urea
Propyl 3-[6-methyl-3-(thien-2-ylcarbonyl)pyrazolo[1,5-a]pyrimidin-7-yl]phenylcarbamate
Butyl 3-[6-methyl-3-(thien-2-ylcarbonyl)pyrazolo[1,5-a]pyrimidin-7-yl]phenylcarbamate
Allyl 3-[6-methyl-3-(thien-2-ylcarbonyl)pyrazolo[1,5-a]pyrimidin-7-yl]phenylcarbamate
Isopropyl 3-[6-methyl-3-(thien-2-ylcarbonyl)pyrazolo[1,5-a]pyrimidin-7-yl]phenylcarbamate
2-Chloroethyl 3-[6-methyl-3-(thien-2-ylcarbonyl)pyrazolo[1,5-a]pyrimidin-7-yl]phenylcarbamate
But-3-enyl 3-[6-methyl-3-(thien-2-ylcarbonyl)pyrazolo[1,5-a]pyrimidin-7-yl]phenylcarbamate
Neopentyl 3-[6-methyl-3-(thien-2-ylcarbonyl)pyrazolo[1,5-a]pyrimidin-7-yl]phenylcarbamate
3-Methyl-N-{3-[6-methyl-3-(thien-2-ylcarbonyl)pyrazolo[1,5-a]pyrimidin-7-yl]phenyl}butanamide
N-{3-[6-Methyl-3-(thien-2-ylcarbonyl)pyrazolo[1,5-a]pyrimidin-7-yl]phenyl}benzamide
N-{3-[6-Methyl-3-(thien-2-ylcarbonyl)pyrazolo[1,5-a]pyrimidin-7-yl]phenyl}cyclopropanecarboxamide
N-{3-[6-Methyl-3-(thien-2-ylcarbonyl)pyrazolo[1,5-a]pyrimidin-7-yl]phenyl}cyclopentanecarboxamide
N-{3-[6-Methyl-3-(thien-2-ylcarbonyl)pyrazolo[1,5-a]pyrimidin-7-yl]phenyl}-2-thien-2-ylacetamide
N-{3-[6-Methyl-3-(thien-2-ylcarbonyl)pyrazolo[1,5-a]pyrimidin-7-yl]phenyl}nicotinamide
N-Butyl-N′-{3-[6-methyl-3-(thien-2-ylcarbonyl)pyrazolo[1,5-a]pyrimidin-7-yl]phenyl}urea
N-Isopropyl-N′-3-[6-methyl-3-(thien-2-ylcarbonyl)pyrazolo[1,5-a]pyrimidin-7-yl]phenyl}urea
2-Thienyl[7-(2-thienyl)pyrazolo[1,5-a]-pyrimidin-3-yl}-methanone
[7-(3-Fluorophenyl)pyrazolo[1,5-a]-pyrimidin-3-yl}-2-thienyl-methanone
1-[3-[3-(2-Furanylcarbonyl)pyrazolo-[1,5-a]pyrimidin-7-yl]phenyl}-2-piperidinone
1-[3-(3-Benzoylpyrazolo[1,5-a]-pyrimidin-7-yl)phenyl]-2-piperidinone
1-[3-[3-(2-Furanylcarbonyl)pyrazolo-[1,5-a]pyrimidin-7-yl]phenyl}-2-pyrrolidinone
1-[3-(3-Benzoylpyrazolo[1,5-a]-pyrimidin-7-yl)phenyl]-2-pyrrolidinone
N-[3-(3-Benzoylpyrazolo-[1,5-a]pyrimidin-7-yl)phenyl]-cyclopropanecarboxamide
[7-[3-(Methylamino)phenyl]pyrazolo[1,5-a]-pyrimidin-3-yl}phenyl-methanone
N-[3-(3-Benzoylpyrazolo-[1,5-a]pyrimidin-7-yl)phenyl]-N-methyl-Cyclobutanecarboxamide
N-[3-[3-(2-Furanylcarbonyl)-pyrazolo[1,5-a]pyrimidin-7-yl]phenyl}-N-methyl-cyclobutanecarboxamide
N-[3-(3-Cyanopyrazolo[1,5-a]-pyrimidin-7-yl)phenyl]-N-methyl-cyclobutanecarboxamide
N-[3-[3-(2-Furanylcarbonvi)-pyrazoio[1,5-a]pyrimidin-7-yl]phenyl}-cyclopropanecarboxamide
N-[3-(3-Benzoylpyrazolo[1,5-a]-pyrimidin-7-yl)phenyl]-N-methyl-cyclopropanecarboxamide
N-methyl-N-[3-[3-(2-thienylcarbonyl)-pyrazolo[1,5-a]pyrimidin-7-yl]phenyl}-propanamide
N-methyl-N-[3-[3-(2-thienylcarbonyl)-pyrazolo[1,5-a]pyrimidin-7-yl]phenyl}-acetamide
Methy [3-[3-(2-thienylcarbonyl)pyrazolo-[1,5-a]pyrimidin-7-yl]phenyl}-carbamic acid
Ethyl Pyrazolo[1,5-a]pyrimidine-3-carboxylic acid,7-[3-[(cyclobutylcarbonyl)amino]phenyl]
N-[3-[3-(2-Furanylcarbonyl)pyrazolo-[1,5-a]pyrimidin-7-yl]phenyl}-2-methyl-propanamide
N-[3-(3-Benzoylpyrazolo[1,5-a]-pyrimidin-7-yl)phenyl]-2,2-dimethyl-propanamide
N-[3-[3-(2-Furanylcarbonyl)pyrazolo-[1,5-a]pyrimidin-7-yl]phenyl}-2,2-dimethyl-propanamide
[7-[3-(1H-Imidazol-1-yl)phenyl]pyrazolo-[1,5-a]pyrimidin-3-yl]-2-thienyl-methanone, [7-[3-(2-Methyl-1H-imidazol-1-yl)phenyl]-pyrazolo[1,5-a]pyrimidin-3-yl]-2-thienyl-methanone[7-(2,4-Dimethoxyphenyl)pyrazolo[1,5-a]-pyrimidin-3-yl}-2-thienyl-methanone[7-[3-(4-Methyl-1H-imidazol-1-yl)phenyl]-pyrazolo[1,5-a]pyrimidin-3-yl]-2-thienyl-methanone
N,N-Diethyl-3-[3-(2-thienylcarbonyl)-pyrazolo[1,5-a]pyrimidin-7-yl]-benzamide
N-[3-[3-(2-Thienylcarbonyl)pyrazolo[1,5-a]pyrimidin-7-yl]phenyl}-cyclopropane-carboxamide
N-[3-[3-(2-Thienylcarbonyl)pyrazolo[1,5-a]pyrimidin-7-yl]phenyl}-acetamide
N-[3-[3-(2-Thienylcarbonyl)pyrazolo[1,5-a]pyrimidin-7-yl]phenyl}-propanamide
N-[3-[3-[(5-Methyl-2-thienyl)carbonyl]pyrazolo[1,5-a]pyrimidin-7-yl]phenyl}-cyclopropanecarboxamide
N-[3-(3-Benzoylpyrazolo[1,5-a]pyrimidin-7-yl)phenyl]-N-ethyl-cyclopropanecarboxamide
N-Ethyl-N-(3-Pyrazolo[1,5-a]pyrimidin-7-ylphenyl)-cyclopropanecarboxamide
N-[3-(3-Chloropyrazolo[1,5-a]pyrimidin-7-yl)phenyl]-N-ethyl-cyclopropanecarboxamide
N-[3-(3-Cyanopyrazolo[1,5-a]pyrimidin-7-yl)phenyl]-N-ethyl-cyclopropanecarboxamide
N-Ethyl-N-[3-[3-(2-furanylcarbonyl)pyrazolo[1,5-a]pyrimidin-7-yl]phenyl}-cyclopropanecarboxamide
Ethyl 7-[3-[(cyclopropylcarbonyl)-2-propynylamino]phenyl]-pyrazolo[1,5-a]pyrimidine-3-carboxylic acid
N-2-Propynyl-N-[3-[3-(2-thienylcarbonyl)pyrazolo[1,5-a]pyrimidin-7-yl]phenyl}-cyciopropanecarboxamide
N-[3-[3-(2-Thienylcarbonyl)pyrazolo[1,5-a}pyrimidin-7-yl]phenyl}-formamide
N-Methyl-N-[3-[3-(2-thienylcarbonyl)pyrazolo[1,5-a]pyrimidin-7-yl]phenyl}-formamide
[7-(3-Aminophenyl)pyrazolo[1,5-a]pyrimidin-3-yl]-2-thienyl-methanone
[7-(3-Ethoxyphenyl)pyrazolo[1,5-a]py-rimidin-3-yl]-2-thienyl-methanone
[7-[3-(2-Methoxyethoxy)phenyl]pyrazolo[1,5-a]pyrimidin-3-yl]-2-thienyl-methanone
[7-[3-(Cyclopentyloxy)phenyl]pyrazolo[1,5-a]pyrimidin-3-yl]-2-thienyl-methanone
[7-[3-(Methylamino)phenyl]pyrazolo[1,5-a]pyrimidin-3-yl]-2-thienyl-methanone
[7-[3-(Propylamino)phenyl]pyrazolo[1,5-a]pyrimidin-3-yl]-2-thienyl-methanone
N-Methyl-N-[3-[3-(4-methylbenzoyl)pyrazolo[1,5-a]pyrimidin-7-yl]phenyl}-cyclopropanecarboxamide
2-Furanyl[7-[3-(1H-pyrrol-1-yl)phenyl]pyrazolo[1,5-a]pyrimidin-3-yl]-methanone
[7-[3-(1H-Pyrrol-1-yl)phenyl]pyrazolo[1,5-a]pyrimidin-3-yl]-2-thienyl-methanon
[7-[4-(Ethylamino)phenyl]pyrazolo[1,5-a]pyrimidin-3-yl]-2-thienyl-methanon
N-{3-[3-(2-Thienylcarbonyl)pyrazolo[1,5-a]pyrimidin-7-yl]phenyl}acrylamide
2-Methyl-N-{3-[3-(2-thienylcarbonyl)pyrazolo[1,5-a]pyrimidin-7-yl]phenyl}propanamide
N-{3-[3-(2-Thienylcarbonyl)pyrazolo[1,5-a]pyrimidin-7-yl]phenyl}-2-butynamide
N-{3-[3-(2-Thienylcarbonyl)pyrazolo[1,5-a]pyrimidin-7-yl]phenyl}cyclobutanecarboxwamide
N-{3-[5-Methyl-3-(2-thienylcarbonyl)pyrazolo[1,5-a]pyrimidin-7-yl]phenyl}cyclopropanecarboxamide
N-{3-[3-(2-Thienylcarbonyl)pyrazolo[1,5-a]pyrimidin-7-yl]phenyl}-2-oxiranecarboxamide
N-{3-[5-Methyl-3-(2-thienylcarbonyl)pyrazolo[1,5-a]pyrimidin-7-yl]phenyl}-2-oxiranecarboxamide
N-{3-[5-Methyl-3-(2-thienylcarbonyl)pyrazolo[1,5-a]pyrimidin-7-yl]phenyl}cyclobutanecarboxamide
N-{3-[3-(2-Thienylcarbonyl)pyrazolo[1,5-a]pyrimidin-7-yl]phenyl}-2,5-bis(trifluoromethyl)-benzamide
N-{3-[3-(2-Thienylcarbonyl)pyrazolo[1,5-a]pyrimidin-7-yl]phenyl}-3-(trifluoromethyl)-benzamide
N-{3-[3-(2-Thienylcarbonyl)pyrazolo[1,5-a]pyrimidin-7-yl]phenyl}cyclohexane-carboxamide
N-{3-[3-(2-Thienylcarbonyl)pyrazolo[1,5-a]pyrimidin-7-yl]phenyl}cyclopentane-carboxamide
N-{3-[3-(2-Thienylcarbonyl)pyrazolo[1,5-a]pyrimidin-7-yl]phenyl}benzamide
N-{3-[3-(2-Thienylcarbonyl)pyrazolo[1,5-a]pyrimidin-7-yl]phenyl}-2-furamide
3-Bromo-N-{3-[3-(2-thienylcarbonyl)pyrazolo[1,5-a]pyrimidin-7-yl]phenyl}benzamide
4-(tert-Butyl)-N-{3-[3-(2-thienylcarbonyl)pyrazolo[1,5-a]pyrimidin-7-yl]phenyl}benzamide
3,5-Dinitro-N-{3-[3-(2-thienylcarbonyl)pyrazolo[1,5-a]pyrimidin-7-yl]phenyl}benzamide
2,4-Dichloro-N-{3-[3-(2-thienylcarbonyl)pyrazolo[1,5-a]pyrimidin-7-yl]phenyl}benzamide
N-{3-[3-(2-Thienylcarbonyl)pyrazolo[1,5-a]pyrimidin-7-yl]phenyl}nicotinamide
N-{3-[3-(2-Thienylcarbonyl)pyrazolo[1,5-a]pyrimidin-7-yl]phenyl}isonicotinamide
N,N′-Dibutyl-N-{3-[3-(2-thienylcarbonyl)pyrazolo[1,5-a]pyrimidin-7-yl]phenyl}-dicarbonimidic diamide
Isopropyl 3-[3-(3-thienylcarbonyl)pyrazolo[1,5-a]pyrimidin-7-yl]phenylcarbamate
Isopropyl 3-(3-benzoylpyrazolo[1,5-a]pyrimidin-7-yl)phenylcarbamate
Isopropyl 3-[3-(cyclopentylcarbonyl)pyrazolo[1,5-a]pyrimidin-7-yl]phenylcarbamate
N-{3-[3-(Cyclopentylcarbonyl)pyrazolo[1,5-a]pyrimidin-7-yl]phenyl}-3-methylbutanamide
3-Methyl-N-{3-[3-(3-thienylcarbonyl)pyrazolo[1,5-a]pyrimidin-7-yl]phenyl}butanamide
N-{3-[3-(2-Furoyl)pyrazolo[1,5-a]pyrimidin-7-yl]phenyl}-N′-isopropylurea
Isopropyl 3-[3-(2-furoyl)pyrazolo[1,5-a]pyrimidin-7-yl]phenylcarbamate
N-{3-[3-(2-Furoyl)pyrazolo[1,5-a]pyrimidin-7-yl]phenyl}benzamide
N-{3-[3-(2-Furoyl)pyrazolo[1,5-a]pyrimidin-7-yl]phenyl}-3-methylbutanamide
N-{3-[3-(2-Furoyl)pyrazolo[1,5-a]pyrimidin-7-yl]phenyl}cyclopentanecarboxamide
2-Cyclopentyl-N-{3-[3-(2-furoyl)pyrazolo[1,5-a]pyrimidin-7-yl]phenyl}acetamide
N-{3-[3-(3-Furoyl)pyrazolo[1,5-a]pyrimidin-7-yl]phenyl}-3-methylbutanamide
Isopropyl 3-[3-(3-furoyl)pyrazolo[1,5-a]pyrimidin-7-yl]phenylcarbamate
Methyl 3-[3-(2-thienylcarbonyl)pyrazolo[1,5-a]pyrimidin-7-yl]benzoate
3-Methyl-N-{3-[2-methyl-3-(2-thienylcarbonyl)pyrazolo[1,5-a]pyrimidin-7-yl]phenyl}butanamide
Isopropyl 3-[2-methyl-3-(2-thienylcarbonyl)pyrazolo[1,5-a]pyrimidin-7-yl]phenylcarbamate
N-{2-Methyl-5-[3-(2-thienylcarbonyl)pyrazolo[1,5-a]pyrimidin-7-yl]phenyl}benzamide
3-Methyl-N-{2-methyl-5-[3-(2-thienylcarbonyl)pyrazolo[1,5-a]pyrimidin-7-yl]phenyl}butanamide
N-Isopropyl-N′-{2-methyl-5-[3-(2-thienylcarbonyl)pyrazolo[1,5-a]pyrimidin-7-yl]phenyl}urea
N-{2-Chloro-5-[3-(2-thienylcarbonyl)pyrazolo[1,5-a]pyrimidin-7-yl]phenyl}benzamide
N-{2-Chloro-5-[3-(2-thienylcarbonyl)pyrazolo[1,5-a]pyrimidin-7-yl]phenyl}-3-methylbutanamide
Isopropyl 2-methyl-5-[3-(2-thienylcarbonyl)pyrazolo[1,5-a]pyrimidin-7-yl]phenylcarbamate
N-{2-Methoxy-5-[3-(2-thienylcarbonyl)pyrazolo[1,5-a]pyrimidin-7-yl]phenyl}benzamide
Isopropyl 2-methoxy-5-[3-(2-thienylcarbonyl)pyrazolo[1,5-a]pyrimidin-7-yl]phenylcarbamate
N-{2-Methoxy-5-[3-(2-thienylcarbonyl)pyrazolo[1,5-a]pyrimidin-7-yl]phenyl}-3-methylbutanamide
N-Isobutyl-3-[3-(2-thienylcarbonyl)pyrazolo[1,5-a]pyrimidin-7-yl]benzamide
N-Butyl-3-[3-(2-thienylcarbonyl)pyrazolo[1,5-a]pyrimidin-7-yl]benzamide
N-Cyclopentyl-3-[3-(2-thienylcarbonyl)pyrazolo[1,5-a]pyrimidin-7-yl]benzamide
N-Phenyl-3-[3-(2-thienylcarbonyl)pyrazolo[1,5-a]pyrimidin-7-yl]benzamide
N-Isopropyl-3-[3-(2-thienylcarbonyl)pyrazolo[1,5-a]pyrimidin-7-yl]benzamide
N-Isopropyl-N′-{2-methoxy-5-[3-(2-thienylcarbonyl)pyrazolo[1,5-a]pyrimidin-7-yl]phenyl}urea
3-Methyl-N-{5-[3-(2-thienylcarbonyl)pyrazolo[1,5-a]pyrimidin-7-yl]-3-pyridinyl}butanamide
7-(3-Nitrophenyl)-3-(3-thienyl)pyrazolo[1,5-a]pyrimidine
3-(3-Bromopyrazolo[1,5-a]pyrimidin-7-yl)aniline
Isobutyl 3-(3-bromopyrazolo[1,5-a]pyrimidin-7-yl)phenylcarbamate
[7-(5-Nitro-3-thienyl)pyrazolo[1,5-a]pyrimidin-3-yl](2-thienyl)methanone
N-Isopropyl-N′-{5-[3-(thien-2-ylcarbonyl)pyrazolo[1,5-a]pyrimidin-7-yl]thien-2-yl}urea
N-[3-(3-Bromopyrazolo[1,5-a]pyrimidin-7-yl)phenyl]-3-methylbutanamide
[7-(5-Aminothien-3-yl)pyrazolo[1,5-a]pyrimidin-3-yl](thien-2-yl)methanone
O-Ethyl 3-[3-(thien-2-ylcarbonyl)pyrazolo[1,5-a]pyrimidin-7-yl]phenylthiocarbamate
3-Methyl-N-[3-(3-thien-2-ylpyrazolo[1,5-a]pyrimidin-7-yl)phenyl]butanamide
N-Phenyl-N′-{3-[3-(thien-2-ylcarbonyl)pyrazolo[1,5-a]pyrimidin-7-yl]phenyl}thiourea
Isopropyl 4-chloro-3-[3-(thien-2-ylcarbonyl)pyrazolo[1,5-a]pyrimidin-7-yl]phenylcarbamate
[7-(2-chloro-5-nitrophenyl)pyrazolo[1,5-a]pyrimidin-3-yl](thien-2-yl)methanone
N-{3-[3-(Pyridin-2-ylcarbonyl)pyrazolo[1,5-a]pyrimidin-7-yl]phenyl}cyclopropanecarboxamide
N-{3-[3-(1,3-Thiazol-2-ylcarbonyl)pyrazolo[1,5-a]pyrimidin-7-yl]phenyl}cyclopropanecarboxamide
N-{4-Chloro-3-[3-(thien-2-ylcarbonyl)pyrazolo[1,5-a]pyrimidin-7-yl]phenyl}-3-methylbutanamide
N-{4-Chloro-3-[3-(thien-2-ylcarbonyl)pyrazolo[1,5-a]pyrimidin-7-yl]phenyl}-N′-isopropylthiourea
N-{4-Chloro-3-[3-(thien-2-ylcarbonyl)pyrazolo[1,5-a]pyrimidin-7-yl]phenyl}benzamide
N-{2-Fluoro-5-[3-(thien-2-ylcarbonyl)pyrazolo[1,5-a]pyrimidin-7-yl]phenyl}-3-methylbutanamide
3-Methyl-N-{5-[3-(thien-2-ylcarbonyl)pyrazolo[1,5-a]pyrimidin-7-yl]thien-2-yl}butanamide
Isopropyl 5-[3-(thien-2-ylcarbonyl)pyrazolo[1,5-a]pyrimidin-7-yl]thien-2-ylcarbamate
[7-(5-Aminothien-2-yl)pyrazolo[1,5-a]pyrimidin-3-yl](thien-2-yl)methanone
4-Chloro-N-{3-[3-(thien-2-ylcarbonyl)pyrazolo[1,5-a]pyrimidin-7-yl]phenyl}butanamide
Isopropyl 2-fluoro-5-[3-(thien-2-ylcarbonyl)pyrazolo[1,5-a]pyrimidin-7-yl]phenylcarbamate
Isopropyl 2-(dimethylamino)-5-[3-(thien-2-ylcarbonyl)pyrazolo[1,5-a]pyrimidin-7-yl]phenylcarbamate
{7-[3-(Benzylamino)phenyl]pyrazolo[1,5-a]pyrimidin-3-yl}(thien-2-yl)methanone
{7-[3-(Isopentylamino)phenyl]pyrazolo[1,5-a]pyrimidin-3-yl}(thien-2-yl)methanone
3-Methyl-N-{4-[3-(thien-2-ylcarbonyl)pyrazolo[1,5-a]pyrimidin-7-yl]thien-2-yl}butanamide
[7-(3-Amino-4-fluorophenyl)pyrazolo[1,5-a]pyrimidin-3-yl](thien-2-yl)methanone
4,4,4-Trichloro-N-{3-[3-(thien-2-ylcarbonyl)pyrazolo[1,5-a]pyrimidin-7-yl]phenyl}butanamide
N-{2-Fluoro-5-[3-(thien-2-ylcarbonyl)pyrazolo[1,5-a]pyrimidin-7-yl]phenyl}-3,3-dimethylbutanamide
4,4,4-Trifluoro-N-{2-fluoro-5-[3-(thien-2-ylcarbonyl)pyrazolo[1,5-a]pyrimidin-7-yl]phenyl}-3-methylbutanamide
Bis(2,2,2-trichloroethyl) 2-fluoro-5-[3-(thien-2-ylcarbonyl)pyrazolo[1,5-a]pyrimidin-7-yl]phenylimidodicarbonate
2-Fluoro-N-{3-[3-(thien-2-ylcarbonyl)pyrazolo[1,5-a]pyrimidin-7-yl]phenyl}benzamide
2,6-Difluoro-N-{3-[3-(thien-2-ylcarbonyl)pyrazolo[1,5-a]pyrimidin-7-yl]phenyl}benzamide
N-{3-[3-(Thien-2-ylcarbonyl)pyrazolo[1,5-a]pyrimidin-7-yl]phenyl}pyridine-2-carboxamide
N-{5-[3-(Thien-2-ylcarbonyl)pyrazolo[1,5-a]pyrimidin-7-yl]thien-2-yl}benzamide
(7-{3-[(2-Phenylethyl)amino]phenyl}pyrazolo[1,5-a]pyrimidin-3-yl)(thien-2-yl)methanone
(7-{3-[(2-Methoxybenzyl)amino]phenyl}pyrazolo[1,5-a]pyrimidin-3-yl)(thien-2-yl)methanone
N-Butyl-N′-{3-[3-(thien-2-ylcarbonyl)pyrazolo[1,5-a]pyrimidin-7-yl]phenyl}thiourea
(7-{3-[(3-Chlorobenzyl)amino]phenyl}pyrazolo[1,5-a]pyrimidin-3-yl)(thien-2-yl)methanone
(7-{3-[(4-Chlorobenzyl)amino]phenyl}pyrazolo[1,5-a]pyrimidin-3-yl)(thien-2-yl)methanone
(7-{3-[(2-Furylmethyl)amino]phenyl}pyrazolo[1,5-a]pyrimidin-3-yl)(thien-2-yl)methanone
(7-{3-[(2-Chlorobenzyl)amino]phenyl}pyrazolo[1,5-a]pyrimidin-3-yl)(thien-2-yl)methanone
(7-{3-[(3-Furylmethyl)amino]phenyl}pyrazolo[1,5-a]pyrimidin-3-yl)(thien-2-yl)methanone
(7-{3-[(2E)-But-2-enylamino]phenyl}pyrazolo[1,5-a]pyrimidin-3-yl)(thien-2-yl)methanone
(7-{3-[(3-Methylbut-2-enyl)amino]phenyl}pyrazolo[1,5-a]pyrimidin-3-yl)(thien-2-yl)methanone
(7-{3-[(3,3-Dimethylbutyl)amino]phenyl}pyrazolo[1,5-a]pyrimidin-3-yl)(thien-2-yl)methanone
[7-(6-Chloropyridin-3-yl)pyrazolo[1,5-a]pyrimidin-3-yl](thien-2-yl)methanone
2,6-Difluoro-N-{2-fluoro-5-[3-(thien-2-ylcarbonyl)pyrazolo[1,5-a]pyrimidin-7-yl]phenyl}benzamide
2-Fluoro-N-{2-fluoro-5-[3-(thien-2-ylcarbonyl)pyrazolo[1,5-a]pyrimidin-7-yl]phenyl}benzamide
2-Chloro-N-{2-fluoro-5-[3-(thien-2-ylcarbonyl)pyrazolo[1,5-a]pyrimidin-7-yl]phenyl}benzamide
(7-{3-[(2-Methylbenzyl)amino]phenyl}pyrazolo[1,5-a]pyrimidin-3-yl)(thien-2-yl)methanone
(7-{3-[(2-Phenylpropyl)amino]phenyl}pyrazolo[1,5-a]pyrimidin-3-yl)(thien-2-yl)methanone
(7-{3-[(Cyclohexylmethyl)amino]phenyl}pyrazolo[1,5-a]pyrimidin-3-yl)(thien-2-yl)methanone
{7-[3-(Butylamino)phenyl]pyrazolo[1,5-a]pyrimidin-3-yl}(thien-2-yl)methanone
2-Chloroethyl 2-fluoro-5-[3-(thien-2-ylcarbonyl)pyrazolo[1,5-a]pyrimidin-7-yl]phenylcarbamate
N-{2-Fluoro-5-[3-(thien-2-ylcarbonyl)pyrazolo[1,5-a]pyrimidin-7-yl]phenyl}-N′-isopropylurea
4-(1H-Pyrrol-1-yl)-N-{3-[3-(thien-2-ylcarbonyl)pyrazolo[1,5-a]pyrimidin-7-yl]phenyl}benzamide
2,6-Dichloro-N-{3-[3-(thien-2-ylcarbonyl)pyrazolo[1,5-a]pyrimidin-7-yl]phenyl}benzamide
N-{3-[3-(Thien-2-ylcarbonyl)pyrazolo[1,5-a]pyrimidin-7-yl]phenyl}pyrazine-2-carboxamide
Phenyl N′-cyano-N-{3-[3-(thien-2-ylcarbonyl)pyrazolo[1,5-a]pyrimidin-7-yl]phenyl}imido-carbamate
1-Methyl-N-{3-[3-(thien-2-ylcarbonyl)pyrazolo[1,5-a]pyrimidin-7-yl]phenyl}-1H-pyrrole-2-carboxamide
N-{2-Fluoro-5-[3-(thien-2-ylcarbonyl)pyrazolo[1,5-a]pyrimidin-7-yl]phenyl}isonicotinamide
N-Isopropyl-N′-{3-[3-(thien-2-ylcarbonyl)pyrazolo[1,5-a]pyrimidin-7-yl]phenyl}thiourea
(7-Chloropyrazolo[1,5-a]pyrimidin-3-yl)(thien-2-yl)methanone
2,6-Dichloro-N-(2,6-dichlorobenzoyl)-N-{3-[3-(thien-2-ylcarbonyl)pyrazolo[1,5-a]pyrimidin-7-yl]phenyl}benzamide tert-Butyl 4-[2-oxo-2-({3-[3-(thien-2-ylcarbonyl)pyrazolo[1,5-a]pyrimidin-7-yl]phenyl}amino)ethyl]piperidine-1-carboxylate
2-Piperidin-4-yl-N-{3-[3-(thien-2-ylcarbonyl)pyrazolo[1,5-a]pyrimidin-7-yl]phenyl}acetamide
{7-[3-(Diethylamino)phenyl]pyrazolo[1,5-a]pyrimidin-3-yl}(thien-2-yl)methanone
[7-(3-{[(2E)-3-(2-Methoxyphenyl)prop-2-enyl]amino}phenyl)pyrazolo[1,5-a]pyrimidin-3-yl](thien-2-yl)methanone
[7-(3-{[(2E)-3-(4-Methoxyphenyl)prop-2-enyl]amino}phenyl)pyrazolo[1,5-a]pyrimidin-3-yl](thien-2-yl)methanone
[7-(3-{[(2E)-2-Methyl-3-phenylprop-2-enyl]amino}phenyl)pyrazolo[1,5-a]pyrimidin-3-yl](thien-2-yl)methanone
[7-(3-{[(2E)-3-(4-Nitrophenyl)prop-2-enyl]amino}phenyl)pyrazolo[1,5-a]pyrimidin-3-yl](thien-2-yl)methanone
(7-{3-[(3,3-Diphenylprop-2-enyl)amino]phenyl}pyrazolo[1,5-a]pyrimidin-3-yl)(thien-2-yl)methanone
(7-{3-[(3-Phenylbutyl)amino]phenyl}pyrazolo[1,5-a]pyrimidin-3-yl)(thien-2-yl)methanone
[7-(3-{[(2E)-3-(4-Hydroxy-3-methoxyphenyl)prop-2-enyl]amino}phenyl)pyrazolo[1,5-a]pyrimidin-3-yl](thien-2-yl)methanone
(7-{3-[(2-Methylbutyl)amino]phenyl}pyrazolo[1,5-a]pyrimidin-3-yl)(thien-2-yl)methanone
(7-{3-[(2-Ethylbutyl)amino]phenyl}pyrazolo[1,5-a]pyrimidin-3-yl)(thien-2-yl)methanone {7-[3-(Isobutylamino)phenyl]pyrazolo[1,5-a]pyrimidin-3-yl}(thien-2-yl)methanone tert-Butyl 4-[({3-[3-(thien-2-ylcarbonyl)pyrazolo[1,5-a]pyrimidin-7-yl]phenyl}amino)-carbonyl]piperidine-1-carboxylate
N,N′-Bis {3-[3-(thien-2-ylcarbonyl)pyrazolo[1,5-a]pyrimidin-7-yl]phenyl}thiourea
Isobutyl 3-((3-thien-2-ylpyrazolo[1,5-a]pyrimidin-7-yl)phenylcarbamate
N-{3-[3-(Thien-2-ylcarbonyl)pyrazolo[1,5-a]pyrimidin-7-yl]phenyl}piperidine-4-carboxamide
[7-(3-{[(2E)-2-Hexyl-3-phenylprop-2-enyl]amino}phenyl)pyrazolo[1,5-a]pyrimidin-3-yl](thien-2-yl)methanone
[7-(3-{[(2E)-2-Pentyl-3-phenylprop-2-enyl]amino}phenyl)pyrazolo[1,5-a]pyrimidin-3-yl](thien-2-yl)methanone
N-Isopropyl-N′-{3-[3-(thien-2-ylcarbonyl)pyrazolo[1,5-a]pyrimidin-7-yl]phenyl}guanidine
[7-(3-{[(2E)-3-Phenylprop-2-enyl]amino}phenyl)pyrazolo[1,5-a]pyrimidin-3-yl](thien-2-yl)methanone
[7-(3-{[(2E)-3-(4-tert-Butylphenyl)-2-methylprop-2-enyl]amino}phenyl)pyrazolo[1,5-a]pyrimidin-3-yl](thien-2-yl)methanone
N′-Cyano-N-isopropyl-N′-{3-[3-(thien-2-ylcarbonyl)pyrazolo[1,5-a]pyrimidin-7-yl]phenyl}guanidine
N-{3-[3-(Thien-2-ylcarbonyl)pyrazolo[1,5-a]pyrimidin-7-yl]phenyl}-1H-imidazole-5-carboxamide
Pyridin-4-ylmethyl 3-[3-(thien-2-ylcarbonyl)pyrazolo[1,5-a]pyrimidin-7-yl]phenylcarbamate
1-Methyl-N-{3-[3-(thien-2-ylcarbonyl)pyrazolo[1,5-a]pyrimidin-7-yl]phenyl}piperidine-4-carboxamide
(7-{3-[(2-Methylpentyl)amino]phenyl}pyrazolo[1,5-a]pyrimidin-3-yl)(thien-2-yl)methanone
(7-{3-[(2-Ethylhexyl)amino]phenyl}pyrazolo[1,5-a]pyrimidin-3-yl)(thien-2-yl)methanone
3-(1,1-Dioxidothiomorpholin-4-yl)propyl 3-[3-(thien-2-ylcarbonyl)pyrazolo[1,5-a]pyrimidin-7-yl]phenylcarbamate
2-Chloro-N-{2-(dimethylamino)-5-[3-(thien-2-ylcarbonyl)pyrazolo[1,5-a]pyrimidin-7-yl]phenyl}benzamide
{7-[3-(Ethylamino)phenyl]pyrazolo[1,5-a]pyrimidin-3-yl}(thien-2-yl)methanone
4-Morpholin-4-yl-N-{3-[3-(thien-2-ylcarbonyl)pyrazolo[1,5-a]pyrimidin-7-yl]phenyl}butanamide
Pyridin-3-ylmethyl 3-[3-(thien-2-ylcarbonyl)pyrazolo[1,5-a]pyrimidin-7-yl]phenylcarbamate
3-Morpholin-4-ylpropyl 3-[3-(thien-2-ylcarbonyl)pyrazolo[1,5-a]pyrimidin-7-yl]phenylcarbamate
[7-(3-{[(2E)-2-Methylpent-2-enyl]amino}phenyl)pyrazolo[1,5-a]pyrimidin-3-yl](thien-2-yl)methanone
2-Pyridin-4-yl-N-{3-[3-(thien-2-ylcarbonyl)pyrazolo[1,5-a]pyrimidin-7-yl]phenyl}acetamide
N-{2-(Dimethylamino)-5-[3-(thien-2-ylcarbonyl)pyrazolo[1,5-a]pyrimidin-7-yl]phenyl}isonicotinamide
Pyridin-2-ylmethyl 3-[3-(thien-2-ylcarbonyl)pyrazolo[1,5-a]pyrimidin-7-yl]phenylcarbamate
(7-{3-[(2,3-Dimethylpentyl)amino]phenyl}pyrazolo[1,5-a]pyrimidin-3-yl)(thien-2-yl)methanone
[7-(3-{[(2E)-2-Methylbut-2-enyl]amino}phenyl)pyrazolo[1,5-a]pyrimidin-3-yl](thien-2-yl)methanone
(7-{3-[(2E)-Pent-2-enylamino]phenyl}pyrazolo[1,5-a]pyrimidin-3-yl)(thien-2-yl)methanone
Thien-2-yl(7-{3-[(3,5,5-trimethylhexyl)amino]phenyl}pyrazolo[1,5-a]pyrimidin-3-yl)methanone
(7-{3-[(2-Methylprop-2-enyl)amino]phenyl}pyrazolo[1,5-a]pyrimidin-3-yl)(thien-2-yl)methanone,
(7-{3-[(2-Ethylprop-2-enyl)amino]phenyl}pyrazolo[1,5-a]pyrimidin-3-yl)(thien-2-yl)methanone,
{7-[4′-(Dimethylamino)-1,1′-biphenyl-3-yl]pyrazolo[1,5-a]pyrimidin-3-yl}(thien-2-yl)methanone;
[7-(3′-Amino-1,1′-biphenyl-3-yl)pyrazolo[1,5-a]pyrimidin-3-yl](thien-2-yl)methanone;
{7-[4′-(Hydroxymethyl)-1,1′-biphenyl-3-yl]pyrazolo[1,5-a]pyrimidin-3-yl}(thien-2-yl)methanone;
{7-[3′-(Dimethylamino)-1,1′-biphenyl-3-yl]pyrazolo[1,5-a]pyrimidin-3-yl}(thien-2-yl)methanone;
{7-[4′-(Dimethylamino)-6-fluoro-1,1′-biphenyl-3-yl]pyrazolo[1,5-a]pyrimidin-3-yl}(thien-2-yl)methanone;
{7-[6-Fluoro-4′-(hydroxymethyl)-1,1′-biphenyl-3-yl]pyrazolo[1,5-a]pyrimidin-3-yl}(thien-2-yl)methanone;
[7-(4′-Ethyl-1,1′-biphenyl-3-yl)pyrazolo[1,5-a]pyrimidin-3-yl](thien-2-yl)methanone;
3-{3′-[3-(Thien-2-ylcarbonyl)pyrazolo[1,5-a]pyrimidin-7-yl]-1,1′-biphenyl-4-yl}propanoic acid;
3′-[3-(Thien-2-ylcarbonyl)pyrazolo[1,5-a]pyrimidin-7-yl]-1,1′-biphenyl-4-carbaldehyde;
{7-[4′-(Morpholin-4-ylmethyl)-1,1′-biphenyl-3-yl]pyrazolo[1,5-a]pyrimidin-3-yl}(thien-2-yl)methanone;
(7-{4′-[(4-Methylpiperazin-1-yl)methyl]-1,1′-biphenyl-3-yl}pyrazolo[1,5-a]pyrimidin-3-yl)(thien-2-yl)methanone;
[7-(4′-{[(2-Morpholin-4-ylethyl)amino]methyl}-1,1′-biphenyl-3-yl)pyrazolo[1,5-a]pyrimidin-3-yl](thien-2-yl)methanone;
(7-{4′-[(Diethylamino)methyl]-1,1′-biphenyl-3-yl}pyrazolo[1,5-a]pyrimidin-3-yl)(thien-2-yl)methanone;
(7-{4′-[(Dimethylamino)methyl]-1,1′-biphenyl-3-yl}pyrazolo[1,5-a]pyrimidin-3-yl)(thien-2-yl)methanone;
N-{3-[3-(3-Aminophenyl)pyrazolo[1,5-a]pyrimidin-7-yl]phenyl}-3-methylbutanamide;
N-(3-{3-[3-(Dimethylamino)phenyl]pyrazolo[1,5-a]pyrimidin-7-yl}phenyl)-3-methylbutanamide;
N-{3-[3-(1H-Indol-5-yl)pyrazolo[1,5-a]pyrimidin-7-yl]phenyl}-3-methylbutanamide;
3-Methyl-N-{3-[3-(3,4,5-trimethoxyphenyl)pyrazolo[1,5-a]pyrimidin-7-yl]phenyl}butanamide;
3-Methyl-N-[3-(3-quinolin-8-ylpyrazolo[1,5-a]pyrimidin-7-yl)phenyl]butanamide;
N-(3-{3-[4-(Hydroxymethyl)phenyl]pyrazolo[1,5-a]pyrimidin-7-yl}phenyl)-3-methylbutanamide;
N-{3-[3-(4-Methoxyphenyl)pyrazolo[1,5-a]pyrimidin-7-yl]phenyl}-3-methylbutanamide;
N-{3-[3-(3-Chlorophenyl)pyrazolo[1,5-a]pyrimidin-7-yl]phenyl}-3-methylbutanamide;
N-{3-[3-(4-Chlorophenyl)pyrazolo[1,5-a]pyrimidin-7-yl]phenyl}-3-methylbutanamide;
N-{3-[3-(2,3-Dichlorophenyl)pyrazolo[1,5-a]pyrimidin-7-yl]phenyl}-3-methylbutanamide;
N-{3-[3-(3,4-Dichlorophenyl)pyrazolo[1,5-a]pyrimidin-7-yl]phenyl}-3-methylbutanamide;
N-{3-[3-(3,5-Dichlorophenyl)pyrazolo[1,5-a]pyrimidin-7-yl]phenyl}-3-methylbutanamide;
N-{3-[3-(3,5-Dimethylphenyl)pyrazolo[1,5-a]pyrimidin-7-yl]phenyl}-3-methylbutanamide;
3-Methyl-N-(3-{3-[3-(trifluoromethyl)phenyl]pyrazolo[1,5-a]pyrimidin-7-yl}phenyl)butanamide;
N-{3-[3-(3-Methoxyphenyl)pyrazolo[1,5-a]pyrimidin-7-yl]phenyl}-3-methylbutanamide;
N-{3-[3-(3-Ethoxyphenyl)pyrazolo[1,5-a]pyrimidin-7-yl]phenyl}-3-methylbutanamide;
3-Methyl-N-{3-[3-(4-phenoxyphenyl)pyrazolo[1,5-a]pyrimidin-7-yl]phenyl}butanamide;
N-{3-[3-(1,3-Benzodioxol-5-yl)pyrazolo[1,5-a]pyrimidin-7-yl]phenyl}-3-methylbutanamide;
N-{3-[3-(3-Cyanophenyl)pyrazolo[1,5-a]pyrimidin-7-yl]phenyl}-3-methylbutanamide;
N-{3-[3-(3-Acetylphenyl)pyrazolo[1,5-a]pyrimidin-7-yl]phenyl}-3-methylbutanamide;
N-{3-[3-(3-Formyl-4-methoxyphenyl)pyrazolo[1,5-a]pyrimidin-7-yl]phenyl}-3-methylbutanamide;
N-{3-[3-(1,1′-Biphenyl-3-yl)pyrazolo[1,5-a]pyrimidin-7-yl]phenyl}-3-methylbutanamide;
N-{3-[3-(1,1′-Biphenyl-4-yl)pyrazolo[1,5-a]pyrimidin-7-yl]phenyl}-3-methylbutanamide;
N-{3-[3-(3-Formylphenyl)pyrazolo[1,5-a]pyrimidin-7-yl]phenyl}-3-methylbutanamide;
3-Methyl-N-(3-{3-[3-(morpholin-4-ylmethyl)phenyl]pyrazolo[1,5-a]pyrimidin-7-yl}phenyl)butanamide;
3-Methyl-N-[3-(3-{3-[(4-methylpiperazin-1-yl)methyl]phenyl}pyrazolo[1,5-a]pyrimidin-7-yl)phenyl]butanamide;
3-[3-(3,5-Dimethylphenyl)pyrazolo[1,5-a]pyrimidin-7-yl]benzonitrile;
3-(3,5-Dimethylphenyl)-7-[3-(1H-tetraazol-5-yl)phenyl]pyrazolo[1,5-a]pyrimidine;
7-{3-[2-(Cyclobutylmethyl)-2H-tetraazol-5-yl]phenyl}-3-(3,5-dimethylphenyl)pyrazolo[1,5-a]pyrimidine;
3-(3,5-Dimethylphenyl)-7-{3-[2-(2-morpholin-4-ylethyl)-2H-tetraazol-5-yl]phenyl}pyrazolo[1,5-a]pyrimidine;
3-(3,5-Dimethylphenyl)-7-{3-[2-(pyridin-3-ylmethyl)-2H-tetrazol-5-yl]phenyl}pyrazolo[1,5-a]pyrimidine;
3-(3,5-Dimethylphenyl)-7-{3-[2-(pyridin-2-ylmethyl)-2H-tetrazol-5-yl]phenyl}pyrazolo[1,5-a]pyrimidine;
3-(3,5-Dimethylphenyl)-7-[3-(2-ethyl-2H-tetrazol-5-yl)phenyl]pyrazolo[1,5-a]pyrimidine;
N-[3-(3-{3-[(Dimethylamino)methyl]phenyl}pyrazolo[1,5-a]pyrimidin-7-yl)phenyl]-3-methylbutanamide;
3-(3,5-Dimethylphenyl)-7-{3-[2-(2-pyrrolidin-1-ylethyl)-2H-tetrazol-5-yl]phenyl}pyrazolo[1,5-a]pyrimidine;
3-(3,5-Dimethylphenyl)-7-{3-[2-(2-piperidin-1-ylethyl)-2H-tetrazol-5-yl]phenyl}pyrazolo[1,5-a]pyrimidine;
3-(3,5-Dimethylphenyl)-7-{3-[2-(pyridin-4-ylmethyl)-2H-tetrazol-5-yl]phenyl}pyrazolo[1,5-a]pyrimidine;
{7-[3-(1H-Tetraazol-5-yl)phenyl]pyrazolo[1,5-a]pyrimidin-3-yl}(thien-2-yl)methanone;
{7-[3-(2-Ethyl-2H-tetraazol-5-yl)phenyl]pyrazolo[1,5-a]pyrimidin-3-yl}(thien-2-yl)methanone;
(7-{3-[2-(Pyridin-3-ylmethyl)-2H-tetraazol-5-yl]phenyl}pyrazolo[1,5-a]pyrimidin-3-yl)(thien-2-yl)methanone;
{7-[3-(2-Isobutyl-2H-tetraazol-5-yl)phenyl]pyrazolo[1,5-a]pyrimidin-3-yl}(thien-2-yl)methanone;
(7-{3-[2-(Cyclobutylmethyl)-2H-tetraazol-5-yl]phenyl}pyrazolo[1,5-a]pyrimidin-3-yl)(thien-2-yl)methanone;
{7-[3-(2-Butyl-2H-tetraazol-5-yl)phenyl]pyrazolo[1,5-a]pyrimidin-3-yl}(thien-2-yl)methanone;
(7-{3-[2-(2-Morpholin-4-ylethyl)-2H-tetraazol-5-yl]phenyl}pyrazolo[1,5-a]pyrimidin-3-yl)(thien-2-yl)methanone;
(7-{3-[1-(Cyclobutylmethyl)-1H-tetraazol-5-yl]phenyl}pyrazolo[1,5-a]pyrimidin-3-yl)(thien-2-yl)methanone;
(7-{3-[2-(Pyridin-2-ylmethyl)-2H-tetraazol-5-yl]phenyl}pyrazolo[1,5-a]pyrimidin-3-yl)(thien-2-yl)methanone;
(7-{3-[2-(Pyridin-4-ylmethyl)-2H-tetraazol-5-yl]phenyl}pyrazolo[1,5-a]pyrimidin-3-yl)(thien-2-yl)methanone;
(7-{3-[2-(2-Pyrrolidin-1-ylethyl)-2H-tetraazol-5-yl]phenyl}pyrazolo[1,5-a]pyrimidin-3-yl)(thien-2-yl)methanone;
(7-{3-[2-(2-Piperidin-1-ylethyl)-2H-tetraazol-5-yl]phenyl}pyrazolo[1,5-a]pyrimidin-3-yl)(thien-2-yl)methanone;
(7-{3-[2-(2-Hydroxyethyl)-2H-tetraazol-5-yl]phenyl}pyrazolo[1,5-a]pyrimidin-3-yl)(thien-2-yl)methanone;
{7-[3-(1H-Pyrazol-3-yl)phenyl]pyrazolo[1,5-a]pyrimidin-3-yl}(thien-2-yl)methanone;
(7-{3-[1-(Cyclobutylmethyl)-1H-pyrazol-3-yl]phenyl}pyrazolo[1,5-a]pyrimidin-3-yl)(thien-2-yl)methanone;
(7-{3-[1-(2-Morpholin-4-ylethyl)-1H-pyrazol-3-yl]phenyl}pyrazolo[1,5-a]pyrimidin-3-yl)(thien-2-yl)methanone;
(7-{3-[1-(Pyridin-2-ylmethyl)-1H-pyrazol-3-yl]phenyl}pyrazolo[1,5-a]pyrimidin-3-yl)(thien-2-yl)methanone;
{7-[3-(1-Ethyl-1H-pyrazol-3-yl)phenyl]pyrazolo[1,5-a]pyrimidin-3-yl}(thien-2-yl)methanone;
(7-{3-[1-(2-Piperidin-1-ylethyl)-1H-pyrazol-3-yl]phenyl}pyrazolo[1,5-a]pyrimidin-3-yl)(thien-2-yl)methanone;
(7-{3-[1-(2-Pyrrolidin-1-ylethyl)-1H-pyrazol-3-yl]phenyl}pyrazolo[1,5-a]pyrimidin-3-yl)(thien-2-yl)methanone;
and pharmaceutically acceptable salts and prodrugs thereof.
